(19) United States
(12) Patent Application Publication
(10) Pub. No.: US 2019/0240293 A1
(43) Pub. Date: Aug. 8, 2019

(54) NEUROMODULATING COMPOSITIONS AND RELATED THERAPEUTIC METHODS FOR THE TREATMENT OF CANCER BY MODULATING AN ANTI-CANCER IMMUNE RESPONSE

(71) Applicant: Flagship Pioneering Innovations V, Inc., Cambridge, MA (US)

(72) Inventors: Erica WEINSTEIN, Boston, MA (US); Jordi MATA-FINK, Baltimore, MD (US); Avak KAHVEJIAN, Lexington, MA (US); Noubar B. AFEYAN, Lexington, MA (US); Laura Kristina JEANBART, Zurich (CH); Alexandra LANTERMANN, Boston, MA (US); Jonathan Barry HUROV, Bedford, MA (US); Manuel Andreas FANKHAUSER, Bern (CH); Chengyi J. SHU, Cambridge, MA (US); Eric Franklin ZHU, Cambridge, MA (US)

(21) Appl. No.: 16/320,342

(22) PCT Filed: Jul. 25, 2017
(86) PCT No.: PCT/US17/43804

§ 371 Date: Jan. 24, 2019

Related U.S. Application Data
(60) Provisional application No. 62/366,773, filed on Jul. 26, 2016.

(51) Int. Cl. A61K 38/22; A61P 25/00; A61P 35/00; A61P 37/02; C07K 16/26; A61K 45/06

(52) U.S. Cl. CPC A61K 38/2271; A61P 25/00; A61P 35/00; C07K 2317/76; A61K 38/225; C07K 16/26; A61K 45/06; A61P 37/02

(57) ABSTRACT

Described herein are methods for treating a subject having or at risk of developing cancer administering a neuromodulating agent.

### BACKGROUND

Cancer is still one of the deadliest threats to human health. In 2012, there were 14 million new cases of cancer worldwide and 8.2 million cancer-related deaths. The number of new cancer cases is expected to rise to 22 million by 2030, and worldwide cancer deaths are project to increase by 60%. Thus, there remains a need in the field for treatments for cancer.

### SUMMARY OF THE INVENTION

The invention relates to the discovery that modulation of neurological signaling pathways can modulate an immune response and, e.g., can be used to modulate an anti-cancer immune response. Accordingly, therapeutic and pharmaceutical compositions (as well as veterinary compositions) comprising neuromodulating agents and related methods are disclosed herein for treatment of cancer. The invention also features methods of modulating an immune response or immune cell activities in a subject or in isolated immune cells.

In a first aspect, the invention provides a method of treating a subject with a disease characterized by immune dysregulation by administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject identified as having a disease characterized by immune dysregulation by administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject with a disease characterized by immune dysregulation by contacting an immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject identified as having a disease characterized by immune dysregulation by contacting an immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of modulating an immune response in a subject by contacting an immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of modulating an immune response in a subject by administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of modulating an immune cell activity by contacting an immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject with cancer by administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject identified as having cancer by administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject with cancer by contacting an immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject identified as having cancer by contacting an immune cell with an effective amount of a neuromodulating agent selected from the group including a neurotransmission modulator, a neuropeptide signaling modulator, a neuronal growth factor modulator, and a neurome gene expression modulator.

In some embodiments of any of the above aspects, the cancer is pancreatic cancer and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is small cell lung cancer (SCLC) and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is non-small cell lung cancer (NSCLC) and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is melanoma and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is prostate cancer and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is breast cancer and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is glioma and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is gastric cancer and the method includes administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject with a T cell-infiltrated tumor by administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject with a T cell-infiltrated tumor by contacting the tumor with an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject with a T cell-infiltrated tumor by contacting a T cell in the tumor with an effective amount of a neuromodulating agent selected from the group including a neurotransmission modulator, a neuropeptide signaling modulator, a neuronal growth factor modulator, and a neurome gene expression modulator.

In some embodiments of any of the above aspects, the method includes contacting an immune cell from column 2 of Table 13 with an effective amount of a neuromodulating agent that modulates a corresponding gene in column 1 of Table 13.

In some embodiments of any of the above aspects, the method includes modulating an immune cell activity.

In some embodiments of any of the above aspects, the method includes modulating lymph node innervation, modulating development of high endothelial venules (HEVs), or modulating the development of ectopic or tertiary lymphoid organs (TLOs).

In some embodiments of any of the above aspects, the immune cell activity is activation, proliferation, phagocytosis, antibody-dependent cell-mediated cytotoxicity (ADCC), antibody-dependent cell-mediated phagocytosis (ADCP), antigen presentation, lymph node homing, lymph node egress, differentiation, degranulation, polarization, cytokine production, recruitment, or migration. In some embodiments, the activation, proliferation, phagocytosis, ADCC, ADCP, antigen presentation, lymph node homing, lymph node egress, differentiation, degranulation, polarization, cytokine production, recruitment, migration, lymph node innervation, development of HEVs, or development of TLOs is increased. In some embodiments, polarization toward an M1 phenotype is increased. In some embodiments, polarization toward an M2 phenotype is increased. In some embodiments, the activation, proliferation, phagocytosis, ADCC, ACCP, antigen presentation, lymph node homing, lymph node egress, differentiation, degranulation, polarization, cytokine production, recruitment, migration, lymph node innervation, development of HEVs, or development of TLOs is decreased. In some embodiments, polarization toward an M1 phenotype is decreased. In some embodiments, polarization toward an M2 phenotype is decreased. In some embodiments, the cytokines are pro-inflammatory cytokines, anti-inflammatory cytokines, or proliferative cytokines. In some embodiments, recruitment or migration is directed toward a site of inflammation or infection. In some embodiments, migration is directed away from a site of inflammation or infection. In some embodiments, recruitment or migration is directed toward a lymph node or secondary lymphoid organ. In some embodiments, migration is directed away from a lymph node or secondary lymphoid organ.

In some embodiments of any of the above aspects, the immune cell is selected from the group including a T cell, a cytotoxic T cell, a monocyte, a peripheral blood hematopoietic stem cell, a macrophage, an antigen presenting cell, a Natural Killer cell, a mast cell, a neutrophil, an eosinophil, a basophil, a Natural Killer T cell, a B cell, a dendritic cell, and a regulatory T cell. In some embodiments of any of the above aspects, the immune cell is a T cell. In some embodiments of any of the above aspects, the immune cell is a macrophage. In some embodiments of any of the above aspects, the immune cell is a Natural Killer (NK) cell. In some embodiments of any of the above aspects, the immune cell is a dendritic cell. In some embodiments of any of the above aspects, the immune cell is a regulatory T cell (Treg).

In another aspect, the invention provides a method of modulating innervation of a lymph node or lymphoid organ by contacting an immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of modulating innervation of a lymph node or lymphoid organ, the method comprising administering an effective amount of a neuromodulating agent selected from the group including a neurotransmission modulator, a neuropeptide signaling modulator, a neuronal growth factor modulator, and a neurome gene expression modulator.

In some embodiments, innervation is increased. In some embodiments, innervation is decreased.

In another aspect, the invention provides a method of modulating development of HEVs or TLOs by contacting an immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of modulating development of HEVs or TLOs by administering with an effective amount of a neuromodulating agent selected from the group including a neurotransmission modulator, a neuropeptide signaling modulator, a neuronal growth factor modulator, and a neurome gene expression modulator. In some embodiments, development of HEVs or TLOs is increased. In some embodiments, development of HEVs or ectopic or TLOs is decreased.

In another aspect, the invention provides a method of modulating T cell cytokine production by contacting a T cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of modulating T cell cytokine production by administering an effective amount of a neuromodulating agent selected from the group including a neurotransmission modulator, a neuropeptide signaling modulator, a neuronal growth factor modulator, and a neurome gene expression modulator.

In some embodiments, T cell cytokine production of pro-inflammatory or pro-survival cytokines is increased. In some embodiments, T cell cytokine production of pro-inflammatory cytokines is decreased. In some embodiments, T cell cytokine production of anti-inflammatory cytokines is increased.

In another aspect, the invention provides a method of modulating macrophage polarization by contacting an immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of modulating macrophage polarization by administering an effective amount of a neuromodulating agent selected from the group including a neurotransmission modulator, a neuropeptide signaling modulator, a neuronal growth factor modulator, and a neurome gene expression modulator.

In some embodiments, macrophages are polarized toward an M2 phenotype. In some embodiments, macrophages are polarized toward an M1 phenotype.

In another aspect, the invention provides a method of increasing the number of immune cells in a tumor by contacting an immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of increasing the number of immune cells in a tumor by administering an effective amount of a neuromodulating agent selected from the group including a neurotransmission modulator, a neuropeptide signaling modulator, a neuronal growth factor modulator, and a neurome gene expression modulator.

In some embodiments of any of the above aspects, the method includes increasing immune cell migration or recruitment to a tumor. In some embodiments, the immune cell is a T cell, γδ T cell, Th1 CD4+ T cell, cytotoxic CD8+ T cell, B cell, macrophage, M1 macrophage, natural killer cell, neutrophil, eosinophil, mast cell, or dendritic cell. In some embodiments, the immune cell is a T cell. In some embodiments, the immune cell is a macrophage. In some embodiments, the immune cell is a dendritic cell. In some embodiments, the immune cell is an NK cell. In some embodiments, the immune cell is a CCR7+ T cell.

In another aspect, the invention provides a method of increasing immune cell homing to a lymph node by administering an effective amount of a neuromodulating agent selected from the group including a neurotransmission modulator, a neuropeptide signaling modulator, a neuronal growth factor modulator, and a neurome gene expression modulator.

In some embodiments, the invention provides a method of increasing immune cell homing to a lymph node, the method comprising contacting an immune cell with an effective amount of a neuromodulating agent selected from the group including a neurotransmission modulator, a neuropeptide signaling modulator, a neuronal growth factor modulator.

In some embodiments of any of the above aspects, the immune cell is a T cell, B cell, macrophage, or dendritic cell. In some embodiments of any of the above aspects, the immune cell is a T cell. In some embodiments of any of the above aspects, the immune cell is a macrophage. In some embodiments of any of the above aspects, the immune cell is a dendritic cell. In some embodiments, the immune cell is a CCR7+ T cell.

In another aspect, the invention provides a method of increasing the number of CCR7+ T cells in a lymph node by contacting a CCR7+ T cell with an effective amount of a dopamine agonist.

In another aspect, the invention provides a method of increasing the number of CCR7+ T cells in a lymph node by administering an effective amount of a dopamine agonist.

In some embodiments of any of the above aspects, the method includes increasing CCR7+ T cell proliferation. In some embodiments of any of the above aspects, the method includes increasing CCR7+ T cell lymph node homing.

In another aspect, the invention provides a method of decreasing immune cell migration to a tumor by contacting an immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing immune cell migration to a tumor by administering an effective amount of a neuromodulating agent selected from the group including a neurotransmission modulator, a neuropeptide signaling modulator, a neuronal growth factor modulator, and a neurome gene expression modulator.

In some embodiments of any of the above aspects, the immune cell is a myeloid-derived suppressor cell (MDSC), regulatory T cell, M2 macrophage, or immature dendritic cell. In some embodiments of any of the above aspects, the immune cell is a regulatory T cell. In some embodiments of any of the above aspects, the immune cell is an M2 macrophage. In some embodiments of any of the above aspects, the immune cell is an immature dendritic cell.

In another aspect, the invention provides a method of increasing pro-inflammatory cytokine levels by administering an effective amount of a neuromodulating agent selected from the group including a neurotransmission modulator, a neuropeptide signaling modulator, a neuronal growth factor modulator, and a neurome gene expression modulator.

In another aspect, the invention provides a method of increasing pro-inflammatory cytokine levels by contacting immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of increasing T cell production of pro-inflammatory or proliferative cytokines by contacting a T cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of increasing T cell production of pro-inflammatory or proliferative cytokines by administering an effective amount of a neuromodulating agent selected from the group including a neurotransmission modulator, a neuropeptide signaling modulator, a neuronal growth factor modulator, and a neurome gene expression modulator.

In some embodiments of any of the above aspects, the pro-inflammatory cytokine is interferon gamma (IFNγ), interleukin-5 (IL-5), IL-6, IL-4, IL-1β, IL-13, or tumor necrosis factor alpha (TNFα).

In some embodiments of any of the above aspects, the pro-inflammatory cytokine is interferon gamma IFNγ. In some embodiments of any of the above aspects, the pro-inflammatory cytokine is TNFα.

In some embodiments of any of the above aspects, the pro-inflammatory cytokine is IL-13. In some embodiments of any of the above aspects, the pro-inflammatory cytokine is IL-4. In some embodiments of any of the above aspects, the pro-inflammatory cytokine is IL-1R.

In another aspect, the invention provides a method of increasing macrophage polarization toward an M1 phenotype by contacting a macrophage with an effective amount of a neuromodulating agent selected from the group including a neurotransmission modulator, a neuropeptide signaling modulator, a neuronal growth factor modulator, and a neurome gene expression modulator.

In another aspect, the invention provides a method of increasing macrophage polarization toward an M1 phenotype by administering an effective amount of a neuromodulating agent selected from the group including a neurotransmission modulator, a neuropeptide signaling modulator, a neuronal growth factor modulator, and a neurome gene expression modulator.

In another aspect, the invention provides a method of increasing immune cell cytotoxicity by contacting an immune cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of increasing immune cell cytotoxicity by administering an effective amount of a neuromodulating agent selected from the group including a neurotransmission modulator, a neuropeptide signaling modulator, a neuronal growth factor modulator, and a neurome gene expression modulator.

In some embodiments of any of the above aspects, the cytotoxicity is antibody-dependent cell-mediated cytotoxicity. In some embodiments of any of the above aspects, the immune cell is an NK cell.

In another aspect, the invention provides a method of increasing Natural Killer (NK) cell activity or restoring NK cell lytic function by contacting an NK cell with an effective amount a neuromodulating agent selected from the group including a neurotransmission modulator, a neuropeptide signaling modulator, a neuronal growth factor modulator, and a neurome gene expression modulator.

In another aspect, the invention provides a method of increasing NK cell activity or restoring NK cell lytic function by administering an effective amount a neuromodulating agent selected from the group including a neurotransmission modulator, a neuropeptide signaling modulator, a neuronal growth factor modulator, and a neurome gene expression modulator.

In another aspect, the invention provides a method of increasing immune cell activation by contacting an immune cell with an effective amount of a neuromodulating agent selected from the group including a neurotransmission modulator, a neuropeptide signaling modulator, a neuronal growth factor modulator, and a neurome gene expression modulator, wherein the neuromodulating agent slows or prevents tumor growth.

In another aspect, the invention provides a method of increasing immune cell activation by administering an effective amount of a neuromodulating agent selected from the group including a neurotransmission modulator, a neuropeptide signaling modulator, a neuronal growth factor modulator, and a neurome gene expression modulator, wherein the neuromodulating agent slows or prevents tumor growth.

In another aspect, the invention provides a method of increasing immune cell polarization toward an M1 phenotype by contacting an immune cell with an effective amount of a neuromodulating agent selected from the group including a neurotransmission modulator, a neuropeptide signaling modulator, a neuronal growth factor modulator, and a neurome gene expression modulator, wherein the neuromodulating agent slows or prevents tumor growth.

In another aspect, the invention provides a method of increasing immune cell polarization toward an M1 phenotype by administering an effective amount of a neuromodulating agent selected from the group including a neurotransmission modulator, a neuropeptide signaling modulator, a neuronal growth factor modulator, and a neurome gene expression modulator, wherein the neuromodulating agent slows or prevents tumor growth.

In some embodiments of any the above aspects, the immune cell is a macrophage. In some embodiments of any the above aspects, the immune cell is a T cell. In some embodiments of any of the above aspects, the immune cell is a dendritic cell. In some embodiments of any of the above aspects, the immune cell is an NK cell. In some embodiments of any of the above aspects, the immune cell is a Treg.

In some embodiments of any of the above aspects, the pro-inflammatory cytokine is IL-1β, IL-5, IL-6, IL-8, IL-10, IL-12, IL-13, IL-18, TNFα, IFNγ, MCP-1, CCL2, or GMCSF.

In some embodiments of any of the above aspects, the pro-survival cytokine is IL-2, IL-4, IL-6, IL-7, or IL-15.

In some embodiments of any of the above aspects, the anti-inflammatory cytokine is IL-4, IL-10, IL-11, IL-13, IFNα, or TGFβ.

In some embodiments of any of the above aspects, the cancer is gastrointestinal cancer, gastric cancer, melanoma, pancreatic cancer, urogenital cancer, prostate cancer, gynecological cancer, ovarian cancer, lung cancer, small cell lung cancer, non-small cell lung cancer, head and neck cancer, esophageal cancer, CNS cancer, glioma, malignant mesothelioma, non-metastatic or metastatic breast cancer, skin cancer, thyroid cancer, bone or soft tissue sarcoma, paraneoplastic cancer, or a hematologic neoplasia.

In some embodiments of any of the above aspects, the neuromodulating agent is a dopamine agonist, adrenergic agonist, nicotinic agonist, muscarinic agonist, serotonin agonist, glutamate receptor agonist, histamine agonist, cannabinoid receptor agonist, purinergic receptor agonist, GABA agonist, neuropeptide Y receptor agonist, somatostatin receptor agonist, CGRP receptor agonist, tachykinin receptor agonist, VIP receptor agonist, opioid agonist, oxytocin receptor agonist, or vasopressin receptor agonist. In some embodiments, the agonist is selected from an agonist listed in Tables 2A-2L. In some embodiments, the agonist is a dopamine agonist listed in Table 2A or 2C. In some embodiments, the dopamine agonist is dopamine, quinpirole dopexamine, bromocriptine, lisuride, pergolide, cabergoline, quinagolide, apomorphine, ropinirole, pramipexole, or piribedil. In some embodiments, the agonist is an adrenergic agonist listed in Table 2A or 2B. In some embodiments, the adrenergic agonist is isoproterenol or metaproterenol.

In some embodiments of any of the above aspects, the neuromodulating agent is a dopamine antagonist, adrenergic antagonist, nicotinic antagonist, muscarinic antagonist, serotonin antagonist, glutamate receptor antagonist, histamine antagonist, cannabinoid receptor antagonist, purinergic receptor antagonist, GABA antagonist, neuropeptide Y receptor antagonist, somatostatin receptor antagonist, CGRP receptor antagonist, tachykinin receptor antagonist, VIP receptor antagonist, opioid antagonist, oxytocin receptor antagonist, or vasopressin receptor antagonist. In some embodiments, the antagonist is selected from an antagonist listed in Tables 2A-2L. In some embodiments, the antagonist is a dopamine antagonist listed in Table 2A or 2C. In some embodiments, the dopamine antagonist is haloperidol or L-741,626. In some embodiments, the antagonist is a beta adrenergic antagonist listed in Table 2A or 2B. In some embodiments, the beta adrenergic antagonist is propranolol or nadolol.

In some embodiments of any of the above aspects, the neuromodulating agent is neuropeptide Y, CGRP, somatostatin, bombesin, cholecystokinin, dynorphin, enkephalin, endorphin, gastrin glucagon, melatonin, motilin, neurokinin A, neurokinin B, orexin, oxytocin, pancreatic peptide, peptide YY, substance P, or vasoactive intestinal peptide. In some embodiments, the neuromodulating agent is neuropeptide Y. In some embodiments, the neuromodulating agent is CGRP.

In some embodiments of any of the above aspects, the neuromodulating agent is a neuropeptide Y, CGRP, somatostatin, bombesin, cholecystokinin, dynorphin, enkephalin, endorphin, gastrin glucagon, melatonin, motilin, neurokinin A, neurokinin B, orexin, oxytocin, pancreatic peptide, peptide YY, substance P, or vasoactive intestinal peptide blocking antibody. In some embodiments, the neuromodulating agent is a neuropeptide Y blocking antibody. In some embodiments, the neuromodulating agent is a CGRP blocking antibody. In some embodiments, the CGRP blocking antibody is an antibody listed in Table 4.

In some embodiments of any of the above aspects, the neuromodulating agent is a neurotransmission modulator. In some embodiments, the neurotransmission modulator is a neurotransmitter listed in Tables 1A-1B a neurotransmitter encoded by a gene in Table 7, an agonist or an antagonist of a neurotransmitter of neurotransmitter receptor listed in Tables 1A-1B or encoded by a gene in Table 7, a neurotransmission modulator listed in Table 2M, a modulator of a biosynthesis, channel, ligand receptor, signaling, structural, synaptic, vesicular, or transporter protein encoded by a gene in Table 7, a channel or transporter protein encoded by a gene in Table 8, or a neurotoxin listed in Table 3. In some embodiments, the agonist or antagonist is an agonist or antagonist listed in Tables 2A-2K.

In some embodiments of any of the above aspects, the neuromodulating agent is a neuropeptide signaling modulator. In some embodiments, the neuropeptide signaling modulator is a neuropeptide listed in Tables 1A-1B or encoded by a gene in Table 7 or analog thereof, an agonist or antagonist of a neuropeptide or neuropeptide receptor listed in Tables 1A-1B or encoded by a gene in Table 7, or a modulator of a biosynthesis, ligand, receptor, or signaling protein encoded by a gene in Table 7. In some embodiments, the neuropeptide has at least 70%, 75%, 80%, 85%, 90%, 95%, 98%, or 99% identity to the neuropeptide sequence referenced by accession number or Entrez Gene ID in Tables 1A-1B or Table 7. In some embodiments, the agonist or antagonist is an agonist or antagonist listed in Tables 2A or 2L.

In some embodiments of any of the above aspects, the neuromodulating agent is a neuronal growth factor modulator. In some embodiments, the neuronal growth factor modulator is a neuronal growth factor listed in Table 10 or encoded by a gene in Table 7 or an analog thereof, or a modulator of a ligand, receptor, structural, synaptic, or signaling protein encoded by a gene in Table 7. In some embodiments, the neuronal growth factor has at least 70%, 75%, 80%, 85%, 90%, 90%, 98%, or 99% identity to the neuronal growth factor sequence referenced by accession number or Entrez Gene ID in Table 10 or Table 7. In some embodiments, the neuronal growth factor modulator is an antibody listed in Table 5. In some embodiments, the neuronal growth factor modulator is an agonist or antagonist listed in Table 6. In some embodiments, the neuronal growth factor modulator is etanercept, thalidomide, lenalidomide, pomalidomide, pentoxifylline, bupropion, DOI, disitertide, or trabedersen.

In some embodiments of any of the above aspects, the neuromodulating agent is a neurome gene expression modulator. In some embodiments, the neurome gene expression modulator increases or decreases the expression of a neurome gene in Table 7.

In some embodiments of any of the above aspects, the neuromodulating agent modulates the expression of a neurome gene in Table 7 or the activity of a protein encoded by a neurome gene in Table 7.

In some embodiments of any of the above aspects, the neuromodulating agent modulates the expression or activity of a chemokine, chemokine receptor, or immune cell trafficking molecule in Tables 10 or 11.

In some embodiments of any of the above aspects, the neuromodulating agent is selected from the group including a neurotransmitter, a neuropeptide, an antibody, a small molecule, a DNA molecule, a RNA molecule, a gRNA, and a viral vector. In some embodiments, the antibody is a blocking or neutralizing antibody. In some embodiments, the RNA molecule is an mRNA or an inhibitory RNA. In some embodiments, the viral vector is selected from the group including an adeno-associated virus (AAV), an adenovirus, a parvovirus, a coronavirus, a rhabdovirus, a paramyxovirus, a picornavirus, an alphavirus, a herpes virus, a poxvirus, and a lentivirus. In some embodiments, the herpes virus is a replication deficient herpes virus.

In some embodiments of any of the above aspects, the neuromodulating agent does not cross the blood brain barrier. In some embodiments, the neuromodulating agent has been modified to prevent blood brain barrier crossing by conjugation to a targeting moiety, formulation in a particulate delivery system, addition of a molecular adduct, or through modulation of its size, polarity, flexibility, or lipophilicity.

In some embodiments of any of the above aspects, the neuromodulating agent does not have a direct effect on the central nervous system or gut.

In some embodiments of any of the above aspects, wherein the neuromodulating agent is administered locally. In some embodiments, the neuromodulating agent is administered to or near a lymph node. In some embodiments, the neuromodulating agent is administered intratumorally.

In some embodiments of any of the above aspects, the method further includes administering a second therapeutic agent. In some embodiments, the second therapeutic agent is a checkpoint inhibitor, a chemotherapeutic agent, a biologic cancer agent, an anti-angiogenic drug, a drug that targets cancer metabolism, an antibody that marks a cancer cell surface for destruction, an antibody-drug conjugate, a cell therapy, a commonly used anti-neoplastic agent, or a non-drug therapy. In some embodiments, the checkpoint inhibitor is an inhibitory antibody, a fusion protein, an agent that interacts with a checkpoint protein, an agent that interacts with the ligand of a checkpoint protein, an inhibitor of CTLA-4, an inhibitor of PD-1, an inhibitor of PD-L1, an inhibitor of PD-L2, or an inhibitor of B7-H3, B7-H4, BTLA, HVEM, TIM3, GAL9, LAGS, VISTA, KIR, 2B4, CD160, CGEN-15049, CHK 1, CHK2, A2aR, or B-7 family ligands. In some embodiments, the biologic cancer agent is an antibody listed in Table 12.

In some embodiments of any of the above aspects, the neuromodulating agent decreases tumor volume, tumor growth, tumor innervation, cancer cell proliferation, cancer cell invasion, or cancer cell metastasis, or increases cancer cell death.

In some embodiments of any of the above aspects, the method further includes measuring one or more of tumor volume, tumor growth, tumor innervation, cancer cell proliferation, cancer cell invasion, cancer cell metastasis, or tumor neurome gene expression after administration of the neuromodulating agent.

In some embodiments of any of the above aspects, the method further includes measuring cytokine levels after administration of the neuromodulating agent.

In some embodiments of any of the above aspects, the method further includes measuring one or more immune cell markers after administration of the neuromodulating agent.

In some embodiments of any of the above aspects, wherein the method further includes measuring the expression of one or more neurome genes in Table 7 after administration of the neuromodulating agent.

In some embodiments of any of the above aspects, wherein the method further includes measuring cytokine levels before administration of the neuromodulating agent.

In some embodiments of any of the above aspects, wherein the method further includes measuring one or more immune cell markers before administration of the neuromodulating agent.

In some embodiments of any of the above aspects, the one or more immune cell markers is a marker listed in Table 9.

In some embodiments of any of the above aspects, the method further includes profiling an immune cell for expression of one or more neurome genes in Table 7 before administration of the neuromodulating agent. In some embodiments, the method further includes selecting a neuromodulating agent based on the profiling results.

In some embodiments of any of the above aspects, the one or more neurome genes in Table 7 is a channel, transporter, neurotransmitter, neuropeptide, neurotrophic, signaling, synaptic, structural, ligand, receptor, biosynthesis, other, or vesicular gene.

In some embodiments of any of the above aspects, the subject is not diagnosed as having a neuropsychiatric disorder.

In some embodiments of any of the above aspects, the subject is not diagnosed as having high blood pressure or a cardiac condition.

In some embodiments of any of the above aspects, the neuromodulating agent is administered in an amount sufficient to increase lymph node innervation, increase tumor innervation, increase nerve activity in a lymph node, increase nerve activity in a tumor, increase the development of HEVs or TLOs, increase immune cell migration, increase immune cell proliferation, increase immune cell recruitment, increase immune cell lymph node homing, increase immune cell lymph node egress, increase immune cell tumor homing, increase immune cell tumor egress, increase immune cell differentiation, increase immune cell activation, increase immune cell polarization, increase immune cell cytokine production, increase immune cell degranulation, increase immune cell maturation, increase immune cell ADCC, increase immune cell ADCP, or increase immune cell antigen presentation.

In some embodiments of any of the above aspects, the neuromodulating agent is administered in an amount sufficient to decrease lymph node innervation, decrease tumor innervation, decrease nerve activity in a tumor, decrease nerve activity in a lymph node, decrease the development of HEVs or TLOs, decrease immune cell migration, decrease immune cell proliferation, decrease immune cell recruitment, decrease immune cell lymph node homing, decrease immune cell lymph node egress, decrease immune cell tumor homing, decrease immune cell tumor egress, decrease immune cell differentiation, decrease immune cell activation, decrease immune cell polarization, decrease immune cell cytokine production, decrease immune cell degranulation, decrease immune cell maturation, decrease immune cell ADCC, decrease immune cell ADCP, or decrease immune cell antigen presentation.

In some embodiments of any of the above aspects, the neuromodulating agent is administered in an amount sufficient to treat the cancer or tumor, cause remission, reduce tumor growth, reduce tumor volume, reduce tumor metastasis, reduce tumor invasion, reduce tumor proliferation, reduce tumor number, increase cancer cell death, increase time to recurrence, or improve survival.

### Definitions

As used herein, “administration” refers to providing or giving a subject a therapeutic agent (e.g., a neuromodulating agent), by any effective route. Exemplary routes of administration are described herein below.

As used herein, the term “agonist” refers to an agent (e.g., a neurotransmitter, neuropeptide, small molecule, or antibody) that increases receptor activity. An agonist may activate a receptor by directly binding to the receptor, by acting as a cofactor, by modulating receptor conformation (e.g., maintaining a receptor in an open or active state). An agonist may increase receptor activity by 10%, 20%, 30%, 40%, 50%, 60%, 70%, 80%, 90%, 95%, 98% or more. An agonist may induce maximal receptor activation or partial activation depending on the concentration of the agonist and its mechanism of action.

As used herein, the term “analog” refers to a protein of similar nucleotide or amino acid composition or sequence to any of the proteins or peptides of the invention, allowing for variations that do not have an adverse effect on the ability of the protein or peptide to carry out its normal function (e.g., bind to a receptor or initiate neurotransmitter or neuropeptide signaling). Analogs may be the same length, shorter, or longer than their corresponding protein or polypeptide. Analogs may have about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to the amino acid sequence of the naturally occurring protein or peptide. An analog can be a naturally occurring protein or polypeptide sequence that is modified by deletion, addition, mutation, or substitution of one or more amino acid residues.

As used herein, the term “antagonist” refers to an agent (e.g., a neurotransmitter, neuropeptide, small molecule, or antibody) that reduces or inhibits receptor activity. An antagonist may reduce receptor activity by directly binding to the receptor, by blocking the receptor binding site, by modulating receptor conformation (e.g., maintaining a receptor in a closed or inactive state). An antagonist may reduce receptor activity by 10%, 20%, 30%, 40%, 50%, 60%, 70%, 80%, 90%, 95%, 98% or more. An antagonist may also completely block or inhibit receptor activity. Antagonist activity may be concentration-dependent or -independent.

As used herein, the term “antibody” comprises at least the variable domain of a heavy chain, and normally comprises at least the variable domains of a heavy chain and of a light chain of an immunoglobulin, which bind to an antigen of interest. Antibodies and antigen-binding fragments, variants, or derivatives thereof include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies. Antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

As used herein, the term “cardiac condition” refers to a medical condition directly affecting the heart or circulatory system. Cardiac conditions include abdominal aortic aneurysm, arrhythmia (e.g., supraventricular tachycardia, inappropriate sinus tachycardia, atrial flutter, atrial fibrillation, ventricular tachycardia, and ventricular fibrillation), angina, atherosclerosis, brugada syndrome, cardiac arrest, cardiomyopathy, cardiovascular disease, congenital heart disease, coronary heart disease, catecholaminergic polymorphic ventricular tachycardia (CVPT), familial hypercholesterolaemia, heart attack, heart failure, heart block, heart valve disease (e.g., heart murmur, valve stenosis, mitral valve prolapse, and heart valve regurgitation), inherited heart conditions, long QT syndrome, progressive cardiac conduction deficit (PCCD), pericarditis, venous thromboembolism, peripheral artery disease, and stroke.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, an agent that “does not cross the blood brain barrier” is an agent that does not significantly cross the barrier between the peripheral circulation and the brain and spinal cord. This can also be referred to as “blood brain barrier impermeable” agent. Agents will have a limited ability to cross the blood brain barrier if they are not lipid soluble or have a molecular weight of over 600 Daltons. Agents that typically cross the blood brain barrier can be modified to become blood brain barrier impermeable based on chemical modifications that increase the size or alter the hydrophobicity of the agent, packaging modifications that reduce diffusion (e.g., packaging an agent within a microparticle or nanoparticle), and conjugation to biologics that direct the agent away from the blood brain barrier (e.g., conjugation to a pancreas-specific antibody). An agent that does not cross the blood brain barrier is an agent for which 30% or less (e.g., 30%, 25%, 20%, 15%, 10%, 5%, 2% or less) of the administered agent crosses the blood brain barrier.

As used herein, an agent that “does not have a direct effect on the central nervous system (CNS) or gut” is an agent that does not directly alter neurotransmission, neuronal numbers, or neuronal morphology in the CNS or gut when administered according to the methods described herein. This may be assessed by administering the agents to animal models and performing electrophysiological recordings or immunohistochemical analysis. An agent will be considered not to have a direct effect on the CNS or gut if administration according to the methods described herein has an effect on neurotransmission, neuronal numbers, or neuronal morphology in the CNS or gut that is 50% or less (e.g., 50%, 45%, 40%, 35%, 30%, 25%, 20%, 15%, 10%, 5%, or less) of the effect observed if the same agent is administered directly to the CNS or gut.

As used herein, the terms “effective amount,” “therapeutically effective amount,” and a “sufficient amount” of composition, vector construct, viral vector or cell described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating cancer it is an amount of the composition, vector construct, viral vector or cell sufficient to achieve a treatment response as compared to the response obtained without administration of the composition, vector construct, viral vector or cell. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, viral vector or cell of the present disclosure is an amount which results in a beneficial or desired result in a subject as compared to a control. As defined herein, a therapeutically effective amount of a composition, vector construct, viral vector or cell of the present disclosure may be readily determined by one of ordinary skill by routine methods known in the art. Dosage regime may be adjusted to provide the optimum therapeutic response.

As used herein, the term “high blood pressure” refers to a chronic medical condition in which the systemic arterial blood pressure is elevated. It is classified as blood pressure above 140/90 mmHg.

As used herein, the terms “increasing” and “decreasing” refer to modulating resulting in, respectively, greater or lesser amounts, of function, expression, or activity of a metric relative to a reference. For example, subsequent to administration of an neuromodulating agent in a method described herein, the amount of a marker of a metric (e.g., T cell polarization) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “innervated” refers to a tissue (e.g., a lymph node or tumor) that contains nerves. “Innervation” refers to the process of nerves entering a tissue.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration and administration to a mucous membrane of the subject, wherein the administration is intended to have a local and not a systemic effect.

As used herein, a “neuromodulating agent” is an agent that affects a nerve impulse, a nerve function, one or more components of a neural pathway, neural structure, function, or activity in a neuron or a cell of an innervated tissue, e.g., in the peripheral nervous system. A neuromodulating agent may, e.g., increase or decrease neurogenesis; potentiate or inhibit the transmission of a nerve impulse; increase or decrease innervation of a tissue or tumor; or increase or decrease adrenergic, dopaminergic, cholinergic, serotonergic, glutamatergic, purinergic, GABAergic, or neuropetidergic signaling in a nerve or cell of an innervated tissue. A neuromodulating agent may be a neuropeptide, a neurotoxin, or a neurotransmitter, and may be any type of agent such as a small molecule (e.g. a neuropeptide or neurotransmitter agonist or antagonist), a peptide, a protein (e.g., an antibody or receptor fusion protein) or a nucleic acid (e.g., a therapeutic mRNA). Neuromodulating agents include neurotransmission modulators, neuropeptide signaling modulators, neuronal growth factor modulators, and neurome gene expression modulators.

As used herein, the term “neurome gene” refers to a gene expressed by a cell or tissue of the nervous system. A list of exemplary neurome genes is provided in Tables 1A-1C, Table 7, and Table 8. Non-nervous system cells and tissues (e.g., immune cells and tumors) can also express neurome genes, and the invention includes methods of profiling non-nervous system cells and tissues for neurome gene expression, modulating neurome gene expression in in non-nervous system cells and tissues, and treating cancer based on neurome gene expression in in non-nervous system cells and tissues.

As used herein, the term “neurome gene expression modulator” refers to a neuromodulating agent that affects gene expression (e.g., gene transcription, gene translation, or protein levels) of one or more neurome genes. A neurome gene expression modulator may increase or decrease gene expression by 10%, 20%, 30%, 40%, 50%, 60%, 70%, 80%, 90%, 95%, 98%, or more. Neurome gene expression modulators may increase gene expression through epigenetic modifications (e.g., demethylation or acetylation), post-translational modifications (e.g., reducing ubiquitination, or altering sumoylation or phosphorylation), by increasing mRNA translation and stability, or through delivery of exogenous genetic material (e.g., a viral vector expressing a gene of interest). Neurome gene expression modulators may decrease gene expression through epigenetic modifications (e.g., methylation or deacetylation), post-translational modifications (e.g., increasing ubiquitination, or altering sumoylation or phosphorylation), or by decreasing mRNA translation and stability (e.g., using miRNA, siRNA, shRNA, or other therapeutic RNAs).

As used herein, the term “neuronal growth factor modulator” refers to a neuromodulating agent that regulates neuronal growth, development, or survival. Neuronal growth factors include proteins that promote neurogenesis, neuronal growth, and neuronal differentiation (e.g., neurotrophic factors NGF, NT3, BDNF, CNTF, and GDNF), proteins that promote neurite outgrowth (e.g., axon or dendrite outgrowth or stabilization), or proteins that promote synapse formation (e.g., synaptogenesis, synapse assembly, synaptic adhesion, synaptic maturation, synaptic refinement, or synaptic stabilization). These processes lead to innervation of tissue, including neural tissue, muscle, lymph nodes and tumors, and the formation of synaptic connections between two or more neurons and between neurons and non-neural cells (e.g., tumor cells). A neuronal growth factor modulator may block one or more of these processes (e.g., through the use of antibodies that block neuronal growth factors or their receptors) or promote one or more of these processes (e.g., through the use of these proteins or analogs or peptide fragments thereof). Exemplary neuronal growth factors are listed in Table 10.

As used herein, the term “neuropeptide signaling modulator” refers to a neuromodulating agent that either induces or increases neuropeptide signaling, or decreases or blocks neuropeptide signaling. Neuropeptide signaling modulators can increase or decrease neuropeptide signaling by 10%, 20%, 30%, 40%, 50%, 60%, 70%, 80%, 90%, 95%, 98% or more. Exemplary neuropeptides and neuropeptide receptors are listed in Tables 1A-1B. Neuropeptide signaling modulators that induce or increase neuropeptide signaling include neuropeptides and analogs and fragments thereof, agents that increase neuropeptide receptor activity (e.g., neuropeptide agonists), and agents that reduce neuropeptide degradation or reuptake. Neuropeptide signaling modulators that decrease or block neuropeptide signaling include agents that reduce or inhibit neuropeptide receptor activity (e.g., neuropeptide antagonists), agents that bind to neuropeptides or block their interaction with receptors (e.g., neuropeptide blocking antibodies), or agents that increase neuropeptide degradation or clearance. Exemplary neuropeptide agonists and antagonists are listed in Tables 2A and 2L.

As used herein, the term “neuropsychiatric disorder” refers to a psychiatric or mental disorder that may cause suffering or an impaired ability to function. A neuropsychiatric disorder is a syndrome characterized by clinically significant disturbance in an individual's cognition, emotion regulation, or behavior that reflects a dysfunction in the psychological, biological, or developmental processes underlying mental functioning. Neuropsychiatric disorders may be diagnosed by psychiatrists, psychologists, neurologists, or physicians. Neuropsychiatric disorders include mood disorders (e.g., depression, bipolar depression, major depressive disorder), psychotic disorders (e.g., schizophrenia, schizoaffective disorder), personality disorders (e.g., borderline personality disorder, obsessive compulsive personality disorder, narcissistic personality disorder), eating disorders, sleep disorders, sexual disorders, anxiety disorders (e.g., generalized anxiety disorder, social anxiety disorder, post-traumatic stress disorder), developmental disorders (e.g., autism, attention deficit disorder, attention deficit hyperactivity disorder), benign forgetfulness, childhood learning disorders, Alzheimer's disease, addiction, and others listed in the Diagnostic and Statistical Manual of Mental Disorders (DSM).

As used herein, the term “neurotransmission modulator” refers to a neuromodulating agent that either induces or increases neurotransmission or decreases or blocks neurotransmission. Neurotransmission modulators can increase or decrease neurotransmission by 10%, 20%, 30%, 40%, 50%, 60%, 70%, 80%, 90%, 95%, 98% or more. Exemplary neurotransmitters and neurotransmitter receptors are listed in Tables 1A-1B. Neurotransmission modulators may increase neurotransmission by increasing neurotransmitter synthesis or release, preventing neurotransmitter reuptake or degradation, increasing neurotransmitter receptor activity, increasing neurotransmitter receptor synthesis or membrane insertion, decreasing neurotransmitter degradation, and regulating neurotransmitter receptor conformation. Neurotransmission modulators that increase neurotransmission include neurotransmitters and analogs thereof and neurotransmitter receptor agonists. Neurotransmission modulators may decrease neurotransmission by decreasing neurotransmitter synthesis or release, increasing neurotransmitter reuptake or degradation, decreasing neurotransmitter receptor activity, decreasing neurotransmitter receptor synthesis or membrane insertion, increasing neurotransmitter degradation, regulating neurotransmitter receptor conformation, and disrupting the pre- or postsynaptic machinery. Neurotransmission modulators that decrease or block neurotransmission include antibodies that bind to or block the function of neurotransmitters, neurotransmitter receptor antagonists, and toxins that disrupt synaptic release.

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, a “pharmaceutical composition” or “pharmaceutical preparation” is a composition or preparation, having pharmacological activity or other direct effect in the mitigation, treatment, or prevention of disease, and/or a finished dosage form or formulation thereof and which is indicated for human use.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “proliferation” refers to an increase in cell numbers through growth and division of cells.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the terms “subject” and “patient” refer to an animal (e.g., a mammal, such as a human). A subject to be treated according to the methods described herein may be one who has been diagnosed with a particular condition, or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

“Treatment” and “treating,” as used herein, refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “activation” refers to the response of an immune cell to a perceived insult. When immune cells become activated, they proliferate, secrete pro-inflammatory cytokines, differentiate, present antigens, become more polarized, and become more phagocytic and cytotoxic. Factors that stimulate immune cell activation include pro-inflammatory cytokines, pathogens, and non-self antigen presentation (e.g., antigens from pathogens presented by dendritic cells, macrophages, or B cells).

As used herein, the terms “antibody-dependent cell mediated cytotoxicity” and “antibody-dependent cellular toxicity” (ADCC) refer to the killing of an antibody-coated target cell by a cytotoxic effector cell through a non-phagocytic process, characterized by the release of the content of cytotoxic granules or by the expression of cell death-inducing molecules. ADCC is triggered through interaction of target-bound antibodies (belonging to IgG or IgA or IgE classes) with certain Fc receptors (FcRs), glycoproteins present on the effector cell surface that bind the Fc region of immunoglobulins (Ig). Effector cells that mediate ADCC include natural killer (NK) cells, monocytes, macrophages, neutrophils, eosinophils and dendritic cells.

As used herein, the terms “antibody-dependent cell mediated phagocytosis” and “antibody-dependent cellular phagocytosis” (ADCP) refer to the phagocytosis (e.g., engulfment) of an antibody-coated target cell by immune cells (e.g., phagocytes). ADCP is triggered through interaction of target-bound antibodies (belonging to IgG or IgA or IgE classes) with certain Fc receptors (FcRs, e.g., FcγRIIa, FcγRIIIa, and FcγRI), glycoproteins present on the effector cell surface that bind the Fc region of immunoglobulins (Ig). Effector cells that mediate ADCP include monocytes, macrophages, neutrophils, and dendritic cells.

As used herein, the term “antigen presentation” refers to a process in which fragments of antigens are displayed on the cell surface of immune cells. Antigens are presented to T cells and B cells to stimulate an immune response. Antigen presenting cells include dendritic cells, B cells, and macrophages. Mast cells and neutrophils can also be induced to present antigens.

As used herein, the term “anti-inflammatory cytokine” refers to a cytokine produced or secreted by an immune cell that reduces inflammation. Immune cells that produce and secrete anti-inflammatory cytokines include T cells (e.g., Th cells) macrophages, B cells, and mast cells. Anti-inflammatory cytokines include IL4, IL-10, IL-11, IL-13, interferon alpha (IFNα) and transforming growth factor-beta (TGFβ).

As used herein, the term “chemokine” refers to a type of small cytokine that can induce directed chemotaxis in nearby cells. Classes of chemokines include CC chemokines, CXC chemokines, C chemokines, and CX3C chemokines. Chemokines can regulate immune cell migration and homing, including the migration and homing of monocytes, macrophages, T cells, mast cells, eosinophils, and neutrophils. Chemokines responsible for immune cell migration include CCL19, CCL21, CCL14, CCL20, CCL25, CCL27, CXCL12, CXCL13, CCR9, CCR10, and CXCR5. Chemokines that can direct the migration of inflammatory leukocytes to sites of inflammation or injury include CCL2, CCL3, CCL5, CXCL1, CXCL2, and CXCL8.

As used herein, the term “cytokine” refers to a small protein involved in cell signaling. Cytokines can be produced and secreted by immune cells, such as T cells, B cells, macrophages, and mast cells, and include chemokines, interferons, interleukins, lymphokines, and tumor necrosis factors.

As used herein, the term “cytokine production” refers to the expression, synthesis, and secretion (e.g., release) of cytokines by an immune cell.

As used herein, the term “cytotoxicity” refers to the ability of immune cells to kill other cells. Immune cells with cytotoxic functions release toxic proteins (e.g., perforin and granzymes) capable of killing nearby cells. Natural killer cells and cytotoxic T cells (e.g., CD8+ T cells) are the primary cytotoxic effector cells of the immune system, although dendritic cells, neutrophils, eosinophils, mast cells, basophils, macrophages, and monocytes have been shown to have cytotoxic activity.

As used herein, the term “differentiation” refers to the developmental process of lineage commitment. A “lineage” refers to a pathway of cellular development, in which precursor or “progenitor” cells undergo progressive physiological changes to become a specified cell type having a characteristic function (e.g., nerve cell, immune cell, or endothelial cell). Differentiation occurs in stages, whereby cells gradually become more specified until they reach full maturity, which is also referred to as “terminal differentiation.” A “terminally differentiated cell” is a cell that has committed to a specific lineage, and has reached the end stage of differentiation (i.e., a cell that has fully matured). By “committed” or “differentiated” is meant a cell that expresses one or more markers or other characteristic of a cell of a particular lineage.

As used herein, the term “degranulation” refers to a cellular process in which molecules, including antimicrobial and cytotoxic molecules, are released from intracellular secretory vesicles called granules. Degranulation is part of the immune response to pathogens and invading microorganisms by immune cells such as granulocytes (e.g., neutrophils, basophils, and eosinophils), mast cells, and lymphocytes (e.g., natural killer cells and cytotoxic T cells). The molecules released during degranulation vary by cell type and can include molecules designed to kill the invading pathogens and microorganisms or to promote an immune response, such as inflammation.

As used herein, the term “immune dysregulation” refers to a condition in which the immune system is disrupted or responding to an insult. Immune dysregulation includes aberrant activation (e.g., autoimmune disease), activation in response to an injury or disease (e.g., disease-associated inflammation), and activation in response to a pathogen or infection (e.g., parasitic infection). Immune dysregulation also includes under-activation of the immune system (e.g., failure to mount an immune response to cancer cells or immunosuppression). Immune dysregulation can be treated using the methods and compositions described herein to direct immune cells to carry out beneficial functions and reduce harmful activities (e.g., promoting activation and cytotoxicity in subjects with cancer and reducing activation and pro-inflammatory cytokine secretion in subjects with autoimmune disease).

As used herein, the term “modulating an immune response” refers to any alteration in a cell of the immune system or any alteration in the activity of a cell involved in the immune response. Such regulation or modulation includes an increase or decrease in the number of various cell types, an increase or decrease in the activity of these cells, or any other changes that can occur within the immune system. Cells involved in the immune response include, but are not limited to, T lymphocytes (T cells), B lymphocytes (B cells), natural killer (NK) cells, macrophages, eosinophils, mast cells, dendritic cells and neutrophils. In some cases, “modulating” the immune response means the immune response is stimulated or enhanced, and in other cases “modulating” the immune response means suppression of the immune system.

As used herein, the term “lymph node egress” refers to immune cell exit from the lymph nodes, which occurs during immune cell recirculation. Immune cells that undergo recirculation include lymphocytes (e.g., T cells, B cells, and natural killer cells), which enter the lymph node from blood to survey for antigen and then exit into lymph and return to the blood stream to perform antigen surveillance.

As used herein, the term “lymph node homing” refers to directed migration of immune cells to a lymph node. Immune cells that return to lymph nodes include T cells, B cells, macrophages, and dendritic cells.

As used herein, the term “migration” refers to the movement of immune cells throughout the body. Immune cells can migrate in response to external chemical and mechanical signals. Many immune cells circulate in blood including peripheral blood mononuclear cells (e.g., lymphocytes such as T cells, B cells, and natural killer cells), monocytes, macrophages, dendritic cells, and polymorphonuclear cells (e.g., neutrophils and eosinophils). Immune cells can migrate to sites of infection, injury, or inflammation, back to the lymph nodes, or to tumors or cancer cells.

As used herein, the term “phagocytosis” refers to the process in which a cell engulfs or ingests material, such as other cells or parts of cells (e.g., bacteria), particles, or dead or dying cells. A cell that capable of performing this function is called a phagocyte. Immune phagocytes include neutrophils, monocytes, macrophages, mast cells, B cells, eosinophils, and dendritic cells.

As used herein, the term “polarization” refers to the ability of an immune cell to shift between different functional states. A cell that is moving toward one of two functional extremes is said to be in the process of becoming more polarized. The term polarization is often used to refer to macrophages, which can shift between states known as M1 and M2. M1, or classically activated, macrophages secrete pro-inflammatory cytokines (e.g., IL-12, TNF, IL-6, IL-8, IL-1B, MCP-1, and CCL2), are highly phagocytic, and respond to pathogens and other environmental insults. M1 macrophages can also be detected by expression of Nos2. M2, or alternatively activated, macrophages secrete a different set of cytokines (e.g., IL-10) and are less phagocytic. M2 macrophages can detected by expression of Arg1, IDO, PF4, CCL24, IL10, and IL4Rα. Cells become polarized in response to external cues such as cytokines, pathogens, injury, and other signals in the tissue microenvironment.

As used herein, the term “pro-inflammatory cytokine” refers to a cytokine secreted from immune cells that promotes inflammation. Immune cells that produce and secrete pro-inflammatory cytokines include T cells (e.g., Th cells) macrophages, B cells, and mast cells. Pro-inflammatory cytokines include interleukin-1 (IL-1, e.g., IL-1β), IL-5, IL-6, IL-8, IL-10, IL-12, IL-13, IL-18, tumor necrosis factor (TNF, e.g., TNFα), interferon gamma (IFNγ), and granulocyte macrophage colony stimulating factor (GMCSF).

As used herein, the term “pro-survival cytokine” refers to a cytokine that promotes the survival of immune cells (e.g., T cells). Pro-survival cytokines include IL-2, IL-4, IL-6, IL-7, and IL-15.

As used herein, the term “recruitment” refers to the re-distribution of immune cells to a particular location (e.g., the site of infection, injury, or inflammation). Immune cells that can undergo this re-distributed and be recruited to sites of injury or disease include monocytes, macrophages, T cells, B cells, dendritic cells, and natural killer cells.

As used herein, the term “cancer” refers to a condition characterized by unregulated or abnormal cell growth. The terms “cancer cell,” “tumor cell,” and “tumor” refer to an abnormal cell, mass or population of cells that result from excessive division that may be malignant or benign and all pre-cancerous and cancerous cells and tissues.

### BRIEF DESCRIPTION OF THE DRAWINGS

FIGS. 1A-1C are a series of graphs showing that dopamine stimulation induces T cell production of pro-inflammatory cytokines. Dopamine was applied to primary human T cells isolated from healthy donors and maintained in culture. Low sub-nanomolar concentrations of dopamine induced an increase in the production of the pro-inflammatory cytokines IFNγ, IL-5, and IL-13 at 72 hours post treatment.

FIGS. 2A-2B are a series of graphs showing that dopamine stimulation induces T cell production of pro-inflammatory cytokines. Dopamine was applied to primary human T cells isolated from healthy donors and maintained in culture. Low sub-nanomolar concentrations of dopamine induced an increase in the production of the pro-inflammatory cytokines IL-6 and IL-10 at 72 hours post treatment.

FIGS. 3A-3B are a series of graphs showing that dopamine stimulation induces T cell production of pro-survival cytokines. Dopamine and the synthetic dopamine agonist quinpirole were applied to primary human T cells isolated from healthy donors and maintained in culture. Stimulation of T cells with dopamine and the synthetic dopamine agonist quinpirole induced an increase in T cell production of the pro-survival cytokine IL-2 (FIGS. 3A-3B). Dopamine induced an increase in IL-2 production at 24 and 48 hours post-treatment, while quinpirole induced an increase at all time points tested.

FIG. 4 is a series of graphs showing that isoproterenol modulates T cell cytokine production. Adrenergic agonist isoproterenol was applied to primary human T cells isolated from healthy donors and maintained in culture. Stimulation of T cells with the adrenergic agonist isoproterenol decreased production of pro-inflammatory cytokine IFNγ in T cells from two different donors at multiple time points.

FIG. 5 is a series of graphs showing that isoproterenol modulates T cell cytokine production. Adrenergic agonist isoproterenol was applied to primary human T cells isolated from healthy donors and maintained in culture. Stimulation of T cells with the adrenergic agonist isoproterenol decreased production of pro-inflammatory cytokine TNFα in T cells from two different donors at multiple time points.

FIGS. 6A-6C are a series of graphs showing that isoproterenol modulates T cell cytokine production. Adrenergic agonist isoproterenol was applied to primary human T cells isolated from healthy donors and maintained in culture. Stimulation of T cells with the adrenergic agonist isoproterenol decreased production of pro-inflammatory cytokines IFNγ (FIG. 6A), TNFα (FIG. 6B), and IL-10 (FIG. 6C) in T cells from two different donors at 72 hours.

FIG. 7 is a graph showing that neuropeptide Y modulates T cell cytokine production. Neuropeptide Y was applied to primary human T cells isolated from healthy donors and maintained in culture. Stimulation of T cells with Neuropeptide Y induced an increase in IL-4 at sub-nanomolar concentrations at 48 hours post-treatment.

FIGS. 8A-8D are a series of graphs showing that hock injection of dopaminergic pathway modulators in mice modulates T cell migration. C57BL/6J mice were injected in each hock with 50 μL of the immunostimulant CpG ODN (0.1 nmol), 50 μL of the dopaminergic agonist quinpirole (0.1 nmol), or with 25 μL dopaminergic antagonist (Haloperidol −48.5 nmol) followed by 25 μL quinpirole (0.1 nmol). Hock injection of dopamine agonist quinpirole increased the number of migratory phenotype CCR7+ T cells in the lymph node (FIGS. 8A-8B), which was inhibited by pre-treatment with dopaminergic antagonist haloperidol. In contrast, treatment with CpG ODN increased the number of inflammatory CD69+ T cells but had no effect on CCR7 expression (FIGS. 8C-8D).

### DETAILED DESCRIPTION

Neuromodulating agents described herein can surprisingly have immune effects, such as effects on T cell polarization, T cell activation, T cell proliferation, cytotoxic T cell activation, circulating monocytes, peripheral blood hematopoietic stem cells, immune cell numbers, macrophage polarization, macrophage phagocytosis, antibody-dependent cell-mediated phagocytosis (ADCP), macrophage activation, macrophage polarization, antigen presentation, antigen presenting cell migration, lymph node immune cell homing and cell egress, NK cell activation, antibody-dependent cell-mediated cytotoxicity (ADCC), mast cell degranulation, neutrophil recruitment, eosinophil recruitment, NKT cell activation, B cell activation, and regulatory T cell differentiation. It has been found that neuromodulating agents thus can have a therapeutic effect on cancer.

I. Neuromodulating Agents

Neuromodulating agents described herein can agonize or inhibit genes or proteins in neuromodulatory signaling pathways, in order to treat cancer. Neuromodulatory signaling pathway genes are listed in Tables 1A-C (column 1). Additional neurome genes (e.g., genes expressed by a nervous system cell or tissue) are listed in Table 7 and Table 8. The level, activity and/or function of such genes and the proteins they encode can be modulated by pharmaceutical compositions comprising agents described herein. Neuromodulating agents also include neurotransmitter and neuropeptide ligands listed in Table 1B and neuronal growth factors listed in Table 10.

Neuromodulating agents can be divided into four major categories: 1) neurotransmission modulators (e.g., agents that increase or decrease neurotransmission, such as neurotransmitter agonists or antagonists or neurotoxins), 2) neuropeptide signaling modulators (e.g., neuropeptides and neuropeptide agonists or antagonists), 3) neuronal growth factor modulators (e.g., neuronal growth factors or agents that agonize or antagonize neuronal growth factor signaling), and 4) neurome gene expression modulators (e.g., agents that modulate expression of a gene listed in Table 7 or Table 8). These classes of neuromodulating are described in more detail herein below.

TABLE 1A
NEUROTRANSMITTER & NEUROPEPTIDE
GENES & PATHWAYS

| | | | | Entrez |
|---|---|---|---|---|
| | | | Accession | Gene |
| Gene | Pathway | Type | Number | ID |
| ABAT | Neurotransmitter | Biosynthesis | P80404 | 18 |
| ACHE | Neurotransmitter | Biosynthesis | P22303 | 43 |
| ADCYAP1 | Neuropeptide | Ligand | P18509 | 116 |
| ADCYAP1R1 | Neuropeptide | Receptor | P41586 | 117 |
| ADIPOQ | Neuropeptide | Ligand | Q15848 | 9370 |
| ADM | Neuropeptide | Ligand | P35318 | 133 |
| ADM2 | Neuropeptide | Ligand | Q7Z4H4 | 79924 |
| ADORA2A | Neurotransmitter | Receptor | P29274 | 135 |
| ADORA2B | Neurotransmitter | Receptor | P29275 | 136 |
| Adra1a | Adrenergic/ | Receptor | P35348 | 148 |
| | Neurotransmitter | | | |
| Adra1b | Adrenergic/ | Receptor | P35368 | 147 |
| | Neurotransmitter | | | |
| Adra1d | Adrenergic/ | Receptor | P25100 | 146 |
| | Neurotransmitter | | | |
| Adra2a | Adrenergic/ | Receptor | P08913 | 150 |
| | Neurotransmitter | | | |
| Adra2b | Adrenergic/ | Receptor | P18089 | 151 |
| | Neurotransmitter | | | |
| Adra2c | Adrenergic/ | Receptor | P18825 | 152 |
| | Neurotransmitter | | | |
| Adrb1 | Adrenergic/ | Receptor | P08588 | 153 |
| | Neurotransmitter | | | |
| Adrb2 | Adrenergic/ | Receptor | P07550 | 154 |
| | Neurotransmitter | | | |
| Adrb3 | Adrenergic/ | Receptor | P13945 | 155 |
| | Neurotransmitter | | | |
| Adrbk1 | Adrenergic | Kinase | P25098 | 156 |
| Adrbk2 | Adrenergic | Kinase | P35626 | 157 |
| AGRN | Neuropeptide | Ligand | O00468 | 375790 |
| AGRP | Neuropeptide | Ligand | O00253 | 181 |
| AGT | Neuropeptide | Ligand | P01019 | 183 |
| AGTR1 | Neuropeptide | Receptor | P30556 | 185 |
| APLN | Neuropeptide | Ligand | Q9ULZ1 | 8862 |
| ASIP | Neuropeptide | Ligand | P42127 | 434 |
| AVP | Neuropeptide | Ligand | P01185 | 551 |
| AVPR1A | Neuropeptide | Receptor | P30560 | 552 |
| AVPR1B | Neuropeptide | Receptor | P47901 | 553 |
| AVPR2 | Neuropeptide | Receptor | P30518 | 554 |
| BACE1 | Neurotransmitter | Biosynthesis | P56817 | 23621 |
| BCHE | Neurotransmitter | Biosynthesis | P06276 | 590 |
| BDKRB2 | Neuropeptide | Receptor | P30411 | 624 |
| BRS3 | Neuromodulator | Receptor | P32247 | P32247 |
| C1QBP | Neuropeptide | Receptor | Q07021 | 708 |
| C4orf48 | Neuropeptide | Ligand | Q5BLP8 | 401115 |
| C6orf89 | Neuromodulator | Receptor | Q6UWU4 | 221477 |
| CALCA | Neuropeptide | Ligand | P06881 | 796 |
| CALCB | Neuropeptide | Ligand | P10092 | 797 |
| CALCR | Neuropeptide | Receptor | P30988 | 799 |
| CALCRL | Neuropeptide | Receptor | Q16602 | 10203 |
| CARTPT | Neuropeptide | Ligand | Q16568 | 9607 |
| CASR | Neuropeptide | Biosynthesis | P41180 | 846 |
| CCK | Neuropeptide | Ligand | P06307 | 885 |
| CCKAR | Neuropeptide | Receptor | P32238 | 886 |
| CCKBR | Neuropeptide | Receptor | P32239 | 887 |
| CCL2 | Neuropeptide | Ligand | P13500 | 6347 |
| CHAT | Neurotransmitter | Biosynthesis | P28329 | 1103 |
| CHGA | Neuropeptide | Ligand | P10645 | 1113 |
| CHGB | Neuropeptide | Ligand | P05060 | 1114 |
| CHRFAM7A | Neurotransmitter | Receptor | Q494W8 | 89832 |
| Chrm1 | Cholinergic/ | Receptor | P11229 | 1128 |
| | Neurotransmitter | | | |
| Chrm2 | Cholinergic/ | Receptor | P08172 | 1129 |
| | Neurotransmitter | | | |
| Chrm3 | Cholinergic/ | Receptor | P20309 | 1131 |
| | Neurotransmitter | | | |
| Chrm4 | Cholinergic/ | Receptor | P08173 | 1132 |
| | Neurotransmitter | | | |
| Chrm5 | Cholinergic/ | Receptor | P08912 | 1133 |
| | Neurotransmitter | | | |
| Chrna1 | Cholinergic/ | Receptor | P02708 | 1134 |
| | Neurotransmitter | | | |
| Chrna10 | Cholinergic/ | Receptor | Q9GZZ6 | 57053 |
| | Neurotransmitter | | | |
| Chrna2 | Cholinergic/ | Receptor | Q15822 | 1135 |
| | Neurotransmitter | | | |
| Chrna3 | Cholinergic/ | Receptor | P32297 | 1136 |
| | Neurotransmitter | | | |
| Chrna4 | Cholinergic/ | Receptor | P43681 | 1137 |
| | Neurotransmitter | | | |
| Chrna5 | Cholinergic/ | Receptor | P30532 | 1138 |
| | Neurotransmitter | | | |
| Chrna6 | Cholinergic/ | Receptor | Q15825 | 8973 |
| | Neurotransmitter | | | |
| Chrna7 | Cholinergic/ | Receptor | P36544 | 1139 |
| | Neurotransmitter | | | |
| Chrna9 | Cholinergic/ | Receptor | Q9UGM1 | 55584 |
| | Neurotransmitter | | | |
| Chrnb1 | Cholinergic/ | Receptor | P11230 | 1140 |
| | Neurotransmitter | | | |
| Chrnb2 | Cholinergic/ | Receptor | P17787 | 1141 |
| | Neurotransmitter | | | |
| Chrnb3 | Cholinergic/ | Receptor | Q05901 | 1142 |
| | Neurotransmitter | | | |
| Chrnb4 | Cholinergic/ | Receptor | P30926 | 1143 |
| | Neurotransmitter | | | |
| Chrnd | Cholinergic/ | Receptor | Q07001 | 1144 |
| | Neurotransmitter | | | |
| Chrne | Cholinergic/ | Receptor | Q04844 | 1145 |
| | Neurotransmitter | | | |
| Chrng | Cholinergic/ | Receptor | P07510 | 1146 |
| | Neurotransmitter | | | |
| CLCF1 | Neuropeptide | Ligand | Q9UBD9 | 23529 |
| CNR1 | Cannabinoid/ | Receptor | P21554 | 1268 |
| | Neurotransmitter | | | |
| CNR2 | Cannabinoid/ | Receptor | P34972 | 1269 |
| | Neurotransmitter | | | |
| CNRIP1 | Neurotransmitter | Receptor | Q96F85 | 25927 |
| COMT | Neurotransmitter | Biosynthesis | P21964 | 1312 |
| CORT | Neuropeptide | Ligand | O00230 | 1325 |
| CPA4 | Neurotransmitter | Biosynthesis | Q9UI42 | 51200 |
| CPE | Neuropeptide/ | Biosynthesis | P16870 | 1363 |
| | Neurotransmitter | | | |
| CRCP | Neuropeptide | Receptor | O75575 | 27297 |
| CREM | Neurotransmitter | Signaling | Q03060 | 1390 |
| CRH | Neuropeptide | Ligand | Q13324 | 1392 |
| CRHBP | Neuropeptide | Receptor | P24387 | 1393 |
| CRHR1 | Neuropeptide | Receptor | P34998 | 1394 |
| CRHR2 | Neuropeptide | Receptor | Q13324 | 1395 |
| CTSH | Neuropeptide | Biosynthesis | P09668 | 1512 |
| CTSV | Neuropeptide | Biosynthesis | O60911 | 1515 |
| CYSLTR1 | Neuropeptide | Receptor | Q9Y271 | 10800 |
| CYSLTR2 | Neuropeptide | Receptor | Q9NS75 | 57105 |
| DAGLA | Neurotransmitter | Biosynthesis | Q9Y4D2 | 747 |
| | (Cannabinoid) | | | |
| DAGLB | Neurotransmitter | Biosynthesis | Q8NCG7 | 221955 |
| | (Cannabinoid) | | | |
| DBH | Neurotransmitter | Biosynthesis | P09172 | 1621 |
| DBI | Neuropeptide | Ligand | P07108 | 1622 |
| DDC | Neurotransmitter | Biosynthesis | P20711 | 1644 |
| DGKI | Neurotransmitter | Biosynthesis | O75912 | 9162 |
| DOPO | Dopaminergic | Receptor | P09172 | 1621 |
| DPP4 | Neurotransmitter | Biosynthesis | P27487 | 1803 |
| Drd1 | Dopaminergic/ | Receptor | P21728 | 1812 |
| | Neurotransmitter | | | |
| Drd2 | Dopaminergic/ | Receptor | P14416 | 1813 |
| | Neurotransmitter | | | |
| Drd3 | Dopaminergic/ | Receptor | P35462 | 1814 |
| | Neurotransmitter | | | |
| Drd4 | Dopaminergic/ | Receptor | P21917 | 1815 |
| | Neurotransmitter | | | |
| Drd5 | Dopaminergic/ | Receptor | P21918 | 1816 |
| | Neurotransmitter | | | |
| ECEL1 | Neurotransmitter | Biosynthesis | O95672 | 9427 |
| EDN1 | Neuropeptide | Ligand | P05305 | 1906 |
| EDN2 | Neuropeptide | Ligand | P20800 | 1907 |
| EDN3 | Neuropeptide | Ligand | P14138 | 1908 |
| EDNRA | Neuropeptide | Receptor | P25101 | 1909 |
| EDNRB | Neuropeptide | Receptor | P24530 | 1910 |
| FAAH | Neurotransmitter | Biosynthesis | O00519 | 2166 |
| FAP | Neuropeptide | Biosynthesis | Q12884 | 2191 |
| FNTA | Neurotransmitter | Signaling | P49354 | 2339 |
| FOLH1 | Neuropeptide | Biosynthesis | Q04609 | 2346 |
| FSHR | Neuropeptide | Receptor | P23945 | 2492 |
| GABARAP | Amine | Receptor | O95166 | 11337 |
| | Neuromodulator/ | | | |
| | Neurotransmitter | | | |
| GABARAPL1 | Amine | Receptor | Q9H0R8 | 23710 |
| | Neuromodulator | | | |
| GABARAPL2 | Amine | Receptor | P60520 | 11345 |
| | Neuromodulator | | | |
| GABBR1 | Amine | Receptor | Q9UBS5 | 2550 |
| | Neuromodulator/ | | | |
| | Neurotransmitter | | | |
| GABBR2 | Amine | Receptor | O75899 | 9568 |
| | Neuromodulator | | | |
| GABRA1 | Amine | Receptor | P14867 | 2554 |
| | Neuromodulator/ | | | |
| | Neurotransmitter | | | |
| GABRA2 | Amine | Receptor | P47869 | 2555 |
| | Neuromodulator/ | | | |
| | Neurotransmitter | | | |
| GABRA3 | Neurotransmitter | Receptor | P34903 | 2556 |
| GABRA4 | Neurotransmitter | Receptor | P48169 | 2557 |
| GABRA5 | Amine | Receptor | P31644 | 2558 |
| | Neuromodulator/NT | | | |
| GABRA6 | Neurotransmitter | Receptor | Q16445 | 2559 |
| GABRB1 | Neurotransmitter | Receptor | P18505 | 2560 |
| GABRB2 | Amine | Receptor | P47870 | 2561 |
| | Neuromodulator/ | | | |
| | Neurotransmitter | | | |
| GABRB3 | Amine | Receptor | P28472 | 2562 |
| | Neuromodulator/ | | | |
| | Neurotransmitter | | | |
| GABRD | Amine | Receptor | O14764 | 2563 |
| | Neuromodulator/ | | | |
| | Neurotransmitter | | | |
| GABRE | Neurotransmitter | Receptor | P78334 | 2564 |
| GABRG1 | Neurotransmitter | Receptor | Q8N1C3 | 2565 |
| GABRG2 | Amine | Receptor | P18507 | 2566 |
| | Neuromodulator/ | | | |
| | Neurotransmitter | | | |
| GABRG3 | Neurotransmitter | Receptor | Q99928 | 2567 |
| GABRP | Neurotransmitter | Receptor | O00591 | 2568 |
| GABRQ | Neurotransmitter | Receptor | Q9UN88 | 55879 |
| GABRR1 | Amine | Receptor | P24046 | 2569 |
| | Neuromodulator/ | | | |
| | Neurotransmitter | | | |
| GABRR2 | Amine | Receptor | P28476 | 2570 |
| | Neuromodulator/ | | | |
| | Neurotransmitter | | | |
| GABRR3 | Neurotransmitter | Receptor | A8MPY1 | 200959 |
| GAD1 | Neurotransmitter | Biosynthesis | Q99259 | 2571 |
| GAD2 | Neurotransmitter | Biosynthesis | Q05329 | 2572 |
| GAL | Neuropeptide | Ligand | P22466 | 51083 |
| GALP | Neuropeptide | Ligand | Q810H5 | 85569 |
| GALR1 | Neuropeptide | Receptor | P47211 | 2587 |
| GALR2 | Neuropeptide | Receptor | O43603 | 8811 |
| GALR3 | Neuropeptide | Receptor | O60755 | 8484 |
| GAST | Neuropeptide | Ligand | P01350 | 2520 |
| GCGR | Secretin | Receptor | P47871 | 2642 |
| GCHFR | Neurotransmitter | Biosynthesis | P30047 | 2644 |
| GH1 | Neuropeptide | Ligand | P01241 | 2688 |
| GHRH | Neuropeptide | Ligand | P01286 | 2691 |
| GHRHR | Neuropeptide | Receptor | Q02643 | 2692 |
| GHRL | Neuropeptide | Ligand | Q9UBU3 | 51738 |
| GIP | Neuropeptide | Ligand | P09681 | 2695 |
| GLRA1 | Neurotransmitter | Receptor | P23415 | 2741 |
| GLRA2 | Neurotransmitter | Receptor | P23416 | 2742 |
| GLRA3 | Neurotransmitter | Receptor | O75311 | 8001 |
| GLRA4 | Neurotransmitter | Receptor | Q5JXX5 | 441509 |
| GLRB | Neurotransmitter | Receptor | P48167 | 2743 |
| GLS | Neurotransmitter | Biosynthesis | O94925 | 2744 |
| GLS2 | Neurotransmitter | Biosynthesis | Q9UI32 | 27165 |
| GluA1 | Amine | Receptor | P42261 | 2890 |
| (GluR1) | Neuromodulator | | | |
| GluK1 | Amine | Receptor | P39086 | 2897 |
| (GluR5) | Neuromodulator | | | |
| GLUL | Neurotransmitter | Biosynthesis | P15104 | 2752 |
| GluN1 | Amine | Receptor | Q05586 | 2902 |
| (NR1) | Neuromodulator | | | |
| GNMT | Neurotransmitter | Biosynthesis | Q14749 | 27232 |
| GNRH1 | Neuropeptide | Ligand | P01148 | 2796 |
| GNRH2 | Neuropeptide | Ligand | O43555 | 2797 |
| GPHN | Neuropeptide | Ligand | Q9NQX3 | 10243 |
| GPER1 | Neurotransmitter | Receptor | Q99527 | 2852 |
| GPR1 | Neurotransmitter | Receptor | P46091 | 2825 |
| GPR139 | Neurotransmitter | Receptor | Q6DWJ6 | 124274 |
| GPR143 | Neurotransmitter | Receptor | P51810 | 4935 |
| GPR149 | Neurotransmitter | Receptor | Q86SP6 | 344758 |
| GPR18 | Neurotransmitter | Receptor | Q14330 | 2841 |
| GPR21 | Neurotransmitter | Receptor | Q99679 | 2844 |
| GPR26 | Neurotransmitter | Receptor | Q8NDV2 | 2849 |
| GPR3 | Neurotransmitter | Receptor | P46089 | 2827 |
| GPR35 | Neurotransmitter | Receptor | Q9HC97 | 2859 |
| GPR52 | Neurotransmitter | Receptor | Q9Y2T5 | 9293 |
| GPR55 | Neurotransmitter | Receptor | Q9Y2T6 | 9290 |
| GPR78 | Neurotransmitter | Receptor | Q96P69 | 27201 |
| GPR83 | Neurotransmitter | Receptor | Q9NYM4 | 10888 |
| GPR84 | Neurotransmitter | Receptor | Q9NQS5 | 53831 |
| GPRASP1 | Neurotransmitter | Receptor | Q5JY77 | 9737 |
| GPR50 | Amine | Receptor | Q13585 | 9248 |
| | Neuromodulator | | | |
| GRIA1 | Amine | Receptor | P42261 | 2890 |
| | Neuromodulator/ | | | |
| | Neurotransmitter | | | |
| GRIA2 | Amine | Receptor | P42262 | 2891 |
| | Neuromodulator/ | | | |
| | Neurotransmitter | | | |
| GRIA3 | Amine | Receptor | P42263 | 2892 |
| | Neuromodulator/ | | | |
| | Neurotransmitter | | | |
| GRIA4 | Amine | Receptor | P48058 | 2893 |
| | Neuromodulator/ | | | |
| | Neurotransmitter | | | |
| GRID1 | Neurotransmitter | Receptor | Q9ULK0 | 2894 |
| GRID2 | Neurotransmitter | Receptor | O43424 | 2895 |
| GRIK1 | Amine | Receptor | P39086 | 2897 |
| | Neuromodulator/ | | | |
| | Neurotransmitter | | | |
| GRIK2 | Amine | Receptor | Q13002 | 2898 |
| | Neuromodulator/ | | | |
| | Neurotransmitter | | | |
| GRIK3 | Amine | Receptor | Q13003 | 2899 |
| | Neuromodulator/ | | | |
| | Neurotransmitter | | | |
| GRIK4 | Amine | Receptor | Q16099 | 2900 |
| | Neuromodulator/ | | | |
| | Neurotransmitter | | | |
| GRIK5 | Amine | Receptor | Q16478 | 2901 |
| | Neuromodulator/ | | | |
| | Neurotransmitter | | | |
| GRIN1 | Amine | Receptor | Q05586 | 2902 |
| | Neuromodulator/ | | | |
| | Neurotransmitter | | | |
| GRIN2A | Amine | Receptor | Q12879 | 2903 |
| | Neuromodulator/ | | | |
| | Neurotransmitter | | | |
| GRIN2B | Amine | Receptor | Q13224 | 2904 |
| | Neuromodulator | | | |
| GRIN2C | Amine | Receptor | Q14957 | 2905 |
| | Neuromodulator/ | | | |
| | Neurotransmitter | | | |
| GRIN2D | Amine | Receptor | O15399 | 2906 |
| | Neuromodulator/ | | | |
| | Neurotransmitter | | | |
| GRIN3A | Amine | Receptor | Q8TCU5 | 116443 |
| | Neuromodulator/ | | | |
| | Neurotransmitter | | | |
| GRIN3B | Amine | Receptor | O60391 | 116444 |
| | Neuromodulator/ | | | |
| | Neurotransmitter | | | |
| GRK2 | Neurotransmitter | Receptor | P25098 | 156 |
| GRK3 | Neurotransmitter | Receptor | P35626 | 157 |
| GRM1 | Neurotransmitter | Receptor | Q13255 | 2911 |
| GRM2 | Neurotransmitter | Receptor | Q14416 | 2912 |
| GRM3 | Neurotransmitter | Receptor | Q14832 | 2913 |
| GRM4 | Neurotransmitter | Receptor | Q14833 | 2914 |
| GRM5 | Neurotransmitter | Receptor | P41594 | 2915 |
| GRM6 | Neurotransmitter | Receptor | O15303 | 2916 |
| GRM7 | Neurotransmitter | Receptor | Q14831 | 2917 |
| GRM8 | Neurotransmitter | Receptor | O00222 | 2918 |
| GRP | Neuropeptide | Ligand | P07492 | 2922 |
| GRPR | Neuropeptide | Receptor | P30550 | 2925 |
| HCRT | Neuropeptide | Ligand | O43612 | 3060 |
| HCRTR1 | Neuropeptide/ | Receptor | O43613 | 3061 |
| | Orexin | | | |
| HCRTR2 | Neuropeptide/ | Receptor | O43614 | 3062 |
| | Orexin | | | |
| HNMT | Neurotransmitter | Biosynthesis | P50135 | 3176 |
| HOMER1 | Neurotransmitter | Receptor | Q86YM7 | 9456 |
| HRH1 | Amine | Receptor | P35367 | 3269 |
| | Neuromodulator/ | | | |
| | Neurotransmitter | | | |
| HRH2 | Amine | Receptor | P25021 | 3274 |
| | Neuromodulator/ | | | |
| | Neurotransmitter | | | |
| HRH3 | Amine | Receptor | Q9Y5N1 | 11255 |
| | Neuromodulator/ | | | |
| | Neurotransmitter | | | |
| HRH4 | Amine | Receptor | Q9H3N8 | 59340 |
| | Neuromodulator/ | | | |
| | Neurotransmitter | | | |
| Htr1a | Serotonin/ | Receptor | P08908 | 3350 |
| | Neurotransmitter | | | |
| Htr1b | Serotonin/ | Receptor | P28222 | 3351 |
| | Neurotransmitter | | | |
| Htr1c | Serotonin | Receptor | P28335 | |
| Htr1d | Serotonin/ | Receptor | P28221 | 3352 |
| | Neurotransmitter | | | |
| Htr1e | Serotonin/ | Receptor | P28566 | 3354 |
| | Neurotransmitter | | | |
| Htr1f | Serotonin/ | Receptor | P30939 | 3355 |
| | Neurotransmitter | | | |
| Htr2a | Serotonin/ | Receptor | P28223 | 3356 |
| | Neurotransmitter | | | |
| Htr2b | Serotonin/ | Receptor | P41595 | 3357 |
| | Neurotransmitter | | | |
| Htr2c | Serotonin/ | Receptor | P28335 | 3358 |
| | Neurotransmitter | | | |
| Htr3a | Serotonin/ | Receptor | P46098 | 3359 |
| | Neurotransmitter | | | |
| Htr3b | Serotonin/ | Receptor | O95264 | 9177 |
| | Neurotransmitter | | | |
| Htr3c | Serotonin/ | Receptor | Q8WXA8 | 170572 |
| | Neurotransmitter | | | |
| Htr3d | Serotonin/ | Receptor | Q70Z44 | 200909 |
| | Neurotransmitter | | | |
| HTR3E | Neurotransmitter | Receptor | A5X5Y0 | 285242 |
| Htr4 | Serotonin/ | Receptor | Q13639 | 3360 |
| | Neurotransmitter | | | |
| Htr5a | Serotonin/ | Receptor | P47898 | 3361 |
| | Neurotransmitter | | | |
| Htr5b | Serotonin | Receptor | P35365 | 79247 |
| HTR5BP | Neurotransmitter | Receptor | | 645694 |
| Htr6 | Serotonin/ | Receptor | P50406 | 3362 |
| | Neurotransmitter | | | |
| Htr7 | Serotonin/ | Receptor | P32305 | 3363 |
| | Neurotransmitter | | | |
| IAPP | Neuropeptide | Ligand | P10997 | 3375 |
| ITPR1 | Neurotransmitter | Signaling | Q14643 | 3708 |
| ITPR2 | Neurotransmitter | Signaling | Q14571 | 3709 |
| ITPR3 | Neurotransmitter | Signaling | Q14573 | 3710 |
| KISS1 | Neuropeptide | Ligand | Q15726 | 3814 |
| KISS1R | Neuropeptide | Receptor | Q969F8 | 84634 |
| LEP | Neuropeptide | Ligand | P41159 | 3952 |
| LHCGR | Neuropeptide | Receptor | P22888 | 3973 |
| LIF | Neuropeptide | Ligand | P15018 | 3976 |
| LTB4R | Neuropeptide | Receptor | Q15722 | 1241 |
| LTB4R2 | Neuropeptide | Receptor | Q9NPC1 | 56413 |
| LYNX1 | Neurotransmitter | Receptor | Q9BZG9 | 66004 |
| MAOA | Neurotransmitter | Biosynthesis | P21397 | 4128 |
| MAOB | Neurotransmitter | Biosynthesis | P27338 | 4129 |
| MC1R | Neuropeptide | Receptor | Q01726 | 4157 |
| MC2R | Neuropeptide | Receptor | Q01718 | 4158 |
| MC3R | Neuropeptide | Receptor | P41968 | 4159 |
| MC4R | Neuropeptide | Receptor | P32245 | 4160 |
| MC5R | Neuropeptide | Receptor | P33032 | 4161 |
| MCHR1 | Neuropeptide | Receptor | Q99705 | 2847 |
| MCHR2 | Neuropeptide | Receptor | Q969V1 | 84539 |
| MLN | Neuropeptide | Ligand | P12872 | 4295 |
| MME | Neuropeptide | Biosynthesis | P08473 | 4311 |
| MRAP | Neuropeptide | Receptor | Q8TCY5 | 56246 |
| MRAP2 | Neuropeptide | Receptor | Q96G30 | 112609 |
| MRGPRF | Neurotransmitter | Receptor | Q96AM1 | 116535 |
| MRGPRX1 | Neuropeptide | Receptor | Q96LB2 | 259249 |
| MRGPRX2 | Neurotransmitter | Receptor | Q96LB1 | 117194 |
| MRGPRX3 | Neuropeptide | Receptor | Q96LB0 | 117195 |
| MRGPRX4 | Neuropeptide | Receptor | Q96LA9 | 117196 |
| MTNR1A | Amine | Receptor | P48039 | 4543 |
| | Neuromodulator/ | | | |
| | Neuropeptide | | | |
| MTNR1B | Amine | Receptor | P49286 | 4544 |
| | Neuromodulator/ | | | |
| | Neuropeptide | | | |
| NAALAD2 | Neuropeptide | Biosynthesis | Q9Y3Q0 | 10003 |
| NAMPT | NT | Biosynthesis | P43490 | 10135 |
| NGF | Neuropeptide | Ligand | P01138 | 4803 |
| NISCH | Amine | Receptor | Q9Y2I1 | 11188 |
| | Neuromodulator/ | | | |
| | Neurotransmitter | | | |
| NMB | Neuropeptide | Ligand | P08949 | 4828 |
| NMBR | Neuropeptide | Receptor | P28336 | 4829 |
| NMS | Neuropeptide | Ligand | Q5H8A3 | 129521 |
| NMU | Neuropeptide | Ligand | P48645 | 10874 |
| NMUR1 | Neuropeptide | Receptor | Q9HB89 | 10316 |
| NMUR2 | Neuropeptide | Receptor | Q9GZQ4 | 56923 |
| NOS1 | Neurotransmitter | Biosynthesis | P29475 | 4842 |
| NPB | Neuropeptide | Ligand | Q8NG41 | 256933 |
| NPBWR1 | Neuropeptide | Receptor | P48145 | 2831 |
| NPBWR2 | Neuropeptide | Receptor | P48146 | 2832 |
| NPFF | Neuropeptide | Ligand | O15130 | 8620 |
| NPFFR1 | Neuropeptide | Receptor | Q9GZQ6 | 64106 |
| NPFFR2 | Neuropeptide | Receptor | Q9Y5X5 | 10886 |
| NPPA | Neuropeptide | Ligand | P01160 | 4878 |
| NPPB | Neuropeptide | Ligand | P16860 | 4879 |
| NPPC | Neuropeptide | Ligand | P23582 | 4880 |
| NPS | Neuropeptide | Ligand | P0C0P6 | 594857 |
| NPSR1 | Neuropeptide | Receptor | Q6W5P4 | 387129 |
| NPTN | Neurotransmitter | Receptor | Q9Y639 | 27020 |
| NPVF | Neuropeptide | Ligand | Q9HCQ7 | 64111 |
| NPW | Neuropeptide | Ligand | Q8N729 | 283869 |
| NPY | Neuropeptide | Ligand | P01303 | 4852 |
| NPY1R | Neuropeptide | Receptor | P25929 | 4886 |
| NPY2R | Neuropeptide | Receptor | P49146 | 4887 |
| NPY4R | Neuropeptide | Receptor | P50391 | 5540 |
| NPY5R | Neuropeptide | Receptor | Q15761 | 4889 |
| NPY6R | Neuropeptide | Receptor | Q61212 | 4888 |
| NTS | Neuropeptide | Ligand | Q6FH20 | 4922 |
| NTSR1 | Neuropeptide | Receptor | P30989 | 4923 |
| NTSR2 | Neuropeptide | Receptor | Q63384 | 23620 |
| NXPH1 | Neuropeptide | Ligand | P58417 | 30010 |
| NXPH2 | Neuropeptide | Ligand | O95156 | 11249 |
| NXPH3 | Neuropeptide | Ligand | O95157 | 11248 |
| NXPH4 | Neuropeptide | Ligand | O95158 | 11247 |
| OGFR | Neuropeptide | Receptor | Q9NZT2 | 11054 |
| OPRD1 | Neuropeptide/ | Receptor | P41143 | 4985 |
| | Opioid | | | |
| OPRK1 | Neuropeptide/ | Receptor | P41145 | 4986 |
| | Opioid | | | |
| OPRL1 | Neuropeptide | Receptor | P41146 | 4987 |
| OPRM1 | Neuropeptide/ | Receptor | P35372 | 4988 |
| | Opioid | | | |
| OXT | Neuropeptide | Ligand | P01178 | 5020 |
| OXTR | Neuropeptide | Receptor | P30559 | 5021 |
| P2RX1 | Neurotransmitter | Receptor | P51575 | 5023 |
| P2RX2 | Neurotransmitter | Receptor | Q9UBL9 | 22953 |
| P2RX3 | Neurotransmitter | Receptor | P56373 | 5024 |
| P2RX4 | Neurotransmitter | Receptor | Q99571 | 5025 |
| P2RX5 | Neurotransmitter | Receptor | Q93086 | 5026 |
| P2RX6 | Neurotransmitter | Receptor | O15547 | 9127 |
| P2RX7 | Neurotransmitter | Receptor | Q99572 | 5027 |
| P2RY11 | Neurotransmitter | Receptor | Q96G91 | 5032 |
| PAH | Neurotransmitter | Biosynthesis | P00439 | 5053 |
| PC | Neurotransmitter | Biosynthesis | P11498 | 5091 |
| PCSK1 | Neuropeptide | Biosynthesis | P29120 | 5122 |
| PCSK1N | Neuropeptide | Ligand/ | Q9UHG2 | 27344 |
| | | Biosynthesis | | |
| PDE1B | Neurotransmitter | Signaling | Q01064 | 5153 |
| PDE4A | Neurotransmitter | Signaling | P27815 | 5141 |
| PDE4D | Neurotransmitter | Signaling | Q08499 | 5144 |
| PDYN | Neuropeptide | Ligand | P01213 | 5173 |
| PENK | Neuropeptide | Ligand | P01211 | 5179 |
| PHOX2A | Neurotransmitter | Biosynthesis | O14813 | 401 |
| PHOX2B | Neurotransmitter | Biosynthesis | Q99453 | 8929 |
| PIK3CA | Neurotransmitter | Signaling | P42336 | 5290 |
| PIK3CB | Neurotransmitter | Signaling | P42338 | 5291 |
| PIK3CG | Neurotransmitter | Signaling | P48736 | 5294 |
| PLCB1 | Neurotransmitter | Signaling | Q9NQ66 | 23236 |
| PLCB2 | Neurotransmitter | Signaling | Q00722 | 5330 |
| PLCB3 | Neurotransmitter | Signaling | Q01970 | 5331 |
| PLCB4 | Neurotransmitter | Signaling | Q15147 | 5332 |
| PLCD1 | Neurotransmitter | Signaling | P51178 | 5333 |
| PLCE1 | Neurotransmitter | Signaling | Q9P212 | 51196 |
| PLCG1 | Neurotransmitter | Signaling | P19174 | 5335 |
| PLCL1 | Neurotransmitter | Signaling | Q15111 | 5334 |
| PLCL2 | Neurotransmitter | Signaling | Q9UPR0 | 23228 |
| PMCH | Neuropeptide | Ligand | P20382 | 5367 |
| PNOC | Neuropeptide | Ligand | Q13519 | 5368 |
| POMC | Neuropeptide | Ligand | P01189 | 5443 |
| PPP1CB | Neurotransmitter | Signaling | P62140 | 5500 |
| PPP1CC | Neurotransmitter | Signaling | P36873 | 5501 |
| PPY | Neuropeptide | Ligand | P01298 | 5539 |
| PPY2P | Neuropeptide | Ligand | Q9NRI7 | 23614 |
| PRIMA1 | Neurotransmitter | Biosynthesis | Q86XR5 | 145270 |
| PRKACG | Neurotransmitter | Signaling | P22612 | 5568 |
| PRKAR2B | Neurotransmitter | Signaling | P31323 | 5577 |
| PRKCG | Neurotransmitter | Signaling | P05129 | 5582 |
| PRKX | Neurotransmitter | Signaling | P51817 | 5613 |
| PRL | Neuropeptide | Ligand | P01236 | 5617 |
| PRLH | Neuropeptide | Ligand | P81277 | 51052 |
| PRLHR | Neuropeptide | Receptor | P49683 | 2834 |
| PRLR | Neuropeptide | Receptor | P16471 | 5618 |
| PROK1 | Neuropeptide | Ligand | P58294 | 84432 |
| PROK2 | Neuropeptide | Ligand | Q9HC23 | 60675 |
| PROKR1 | Neuropeptide | Receptor | Q8TCW9 | 10887 |
| PROKR2 | Neuropeptide | Receptor | Q8NFJ6 | 128674 |
| PTGDR | Neuropeptide | Receptor | Q13258 | 5729 |
| PTGDR2 | Neuropeptide | Receptor | Q9Y5Y4 | 11251 |
| PTGER1 | Neuropeptide | Receptor | P34995 | 5731 |
| PTGER2 | Neuropeptide | Receptor | P43116 | 5732 |
| PTGER3 | Neuropeptide | Receptor | P43115 | 5733 |
| PTGER4 | Neuropeptide | Receptor | P35408 | 5734 |
| PTGFR | Neuropeptide | Receptor | P43088 | 5737 |
| PTGIR | Neuropeptide | Receptor | P43119 | 5739 |
| PTGS2 | Neuropeptide | Biosynthesis | P35354 | 5743 |
| PTH | Neuropeptide | Ligand | P01270 | 5741 |
| PTH1R | Neuropeptide | Receptor | Q03431 | 5745 |
| PTH2 | Neuropeptide | Ligand | Q9Y3E5 | 113091 |
| PTH2R | Neuropeptide | Receptor | P49190 | 5746 |
| PTHLH | Neuropeptide | Ligand | P12272 | 5744 |
| PTK2 | Neuropeptide | Signaling | Q05397 | 5747 |
| PTK2B | Neuropeptide | Signaling | Q14289 | 2185 |
| PYY | Neuropeptide | Ligand | P10082 | 5697 |
| PYY2 | Neuropeptide | Ligand | Q9NRI6 | 23615 |
| PYY3 | Neuropeptide | Ligand | Q5JQD4 | 644059 |
| QRFP | Neuropeptide | Ligand | P83859 | 347148 |
| QRFPR | Neuropeptide | Receptor | Q96P65 | 84109 |
| RAMP1 | Neuropeptide | Receptor | O60894 | 10267 |
| RAMP2 | Neuropeptide | Receptor | O60895 | 10266 |
| RAMP3 | Neuropeptide | Receptor | O60896 | 10268 |
| RIC3 | Neurotransmitter | Receptor | Q7Z5B4 | 79608 |
| RLN1 | Neuropeptide | Ligand | P04808 | 6013 |
| RLN2 | Neuropeptide | Ligand | P04090 | 6019 |
| RLN3 | Neuropeptide | Ligand | Q8WXF3 | 117579 |
| RXFP1 | Neuropeptide | Receptor | Q9HBX9 | 59350 |
| RXFP2 | Neuropeptide | Receptor | Q8WXD0 | 122042 |
| RXFP3 | Neuropeptide | Receptor | Q9NSD7 | 51289 |
| RXFP4 | Neuropeptide | Receptor | Q8TDU9 | 339403 |
| S1PR4 | Neuropeptide | Receptor | O95977 | 8698 |
| SCG2 | Neuropeptide | Ligand/ | P13521 | 7857 |
| | | Vesicles | | |
| SCG3 | Neuropeptide | Ligand/ | Q8WXD2 | 29106 |
| | | Vesicles | | |
| SCG5 | Neuropeptide | Ligand/ | P05408 | 6447 |
| | | Vesicles | | |
| SCT | Neuropeptide | Ligand | P09683 | 6343 |
| SCTR | Secretin | Receptor | P47872 | 6344 |
| SHANK3 | Neurotransmitter | Signaling | Q9BYB0 | 85358 |
| SLC6A1 | Amine | Transferase | P30531 | 6529 |
| | Neuromodulator | | | |
| SLC6A13 | Amine | Transferase | Q9NSD5 | 6540 |
| | Neuromodulator | | | |
| Slc6a4 | Serotonin | Transporter | P31645 | 6532 |
| SNX13 | Neurotransmitter | Signaling | Q9Y5W8 | 23161 |
| SPX | Neuropeptide | Ligand | Q9BT56 | 80763 |
| SST | Neuropeptide | Ligand | P61278 | 6750 |
| SSTR1 | Neuropeptide | Receptor | P30872 | 6751 |
| SSTR2 | Neuropeptide | Receptor | P30874 | 6752 |
| SSTR3 | Neuropeptide | Receptor | P32745 | 6753 |
| SSTR4 | Neuropeptide | Receptor | P31391 | 6754 |
| SSTR5 | Neuropeptide | Receptor | P35346 | 6755 |
| TAAR1 | Amine | Receptor | Q96RJ0 | 134864 |
| | Neuromodulator | | | |
| TAAR2 | Amine | Receptor | Q9P1P5 | 9287 |
| | Neuromodulator | | | |
| TAAR5 | Neurotransmitter | Receptor | O14804 | 9038 |
| TAC1 | Neuropeptide | Ligand | P20366 | 6863 |
| TAC3 | Neuropeptide | Ligand | Q9UHF0 | 6866 |
| TAC4 | Neuropeptide | Ligand | Q86UU9 | 255061 |
| TACR1 | Neuropeptide | Receptor | P25103 | 6869 |
| TACR2 | Neuropeptide | Receptor | P21452 | 6865 |
| TACR3 | Neuropeptide | Receptor | P29371 | 6870 |
| TBXA2R | Neuropeptide | Receptor | P21731 | 6915 |
| TH | Neurotransmitter | Biosynthesis | P07101 | 7054 |
| TPH1 | Neurotransmitter | Biosynthesis | P17752 | 7166 |
| TPH2 | Neurotransmitter | Biosynthesis | Q8IWU9 | 121278 |
| TRHDE | Neurotransmitter | Biosynthesis | Q9UKU6 | 29953 |
| TRH | Neuropeptide | Ligand | P20396 | 7200 |
| TRHR | Neuropeptide | Receptor | P34981 | 7201 |
| TSHR | Neuropeptide | Receptor | P16473 | 7253 |
| UCN | Neuropeptide | Ligand | P55089 | 7349 |
| UCN2 | Neuropeptide | Ligand | Q96RP3 | 90226 |
| UCN3 | Neuropeptide | Ligand | Q969E3 | 114131 |
| UTS2 | Neuropeptide | Ligand | O95399 | 10911 |
| UTS2B | Neuropeptide | Ligand | Q756I0 | 257313 |
| UTS2R | Neuropeptide | Receptor | Q9UKP6 | 2837 |
| VIP | Neuropeptide | Ligand | P01282 | 7432 |
| VIPR1 | Neuropeptide | Receptor | P32241 | 7433 |
| VIPR2 | Neuropeptide | Receptor | P41587 | 7434 |

TABLE 1B
NEUROTRANSMITTERS & NEUROPEPTIDE LIGANDS

| | | | Accession |
|---|---|---|---|
| Ligand | Pathway | Type | Number |
| 2-Arachidonoylglycerol | Endocannabinoid | Ligand | |
| 2-Arachidonyl glyceryl ether | Endocannabinoid | Ligand | |
| 3-methoxytyramine | Amines | Ligand | |
| Acetylcholine | Amino Acids | Ligand | |
| Adenosine | Purine | Ligand | |
| Adenosine triphosphate | Purine | Ligand | |
| Agmatine | Amino Acids | Ligand | |
| Anandamide | Endocannabinoid | Ligand | |
| Aspartate | Amino Acids | Ligand | |
| Bombesin | Other | Ligand | |
| Carbon monoxide | Gas | Ligand | |
| Cholecystokinin | Gastrins | Ligand | P06307 |
| Cocaine | Other | Ligand | |
| Corticotropin | Opioids | Ligand | |
| D-serine | Amino Acids | Ligand | |
| Dopamine | Monoamines | Ligand | |
| Dynorphin | Opioids | Ligand | |
| Endorphin | Opioids | Ligand | |
| Enkephaline | Opioids | Ligand | |
| Epinephrine | Monoamines | Ligand | |
| Gamma-aminobutyric acid | Amino Acids | Ligand | |
| Gastrin | Gastrins | Ligand | P01350 |
| Gastrin releasing peptide | Other | Ligand | P07492 |
| Glucagon | Secretins | Ligand | |
| Glutamate | Amino Acids | Ligand | |
| Glycine | Amino Acids | Ligand | |
| Growth hormone-releasing | Secretins | Ligand | Q9UBU3 |
| factor | | | |
| Histamine | Monoamines | Ligand | |
| Melatonin | Monoamines | Ligand | |
| Motilin | Secretins | Ligand | P12872 |
| N-Acetylaspartylglutamate | Neuropeptides | Ligand | |
| N-Arachidonoyl dopamine | Endocannabinoid | Ligand | |
| N-methylphenethylamine | Amines | Ligand | |
| N-methyltryptamine | Amines | Ligand | |
| Neurokinin A | Tachykinins | Ligand | P20366 |
| Neurokinin B | Tachykinins | Ligand | Q334E7 |
| Neuropeptide Y | Neuropeptides | Ligand | P01303 |
| Neurophysin I | Neurohypophyseals | Ligand | P01178 |
| Neurophysin II | Neurohypophyseals | Ligand | P01185 |
| Nitric oxide | Gas | Ligand | |
| Norepinephrine | Monoamines | Ligand | |
| Octopamine | Amines | Ligand | |
| Orexin A | Orexins | Ligand | O43612 |
| Orexin B | Orexins | Ligand | O43613 |
| Oxytocin | Neurohypophyseals | Ligand | |
| Pancreatic polypeptide | Neuropeptides | Ligand | P01298 |
| Peptide YY | Neuropeptides | Ligand | P10082 |
| Phenethylamine | Amines | Ligand | |
| Serotonin | Monoamines | Ligand | |
| Somatostatin | Somatostatins | Ligand | P61278 |
| Substance P | Neuropeptides | Ligand | |
| Synephrine | Amines | Ligand | |
| Tryptamine | Amines | Ligand | |
| Tyramine | Amines | Ligand | |
| Vasoactive intestinal | Secretins | Ligand | P01282 |
| peptide | | | |
| Vasopressin | Neurohypophyseals | Ligand | |
| Virodhamine | Endocannabinoid | Ligand | |

TABLE 1C
NEURONAL GROWTH FACTORS

| | | Accession | Entrez |
|---|---|---|---|
| Gene | Type | Number | Gene ID |
| ARTN | Ligand | Q5T4W7 | 9048 |
| BDNF | Ligand | P23560 | 627 |
| BDNF-AS | Ligand | | 497258 |
| BEX1 | Signaling | Q9HBH7 | 55859 |
| BEX3 | Signaling | Q00994 | 27018 |
| CD34 | Receptor | P28906 | 947 |
| CDNF | Ligand | Q49AH0 | 441549 |
| CNTF | Ligand | P26441 | 1270 |
| CNTFR | Receptor | P26992 | 1271 |
| CRLF1 | Receptor | O75462 | 9244 |
| CSPG5 | Ligand | O95196 | 10675 |
| DCLK1 | Signaling | O15075 | 9201 |
| DISC1 | Signaling | Q9NRI5 | 27185 |
| DNAJC5 | Signaling | Q9H3Z4 | 80331 |
| DPYSL2 | Signaling | Q16555 | 1808 |
| DVL1 | Signaling | O14640 | 1855 |
| EFNA5 | Ligand | P52803 | 1946 |
| EGR3 | Signaling | Q06889 | 1960 |
| ENO2 | Signaling | P09104 | 2026 |
| EphA1 | Receptor | P21709 | 2041 |
| EphA10 | Receptor | Q5JZY3 | 284656 |
| EphA2 | Receptor | P29317 | 1969 |
| EphA3 | Receptor | P29320 | 2042 |
| EphA4 | Receptor | P29317 | 2043 |
| EphA5 | Receptor | P54756 | 2044 |
| EphA6 | Receptor | Q9UF33 | 285220 |
| EphA7 | Receptor | Q15375 | 2045 |
| EphA8 | Receptor | P29322 | 2046 |
| EphB1 | Receptor | P54762 | 2047 |
| EphB2 | Receptor | P29323 | 2048 |
| EphB3 | Receptor | P54753 | 2049 |
| EphB4 | Receptor | P54760 | 2050 |
| EphB6 | Receptor | O15197 | 2051 |
| ETBR2 | Receptor | O60883 | 9283 |
| FSTL4 | Receptor | Q6MZW2 | 23105 |
| GDNF | Ligand | P39905 | 2668 |
| GFRA1 | Receptor | P56159 | 2674 |
| GFRA2 | Receptor | O00451 | 2675 |
| GFRA3 | Receptor | O60609 | 2676 |
| GFRA4 | Receptor | Q9GZZ7 | 64096 |
| GPR37 | Receptor | O15354 | 2861 |
| GPRIN1 | Signaling | Q7Z2K8 | 114787 |
| GPRIN2 | Signaling | O60269 | 9721 |
| GPRIN3 | Signaling | Q6ZVF9 | 285513 |
| GRB2 | Signaling | P62993 | 2885 |
| GZF1 | Signaling | Q9H116 | 64412 |
| IFNA1 | Ligand | P01562 | 3439 |
| IGF1 | Ligand | P05019 | 3479 |
| IGF2 | Ligand | P01344 | 3481 |
| IL11RA | Receptor | Q14626 | 3590 |
| IL1B | Ligand | P01584 | 3553 |
| IL3 | Ligand | P08700 | 3562 |
| IL4 | Ligand | P05112 | 3565 |
| IL6 | Ligand | P05231 | 3569 |
| IL6R | Receptor | P08887 | 3570 |
| IL6ST | Signaling | P40189 | 3572 |
| INS | Ligand | P01308 | 3630 |
| L1CAM | Signaling | P32004 | 3897 |
| LIF | Ligand | P15018 | 3976 |
| LIFR | Receptor | P42702 | 3977 |
| MAGED1 | Signaling | Q9Y5V3 | 9500 |
| MANF | Ligand | P55145 | 7873 |
| NDNF | Ligand | Q8TB73 | 79625 |
| NENF | Ligand | Q9UMX5 | 29937 |
| NENFP1 | Ligand | | 106480294 |
| NENFP2 | Ligand | | 100129880 |
| NENFP3 | Ligand | | 106481703 |
| NGF | Ligand | P01138 | 4803 |
| NGFR | Receptor | P08138 | 4804 |
| NRG1 | Ligand | Q02297 | 3084 |
| NRP1 | Receptor | O14786 | 8829 |
| NRTN | Ligand | Q99748 | 902 |
| NTF3 | Ligand | P20783 | 4908 |
| NTF4 | Ligand | P34130 | 4909 |
| NTRK1 | Receptor | P04629 | 4914 |
| NTRK2 | Receptor | Q16620 | 4915 |
| NTRK3 | Receptor | Q16288 | 4916 |
| PDPK1 | Signaling | O15530 | 5170 |
| PEDF | Ligand | P36955 | 5176 |
| PLEKHH3 | Signaling | Q7Z736 | 79990 |
| PSAP | Ligand | P07602 | 5660 |
| PSEN1 | Signaling | P49768 | 5663 |
| PSPN | Ligand | O70300 | 5623 |
| PTN | Ligand | P21246 | 5764 |
| RELN | Ligand | P78509 | 5649 |
| RET | Signaling | P07949 | 5979 |
| ROR1 | Receptor | Q01973 | 4919 |
| ROR2 | Receptor | Q01974 | 4920 |
| RPS6KA3 | Signaling | P51812 | 6197 |
| SDC3 | Receptor | O75056 | 9672 |
| SEMA3E | Ligand | O15041 | 9723 |
| SERPINE2 | Ligand | P07093 | 5270 |
| SERPINF1 | Ligand | P36955 | 5176 |
| SHC1 | Signaling | P51812 | 6464 |
| SNTG1 | Biosynthesis | P07602 | 54212 |
| SORCS1 | Receptor | O75056 | 114815 |
| SORCS2 | Receptor | O15041 | 57537 |
| SORCS3 | Receptor | P07093 | 22986 |
| SORT1 | Receptor | Q99523 | 6272 |
| SULF1 | Signaling | Q8IWU6 | 23213 |
| SULF2 | Signaling | Q8IWU5 | 55959 |
| TGFB1 | Ligand | P01137 | 7040 |
| TGFB2 | Ligand | P61812 | 7042 |
| TGFB3 | Ligand | P10600 | 7043 |
| TMEM158 | Receptor | Q8WZ71 | 25907 |
| TNF | Ligand | P01375 | 7124 |
| TPM3 | Receptor | P06753 | 7170 |
| VEGFA | Ligand | P15692 | 7422 |
| VEGFB | Ligand | P49765 | 7423 |
| VGF | Ligand | O15240 | 7425 |
| XCR1 | Receptor | P46094 | 2829 |
| ZN274 | Signaling | Q96GC6 | 10782 |

Neurotransmission Modulators

In some embodiments, the neuromodulating agent is a neurotransmission modulator (e.g., an agent that increases or decreases neurotransmission). For example, in some embodiments, the neuromodulating agent is a neurotransmitter or neurotransmitter receptor listed in Table 1A, 1B, Table 7, or Table 8, a modulator of a channel or transporter encoded by a gene in Table 7, or an agonist or antagonist listed in Tables 2A-2K for a corresponding neurotransmitter pathway member. In some embodiments, the neurotransmission modulator is a neurotransmission modulator listed in Table 2M. Neuromodulating agents that increase neurotransmission include neurotransmitters and neurotransmitter receptors listed in Tables 1A, 1B, Table 7, and Table 8 and analogs thereof, and neurotransmitter agonists (e.g., small molecules that agonize a neurotransmitter receptor listed in Table 1A or encoded by a gene in Table 7 or Table 8). Exemplary agonists are listed in Tables 2A-2K. In some embodiments, neurotransmission is increased via administration, local delivery, or stabilization of neurotransmitters (e.g., ligands listed in Tables 1A, 1B, and Table 7). Neurotransmission modulators that increase neurotransmission also include agents that increase neurotransmitter synthesis or release (e.g., agents that increase the activity of a biosynthetic protein encoded by a gene in Table 1A or Table 7 via stabilization, overexpression, or upregulation, or agents that increase the activity of a synaptic or vesicular protein encoded by a gene in Table 7 via stabilization, overexpression, or upregulation), prevent neurotransmitter reuptake or degradation (e.g., agents that block or antagonize transporters encoded by a gene in Table 7 or Table 8 that remove neurotransmitter from the synaptic cleft), increase neurotransmitter receptor activity (e.g., agents that increase the activity of a signaling protein encoded by a gene in Table 1A or Table 7 via stabilization, overexpression, agonism, or upregulation, or agents that upregulate, agonize, or stabilize a neurotransmitter receptor listed in Table 1A or encoded by a gene in Table 7 or Table 8), increase neurotransmitter receptor synthesis or membrane insertion, decrease neurotransmitter degradation, and regulate neurotransmitter receptor conformation (e.g., agents that bind to a receptor and keep it in an “open” or “primed” conformation). In some embodiments, the neurotransmitter receptor is a channel (e.g., a ligand or voltage gated ion channel listed in Table 7 or Table 8), the activity of which can be increased by agonizing, opening, stabilizing, or overexpressing the channel. Neurotransmission modulators that increase neurotransmission further include agents that stabilize a structural protein encoded by a gene in Table 7. Neurotransmission modulators can increase neurotransmission by 10%, 20%, 30%, 40%, 50%, 60%, 70%, 80%, 90%, 95%, 98% or more. Exemplary neurotransmission modulators are listed in Table 2M.

Neuromodulating agents that decrease neurotransmission include neurotransmitter antagonists (e.g., small molecules that antagonize a neurotransmitter receptor listed in Table 1A or Table 7 or Table 8). Exemplary antagonists are listed herein below and in Tables 2A-2K. Neurotransmission modulators that decrease neurotransmission also include agents that decrease neurotransmitter synthesis or release (e.g., agents that decrease the activity of a biosynthetic protein encoded by a gene in Table 1A or Table 7 via inhibition or downregulation, or agents that decrease the activity of a synaptic or vesicular protein encoded by a gene in Table 7 via blocking, disrupting, or downregulating, or antagonizing the protein), increase neurotransmitter reuptake or degradation (e.g., agents that agonize, open, or stabilize transporters encoded by a gene in Table 7 or Table 8 that remove neurotransmitter from the synaptic cleft), decrease neurotransmitter receptor activity (e.g., agents that decrease the activity of a signaling protein encoded by a gene in Table 1A or Table 7 via blocking or antagonizing the protein, or agents that block, antagonize, or downregulate a neurotransmitter receptor listed in Table 1A or encoded by a gene in Table 7 or Table 8), decrease neurotransmitter receptor synthesis or membrane insertion, increase neurotransmitter degradation, regulate neurotransmitter receptor conformation (e.g., agents that bind to a receptor and keep it in a “closed” or “inactive” conformation), and disrupt the pre- or postsynaptic machinery (e.g., agents that block or disrupt a structural protein encoded by a gene in Table 7, or agents that block, disrupt, downregulate, or antagonize a synaptic or vesicular protein encoded by a gene in Table 7). In some embodiments, the neurotransmitter receptor is a channel (e.g., a ligand or voltage gated ion channel listed in Table 7 or Table 8), the activity of which can be decreased by blockade, antagonism, or inverse agonism of the channel. Neurotransmission modulators that decrease neurotransmission further include agents that sequester, block, antagonize, or degrade a neurotransmitter listed in Tables 1A, 1B, or encoded by a gene in Table 7. Neurotransmission modulators that decrease or block neurotransmission include antibodies that bind to or block the function of neurotransmitters, neurotransmitter receptor antagonists, and toxins that disrupt synaptic release. Neurotransmission modulators can decrease neurotransmission by 10%, 20%, 30%, 40%, 50%, 60%, 70%, 80%, 90%, 95%, 98% or more.

In some embodiments, the neuromodulating agent is an adrenergic receptor pathway modulator (e.g., a blocker or agonist of an adrenergic receptor listed in Table 1A or Table 7, e.g., an adrenergic blocker or agonist listed in Table 2A or Table 2B); a cholinergic receptor pathway modulator (e.g., a blocker or agonist of a cholinergic receptor listed in Table 1A or Table 7, e.g., a cholinergic blocker or agonist listed in Table 2A, 2E, or 2F); a dopamine receptor pathway modulator (e.g., a blocker or agonist of a dopamine receptor listed in Table 1A or Table 7, e.g., a dopamine blocker or agonist listed in Table 2A or 2C); a serotonin receptor pathway modulator (e.g., a blocker or agonist of a serotonin receptor listed in Table 1A, Table 7, or Table 8, e.g., a serotonin blocker or agonist listed in Table 2A or 2G); a GABA receptor pathway modulator (e.g., a blocker or agonist of a GABA receptor listed in Table 1A, Table 7, or Table 8, e.g., a GABA blocker or agonist listed in Table 2A or 2D); a glutamate receptor pathway modulator (e.g., a blocker or agonist of a glutamate receptor listed in Table 1A, Table 7, or Table 8, e.g., a glutamate blocker or agonist listed in Table 2A or 2H).

TABLE 2B
ADRENERGIC AGONISTS AND ANTAGONISTS

| Receptor | Agonist | Antagonist |
|---|---|---|
| Non-selective | Adrenaline (epinephrine), | Carvedilol, arotinolol, and |
| | Noradrenaline (norepinephrine), | labetalol |
| | Isoprenaline (isoproterenol), | |
| | dopamine, caffeine, nicotine, | |
| | tyramine, methylphenidate, | |
| | ephedrine and pseudophedrine. | |
| α1 selective | Phenylephrine, methoxamine, | Acepromazine, alfuzosin, |
| (ADRA1A, ADRA1B, | midodrine, cirazoline, | doxazosin, labetalol, |
| ADRA1D) | Xylometazoline, metaraminol | phenoxybenzamine, KW3902, |
| | Chloroehtylclonidine, | phentolamine, prazosin, |
| | oxymetazoline | tamsulosin, terazosin, tolazoline, |
| | | trazodone, amitriptyline, |
| | | silodosin, clomipramine, doxepin, |
| | | trimipramine, typical and atypical |
| | | antipsychotics, and |
| | | antihistamines, such as |
| | | hyroxyzine |
| α2 selective | A-methyl dopa, clonidine, | Phentolamine, |
| (ADRA2A, ADRA2B, | Brimonidine, agmatine, | phenoxybenzamine, yohimbine, |
| ADRA2C) | Dexmedetomidine, | idazoxan, atipamezole, |
| | Medetomidine, romifidine | mirtazapine, tolazoline, |
| | Chloroethylclonidine, | trazodone, and typical and |
| | Detomidine, lofexidine, xylazine, | atypical antipsychotics |
| | Tizanidine, guanfacine, and | |
| | amitraz | |
| β1 selective | Dobutamine | Metroprolol, atenolol, acebutolol, |
| (ADRB1) | | bisoprolol, betaxolol, |
| | | levobetaxolol, esmolol, celiprolol, |
| | | carteolol, landiolol, oxprenolol, |
| | | propanolol, practolol, penbutolol, |
| | | timolol, labetalol, nebivolol, |
| | | levobunolol, nadolol, pindolol, |
| | | sotalol, metipranolol, tertatolol, |
| | | vortioxene |
| β2 selective | Salbutamol, albuterol, bitolterol | Butaxamine, acebutolol, timolol, |
| (ADRB2) | mesylate, levabuterol, ritodrine, | propanolol, levobunolol, |
| | metaproterenol, terbutaline, | carteolol, labetalol, pindolol, |
| | salmeterol, formoterol, and | oxprenolol, nadolol, metipranolol, |
| | pirbuterol | penbutolol, tertatolol, sotalol |
| β3 selective | L-796568, amibegron, | SR 59230A, arotinolol |
| (ADRB3) | solabegron, mirabegron | |

TABLE 2C
DOPAMINE AGONISTS AND ANTAGONISTS

| Receptor | Agonist | Antagonist |
|---|---|---|
| Non-selective | Pramipexole, ropinirole, | Haloperidol, paliperidone, |
| | rotigotine, apomorphine, | clozapine, risperidone, |
| | propylnorapomorphine, | olanzapine, quetiapine, |
| | bromocriptine, cabergoline, | ziprasidone, metoclopramide, |
| | ciladopa, dihydrexidine, | droperidol, dromperidone, |
| | dinapsoline, doxamthrine, | amoxapine, clomipramine, |
| | epicriptine, lisuride, pergolide, | trimipramine, choline, melatonin, |
| | piribedil, quinagolide, roxindole, | acepromazine, amisulpride, |
| | dopamine | asenapine, azaperone, |
| | | benperidol, bromopride, |
| | | butaclamol, chlorpromazine, |
| | | chlorprothixene, clopenthixol, |
| | | eticlopride, flupenthixol, |
| | | fluphenazine, fluspirilene, |
| | | hydroxyzine, iodobenzamide, |
| | | levomepromazine, loxapine, |
| | | mesoridazine, nafadotride, |
| | | nemonapride, penfluridol, |
| | | perazine, perphenazine, |
| | | pimozide, prochlorperazine, |
| | | promazine, raclopride, |
| | | remoxipride, spiperone, |
| | | spiroxatrine, stepholidine, |
| | | sulpiride, suitopride, |
| | | tetrahydropalmatine, |
| | | thiethylperazine, thioridazine, |
| | | thiothixene, tiapride, |
| | | trifluoperazine, trifluperidol, |
| | | triflupromazine, and ziprasidone |
| D1 (DRD1) | Fenoldopam, A-86929, | Sch-23,390, skf-83,959, |
| | dihydrexidine, dinapsoline, | ecopipam |
| | dinoxyline, doxanthrine, SKF- | |
| | 81297, SKF-82958, SKF-38393, | |
| | G-BR-APB, dopexamine | |
| D2 (DRD2) | Cabergoline, pergolide, | Chloroethylnorapomorphine, |
| | quinelorane, sumanirole, | desmethoxyfallypride, |
| | talipexole, piribedil, quinpirole, | domperidone, eticlopride, |
| | quinelorane, dinoxyline, | fallypride, hydroxyzine, itopride, |
| | dopexamine | L-741,626, SV 293, yohimbine, |
| | | raclopride, sulpiride, |
| D3 (DRD3) | Piribedil, quinpirole, captodiame, | Domperidone, FAUC 365, |
| | compound R, R-16, FAUC 54, | nafadotride, raclopride, PNU- |
| | FAUC 73, PD-128,907, PF- | 99,194, SB-277011-A, sulpiride, |
| | 219,061, PF-592,379, CJ-1037, | risperidone, YQA14, U99194, SR |
| | FAUC 460, FAUC 346, | 21502 |
| | cariprazine | |
| D4 (DRD4) | Way-100635, a-412,997, abt- | A-381393, FAUC 213, L- |
| | 724, abt-670, fauc 316, pd-168, | 745,870, L-570,667, ML-398, |
| | 077, cp-226,269 | fananserin, clozapine |
| D5 (DRD5) | Dihydrexidine, rotigotine, SKF- | Sch 23390 |
| | 83,959, fenoldopam, | |
| Partial | Aplindore, brexpiprazole, | |
| | aripiprazole, CY-208,243, | |
| | pardoprunox, phencyclidine, and | |
| | salvinorin A | |

TABLE 2D
GABA AGONISTS AND ANTAGONISTS

| Receptor | Agonist | Antagonist |
|---|---|---|
| GABA_{A} | Barbiturates (e.g., allobarbital, | Bicuculline, gabazine, hydrastine, |
| | amobarbital, aprobarbital, | pitrazepin, sinomenine, tutin, |
| | alphenal, barbital, brallobarbital, | thiocolchicoside, metrazol, |
| | phenobarbital, secobarbital, | securinine, gabazine |
| | thiopental), bamaluzole, gaba, | |
| | gabob, gaboxadol, ibotenic acid, | |
| | isoguvacine, isonipecotic acid, | |
| | muscimol, phenibut, picamilon, | |
| | progabide, quisqualamine, sl | |
| | 75102, thiomuscimol, positive | |
| | allosteric modulators (pams) | |
| | (e.g., alcohols, such as ethanol | |
| | and isopropanol; avermectins, | |
| | such as ivermectin; | |
| | benzodiazepines, such as | |
| | diazepam, alprazolam, | |
| | chlordiazepoxide, clonazepam, | |
| | flunitrazepam, lorazepam, | |
| | midazolam, oxazepam, | |
| | prazepam, brotizolam, triazolam, | |
| | estazolam, lormetazepam, | |
| | nitrazepam, temazepam, | |
| | flurazepam, clorazepate | |
| | halazepam, prazepam, | |
| | nimetazapem, adinazolam, and | |
| | climazolam; bromides, such as | |
| | potassium bromide; carbamates, | |
| | such as meprobamate and | |
| | carisoprodol; chloralose; | |
| | chlormezanone; chlomethiazole; | |
| | dihydroergolines, such as | |
| | ergoloid; etazepine; etifoxine; | |
| | imidazoles, such as etomidate; | |
| | imidazopyridines, such as | |
| | alpidem and necopdiem; | |
| | kavalactones; loreclezole; | |
| | neuroactive steroids, such as | |
| | allogregnanolone, pregnanolone, | |
| | dihydrodeoxycorticosterone, | |
| | tetrahydrodeoxycortisosterone, | |
| | androstenol, androsterone, | |
| | etiocholanolone, 3α- | |
| | androstanediol, 5α, 5β, or 3α- | |
| | dihydroprogesterone, and | |
| | ganaxolone; | |
| | nonbenzodiazepines, such as | |
| | zalepon, zolpidem, zopiclone, | |
| | and eszopiclone; petrichloral; | |
| | phenols, such as propofol; | |
| | piperidinediones, such as | |
| | glutethimide and methyprylon; | |
| | propanidid; pyrazolopyridines, | |
| | such as etazolate; | |
| | pyrazolopyrimidines, such as | |
| | divapln and fasiplon; | |
| | cyclopyrrolones, sush as | |
| | pagoclone and suproclone; β- | |
| | cabolines, such as abecarnil and | |
| | geodecarnil; quinazolinones, | |
| | such as methaqualone; | |
| | scutellaria constituents; | |
| | stiripentol; sulfonylalkanes, such | |
| | as sulfonomethane, teronal, and | |
| | trional; valerian constituents, | |
| | such as valeric acid and | |
| | valerenic acid; and gases, such | |
| | as chloral hydrate, chloroform, | |
| | homotaurine, diethyl ether, and | |
| | sevoflurane. | |
| GABA_{B} | 1,4-butanediol, baclofen, GABA, | CGP-35348, homotaurine, |
| | Gabamide, GABOB, gamma- | phaclofen, saclofen, and SCH- |
| | butyrolactone, gamma- | 50911 |
| | hydroxybutyric acid, gamma- | |
| | hyrdoxyvaleric acid, gamma- | |
| | valerolactone, isovaline, | |
| | lesogaberan, phenibut, | |
| | picamilon, progabide, | |
| | homotaurine, SL-75102, | |
| | tolgabide | |
| GABA_{A}-ρ | CACA, CAMP, GABA, GABOB, | Gabazine, gaboxadol, |
| | N4-chloroacetylcytosine | isonipecotic acid, SKF-97,541, |
| | arabinoside, picamilon, | and (1,2,5,6-Tetrahydropyridin-4- |
| | progabide, tolgabide, and | yl)methylphosphinic acid |
| | neuroactive steroids, such as | |
| | allopregnanolone, THDOC, and | |
| | alphaxolone | |

TABLE 2E
MUSCARINC AGONISTS AND ANTAGONISTS

| Receptor | Agonist | Antagonist |
|---|---|---|
| Chrm1 | AF102B, AF150(S), AF267B, | Atropine, dicycloverine, |
| | acetylcholine, carbachol, | hyoscyamine, ipratropium, |
| | cevimeline, muscarine, | mamba toxin muscarinic toxin 7 |
| | oxotremorine, pilocarpine, | (MT7)^{,} olanzapine, oxybutynin, |
| | vedaclidine, 77-LH-28-1, CDD- | pirenzepine, telenzepine, and |
| | 0097, mcn-A-343, L689,660, and | tolterodine |
| | xanomeline | |
| Chrm2 | Acetylcholine, methacholine, | Atropine, dicycloverine, |
| | iper-8-naph, berbine, and | hyoscyamine, otenzepad, AQRA- |
| | (2S,2′R,3′S,5′R)-1-methyl-2-(2- | 741, AFDX-384, thorazine, |
| | methyl-1,3-oxathiolan-5- | diphenhydramine, |
| | yl)pyrrolidine 3-sulfoxide methyl | dimenhydrinate, ipratropium, |
| | iodide | oxybutynin, pirenzepine, |
| | | methoctramine, tripitramine, |
| | | gallamine, and tolterodine |
| Chrm3 | Acetylcholine, bethanechol, | Atropine, dicycloverine, |
| | carbachol, L689, 660, | hyoscyamine, alcidium bromide, |
| | oxotremorine, pilocarpine, | 4-DAMP, darifenacin, DAU-5884, |
| | aceclidine, arecoline, and | HL-031,120, ipratropium, J- |
| | cevimeline | 104,129, oxybutynin, tiotropium, |
| | | zamifenacin, and tolterodine |
| Chrm4 | Acetylcholine, carbachol, and | AFDX-384, dicycloverine, |
| | oxotremorine), and Chrm5 | himbacine, mamba toxin 3, PD- |
| | agonists (e.g., acetylcholine, | 102,807, PD-0298029, and |
| | milameline, sabcomeline | tropicamide |
| Chrm5 | Acetylcholine, milameline, | VU-0488130, xanomeline |
| | sabcomeline | |
| Non-selective | | Scopolamine, hydroxyzine, |
| | | doxylamine, dicyclomine, |
| | | flavoxate, cyclopentolate, |
| | | atropine methonitrate, |
| | | trihexyphenidyl/benzhexol, |
| | | solifenacin, benzatropine, |
| | | mebeverine, and procyclidine |

TABLE 2F
NICOTINIC AGONISTS AND ANTAGONISTS

| Receptor | Agonist | Antagonist |
|---|---|---|
| Chrna receptors | Choline, acetylcholine, | Turbocurarine, bupropion, |
| | carbachol, methacholine, | mecamylamine, 18- |
| | nicotine, varenicline tartrate, | methozycoronaridine, |
| | galantamine hydrobromide, | hexamethonium, trimethaphan, |
| | suxamethonium chloride | atraciurium, doxacurium, |
| | (succinylcholine chloride), | mivacurium, pancuronium, |
| | epibatidine, iobeline, | vecuronium, succinylcholine, |
| | decamethonium, | dextromethorphan, neramexane, |
| | isopronicline/TC-1734/AZD3480 | dextrophan, and 3- |
| | (TC-1734), AZD1446 (TC-6683), | methoxymorphinan |
| | TC-5619, TC-5214, MEM 3454 | |
| | (RG3487), ABT-894, ABT-560, | |
| | EVP-6124, EVP-4473, PNU- | |
| | 282987, AR-R17779, SSR | |
| | 189711, JN403, ABBF, PHA- | |
| | 543613, SEN12333, GTS- | |
| | 21/DMXB-A, AZD0328, A- | |
| | 582941, ABT-418, 5-iodo-A- | |
| | 85380, SIB-1765F, ABT-089, and | |
| | ABT-594 | |

TABLE 2G
SEROTONIN AGONISTS AND ANTAGONISTS

| Receptor | Agonist | Antagonist |
|---|---|---|
| 5-HT_{1A} | Azapirones, such as alnespirone, | Pindolol, tertatolol, alprenolol, |
| | binosperone, buspirone, | AV-965, BMY-7,378, |
| | enilospirone, etapirone, geprione, | cyanopindolol, dotarizine, |
| | ipsaprione, revospirone, | flopropione, GR-46,611, |
| | zalospirone, perospirone, | iodocyanopindolol, isamoltane, |
| | tiosperone, umespirone, and | lecozotan, mefway, methiothepin, |
| | tandospirone; 8-OH-DPAT, | methysergide, MPPF, NAN-190, |
| | befiradol, F-15,599, lesopitron, | oxprenolol, pindobind, |
| | MKC-242, LY-283,284, | propanolol, risperidone, |
| | osemozotan, repinotan, U- | robalzotan, SB-649,915, SDZ- |
| | 92,016-A, RU-24969, 2C-B, 2C- | 216,525, spiperone, spiramide, |
| | E, 2C-T-2, aripiprazole, | spiroxatrine, UH-301, WAY- |
| | asenapine, bacoside, befiradol, | 100,135, WAY-100,635, and |
| | brexpiprazole, bufotenin, | xylamidine |
| | cannabidiol, and fibanserin | |
| 5-HT_{1B} | Triptans, such as sumatriptan, | Methiothepin, yohimbine, |
| | rizatriptan, eletriptan, donitripatn, | metergoline, aripiprazole, |
| | almotriptan, frovatriptan, | isamoltane, AR-A000002, SB- |
| | avitriptan, zolmitriptan, and | 216,641, SB-224,289, GR- |
| | naratriptan; ergotamine, 5- | 127,935, SB-236,057 |
| | carboxamidotryptamine, CGS- | |
| | 12066A, CP-93,129, CP-94,253, | |
| | CP-122,288, CP-135,807, RU- | |
| | 24969, vortioxetine, ziprasidone, | |
| | and asenapine | |
| 5-HT_{1D} | Triptans, such as sumatriptan, | Ziprasidone, methiothepin, |
| | rizatriptan, and naratriptan; | yohimbine, metergoline, |
| | ergotamine, 5- | ergotamine, BRL-15572, |
| | (nonyloxy)tryptaime, 5-(t-butyl)- | vortioxetine, GR-127,935, LY- |
| | N-methyltryptamine, CP-286,601, | 310,762, LY-367,642, LY- |
| | PNU-109,291, PNU-142,633, | 456,219, and LY-456,220 |
| | GR-46611, L-694,247, L- | |
| | 772,405, CP-122,288, and CP- | |
| | 135,807 | |
| 5-HT_{1E} | BRL-54443, eletriptan | |
| 5-HT_{1F} | LY-334,370, 5-n-butyryloxy-DMT, | |
| | BRL-54443, eletriptan, LY- | |
| | 344,864, naratriptan, and | |
| | lasmiditan | |
| 5-HT_{2A} | 25I-NBOH, 25I-nbome, (R)-DOI, | Cyproheptadine, methysergide, |
| | TCB-2, mexamine, O-4310, | quetiapine, nefazodone, |
| | PHA-57378, OSU-6162, 25CN- | olanzapine, asenapine, pizotifen, |
| | NBOH, juncosamine, efavirenz, | LY-367,265, AMDA, hydroxyzine, |
| | mefloquine, lisuride, and 2C-B | 5-meo-nbpbrt, and niaprazine |
| 5-HT_{2B} | Fenfluramine, pergolide, | Agomelatine, aripiprazole, |
| | cabergoline, mefloquine, BW- | sarpogrelate, lisuride, tegaserod, |
| | 723C86, Ro60-0175, VER-3323, | metadoxine, RS-127,445, SDZ |
| | 6-APB, guanfacine, | SER-082, EGIS-7625, PRX- |
| | norfenfluramine, 5-meo-DMT, | 08066, SB-200,646, SB-204,741, |
| | DMT, mcpp, aminorex, | SB-206,553, SB-215,505, SB- |
| | chlorphentermine, MEM, MDA, | 228,357, LY-266,097, and LY- |
| | LSD, psilocin, MDMA | 272,015 |
| 5-HT_{2C} | Lorcaserin, lisuride, A-372,159, | Agomelatine, CPC, eltoprazine, |
| | AL-38022A, CP-809,101, | etoperidone, fluoxetine, FR- |
| | fenfluramine, mesulergine, MK- | 260,010, LU AA24530, |
| | 212, naphthyllisopropylamine, | methysergide, nefazodone, |
| | norfenfluramine, ORG-12,962, | norfluoxetine, O- |
| | ORG-37,684, oxaflozane, PNU- | desmethyltramadol, RS-102,221, |
| | 22395, PNU-181731, | SB-200,646, SB-221,284, SB- |
| | lysergamides, phenethylamines, | 242,084, SDZ SER-082, |
| | piperazines, tryptamines, Ro60- | tramadol, and trazodone |
| | 0175, vabicaserin, WAY-629, | |
| | WAY-161,503, WAY-163,909, | |
| | and YM-348 | |
| 5-HT_{2A/2C} | | Ketanserin, risperidone, |
| | | trazodone, mirtazapine, |
| | | clozapine |
| 5-HT₃ | 2-methyl-5-HT, alpha- | Dolasetron, granisetron, |
| | methyltryptamine, bufotenin, | ondansetron, palonosetron, |
| | chlorophenylbiguanide, ethanol, | tropisetron, alosetron, |
| | ibogaine, phenylbiguanide, | cilanosetron, mirtazapine, AS- |
| | quipazine, RS-56812, SR-57227, | 8112, bantopride, |
| | varenicline, and YM-31636 | metroclopramide, renzapride, |
| | | zacopride, mianserin, |
| | | vortioxetine, clozapine, |
| | | olanzapine, quetiapine, menthol, |
| | | thujone, lamotigrine, and 3- |
| | | tropanyl indole-3-carboxylate |
| 5-HT₄ | Cisapride, tegaserod, | Piboserod, GR-113,808, GR- |
| | prucalopride, BIMU-8, CJ- | 125,487, RS-39604, SB-203,186, |
| | 033,466, ML-10302, mosapride, | SB-204,070, and chamomile |
| | renzapride, RS-67506, RS- | |
| | 67333, SL65.1055, zacopride, | |
| | metoclopramide, and sulpride | |
| 5-HT_{5A} | Valeronic acid | ASP-5736, AS-2030680, AS- |
| | | 2674723, latrepiridine, |
| | | risperidone, and SB-699,551 |
| 5-HT₆ | EMDT, WAY-181,187, WAY- | ALX-1161, AVN-211, BVT-5182, |
| | 208,466, N-(inden-5- | BVT-74316, cerlapiridine, EGIS- |
| | yl)imidazothiazole-5-sulfonamide, | 12233, idalopiridine, interpridine, |
| | E-6837, E-6801, and EMD- | latrepiridine, MS-245, PRX- |
| | 386,088 | 07034, SB-258,585, SB-271,046, |
| | | SB-357,134, SB-339,885, Ro 04- |
| | | 6790, Ro-4368554, sertindole, |
| | | olanzapine, asenapine, |
| | | clozapine, rosa rugosa extract, |
| | | and WAY-255315 |
| 5-HT₇ | AS-19, 5-CT, 5-meot, 8-OH- | Amisulpride, amitriptyline, |
| | DAPT, aripiprazole, E-55888, E- | amoxapine, clomipramine, |
| | 57431, LP-12, LP-44, MSD-5a, | clozapine, DR-4485, |
| | RA-7, and N,N- | fluphenazine, fluperlapine, ICI |
| | Dimethyltryptamine | 169,369, imipramine, |
| | | ketanserine, JNJ-18038683, |
| | | loxapine, lurasidone, LY- |
| | | 215,840, maprotiline, |
| | | methysergide, mesulergine, |
| | | mianserin, olanzepine, pimozide, |
| | | ritanserin, SB-258,719, SB- |
| | | 258,741, SB-269,970, SB- |
| | | 656,104-A, SB-691,673, |
| | | sertindole, spiperone, tenilapine, |
| | | TFMPP, vortioxetine, |
| | | trifluoperazine, ziprasidone, and |
| | | zotepine |
| Non-selective | | Chlorpromazine, cyproheptadine, |
| 5-HT antagonists | | pizotifen, oxetorone, spiperone, |
| | | ritanserin, |
| | | parachlorophenylalanine, |
| | | metergoline, propranolol, |
| | | mianserin, carbinoxamine, |
| | | methdilazine, promethazine, |
| | | pizotifen, oxatomide, feverfew, |
| | | fenclonin, and reserpine |

TABLE 2H
GLUATAMATE RECEPTOR AGONISTS AND ANTAGONISTS

| Receptor | Agonist | Antagonist |
|---|---|---|
| Ionotropic | AMPA, glutamic acid, ibotenic | AP5, AP7, cppene, selfotel, HU- |
| (GRIA-14, GRIK1-5, | acid, kainic acid, NMDA, | 211, Huperzine A, gabapentin, |
| and GRIN1-3B) | quisqualic acid | remacemide, amantadine, |
| | | atomoxetine, AZD6765, |
| | | agmatine, chloroform, |
| | | dextrallorphan, |
| | | dextromethorphan, dextrorphan, |
| | | diphenidine, dizocilpine (MK- |
| | | 801), ethanol, eticyclidine, |
| | | gacyclidine, ibogaine, ifenprodil, |
| | | ketamine, kynurenic acid, |
| | | memantine, magnesium, |
| | | methoxetamine, nitromemantine, |
| | | nitrous oxide, PD-137889, |
| | | perampanel, phencyclidine, |
| | | rolicyclidine, tenocyclidine, |
| | | methoxydine, tiletamine, |
| | | neramexane, eliprodil, etoxadrol, |
| | | dexoxadrol, WMS-2539, NEFA, |
| | | delucemine, 8A-PDHQ, |
| | | aptiganel, rhynchophylline |
| Metabotropic | L-ap4, acpd, l-qa, chpg, ly- | AIDA, fenobam, MPEP, LY- |
| (GRM1-8) | 379,268, ly-354,740, acpt, vu | 367,385, EGLU, CPPG, MAP4, |
| | 0155041 | MSOP, LY-341,495 |
| Glycine | | Rapastinel, NRX-1074, 7- |
| antagonists | | chlorokynurenic acid, 4- |
| | | chlorokynurenine, 5,7- |
| | | dichlorokynurenic acid, kynurenic |
| | | acid, TK-40, 1- |
| | | aminocyclopropanecarboxylic |
| | | acid (ACPC), L-phenylalanine, |
| | | and xenon |

TABLE 2I
HISTAMINE AGONISTS AND ANTAGONISTS

| Receptor | Agonist | Antagonist |
|---|---|---|
| Non-selective | Histamine dihydrochloride, HTMT | |
| | dimaleate, 2-pyridylethlyamine | |
| | dihydrochloride | |
| H₁ | | Acrivastine, azelastine, |
| | | astemizole, bilastine, |
| | | bromodiphenhydramine, |
| | | brompheniramine, buclizine, |
| | | carbinoxamine, cetirizine, |
| | | cetirizine dihydrochloride, |
| | | clemastine fumarate, clemizole |
| | | hydrochloride, |
| | | chlorodiphenhydramine, |
| | | chlorphenamine, chlorpromazine, |
| | | clemastine, cyclizine, |
| | | cyproheptadine, |
| | | dexbrompheniramine, |
| | | dexchlorpheniramine, |
| | | dimenhydrinate, dimethindene |
| | | maleate, dimetindene, |
| | | diphenhydramine, |
| | | diphenhydramine hydrochloride, |
| | | doxepin hydrochloride, |
| | | doxylamine, ebastine, |
| | | embramine, fexofenadine, |
| | | fexofenadine hydrochloride, |
| | | hydroxyzine, ketotifen fumarate, |
| | | loratadine, meclizine, meclizine |
| | | dihydrochloride, mepyramine |
| | | maleate, mirtazapine, |
| | | olopatadine, olopatadine |
| | | hydrochloride, orphenadrine, |
| | | phenindamine, pheniramine, |
| | | phenyltoloxamine, promethazine, |
| | | quetiapine, rupatadine, |
| | | terfenadine, tripelennamine, |
| | | zotepine, trans- triprolidine |
| | | hydrochloride, and triprolidine |
| H₁ inverse agonists | | Cetirizine, levocetirizine, |
| | | desloratadine, and pyrilamine |
| H₂ | Betazole, impromidine, dimaprit | Aminopotentidine, cimetidine, |
| | dihydrochloride, and amthamine | famotidine, ICI 162,846, |
| | dihyrdobromide | lafutidine, nizatidine, ranitidine, |
| | | ranitidine hyrdochloride, |
| | | roxatidine, zolantadine |
| | | dimaleate, and toitidine |
| H₃ | Imetit dihydropbromide, immepip | Clobenpropit, clobenpropit |
| | dihyrdrobromide, immethridine | dihydrobromide, A 3314440 |
| | dihydrobromide, α- | dihyrdochloride, BF 2649 |
| | Methylhistamine dihydrobromide, | hydrochloride, carcinine |
| | N-methylhistamine | ditrifluoroacetate, ABT-239, |
| | dihydrochloride, proxyfan | ciprofaxin, conessine, GT 2016, |
| | oxalate, and betahistine | A-349,821, impentamine |
| | | dihydrobromide, iodophenpropit |
| | | dihydrobromide, JNJ 10181457 |
| | | dihydrochloride, JNJ 5207852 |
| | | dihydrochloride, ROS 234 |
| | | dioxalate, SEN 12333, VUF 5681 |
| | | dihydrobromide, and |
| | | thioperamide |
| H₄ | Imetit dihydropbromide, immepip | Thioperamide, JNJ 7777120, A |
| | dihyrdrobromide, 4- | 943931 dihydrochloride, A |
| | methylhistamine dihydrochloride, | 987306, JNJ 10191584 maleate, |
| | clobenpropit dihydrobromide, | and VUF-6002 |
| | VUF 10460, and VUF 8430 | |
| | dihydrobromide | |

TABLE 2J
CANNABINOID AGONISTS AND ANTAGONISTS

| Receptor | Agonist | Antagonist |
|---|---|---|
| Cannabinoid receptor (non- | Anandamide, N-Arachidonoyl | |
| selective) | dopamine, 2- | |
| | Arachidonoylglycerol (2-AG), 2- | |
| | Arachidonyl glyceryl ether, Δ-9- | |
| | Tetrahydrocannabinol, EGCG, | |
| | Yangonin, AM-1221, AM-1235, | |
| | AM-2232, UR-144, JWH-007, | |
| | JWH-015, JWH-018, ACEA, | |
| | ACPA, arvanil, CP 47497, DEA, | |
| | leelamine, methanandamide, | |
| | NADA, noladin ether, oleamide, | |
| | CB 65, GP-1a, GP-2a, GW | |
| | 405833, HU 308, JWH-133, L- | |
| | 759,633, L-759,656, LEI 101, | |
| | MDA 19, and SER 601 | |
| CB₁ receptor | ACEA, ACPA, RVD-Hpα, (R)-(+)- | Rimonabant, cannabidiol, Δ⁹- |
| | methanandamide | tetrahydrocannabivarin (THCV), |
| | | taranabant, otenabant, |
| | | surinabant, rosonabant, SLV- |
| | | 319, AVE1625, V24343, AM 251, |
| | | AM 281, AM 6545, hemopressin, |
| | | LY 320135, MJ 15, CP 945598, |
| | | NIDA 41020, PF 514273, SLV |
| | | 319, SR 1141716A, and TC-C |
| | | 14G |
| CB₂ receptor | CB 65, GP 1a, GP 2a, GW | Cannabidiol, Δ⁹- |
| | 405833, HU 308, JWH 133, L- | tetrahydrocannabivarin (THCV), |
| | 759,656, L-759,633, SER 601, | AM 630, COR 170, JTE 907, and |
| | LEI 101 | SR 144528 |

TABLE 2K
PURINERGIC RECEPTOR AGONISTS AND ANTAGONISTS

| Receptor | Agonist | Antagonist |
|---|---|---|
| ADORA1 (P1 adenosine | Adenosine, N6- | Caffeine, theophylline, 8- |
| receptor) | Cyclopentyladenosine, N6-3- | Cyclopentyl-1,3-dimethylxanthine |
| | methoxyl-4-hydroxybenzyl | (CPX), 8-Cyclopentyl-1,3- |
| | adenine riboside (B2), CCPA, | dipropylxanthine (DPCPX), 8- |
| | tecadenoson, selodenoson, | Phenyl-1,3-dipropylxanthine, |
| | Certain Benzodiazepines and | bamifylline, BG-9719, BG09928, |
| | Barbiturates, 2′-meccpa, GR | FK-453, FK838, rolofylline, N- |
| | 79236, and SDZ WAG 994 | 0861, and PSB 36 |
| ADORA2A (P1 adenosine | Adenosine, N6-3-methoxyl-4- | Caffeine, theophylline, |
| receptor) | hydroxybenzyl adenine riboside | istradefylline, SCH-58261, SCH- |
| | (B2), YT-146, DPMA, UK- | 442,416, ATL-444, MSX-3, |
| | 423,097, limonene, NECA, CV- | preladenant, SCH-412,348, VER- |
| | 3146, binodenoson, ATL-146e, | 6623, VER-6947, VER-7835, |
| | CGS-21680, and Regadenoson | vipadenant, and ZM-241,385 |
| ADORA2B (P1 adenosine | Adenosine, 5′-N- | Caffeine, theophylline, CVT- |
| receptor) | ethylcarboxamidoadenosine, | 6883, ATL-801, compound 38, |
| | BAY 60-6583, LUF-5835, NECA, | MRS-1706, MRS-1754, OSIP- |
| | (S)-PHPNECA, and LUF-5845 | 339,391, PSB-603, PSB-0788, |
| | | and PSB-1115 |
| ADORA3 (P1 adenosine | Adenosine, 2-(1-Hexynyl)-N- | Caffeine, theophylline, MRS- |
| receptor) | methyladenosine, CF-101 (IB- | 1191, MRS-1220, MRS-1334, |
| | MECA), CF-102, 2-Cl-IB-MECA, | MRS-1523, MRS-3777, |
| | CP-532,903, inosine, LUF-6000, | MRE3008F20, MRE3005F20, |
| | and MRS-3558 | OT-7999, SSR161421, KF- |
| | | 26777, PSB-10, PSB-11, and |
| | | VUF-5574 |
| P2Y receptor | ATP, ADP, UTP, UDP, UDP- | Clopidogrel, elinogrel, prasugrel, |
| | glucose, 2-methylthioladenosine | ticlopidine, ticagrelor, AR-C |
| | 5′ diphosphate (2-mesadp), | 118925XX, AR-C 66096, AR-C |
| | lysophosphatidic acid, PSB 1114, | 69931, AZD 1283, MRS 2179, |
| | PSB 0474, NF 546, MRS 2365, | MRS 2211, MRS 2279, MRS |
| | MRS 2690, MRS 2693, MRS | 2500, MRS 2578, NF 157, NF |
| | 2768, MRS 2905, MRS 2957, | 340, PPADS, PPTN |
| | MRS 4062, and denufosol (P2Y₂ | hydrochloride, PSD 0739, SAR |
| | agonist) | 216471, and suramin |
| P2X receptor | Atp | A 438079, A 740003, A 804598, |
| | | A 839977, AZ 10606120, AZ |
| | | 11645373, 5-BDBD, BX 430, |
| | | Evans Blue, JNJ 47965567, KN- |
| | | 62, NF 023, NF 110, NF 157, NF |
| | | 279, NF 449, PPADS, iso- |
| | | PPADS, PPNDS, Ro 0437626, |
| | | Ro 51, RO-3, TC-P 262, suramin, |
| | | TNP-ATP, and P2X₇ antagonists |
| | | NF279, calmidazolium, and KN- |
| | | 62 |

TABLE 2L
NEUROPEPTIDE AGONISTS AND ANTAGONISTS

| Gene | Agonist | Antagonist |
|---|---|---|
| Neuropeptide Y receptor (non- | Neuropeptide Y, pancreatic | |
| selective) | polypeptide, BWX-46, and | |
| | Peptide YY | |
| Neuropeptide Y₁ receptor | | BVD-10, GR-231,118, BIBO- |
| | | 3304, BIBP-3226, PD-160,170, |
| | | and BMS 193885 |
| Neuropeptide Y₂ receptor | | BIIE-0246, CYM 9484, JNJ |
| | | 5207787, and SF 11 |
| Neuropeptide Y₄ receptor | | UR-AK49 and GR-231,118 |
| Neuropeptide Y₅ receptor | | Lu AA-33810, CGP 71683 |
| | | hydrochloride, GW 438014A, L- |
| | | 152,804, NPY 5RA972, NTNCB |
| | | hydrochloride, velneperit, and S |
| | | 25585 |
| Somatostatin receptor | Somatostatin, cortistatin, | Cyclomastatin and CYN 154806 |
| | octreotide, lanreotide CH 275, TT | |
| | 232, TC-G 1003, seglitide, RC | |
| | 160, NNC 26-9100, L-817,818, L- | |
| | 803,087 trifluoroacetate, and | |
| | (1R,1′S,3′R/1R,1′R,3′S)-L- | |
| | 054,264 | |
| CGRP receptor (calcitonin gene- | A-CGRP, β-CGRP, calcitonin, | Telcagepant, sumatriptan |
| related peptide receptor) | PHM 27, amylin, pramlintide, | (decreases CGRP promoter |
| | CRSP-1, and SUN-B 8155 | activity), MK-3207, and BIBN |
| | | 4096 BS, MK-0974 |
| Tachykinin 1 receptor (NK₁ | Substance P, GR-73632, and | Aprepitant, Casopitant, |
| receptor) | C14TKL-1 | Ezlopitant, Fosaprepitant, |
| | | Lanepitant, Maropitant, |
| | | Vestipitant, L-733,060, L- |
| | | 741,671, L-742,694, RP-67580, |
| | | RPR-100,893, CP-96345, CP- |
| | | 99994, GR-205,171, TAK-637, |
| | | FK 888, GR 82334, L-760,735, L- |
| | | 732,138, L-733,060, SDZ NKT |
| | | 343, Spantide 1, SR 140333, and |
| | | T-2328 |
| Tachykinin 2 receptor (NK₂ | Neurokinin A and GR-64349 | Ibodutant, saredutant, GR- |
| receptor) | | 159,897, GR 94800, MDL |
| | | 29,913, and MEN 11420, MEN- |
| | | 10376 |
| Tachykinin 3 receptor (NK₃ | Neurokinin B and senktide | Fezolinetant, MLE-4901, |
| receptor) | | Osanetant, Talnetant, SB- |
| | | 222,200, SSR 146977, and SB- |
| | | 218,795 |
| Vasoactive intestinal polypeptide | VIP, PACAP, PACAP-38, | VIP (6-28), [D-p-Cl-Phe⁶,Leu¹²]- |
| receptor 1 (VIPR1/VPAC1) and | PACAP-27, peptide histidine | VIP, and AC-Tyr¹,D-Phe²]GRF 1- |
| Vasoactive intestinal polypeptide | isoleucineamide (PHI), peptide | 29 amide |
| receptor 2 (VIPR2/VPAC2) | histidine methionineamide | |
| | (PHM), peptide histidine valine | |
| | (PHV), and Bay 55-9837 | |
| Opioid receptor (non-selective) | Dynorphins, enkephalins, | |
| | endorphins, endomorphins, and | |
| | nociceptin | |
| μ-opioid receptor | DAMAGO, endomorphin-1, | Naloxone, naltrexone, |
| | endomorphin-2, fentanyl, | nalmefene, diprenorphine, |
| | loperamide, meptazinol, | nalorphine, nalorphine |
| | oxycodone, PL 017, sinomenine, | dinicotinate, levallorphan, |
| | and buprenorphine (partial) | samidorphan, nalodeine, |
| | | alvimopan, methylnaltrexone, |
| | | naloxegol, 6β-naltrexol, |
| | | axelopran, bevenopran, |
| | | methylsamidorphan, |
| | | naldemedine, buprenorphine, |
| | | dezocine, eptazocine, CTAP, |
| | | CTOP, cyprodime, clocinnamox |
| | | mesylate, naloxonazine, |
| | | funaltrexamine, and cyprodamine |
| κ-opioid receptor | Alazocine, Bremazocine, 8- | 5′-Acetamidinoethylnaltrindole, |
| | Carboxamidocyclazocine, | 5′-Guanidinonaltrindole, 6′- |
| | Cyclazocine, Ketazocine, | Guanidinonaltrindole, |
| | Metazocine, Pentazocine, | Amentoflavone, AT-076, |
| | Phenazocine, Morphinans (e.g., | Binaltorphimine, BU09059, |
| | 6′-Guanidinonaltrindole, | Buprenorphine, CERC-501, |
| | Butorphan, Butorphanol, | Dezocine, DIPPA, jdtic, LY- |
| | Cyclorphan, Diprenorphine, | 255582, LY-2459989, LY- |
| | Etorphine, Levallorphan, | 2795050, Methylnaltrexone, |
| | Levomethorphan, Levorphanol, | ML190, ML350, MR-2266, |
| | Morphine, Nalbuphine, | Naloxone, Naltrexone, |
| | Nalfurafine, Nalmefene, | Noribogaine, Norbinaltorphimine, |
| | Nalodeine, Nalorphine, | Pawhuskin A, PF-4455242, |
| | Norbuprenorphine, | Quadazocine, RB-64, and |
| | Norbuprenorphine-3-glucuronide, | Zyklophin |
| | Oxilorphan, Oxycodone, | |
| | Proxorphan, Samidorphan, and | |
| | Xorphanol), Arylacetamides (e.g., | |
| | Asimadoline, BRL-52537, | |
| | Eluxadoline, Enadoline, GR- | |
| | 89696, ICI-204,448, ICI-199,441, | |
| | LPK-26, MB-1C-OH, Niravoline, | |
| | N-MPPP, Spiradoline, U-50,488, | |
| | U-54,494A, and U-69,593), | |
| | CR665, Difelikefalin (CR845), | |
| | Dynorphins (dynorphin A, | |
| | dynorphin B, big dynorphin), | |
| | Terpenoids (e.g., Collybolide, | |
| | Erinacine E, Mentholm RB-64, | |
| | Salvinorin A, and 2- | |
| | Methoxymethyl salvinorin B), | |
| | Apadoline, HS665, HZ-2, | |
| | Ibogaine, Ketamine, Noribogaine, | |
| | Pentazocine, Tifluadom, and | |
| | Nalfurafine | |
| δ-opioid receptor | Leu-enkephalin, Met-enkephalin, | Buprenorphine, naltriben, |
| | Deltorphins I and II, DADLE, AR- | naltrindole, SDM25N, ICI- |
| | M 100390, 7- | 174,864, ICI-154,129, BNTX, and |
| | Spiroindanyloxymorphone, N- | benzylnatrindole |
| | Phenethyl-14-ethoxymetopon, | |
| | ADL-5859, BU-48, SNC-80, | |
| | SNC-162, FIT, 6′-GTI, DPDPE, | |
| | BW373U86, DPI-221, DPI-287, | |
| | DPI-3290, TAN-67, RWJ- | |
| | 394674, Norbuprenorphine, | |
| | Cannabidiol, | |
| | Tetrahydrocannabinol, | |
| | Xorphanol, Mitragynine, and | |
| | Mitragynine pseudoindoxyl | |
| NOP receptor (opioid) | Orphanin, SCH-221510, NNC | UFP-101, Trap 101, nocistatin, |
| | 63-0532, MCOPPB, and Ac- | BAN ORL 24, J 113397, JTC |
| | RYYRWK-NH₂ | 801, and SB 612111 |
| Oxytocin receptor | Carbetocin, demoxytocin, lipo- | Atosiban, barusiban, epelsiban, |
| | oxytocin-1, merotocin, oxytocin, | L-368,899, L-371,257, L-372- |
| | TC OT 39, and WAY-267,464 | 662, retosiban, SSR-126,768, |
| | | and WAY-162,720 |
| Vasopressin receptor | Vasopressin and desmopressin | TC OT 39, OPC 21268, SR |
| | | 49059, TASP 0390325, |
| | | conivaptan, tolvaptan, |
| | | mozavaptan, lixivaptan, |
| | | satavaptan, relcovaptan, |
| | | nelivaptan, demeclocycline, and |
| | | lithium |

TABLE 2M
NEUROTRANSMISSION MODULATORS

| Type | Modulators |
|---|---|
| Norepinephrine reuptake inhibitors | Amedalin, atomoxetine, CP-39,332, daledalin, |
| (increase adrenergic neurotransmission) | edivoxetine, esreboxetine, lortalamine, nisoxetine, |
| | reboxetine, talopram, talsupram, tandamine, |
| | viloxazine, bupropion, ciclazindol, manifaxine, |
| | maprotiline, radafaxine, tapentadol, teniloxazine, |
| | protriptyline, nortriptyline, and desipramine |
| Norepineprhine-dopamine reuptake inhibitors | Amineptine, bupropion, desoxypipradrol, |
| (increase adrenergic and dopamine | dexmethylphenidate, difemetorex, diphenylprolinol, |
| neurotransmission) | ethylphenidate, fencamfamine, fencamine, |
| | lefetamine, methylenedioxypyrovalerone, |
| | methylphenidate, nomifensine, O-2172, oxolinic |
| | acid, pipradrol, prolintane, pyrovalerone, |
| | tametraline, and WY-46824 |
| Serotonin-norepinephrine-dopamine reuptake | Mazindol, nefazodone, sibutramine, venlafaxine, |
| inhibitors (SNDRIs) and serotonin-norepinephrine | esketamine, duloxetine, ketamine, phencyclidine, |
| reuptake inhibitors (SNRIs) | tripelennamine, mepiprazole, amitifadine, AN788, |
| (increase adrengergic, dopamine, and serotonin | ansofaxine, centanafadine, atomoxetine, |
| neurotransmission) | desvenlafaxine, milnacipran, levomilnacipran, |
| | dasotraline, Lu AA34893, Lu AA37096, NS-2360, |
| | tedatioxetine, tesofensine, bicifadine, BMS- |
| | 866,949, brasofensine, diclofensine, DOV-216,303, |
| | EXP-561, liafensine, NS-2359, RG-7166, SEP- |
| | 227,162, SEP-228,425, SEP-228,432, naphyrone, |
| | 3,3-Diphenylcyclobutanamine, 3,4- |
| | Dichlorotametraline, D-161, desmethylsertraline, |
| | DMNPC, DOV-102,677, fezolamine, |
| | GSK1360707F, indatraline, JNJ-7925476, JZ-IV- |
| | 10, JZAD-IV-22, LR-5182, methylnaphthidate, MI-4, |
| | PRC200-SS, PRC050, PRC025, SKF-83,959, TP1, |
| | phenyltropanes (e.g., WF-23, dichloropane, and |
| | RTI-55), Ginkgo biloba extract, St John's Wort, |
| | hyperforin, adhyperforin, and uliginosin B |
| Dopamine reuptake inhibitors | Dopamine reuptake inhbiitors (e.g., altropane, |
| (increase dopamine neurotransmission) | amfonelic acid, amineptine, BTCP, 3C-PEP, DBL- |
| | 583, difluoropine, GBR-12783, GBR-12935, GBR- |
| | 13069, GBR-13098, GYKI-52895, lometopane, |
| | methylphenidate, ethylphenidate, modafinil, |
| | armodafinil, RTI-229, vanoxerine, adrafinil, |
| | benztropine, bupropion, fluorenol, medifoxamine, |
| | metaphit, rimcazole, venlafaxine, Chaenomeles |
| | speciosa, and oroxylin A), dopamine releasing |
| | agents (e.g., p-Tyramine), dextroamphetamine, |
| | lisdexamfetamine, dexmethylphenidate, and |
| | cathinone |
| Dopamine prodrugs | Levopoda, docarpamine |
| (increase dopamine neurotransmission) | |
| GABA reuptake inhibitors | CL-996, deramciclane, gabaculine, guvacine, |
| (increase GABA neurotransmission) | nipecotic acid, NNC-711, NNC 05-2090, SKF- |
| | 89976A, SNAP-5114, tiagabine, and hyperforin |
| GABA analogs | Gabapentin, butyric acid, valproic acid, valpromide, |
| (increase GABA neurotransmission) | valnoctamide, 3-hydroxybutanal, GHB, sodium, |
| | oxybate, aceburic acid, GBL, GHBAL, GHV, GVL, |
| | GHC, GCL, HOCPCA, UMB68, pregabalin, tolibut, |
| | phaclofen, sacolfen, arecaidine, gaboxadol, |
| | isonipecotic acid, 3-Methyl-GABA, AABA, BABA, |
| | DAVA, GAVA, Glutamic acid, hopantenic acid, |
| | piracetam, and vigabatrin |
| GABA prodrugs | L-Glutamine, N-Isonicotinoyl-GABA, picamilon, |
| (increase GABA neurotransmission) | progabide, tolgabide |
| Acetylcholinesterase inhibitors | Carbamates, physostigmine, neostigmine, |
| (increase nicotinic and muscarinic | pyridostigmine, ambenonium, demecarium, |
| neurotransmission) | rivastigmine, phenanthrene derivatives, |
| | galantamine, caffeine, rosmarinic acid, alpha- |
| | pinene, piperidines, donepezil, tacrine, |
| | edrophonium, Huperzine A, ladostigil, ungeremine, |
| | lactucopicrin, dyflos, echothiophate, parathion, and |
| | quasi-irreversible acetylcholinesterase inhibitors |
| Serotonin reuptake inhibitors | Alaproclate, cericlamine, citalopram, dapoxetine, |
| (increase serotonin neurotransmission) | escitalopram, femoxetine, fluoxetine, fluvoxamine, |
| | ifoxetine, indalpine, omiloxetine, panuramine, |
| | paroxetine, pirandamine, RTI-353, sertraline, |
| | zimelidine, desmethylcitalopram, |
| | didesmethylcitalopram, seproxetine ((S)- |
| | norfluoxetine), desvenlafaxine, cianopramine, |
| | litoxetine, lubazodone, SB-649,915, trazodone, |
| | vilazodone, vortioxetine, dextromethorphan, |
| | dextropropoxyphene, dimenhydrinate, |
| | diphenhydramine, mepyramine (pyrilamine), |
| | mifepristone, delucemine, mesembrenone, |
| | mesembrine, roxindole, duloxetine, |
| | levomilnacipran, milnacipran, dapoxetine, |
| | sibutramine, chlorpheniramine, |
| | dextropmethorphan, and methadone |
| Serotonin releasing agents | Chlorphentermine, cloforex, dexfenfluramine, |
| (increase serotonin neurotransmission) | etolorex, fenfluramine, flucetorex, indeloxazine, |
| | levofenfluramine, tramadol, carbamazepine, |
| | amiflamine (FLA-336), viqualine (PK-5078), 2- |
| | Methyl-3,4-methylenedioxyamphetamine (2-Methyl- |
| | MDA), 3-Methoxy-4-methylamphetamine (MMA), 3- |
| | Methyl-4,5-methylenedioxyamphetamine (5-Methyl- |
| | MDA), 3,4-Ethylenedioxy-N-methylamphetamine |
| | (EDMA), 4-Methoxyamphetamine (PMA), 4- |
| | Methoxy-N-ethylamphetamine (PMEA), 4-Methoxy- |
| | N-methylamphetamine (PMMA), 4- |
| | Methylthioamphetamine (4-MTA), 5-(2- |
| | Aminopropyl)-2,3-dihydrobenzofuran (5-APDB), 5- |
| | Indanyl-2-aminopropane (IAP), 5-Methoxy-6- |
| | methylaminoindane (MMAI), 5-Trifluoromethyl-2- |
| | aminoindane (TAI), 5,6-Methylenedioxy-2- |
| | aminoindane (MDAI), 5,6-Methylenedioxy-N- |
| | methyl-2-aminoindane (MDMAI), 6-Chloro-2- |
| | aminotetralin (6-CAT), 6-Tetralinyl-2-aminopropane |
| | (TAP), 6,7-Methylenedioxy-2-aminotetralin (MDAT), |
| | 6,7-Methylenedioxy-N-methyl-2-aminotetralin |
| | (MDMAT), N-Ethyl-5-trifluoromethyl-2-aminoindane |
| | (ETAI), N-Methyl-5-indanyl-2-aminopropane, |
| | aminorex, MDMA, MDEA, MDA, MBDB, and |
| | tryptamines, such as DMT, αmt, 5meo-NMT, NMT, |
| | NETP, Dimethyl-Serotonin, 5meo-NET, αet and |
| | αmt |
| Excitatory amino acid reuptake inhibitors | Didydrokanic acid, WAY-213,613, L-trans-2,4-PDC, |
| (increase Glutamate receptor neurotransmission) | amphetamine, and L-Theanine |
| Glycine reuptake inhibitors | Bitopertin, Org 24598, Org 25935, ALX-5407, |
| (increase Glutamate receptor neurotransmission) | sacrosine, Org 25543, and N-arachidonylglycerine |
| Histidine decarboxylase inhibitors | Tritoqualine, catechin |
| (decrease histamine neurotransmission) | |
| Endocannabinoid enhancers | AM404, fatty acid amide hydrolase inhibitors (e.g., |
| (increase cannabinoid neurotransmission) | AM374, ARN2508, BIA 10-2472, BMS-469908, |
| | CAY-10402, JNJ-245, JNJ-1661010, JNJ- |
| | 28833155, JNJ-40413269, JNJ-42119779, JNJ- |
| | 42165279, MK-3168, MK-4409, MM-433593, OL- |
| | 92, OL-135, PF-622, PF-750, PF-3845, PF- |
| | 04457845, PF-04862853, RN-450, SA-47, SA-73, |
| | SSR-411298, ST-4068, TK-25, URB524, URB597, |
| | URB694, URB937, VER-156084, and V-158866 |
| Monoacylglycerol lipase inhibitors | N-arachidonoyl maleimide, JZL184 |
| (increase cannabinoid neurotransmission) | |
| Endocannabinoid transporter inhibitors | Sb-fi-26 |
| (increase cannabinoid neurotransmission) | |
| Endocannabinoid reuptake inhibitors | AM404, AM1172, LY-2183240, O-2093, OMDM-2, |
| (increase cannabinoid neurotransmission) | UCM-707, VDM-11, guineensine, ETI-T-24_B_I, |
| | WOBE437, and RX-055 |
| Adenosine uptake inhibitors | Cilostazol, dilazep, and dipyramidole |
| (increase purinergic neurotransmission) | |
| Nucleoside transporter inhibitors | 8MDP, Decynium 22, 5-iodotubercidin, NBMPR, |
| (increase purinergic neurotransmission) | and TC-T 6000 |

Neurotoxins

In some embodiments, the neurotransmission modulator is a neurotoxin (e.g., a neurotoxin listed in Table 3), or a functional fragment or variant thereof. Neurotoxins include, without limitation, convulsants, nerve agents, parasympathomimetics, and uranyl compounds. Neurotoxins may be bacterial in origin, or fungal in origin, or plant in origin, or derived from a venom or other natural product. Neurotoxins may be synthetic or engineered molecules, derived de novo or from a natural product. Suitable neurotoxins include but are not limited to botulinum toxin and conotoxin. Exemplary neurotoxins are listed in Table 3.

TABLE 3
NEUROTOXINS
NEUROTOXINS

2,4,5-Trihydroxyamphetamine
2,4,5-Trihydroxymethamphetamine
3,4-Dichloroamphetamine
5,7-Dihydroxytryptamine
5-Iodowillardiine
Ablomin
Aconitine
Aconitum
Aconitum anthora
AETX
Agelenin
Agitoxin
Aldrin
Alpha-Methyldopamine
Alpha-neurotoxin
Altitoxin
Anatoxin-a
Androctonus australis hector insect toxin
Anisatin
Anthopleurin
Antillatoxin
Anuroctoxin
Apamin
Arum italicum
Arum maculatum
Babycurus toxin 1
Batrachotoxin
BDS-1
Bestoxin
Beta-Methylamino-L-alanine
BgK
Birtoxin
BmKAEP
BmTx3
BotlT2
BotlT6
Botulinum toxin
Brevetoxin
Bukatoxin
Butantoxin
Calcicludine
Calciseptine
Calitoxin
Caramboxin
Carbon disulfide
CgNa toxin
Charybdotoxin
Cicutoxin
Ciguatoxin
Cll1
Clostridium botulinum
Conantokins
Conhydrine
Coniine
Conotoxin
Contryphan
CssII
CSTX
Curare
Cyanide poisoning
Cylindrospermopsin
Cypermethrin
Delta atracotoxin
Dendrotoxin
Dieldrin
Diisopropyl fluorophosphates
Dimethylmercury
Discrepin
Domoic acid
Dortoxin
DSP-4
Ergtoxin
Falcarinol
Fenpropathrin
Gabaculine
Ginkgotoxin
Grammotoxin
Grayanotoxin
Hainantoxin
Halcurin
Hefutoxin
Helothermine
Heteroscodratoxin-1
Histrionicotoxin
Homoquinolinic acid
Hongotoxin
Huwentoxin
Ibotenic acid
Ikitoxin
Inhibitor cystine knot
Jingzhaotoxin
Kainic acid
Kaliseptine
Kappa-bungarotoxin
Kodaikanal mercury poisoning
Kurtoxin
Latrotoxin
Lq2
Maitotoxin
Margatoxin
Maurotoxin
Mercury (element)
Methanol
Methiocarb
MPP+
MPTP
Nemertelline
Neosaxitoxin
Nicotine
N-Methylconiine
Oenanthotoxin
Oxalyldiaminopropionic acid
Oxidopamine
Oxotoxin
Pahutoxin
Palytoxin
Pandinotoxin
Para-Bromoamphetamine
Para-Chloroamphetamine
Para-Chloromethamphetamine
Para-Iodoamphetamine
Penitrem A
Phaiodotoxin
Phenol
Phoneutria nigriventer toxin-3
Phrixotoxin
Polyacrylamide
Poneratoxin
Psalmotoxin
Pumiliotoxin
Quinolinic acid
Raventoxin
Resiniferatoxin
Samandarin
Saxitoxin
Scyllatoxin
Sea anemone neurotoxin
Slotoxin
SNX-482
Stichodactyla toxin
Taicatoxin
Taipoxin
Tamapin
Tertiapin
Tetanospasmin
Tetraethylammonium
Tetramethylenedisulfotetramine
Tetrodotoxin
Tityustoxin
Tricresyl phosphate
TsIV
Vanillotoxin
Veratridine

Neurotransmission modulators also include antibodies that bind to neurotransmitters or neurotransmitter receptors listed in Tables 1A, 1B, Table 7, and Table 8 and decrease neurotransmission. These antibodies include blocking and neutralizing antibodies. Antibodies to neurotransmitters or neurotransmitter receptors listed in Tables 1A, 1B, Table 7, and Table 8 can be generated by those of skill in the art using well established and routine methods.

Neuropeptide Signaling Modulators

In some embodiments, a neuromodulating agent is a neuropeptide signaling modulator (e.g., an agent that increases or decreases neuropeptide signaling), such as a blocker or agonist of a neuropeptide receptor listed in Table 1A. Neuromodulating agents that increase neuropeptide signaling include neuropeptides and neuropeptide receptors (e.g., a neuropeptide (ligand) listed in Table 1A, Table 1B, or Table 7, e.g., a neuropeptide having the sequence referenced by accession number or Entrez Gene ID of a neuropeptide listed in Table 1A, Table 1B, or Table 7, or an analog thereof, e.g., a sequence having at least 70%, 75%, 80%, 85%, 90%, 95%, 98%, 99% identity to the sequence referenced by accession number or Entrez Gene ID. The neuromodulating agent can be an endocannabinoid, amine, amino acid, purine, gas, gastrin, opioid, monoamine, secretin, tachykinin, neuropeptide, neurohypophyseal, orexin, or somatostatin, e.g., listed in Table 1B. In some embodiments, neuropeptide signaling is increased by administering, locally delivering, or stabilizing a neuropeptide listed in Tables 1A, 1B, or encoded by a gene in Table 7. Neuromodulating agents that increase neuropeptide signaling also include agents that increase neuropeptide receptor activity (e.g., neuromodulating agents that increase the activity of a neuropeptide receptor or signaling protein listed in Table 1A or encoded by a gene in Table 7 via upregulation, stabilization, agonism, or overexpression). Exemplary neuropeptide agonists are listed in Table 2A and 2L. Neuromodulating agents that increase neuropeptide signaling also include agents that reduce neuropeptide degradation or reuptake, agents that increase neuropeptide synthesis or release (e.g., agents that increase the activity of a biosynthetic protein encoded by a gene in Table 1A or Table 7 via stabilization, overexpression, or upregulation), increase neuropeptide receptor synthesis or membrane insertion, and regulate neuropeptide receptor conformation (e.g., agents that bind to a receptor and keep it in an “open” or “primed” conformation). Neuropeptide signaling modulators can increase neuropeptide signaling by 10%, 20%, 30%, 40%, 50%, 60%, 70%, 80%, 90%, 95%, 98% or more.

Neuromodulating agents that decrease neuropeptide signaling include agents that decrease neuropeptide receptor activity (e.g., neuromodulating agents that decrease the activity of a neuropeptide receptor or signaling protein listed in Table 1A or encoded by a gene in Table 7 via blockade, antagonism, or downregulation). Exemplary neuropeptide antagonists are listed in Table 2A or 2L. Neuromodulating agents that decrease neuropeptide signaling also include agents that bind to neuropeptides or block their interaction with receptors (e.g., neuropeptide blocking or neutralizing antibodies), agents that increase neuropeptide degradation or clearance, agents that decrease neuropeptide synthesis or release (e.g., agents that decrease the activity of a biosynthetic protein encoded by a gene in Table 1A or Table 7 via inhibition or downregulation), decrease neuropeptide receptor synthesis or membrane insertion, and regulate neuropeptide receptor conformation (e.g., agents that bind to a receptor and keep it in a “closed” or “inactive” conformation). In some embodiments, neuropeptide signaling is decreased by sequestering, blocking, antagonizing, or degrading a neuropeptide listed in Tables 1A, 1B, or encoded by a gene in Table 7. Neuropeptide signaling modulators can decrease neuropeptide signaling by 10%, 20%, 30%, 40%, 50%, 60%, 70%, 80%, 90%, 95%, 98% or more.

Neuropeptide signaling modulators also include antibodies that bind to neuropeptides or neuropeptide receptors listed in Tables 1A, 1B, and Table 7 and decrease neuropeptide signaling. These antibodies include blocking and neutralizing antibodies. Exemplary neuropeptide signaling blocking and neutralizing antibodies are listed below in Table 4. Antibodies to neuropeptides and neuropeptide receptors listed in Tables 1A, 1B, and Table 7 can also be generated by those of skill in the art using well established and routine methods.

TABLE 4
NEUROPEPTIDE AND NEUROPEPTIDE
RECEPTOR ANTIBODIES

| Neuropeptide or | | |
|---|---|---|
| Neuropeptide | | |
| Receptor | Antibody | Company |
| CGRP | LY2951742 | Eli Lilly |
| | (Galcanezumab) | |
| CGRP | ALD-403 | Alder Biopharmaceuticals |
| CGRP | TEV-48125 (LBR-101) | Teva Pharmaceuticals |
| CGRP | AMG 334 | Amgen |
| Glucagon | REGN1193 | Regeneron |

Neuronal Growth Factor Modulators

In some embodiments, a neuromodulating agent is a neuronal growth factor modulator (e.g., an agent that decreases or increases neurogenic/axonogenic signals, e.g., a neuronal growth factor or neuronal growth factor mimic, or an agonist or antagonist of a neuronal growth factor or neuronal growth factor receptor). For example, the neuromodulating agent is a neuronal growth factor listed in Table 10 or Table 7, e.g., a neuronal growth factor having the sequence referenced by accession number or Entrez Gene ID in Table 10 or Table 7, or an analog thereof, e.g., a sequence having at least 75%, 80%, 85%, 90%, 90%, 98%, 99% identity to the sequence referenced by accession number or Entrez Gene ID in Table 10 or Table 7. Neuronal growth factor modulators also include agonists and antagonists of neuronal growth factors and neuronal growth factor receptors listed in Table 10 or Table 7. A neuronal growth factor modulator may increase or decrease neurogenesis, neuronal growth, neuronal differentiation, neurite outgrowth, synapse formation, synaptic maturation, synaptic refinement, or synaptic stabilization. Neuronal growth factor modulators regulate innervation and the formation of synaptic connections between two or more neurons and between neurons and non-neural cells. A neuronal growth factor modulator may block one or more of these processes (e.g., through the use of antibodies that block neuronal growth factors or their receptors) or promote one or more of these processes (e.g., through the use of neuronal growth factors or analogs thereof). Neuronal growth factor modulators can increase or decrease one of the above mentioned processes by 10%, 20%, 30%, 40%, 50%, 60%, 70%, 80%, 90%, 95%, 98%, 200%, 500% or more.

In some embodiments, the neuromodulating agent decreases neurogenic/axonogenic signals, e.g., the method includes administering to the subject or contacting a cell with a neuromodulating agent (e.g., a neuronal growth factor modulator) in an amount and for a time sufficient to decrease neurogenesis or axonogenesis. For example, the neuromodulating agent that leads to a decrease in neurogenesis or axonogenesis is a blocking or neutralizing antibody against a neurotrophic factor. Relevant neurotrophic factors include NGF, BDNF, ProNGF, Sortilin, TGFβ and TGFβ family ligands and receptors (e.g., TGFβR1, TGFβR2, TGFβ1, TGFβ2 TGFβ4), GFRα family ligands and receptors (e.g., GFRα1, GFRα2, GFRα3, GFRα4, GDNF), CNTF, LIF, neurturin, artemin, persephin, neurotrophin, chemokines, cytokines, and others listed in Table 10 or Table 7. Receptors for these factors can also be targeted, as well as downstream signaling pathways including Jak-Stat inducers, and cell cycle and MAPK signaling pathways. In some embodiments, the neuronal growth factor modulator decreases neurogenesis, axonogenesis or any of the processes mentioned above by sequestering, blocking, antagonizing, degrading, or downregulating a neuronal growth factor or a neuronal growth factor receptor listed in Table 10 or encoded by a gene in Table 7. In some embodiments, the neuronal growth factor modulator decreases neurogenesis, axonogenesis or any of the processes mentioned above by blocking or antagonizing a signaling protein encoded by a gene in Table 7 that is downstream of a neuronal growth factor. In some embodiments, the neuronal growth factor modulator decreases neurogenesis, axonogenesis or any of the processes mentioned above by blocking, disrupting, or antagonizing a synaptic or structural protein encoded by a gene in Table 7. Neurogenesis, axonogenesis, neuronal growth, neuronal differentiation, neurite outgrowth, synapse formation, synaptic maturation, synaptic refinement, or synaptic stabilization can be decreased in the subject at least 1%, 2%, 5%, 10%, 15%, 20%, 25%, 30%, 35%, 40%, 50%, 60%, 70%, 80% or more, compared to before the administration. Neurogenesis, axonogenesis, neuronal growth, neuronal differentiation, neurite outgrowth, synapse formation, synaptic maturation, synaptic refinement, or synaptic stabilization can be decreased in the subject between 5-20%, between 5-50%, between 10-50%, between 20-80%, between 20-70%.

In some embodiments, the neuromodulating agent is one that increases neurogenic/axonogenic signals, e.g., the method includes administering to the subject or contacting a cell with a neuromodulating agent (e.g., a neuronal growth factor modulator) in an amount and for a time sufficient to increase neurogenesis or axonogenesis. For example, the neuromodulating agent that leads to an increase in neurogenesis or axonogenesis is a neurotrophic factor. Relevant neurotrophic factors include NGF, BDNF, ProNGF, Sortilin, TGFβ and TGFβ family ligands and receptors (e.g., TGFβR1, TGFβR2, TGFβ1, TGFβ2 TGFβ4), GFRα family ligands and receptors (e.g., GFRα1, GFRα2, GFRα3, GFRα4, GDNF), CNTF, LIF, neurturin, artemin, persephin, neurotrophin, chemokines, cytokines, and others listed in Table 1C or Table 7. Receptors for these factors may also be targeted, as well as downstream signaling pathways including Jak-Stat inducers, and cell cycle and MAPK signaling pathways. In some embodiments, the neuronal growth factor modulator increases neurogenesis, axonogenesis or any of the processes mentioned above by administering, locally delivering, or stabilizing a neuronal growth factor listed in Table 10 or encoded by a gene in Table 7, or by upregulating, agonizing, or stabilizing a neuronal growth factor receptor listed in Table 10 or encoded by a gene in Table 7. In some embodiments, the neuronal growth factor modulator increases neurogenesis, axonogenesis or any of the processes mentioned above by stabilizing, agonizing, overexpressing, or upregulating a signaling protein encoded by a gene in Table 7 that is downstream of a neuronal growth factor. In some embodiments, the neuronal growth factor modulator increases neurogenesis, axonogenesis or any of the processes mentioned above by stabilizing, overexpressing, or upregulating a synaptic or structural protein encoded by a gene in Table 7. Neurogenesis, axonogenesis, neuronal growth, neuronal differentiation, neurite outgrowth, synapse formation, synaptic maturation, synaptic refinement, or synaptic stabilization can be increased in the subject at least 1%, 2%, 5%, 10%, 15%, 20%, 25%, 30%, 35%, 40%, 50%, 60%, 70%, 80% or more, compared to before the administration. Neurogenesis, axonogenesis, neuronal growth, neuronal differentiation, neurite outgrowth, synapse formation, synaptic maturation, synaptic refinement, or synaptic stabilization can be increased in the subject between 5-20%, between 5-50%, between 10-50%, between 20-80%, between 20-70%.

In some embodiments, the neuromodulating agent that increases or decreases the number of nerves in an affected tissue. For example, the subject has cancer (e.g., the subject has a highly innervated tumor). For example, the neuromodulating agent is administered in an amount and for a time sufficient to decrease neurogenesis/axonogenesis. The neuromodulating agent can be, e.g., an inhibitor of neuronal growth factor signaling such as a blocking antibody directed to a neuronal growth factor or neuronal growth factor receptor.

Neuronal growth factor modulators also include antibodies that bind to neuronal growth factors or neuronal growth factor receptors and decrease their signaling (e.g., blocking antibodies). Exemplary neuronal growth factor blocking antibodies are listed below in Table 5. Antibodies to neuronal growth factors listed in Table 10 and Table 7 can also be generated by those of skill in the art using well established and routine methods.

TABLE 5
NEURONAL GROWTH FACTOR ANTIBODIES

| Neuronal | | |
|---|---|---|
| Growth | | |
| Factor | Antibody | Company |
| BDNF | 38B8 (agonist antibody) | Pfizer |
| BDNF | 29D7 (agonist antibody) | Pfizer |
| EphA3 | KB004 | KaloBios Pharmaceuticals, |
| | | Inc. |
| IFNA1 | Faralimomab | Creative Biolabs |
| IFNA1 | Sifalimumab (MEDI-545) | MedImmune |
| IFNA1 | Rontalizumab | Genentech |
| IGF | Figitumumab (CP-751,871) - | Pfizer |
| | an IGR-1R MAb | |
| IGF | SCH717454 (Robatumamab, | Merck |
| | inhibits IGF initiated | |
| | phosphorylation) | |
| IGF | Cixutumumab (IGF-1R | Eli Lilly |
| | antibody) | |
| IGF | Teprotumumab (IGF-1R | Genmab/Roche |
| | blocking antibody) | |
| IGF-2 | Dusigitumab | MedImmune/AstraZeneca |
| IGF-2 | DX-2647 | Dyax/Shire |
| IGF | Xentuzumab | Boehringer Ingelheim/Eli |
| | | Lilly |
| IGF | Dalotuzumab (IGFR1 | Merck & Co. |
| | blocking antibody) | |
| IGF | Figitumumab (IGFR1 | Pfizer |
| | blocking antibody) | |
| IGF | Ganitumab (IGFR1 | Amgen |
| | blocking antibody) | |
| IGF | Robatumumab (IGFR1 | Roche/Schering-Plough |
| | blocking antibody) | |
| IL1B | Canakinumab | Novartis |
| IL1B | APX002 | Apexigen |
| IL1B | Gevokizumab | XOMA |
| IL4 | Pascolizumab | GlaxoSmithKline |
| IL4 | Dupilumab | Regeneraon/Sanofi |
| IL6 | Siltuximab | Janssen Biotech, Inc. |
| IL6 | Olokizumab | UCB/R-Pharm |
| IL6 | Elsilimomab | Orphan Pharma |
| | | International |
| IL6 | Sirukumab | Centocor |
| IL6 | Clazakizumab | Bristol Myers Squib/Alder |
| | | Biopharmaceuticals |
| IL6 | Gerilimzumab (ARGX-109) | arGEN-X/RuiYi |
| IL6 | FE301 | Ferring Pharmaceuticals |
| IL6 | FM101 | Femta Pharmaceuticals |
| IL-6R | Sarilumab (directed against | Regeneron/Sanofi |
| | IL6R) | |
| IL-6R | Tocilizumab | Hoffmann-La Roche/Chugai |
| IL-6R | Sapelizumab | Chugai |
| IL-6R | Vobarilizumab | Ablynx |
| L1CAM | AB417 | Creative biolabs |
| L1CAM | L1-9.3 | Creative biolabs |
| L1CAM | L1-14.10 | Biolegend |
| NGF | Tanezumab | Pfizer |
| NGF | Fulranumab (JNJ-42160443), | Amgen |
| NGF | MNAC13 (anti-TrkA, the | Creative Biolabs |
| | NGF receptor) | |
| NGF | mAb 911 | Rinat/Pfizer |
| NGF | Fasinumab | Regeneron/Teva |
| NRG1 | 538.24 | Hoffman-La Roche |
| NRP1 | Vesencumab | Genentech/Roche |
| ROR1 | Cirmtuzumab | Oncternal Therapeutics |
| SAP | GSK2398852 | GlaxoSmithKline |
| TGFβ | Fresolimumab (pan-TGFβ | Genzyme/Aventis |
| | antibody) | |
| TGFβ | IMC-TR1 (LY3022859) | Eli Lilly |
| | (MAb against TGFβRII) | |
| TGFβ | TβM1 (anti-TGFβ1 MAb) | Eli Lilly |
| TGFβ2 | Lerdelimumab (CAT-152) | Genzyme |
| TGFβ1 | Metelimumab | Genzyme |
| TGFβ1 | LY2382770 | Eli Lilly |
| TGFβ | PF-03446962 (MAb against | Pfizer |
| | TGFβRI) | |
| TNF | Infliximab | Janssen Biotech, Inc. |
| TNF | Adalimumab | AbbVie Inc. |
| TNF | Certolizumab pegol | UCB |
| TNF | Golimumab | Janssen Biotech, Inc. |
| TNF | Afelimomab | |
| TNF | Placulumab | Teva Pharmaceutical |
| | | Industries, Inc. |
| TNF | Nerelimomab | Chiron/Celltech |
| TNF | Ozoralizumab | Pfizer/Ablynx |
| VEGFA | Bevacizumab | Genzyme |
| VEGFA | Ranibizumab | Lucentis |
| VEGF | Alacizumab pegol (anti- | UCB |
| | VEGFR2) | |
| VEGFA | Brolucizumab | Novartis |
| VEGF | Icrucumab (anti-VEGFR1) | Eli Lilly |
| VEGF | Ramucirumab (anti-VEGFR2) | Eli Lilly |

Neuronal growth factor modulators also include agents that agonize or antagonize neuronal growth factors and neuronal growth factor receptors. For example, neuronal growth factor modulators include TNF inhibitors (e.g., etanercept, thalidomide, lenalidomide, pomalidomide, pentoxifylline, bupropion, and DOI), TGFβ1 inhibitors, (e.g., disitertide (P144)), TGFβ2 inhibitors (e.g., trabedersen (AP12009)). Exemplary neuronal growth factor agonists and antagonists are listed in Table 6.

TABLE 6
NEURONAL GROWTH FACTOR AGONISTS AND ANTAGONISTS

| | Agonist | Antagonist |
|---|---|---|
| TrkA | NGF, amitriptyline, and gambogic | Ale-0540 |
| | amide, gambogic acid | |
| TrkB | BDNF, NT3, NT4, 3,7- | ANA-12, cyclotraxin B, and |
| | Dihydroxyflavone, 3,7,8,2′- | gossypetin |
| | Tetrahydroxyflavone, 4′- | |
| | Dimethylamino-7,8- | |
| | dihydroxyflavone, 7,3′- | |
| | Dihydroxyflavone, 7,8- | |
| | Dihydroxyflavone, 7,8,2′- | |
| | Trihydroxyflavone, 7,8,3′- | |
| | Trihydroxyflavone, Amitriptyline^{,} | |
| | Deoxygedunin, Diosmetin, HIOC, | |
| | LM22A-4, N-Acetylserotonin, | |
| | Norwogonin (5,7,8-THF), R7, | |
| | LM22A4, and TDP6 | |
| Pan-Trk receptor | | Entrectinib (RXDX-101), AG 879, |
| | | GNF 5837, GW 441756, and PF |
| | | 06273340 |
| GFRα1R | GDNF and XIB4035 | |
| VEGF receptor | | AEE 788, AG 879, AP 24534, |
| | | axitinib, DMH4, GSK 1363089, Ki |
| | | 8751, RAF 265, SU 4312, SU |
| | | 5402, SU 5416, SU 6668, |
| | | sunitinib, toceranib, vatalanib, XL |
| | | 184, ZM 306416, and ZM 323881 |
| TGFβRI | | Galunisertib (LY2157299), TEW- |
| | | 7197, SB-431542, A 83-01, D |
| | | 4476, GW 788388, LY 364947, R |
| | | 268712, repsox, SB 505124, SB |
| | | 525334, and SD 208 |

Modulators of Gene Expression

In some embodiments, a neuromodulating agent is a neurome gene expression modulator (e.g., an agent that affects the expression of a neurome gene listed in Table 7 or Table 8, e.g., a channel, transporter, neuropeptide, neurotransmitter, neurotrophic, signaling, synaptic, biosynthesis, ligand, receptor, structural, or vesicular gene). A neurome gene expression modulator can affect gene expression through modulation of gene transcription, gene translation, or protein levels. Neurome gene expression modulators may increase gene expression through epigenetic modifications (e.g., demethylation or acetylation), post-translational modifications (e.g., reducing ubiquitination, or altering sumoylation or phosphorylation), by increasing mRNA translation and stability, or through delivery of exogenous genetic material (e.g., a viral vector expressing a gene of interest). In some embodiments, the neurome gene expression modulator increases neurome gene expression by stabilizing, upregulating, or promoting overexpression of a biosynthesis, channel, ligand, receptor, signaling, structural, synaptic, transporter, vesicular, neuropeptide, neurotransmitter, or neurotrophic gene in Table 7 or a channel or transporter gene in Table 8. Neurome gene expression modulators may decrease gene expression through epigenetic modifications (e.g., methylation or deacetylation), post-translational modifications (e.g., increasing ubiquitination, or altering sumoylation or phosphorylation), or by decreasing mRNA translation and stability (e.g., using miRNA, siRNA, shRNA, or other therapeutic RNAs). In some embodiments, the neurome gene expression modulator decreases neurome gene expression by downregulating, inhibiting, or disrupting expression of a biosynthesis, channel, ligand, receptor, signaling, structural, synaptic, transporter, vesicular, neuropeptide, neurotransmitter, or neurotrophic gene in Table 7 or a channel or transporter gene in Table 8. A neurome gene expression modulator may increase or decrease gene expression by 10%, 20%, 30%, 40%, 50%, 60%, 70%, 80%, 90%, 95%, 98%, or more.

In some embodiments, a neurome gene expression modulator increases or decreases the expression of a neurome gene listed in Table 13 or Table 7 to treat cancer (e.g., through altering the activity of the immune cell expressing the modulated gene). The neurome gene expression modulator can be introduced systemically (e.g., injected intravenously into the blood stream), or administered locally (e.g., administered to or near a lymph node, secondary lymphoid organ, or tumor). The neurome gene expression modulator can also be used to contact an immune cell in vitro before administering the cell to a subject (e.g., a human subject or animal model).

TABLE 7
NEUROME GENES

| Approved | | Entrez | Gene type/ | |
|---|---|---|---|---|
| Symbol | Approved name | Gene ID | family | Category |
| ABAT | 4-aminobutyrate | 18 | Neurotransmitter | Biosynthesis |
| | aminotransferase | | | |
| ACHE | Acetylcholinesterase | 43 | Neurotransmitter | Biosynthesis |
| ACTR1A | ARP1 actin-related protein 1 | 10121 | Synaptic | Structural |
| | homolog A, centractin alpha | | | |
| ACTR1B | ARP1 actin-related protein 1 | 10120 | Synaptic | Structural |
| | homolog B, centractin beta | | | |
| ADCY1 | Adenylate cyclase 1 | 107 | Signaling | Signaling |
| ADCY10 | Adenylate cyclase 10, soluble | 55811 | Signaling | Signaling |
| ADCY2 | Adenylate cyclase 2 | 108 | Signaling | Signaling |
| ADCY3 | Adenylate cyclase 3 | 109 | Signaling | Signaling |
| ADCY4 | Adenylate cyclase 4 | 196883 | Signaling | Signaling |
| ADCY5 | Adenylate cyclase 5 | 111 | Signaling | Signaling |
| ADCY6 | Adenylate cyclase 6 | 112 | Signaling | Signaling |
| ADCY7 | Adenylate cyclase 7 | 113 | Signaling | Signaling |
| ADCY8 | Adenylate cyclase 8 | 114 | Signaling | Signaling |
| ADCY9 | Adenylate cyclase 9 | 115 | Signaling | Signaling |
| ADCYAP1 | Adenylate cyclase activating | 116 | Signaling | Signaling |
| | polypeptide 1 | | | |
| ADCYAP1R1 | ADCYAP receptor type I | 117 | Neurotransmitter | Receptor |
| ADIPOQ | Adiponectin, C1Q and | 9370 | Neuropeptide | Ligand |
| | collagen domain containing | | | |
| ADK | Adenosine kinase | 132 | Signaling | Signaling |
| ADM | Adrenomedullin | 133 | Neuropeptide | Ligand |
| ADM2 | Adrenomedullin 2 | 79924 | Neuropeptide | Ligand |
| ADNP | Activity dependent | 23394 | Signaling | Structural |
| | neuroprotector homeobox | | | |
| ADORA2A | Adenosine A2a receptor | 135 | Neurotransmitter | Receptor |
| ADORA2B | Adenosine A2b receptor | 136 | Neurotransmitter | Receptor |
| ADRA1A | Adrenoceptor alpha 1A | 148 | Neurotransmitter | Receptor |
| ADRA1B | Adrenoceptor alpha 1B | 147 | Neurotransmitter | Receptor |
| ADRA1D | Adrenoceptor alpha 1D | 146 | Neurotransmitter | Receptor |
| ADRA2A | Adrenoceptor alpha 2A | 150 | Neurotransmitter | Receptor |
| ADRA2B | Adrenoceptor alpha 2B | 151 | Neurotransmitter | Receptor |
| ADRA2C | Adrenoceptor alpha 2C | 152 | Neurotransmitter | Receptor |
| ADRB1 | Adrenoceptor beta 1 | 153 | Neurotransmitter | Receptor |
| ADRB2 | Adrenoceptor beta 2 | 154 | Neurotransmitter | Receptor |
| ADRB3 | Adrenoceptor beta 3 | 155 | Neurotransmitter | Receptor |
| AGRN | Agrin | 375790 | Neuropeptide | Ligand |
| AGRP | Agouti related neuropeptide | 181 | Neuropeptide | Ligand |
| AGT | Angiotensinogen | 183 | Neuropeptide | Ligand |
| AGTR1 | Angiotensin II receptor type 1 | 185 | Neuropeptide | Receptor |
| AKAP1 | A-kinase anchoring protein 1 | 8165 | Signaling | Signaling |
| AKAP10 | A-kinase anchoring protein 10 | 11216 | Signaling | Signaling |
| AKAP11 | A-kinase anchoring protein 11 | 11215 | Signaling | Signaling |
| AKAP12 | A-kinase anchoring protein 12 | 9590 | Signaling | Signaling |
| AKAP13 | A-kinase anchoring protein 13 | 11214 | Signaling | Signaling |
| AKAP14 | A-kinase anchoring protein 14 | 158798 | Signaling | Signaling |
| AKAP17A | A-kinase anchoring protein | 8227 | Signaling | Signaling |
| | 17A | | | |
| AKAP2 | A-kinase anchoring protein 2 | 11217 | Signaling | Signaling |
| AKAP3 | A-kinase anchoring protein 3 | 10566 | Signaling | Signaling |
| AKAP4 | A-kinase anchoring protein 4 | 8852 | Signaling | Signaling |
| AKAP5 | A-kinase anchoring protein 5 | 9495 | Signaling | Signaling |
| AKAP6 | A-kinase anchoring protein 6 | 9472 | Signaling | Signaling |
| AKAP7 | A-kinase anchoring protein 7 | 9465 | Signaling | Signaling |
| AKAP8 | A-kinase anchoring protein 8 | 10270 | Signaling | Signaling |
| AKAP9 | A-kinase anchoring protein 9 | 10142 | Signaling | Signaling |
| AKT1 | AKT serine/threonine kinase 1 | 207 | Signaling | Signaling |
| AKT2 | AKT serine/threonine kinase 2 | 208 | Signaling | Signaling |
| AKT3 | AKT serine/threonine kinase 3 | 10000 | Signaling | Signaling |
| ALDH5A1 | Aldehyde dehydrogenase 5 | 7915 | Other | Miscelaneous |
| | family member A1 | | | |
| ALDH9A1 | Aldehyde dehydrogenase 9 | 223 | Other | Miscelaneous |
| | family member A1 | | | |
| ALOX15 | Arachidonate 15-lipoxygenase | 246 | Other | Miscelaneous |
| ALPL | Alkaline phosphatase, | 249 | Other | Miscelaneous |
| | liver/bone/kidney | | | |
| ANXA1 | Annexin A1 | 301 | Other | Miscelaneous |
| AP3B2 | Adaptor related protein | 8120 | Vesicular | Vesicles |
| | complex 3 beta 2 subunit | | | |
| AP3D1 | Adaptor related protein | 8943 | Vesicular | Vesicles |
| | complex 3 delta 1 subunit | | | |
| APLN | Apelin | 8862 | Neuropeptide | Ligand |
| APOE | Apolipoprotein E | 348 | Other | Miscelaneous |
| ARID1B | AT-rich interaction domain 1B | 57492 | Other | Miscelaneous |
| ARPP19 | Camp regulated | 10776 | Signaling | Signaling |
| | phosphoprotein 19 | | | |
| ARR3 | Arrestin 3 | 407 | Signaling | Signaling |
| ARRB1 | Arrestin beta 1 | 408 | Signaling | Signaling |
| ARRB2 | Arrestin beta 2 | 409 | Signaling | Signaling |
| ARTN | Artemin | 9048 | Neurotrophic | Ligand |
| ASIC1 | Acid sensing ion channel | 41 | Channel or | Channel |
| | subunit 1 | | transporter | |
| ASIC2 | Acid sensing ion channel | 40 | Channel or | Channel |
| | subunit 2 | | transporter | |
| ASIC3 | Acid sensing ion channel | 9311 | Channel or | Channel |
| | subunit 3 | | transporter | |
| ASIP | Agouti signaling protein | 434 | Neuropeptide | Ligand |
| ATP10A | Atpase phospholipid | 57194 | Channel or | Transporter |
| | transporting 10A (putative) | | transporter | |
| ATP10B | Atpase phospholipid | 23120 | Channel or | Transporter |
| | transporting 10B (putative) | | transporter | |
| ATP10D | Atpase phospholipid | 57205 | Channel or | Transporter |
| | transporting 10D (putative) | | transporter | |
| ATP11A | Atpase phospholipid | 23250 | Channel or | Transporter |
| | transporting 11A | | transporter | |
| ATP11B | Atpase phospholipid | 23200 | Channel or | Transporter |
| | transporting 11B (putative) | | transporter | |
| ATP11C | Atpase phospholipid | 286410 | Channel or | Transporter |
| | transporting 11C | | transporter | |
| ATP12A | Atpase H+/K+ transporting | 479 | Channel or | Transporter |
| | non-gastric alpha2 subunit | | transporter | |
| ATP1A1 | Atpase Na+/K+ transporting | 476 | Channel or | Transporter |
| | subunit alpha 1 | | transporter | |
| ATP1A2 | Atpase Na+/K+ transporting | 477 | Channel or | Transporter |
| | subunit alpha 2 | | transporter | |
| ATP1A3 | Atpase Na+/K+ transporting | 478 | Channel or | Transporter |
| | subunit alpha 3 | | transporter | |
| ATP1A4 | Atpase Na+/K+ transporting | 480 | Channel or | Transporter |
| | subunit alpha 4 | | transporter | |
| ATP1B1 | Atpase Na+/K+ transporting | 481 | Channel or | Transporter |
| | subunit beta 1 | | transporter | |
| ATP1B2 | Atpase Na+/K+ transporting | 482 | Channel or | Transporter |
| | subunit beta 2 | | transporter | |
| ATP1B3 | Atpase Na+/K+ transporting | 483 | Channel or | Transporter |
| | subunit beta 3 | | transporter | |
| ATP2A1 | Atpase | 487 | Channel or | Transporter |
| | sarcoplasmic/endoplasmic | | transporter | |
| | reticulum Ca2+ transporting 1 | | | |
| ATP2A2 | Atpase | 488 | Channel or | Transporter |
| | sarcoplasmic/endoplasmic | | transporter | |
| | reticulum Ca2+ transporting 2 | | | |
| ATP2A3 | Atpase | 489 | Channel or | Transporter |
| | sarcoplasmic/endoplasmic | | transporter | |
| | reticulum Ca2+ transporting 3 | | | |
| ATP2B1 | Atpase plasma membrane | 490 | Channel or | Transporter |
| | Ca2+ transporting 1 | | transporter | |
| ATP2B2 | Atpase plasma membrane | 491 | Channel or | Transporter |
| | Ca2+ transporting 2 | | transporter | |
| ATP2B3 | Atpase plasma membrane | 492 | Channel or | Transporter |
| | Ca2+ transporting 3 | | transporter | |
| ATP2B4 | Atpase plasma membrane | 493 | Channel or | Transporter |
| | Ca2+ transporting 4 | | transporter | |
| ATP2C1 | Atpase secretory pathway | 27032 | Channel or | Transporter |
| | Ca2+ transporting 1 | | transporter | |
| ATP2C2 | Atpase secretory pathway | 9914 | Channel or | Transporter |
| | Ca2+ transporting 2 | | transporter | |
| ATP4A | Atpase H+/K+ transporting | 495 | Channel or | Transporter |
| | alpha subunit | | transporter | |
| ATP4B | Atpase H+/K+ transporting | 496 | Channel or | Transporter |
| | beta subunit | | transporter | |
| ATP5A1 | ATP synthase, H+ | 498 | Channel or | Transporter |
| | transporting, mitochondrial F1 | | transporter | |
| | complex, alpha subunit 1, | | | |
| | cardiac muscle | | | |
| ATP5B | ATP synthase, H+ | 506 | Channel or | Transporter |
| | transporting, mitochondrial F1 | | transporter | |
| | complex, beta polypeptide | | | |
| ATP5C1 | ATP synthase, H+ | 509 | Channel or | Transporter |
| | transporting, mitochondrial F1 | | transporter | |
| | complex, gamma polypeptide | | | |
| | 1 | | | |
| ATP5D | ATP synthase, H+ | 513 | Channel or | Transporter |
| | transporting, mitochondrial F1 | | transporter | |
| | complex, delta subunit | | | |
| ATP5E | ATP synthase, H+ | 514 | Channel or | Transporter |
| | transporting, mitochondrial F1 | | transporter | |
| | complex, epsilon subunit | | | |
| ATP5F1 | ATP synthase, H+ | 515 | Channel or | Transporter |
| | transporting, mitochondrial Fo | | transporter | |
| | complex subunit B1 | | | |
| ATP5H | ATP synthase, H+ | 10476 | Channel or | Transporter |
| | transporting, mitochondrial Fo | | transporter | |
| | complex subunit D | | | |
| ATP5I | ATP synthase, H+ | 521 | Channel or | Transporter |
| | transporting, mitochondrial Fo | | transporter | |
| | complex subunit E | | | |
| ATP5J | ATP synthase, H+ | 522 | Channel or | Transporter |
| | transporting, mitochondrial Fo | | transporter | |
| | complex subunit F6 | | | |
| ATP5J2 | ATP synthase, H+ | 9551 | Channel or | Transporter |
| | transporting, mitochondrial Fo | | transporter | |
| | complex subunit F2 | | | |
| ATP5L2 | ATP synthase, H+ | 267020 | Channel or | Transporter |
| | transporting, mitochondrial Fo | | transporter | |
| | complex subunit G2 | | | |
| ATP6V0A1 | Atpase H+ transporting V0 | 535 | Channel or | Transporter |
| | subunit a1 | | transporter | |
| ATP6V0A2 | Atpase H+ transporting V0 | 23545 | Channel or | Transporter |
| | subunit a2 | | transporter | |
| ATP6V0A4 | Atpase H+ transporting V0 | 50617 | Channel or | Transporter |
| | subunit a4 | | transporter | |
| ATP6V0B | Atpase H+ transporting V0 | 533 | Channel or | Transporter |
| | subunit b | | transporter | |
| ATP6V0C | Atpase H+ transporting V0 | 527 | Channel or | Transporter |
| | subunit c | | transporter | |
| ATP6V0D1 | Atpase H+ transporting V0 | 9114 | Channel or | Transporter |
| | subunit d1 | | transporter | |
| ATP6V0D2 | Atpase H+ transporting V0 | 245972 | Channel or | Transporter |
| | subunit d2 | | transporter | |
| ATP6V0E1 | Atpase H+ transporting V0 | 8992 | Channel or | Transporter |
| | subunit e1 | | transporter | |
| ATP6V0E2 | Atpase H+ transporting V0 | 155066 | Channel or | Transporter |
| | subunit e2 | | transporter | |
| ATP6V1A | Atpase H+ transporting V1 | 523 | Channel or | Transporter |
| | subunit A | | transporter | |
| ATP6V1B1 | Atpase H+ transporting V1 | 525 | Channel or | Transporter |
| | subunit B1 | | transporter | |
| ATP6V1B2 | Atpase H+ transporting V1 | 526 | Channel or | Transporter |
| | subunit B2 | | transporter | |
| ATP6V1C1 | Atpase H+ transporting V1 | 528 | Channel or | Transporter |
| | subunit C1 | | transporter | |
| ATP6V1C2 | Atpase H+ transporting V1 | 245973 | Channel or | Transporter |
| | subunit C2 | | transporter | |
| ATP6V1D | Atpase H+ transporting V1 | 51382 | Channel or | Transporter |
| | subunit D | | transporter | |
| ATP6V1E1 | Atpase H+ transporting V1 | 529 | Channel or | Transporter |
| | subunit E1 | | transporter | |
| ATP6V1E2 | Atpase H+ transporting V1 | 90423 | Channel or | Transporter |
| | subunit E2 | | transporter | |
| ATP6V1F | Atpase H+ transporting V1 | 9296 | Channel or | Transporter |
| | subunit F | | transporter | |
| ATP6V1G1 | Atpase H+ transporting V1 | 9550 | Channel or | Transporter |
| | subunit G1 | | transporter | |
| ATP6V1G2 | Atpase H+ transporting V1 | 534 | Channel or | Transporter |
| | subunit G2 | | transporter | |
| ATP6V1G3 | Atpase H+ transporting V1 | 127124 | Channel or | Transporter |
| | subunit G3 | | transporter | |
| ATP6V1H | Atpase H+ transporting V1 | 51606 | Channel or | Transporter |
| | subunit H | | transporter | |
| ATP7A | Atpase copper transporting | 538 | Channel or | Transporter |
| | alpha | | transporter | |
| ATP7B | Atpase copper transporting | 540 | Channel or | Transporter |
| | beta | | transporter | |
| ATP8A1 | Atpase phospholipid | 10396 | Channel or | Transporter |
| | transporting 8A1 | | transporter | |
| ATP8A2 | Atpase phospholipid | 51761 | Channel or | Transporter |
| | transporting 8A2 | | transporter | |
| ATP8B1 | Atpase phospholipid | 5205 | Channel or | Transporter |
| | transporting 8B1 | | transporter | |
| ATP8B2 | Atpase phospholipid | 57198 | Channel or | Transporter |
| | transporting 8B2 | | transporter | |
| ATP8B3 | Atpase phospholipid | 148229 | Channel or | Transporter |
| | transporting 8B3 | | transporter | |
| ATP8B4 | Atpase phospholipid | 79895 | Channel or | Transporter |
| | transporting 8B4 (putative) | | transporter | |
| ATP9A | Atpase phospholipid | 10079 | Channel or | Transporter |
| | transporting 9A (putative) | | transporter | |
| ATP9B | Atpase phospholipid | 374868 | Channel or | Transporter |
| | transporting 9B (putative) | | transporter | |
| AVP | Arginine vasopressin | 551 | Neuropeptide | Ligand |
| AVPR1A | Arginine vasopressin receptor | 552 | Neuropeptide | Receptor |
| | 1A | | | |
| AVPR1B | Arginine vasopressin receptor | 553 | Neuropeptide | Receptor |
| | 1B | | | |
| AVPR2 | Arginine vasopressin receptor | 554 | Neuropeptide | Receptor |
| | 2 | | | |
| BACE1 | Beta-secretase 1 | 23621 | Neurotransmitter | Biosynthesis |
| BAG2 | BCL2 associated athanogene | 9532 | Signaling | Signaling |
| | 2 | | | |
| BAIAP3 | BAI1 associated protein 3 | 8938 | Signaling | Signaling |
| BCHE | Butyrylcholinesterase | 590 | Neurotransmitter | Biosynthesis |
| BCL2 | BCL2, apoptosis regulator | 596 | Signaling | Signaling |
| BDKRB2 | Bradykinin receptor B2 | 624 | Neuropeptide | Receptor |
| BDNF | Brain derived neurotrophic | 627 | Neurotrophic | Ligand |
| | factor | | | |
| BDNF-AS | BDNF antisense RNA | 497258 | Neurotrophic | Ligand |
| BEX1 | Protein BEX1 (Brain- | 55859 | Neurotrophic | Signaling |
| | expressed X-linked protein 1) | | | |
| BEX3 | Protein BEX3 (Brain- | 27018 | Neurotrophic | Signaling |
| | expressed X-linked protein 3) | | | |
| BRAF | B-Raf proto-oncogene, | 673 | Signaling | Signaling |
| | serine/threonine kinase | | | |
| BRSK1 | BR serine/threonine kinase 1 | 84446 | Vesicular | Vesicles |
| BSN | Bassoon presynaptic | 8927 | Other | Miscelaneous |
| | cytomatrix protein | | | |
| BTBD9 | BTB domain containing 9 | 114781 | Vesicular | Vesicles |
| C1QBP | Complement C1q binding | 708 | Neuropeptide | Receptor |
| | protein | | | |
| C2CD4A | C2 calcium dependent domain | 145741 | Other | Miscelaneous |
| | containing 4A | | | |
| C2CD4B | C2 calcium dependent domain | 388125 | Other | Miscelaneous |
| | containing 4B | | | |
| C2CD4C | C2 calcium dependent domain | 126567 | Other | Miscelaneous |
| | containing 4C | | | |
| C2CD4D | C2 calcium dependent domain | 1001910 | Other | Miscelaneous |
| | containing 4D | 40 | | |
| C4orf48 | Chromosome 4 open reading | 401115 | Neuropeptide | Ligand |
| | frame 48 | | | |
| CACNA1A | Calcium voltage-gated | 773 | Channel or | Channel |
| | channel subunit alpha1 A | | transporter | |
| CACNA1B | Calcium voltage-gated | 774 | Channel or | Channel |
| | channel subunit alpha1 B | | transporter | |
| CACNA1C | Calcium voltage-gated | 775 | Channel or | Channel |
| | channel subunit alpha1 C | | transporter | |
| CACNA1D | Calcium voltage-gated | 776 | Channel or | Channel |
| | channel subunit alpha1 D | | transporter | |
| CACNA1E | Calcium voltage-gated | 777 | Channel or | Channel |
| | channel subunit alpha1 E | | transporter | |
| CACNA1F | Calcium voltage-gated | 778 | Channel or | Channel |
| | channel subunit alpha1 F | | transporter | |
| CACNA1G | Calcium voltage-gated | 8913 | Channel or | Channel |
| | channel subunit alpha1 G | | transporter | |
| CACNA1H | Calcium voltage-gated | 8912 | Channel or | Channel |
| | channel subunit alpha1 H | | transporter | |
| CACNA1I | Calcium voltage-gated | 8911 | Channel or | Channel |
| | channel subunit alpha1 I | | transporter | |
| CACNA1S | Calcium voltage-gated | 779 | Channel or | Channel |
| | channel subunit alpha1 S | | transporter | |
| CACNB1 | Calcium voltage-gated | 782 | Channel or | Channel |
| | channel auxiliary subunit beta | | transporter | |
| | 1 | | | |
| CACNB2 | Calcium voltage-gated | 783 | Channel or | Channel |
| | channel auxiliary subunit beta | | transporter | |
| | 2 | | | |
| CACNB4 | Calcium voltage-gated | 785 | Channel or | Channel |
| | channel auxiliary subunit beta | | transporter | |
| | 4 | | | |
| CALCA | Calcitonin related polypeptide | 796 | Neuropeptide | Ligand |
| | alpha, CGRP | | | |
| CALCB | Calcitonin related polypeptide | 797 | Neuropeptide | Ligand |
| | beta | | | |
| CALCR | Calcitonin receptor | 799 | Neuropeptide | Receptor |
| CALCRL | Calcitonin receptor like | 10203 | Neuropeptide | Receptor |
| | receptor | | | |
| CALM1 | Calmodulin 1 | 801 | Signaling | Signaling |
| CALM2 | Calmodulin 2 | 805 | Signaling | Signaling |
| CALM3 | Calmodulin 3 | 808 | Signaling | Signaling |
| CALY | Calcyon neuron specific | 50632 | Other | Miscelaneous |
| | vesicular protein | | | |
| CAMK2A | Calcium/calmodulin | 815 | Signaling | Signaling |
| | dependent protein kinase II | | | |
| | alpha | | | |
| CAMK2B | Calcium/calmodulin | 816 | Signaling | Signaling |
| | dependent protein kinase II | | | |
| | beta | | | |
| CAMK4 | Calcium/calmodulin | 814 | Signaling | Signaling |
| | dependent protein kinase IV | | | |
| CARF | Calcium responsive | 79800 | Signaling | Signaling |
| | transcription factor | | | |
| CARTPT | CART prepropeptide | 9607 | Neuropeptide | Ligand |
| CASK | Calcium/calmodulin | 8573 | Signaling | Signaling |
| | dependent serine protein | | | |
| | kinase | | | |
| CASR | Calcium sensing receptor | 846 | Neuropeptide | Biosynthesis |
| CATSPER1 | Cation channel sperm | 117144 | Channel or | Channel |
| | associated 1 | | transporter | |
| CATSPER2 | Cation channel sperm | 117155 | Channel or | Channel |
| | associated 2 | | transporter | |
| CATSPER3 | Cation channel sperm | 347732 | Channel or | Channel |
| | associated 3 | | transporter | |
| CATSPER4 | Cation channel sperm | 378807 | Channel or | Channel |
| | associated 4 | | transporter | |
| CBLN1 | Cerebellin 1 precursor | 869 | Other | Miscelaneous |
| CBLN2 | Cerebellin 2 precursor | 147381 | Other | Miscelaneous |
| CBLN3 | Cerebellin 3 precursor | 643866 | Other | Miscelaneous |
| CBLN4 | Cerebellin 4 precursor | 140689 | Other | Miscelaneous |
| CCK | Cholecystokinin | 885 | Neuropeptide | Ligand |
| CCKAR | Cholecystokinin A receptor | 886 | Neuropeptide | Receptor |
| CCKBR | Cholecystokinin B receptor | 887 | Neuropeptide | Receptor |
| CCL2 | C-C motif chemokine ligand 2 | 6347 | Neuropeptide | Ligand |
| CCR1 | C-C motif chemokine receptor | 1230 | Other | Miscelaneous |
| | 1 | | | |
| CD34 | Hematopoietic progenitor cell | 947 | Neurotrophic | Receptor |
| | antigen CD34 (CD antigen | | | |
| | CD34) | | | |
| CD38 | CD38 molecule | 952 | Signaling | Signaling |
| CDH2 | Cadherin 2 | 1000 | Signaling | Signaling |
| CDK5 | Cyclin dependent kinase 5 | 1020 | Signaling | Signaling |
| CDK5R1 | Cyclin dependent kinase 5 | 8851 | Signaling | Signaling |
| | regulatory subunit 1 | | | |
| CDKN1A | Cyclin dependent kinase | 1026 | Signaling | Signaling |
| | inhibitor 1A | | | |
| CDNF | Cerebral dopamine | 441549 | Neurotrophic | Ligand |
| | neurotrophic factor | | | |
| CHAT | Choline O-acetyltransferase | 1103 | Neurotransmitter | Biosynthesis |
| CHGA | Chromogranin A | 1113 | Neuropeptide | Ligand |
| CHGB | Chromogranin B | 1114 | Neuropeptide | Ligand |
| CHMP4A | Charged multivesicular body | 29082 | Vesicular | Vesicles |
| | protein 4A | | | |
| CHMP4B | Charged multivesicular body | 128866 | Vesicular | Vesicles |
| | protein 4B | | | |
| CHRFAM7A | CHRNA7 (exons 5-10) and | 89832 | Neurotransmitter | Receptor |
| | FAM7A (exons A-E) fusion | | | |
| CHRM1 | Cholinergic receptor | 1128 | Neurotransmitter | Receptor |
| | muscarinic 1 | | | |
| CHRM2 | Cholinergic receptor | 1129 | Neurotransmitter | Receptor |
| | muscarinic 2 | | | |
| CHRM3 | Cholinergic receptor | 1131 | Neurotransmitter | Receptor |
| | muscarinic 3 | | | |
| CHRM4 | Cholinergic receptor | 1132 | Neurotransmitter | Receptor |
| | muscarinic 4 | | | |
| CHRM5 | Cholinergic receptor | 1133 | Neurotransmitter | Receptor |
| | muscarinic 5 | | | |
| CHRNA1 | Cholinergic receptor nicotinic | 1134 | Neurotransmitter | Receptor |
| | alpha 1 subunit | | | |
| CHRNA10 | Cholinergic receptor nicotinic | 57053 | Neurotransmitter | Receptor |
| | alpha 10 subunit | | | |
| CHRNA2 | Cholinergic receptor nicotinic | 1135 | Neurotransmitter | Receptor |
| | alpha 2 subunit | | | |
| CHRNA3 | Cholinergic receptor nicotinic | 1136 | Neurotransmitter | Receptor |
| | alpha 3 subunit | | | |
| CHRNA4 | Cholinergic receptor nicotinic | 1137 | Neurotransmitter | Receptor |
| | alpha 4 subunit | | | |
| CHRNA5 | Cholinergic receptor nicotinic | 1138 | Neurotransmitter | Receptor |
| | alpha 5 subunit | | | |
| CHRNA6 | Cholinergic receptor nicotinic | 8973 | Neurotransmitter | Receptor |
| | alpha 6 subunit | | | |
| CHRNA7 | Cholinergic receptor nicotinic | 1139 | Neurotransmitter | Receptor |
| | alpha 7 subunit | | | |
| CHRNA9 | Cholinergic receptor nicotinic | 55584 | Neurotransmitter | Receptor |
| | alpha 9 subunit | | | |
| CHRNB1 | Cholinergic receptor nicotinic | 1140 | Neurotransmitter | Receptor |
| | beta 1 subunit | | | |
| CHRNB2 | Cholinergic receptor nicotinic | 1141 | Neurotransmitter | Receptor |
| | beta 2 subunit | | | |
| CHRNB3 | Cholinergic receptor nicotinic | 1142 | Neurotransmitter | Receptor |
| | beta 3 subunit | | | |
| CHRNB4 | Cholinergic receptor nicotinic | 1143 | Neurotransmitter | Receptor |
| | beta 4 subunit | | | |
| CHRND | Cholinergic receptor nicotinic | 1144 | Neurotransmitter | Receptor |
| | delta subunit | | | |
| CHRNE | Cholinergic receptor nicotinic | 1145 | Neurotransmitter | Receptor |
| | epsilon subunit | | | |
| CHRNG | Cholinergic receptor nicotinic | 1146 | Neurotransmitter | Receptor |
| | gamma subunit | | | |
| CLCF1 | Cardiotrophin-like cytokine | 23529 | Neuropeptide | Ligand |
| | factor 1 | | | |
| CLCN1 | Chloride voltage-gated | 1180 | Channel or | Channel |
| | channel 1 | | transporter | |
| CLCN2 | Chloride voltage-gated | 1181 | Channel or | Channel |
| | channel 2 | | transporter | |
| CLCN3 | Chloride voltage-gated | 1182 | Channel or | Channel |
| | channel 3 | | transporter | |
| CLCN4 | Chloride voltage-gated | 1183 | Channel or | Channel |
| | channel 4 | | transporter | |
| CLCN5 | Chloride voltage-gated | 1184 | Channel or | Channel |
| | channel 5 | | transporter | |
| CLCN6 | Chloride voltage-gated | 1185 | Channel or | Channel |
| | channel 6 | | transporter | |
| CLCN7 | Chloride voltage-gated | 1186 | Channel or | Channel |
| | channel 7 | | transporter | |
| CLCNKA | Chloride voltage-gated | 1187 | Channel or | Channel |
| | channel Ka | | transporter | |
| CLCNKB | Chloride voltage-gated | 1188 | Channel or | Channel |
| | channel Kb | | transporter | |
| CLIC6 | Chloride intracellular channel | 54102 | Channel or | Channel |
| | 6 | | transporter | |
| CLN3 | CLN3, battenin | 1201 | Other | Miscelaneous |
| CNGA1 | Cyclic nucleotide gated | 1259 | Channel or | Channel |
| | channel alpha 1 | | transporter | |
| CNGA2 | Cyclic nucleotide gated | 1260 | Channel or | Channel |
| | channel alpha 2 | | transporter | |
| CNGA3 | Cyclic nucleotide gated | 1261 | Channel or | Channel |
| | channel alpha 3 | | transporter | |
| CNGA4 | Cyclic nucleotide gated | 1262 | Channel or | Channel |
| | channel alpha 4 | | transporter | |
| CNGB1 | Cyclic nucleotide gated | 1258 | Channel or | Channel |
| | channel beta 1 | | transporter | |
| CNGB3 | Cyclic nucleotide gated | 54714 | Channel or | Channel |
| | channel beta 3 | | transporter | |
| CNR1 | Cannabinoid receptor 1 | 1268 | Neurotransmitter | Receptor |
| CNR2 | Cannabinoid receptor 2 | 1269 | Neurotransmitter | Receptor |
| CNRIP1 | Cannabinoid receptor | 25927 | Neurotransmitter | Receptor |
| | interacting protein 1 | | | |
| CNTF | Ciliary neurotrophic factor | 1270 | Neurotrophic | Ligand |
| CNTFR | Ciliary neurotrophic factor | 1271 | Neurotrophic | Receptor |
| | receptor | | | |
| CNTNAP4 | Contactin associated protein | 85445 | Vesicular | Vesicles |
| | like 4 | | | |
| COMT | Catechol-O-methyltransferase | 1312 | Neurotransmitter | Biosynthesis |
| CORT | Cortistatin | 1325 | Neuropeptide | Ligand |
| CPA4 | Carboxypeptidase A4 | 51200 | Neurotransmitter | Biosynthesis |
| CPE | Carboxypeptidase E | 1363 | Neurotransmitter | Biosynthesis |
| CPLX1 | Complexin 1 | 10815 | Vesicular | Vesicles |
| CPLX2 | Complexin 2 | 10814 | Vesicular | Vesicles |
| CPLX3 | Complexin 3 | 594855 | Vesicular | Vesicles |
| CPLX4 | Complexin 4 | 339302 | Vesicular | Vesicles |
| CRCP | CGRP receptor component | 27297 | Neuropeptide | Receptor |
| CREB1 | Camp responsive element | 1385 | Signaling | Signaling |
| | binding protein 1 | | | |
| CREM | Camp responsive element | 1390 | Neurotransmitter | Signaling |
| | modulator | | | |
| CRH | Corticotropin releasing | 1392 | Neuropeptide | Ligand |
| | hormone | | | |
| CRHR1 | Corticotropin releasing | 1394 | Neuropeptide | Receptor |
| | hormone receptor 1 | | | |
| CRHR2 | Corticotropin releasing | 1395 | Neuropeptide | Receptor |
| | hormone receptor 2 | | | |
| CRLF1 | Cytokine receptor like factor 1 | 9244 | Neurotrophic | Receptor |
| CSK | C-src tyrosine kinase | 1445 | Signaling | Signaling |
| CSNK1E | Casein kinase 1 epsilon | 1454 | Signaling | Signaling |
| CSPG5 | Chondroitin sulfate | 10675 | Neurotrophic | Ligand |
| | proteoglycan 5 | | | |
| CST3 | Cystatin C | 1471 | Other | Miscelaneous |
| CTAGE15 | CTAGE family member 15 | 441294 | Other | Miscelaneous |
| CTAGE4 | CTAGE family member 4 | 100128553 | Other | Miscelaneous |
| CTAGE6 | CTAGE family member 6 | 340307 | Other | Miscelaneous |
| CTAGE8 | CTAGE family member 8 | 100142659 | Other | Miscelaneous |
| CTAGE9 | CTAGE family member 9 | 643854 | Other | Miscelaneous |
| CTBP2 | C-terminal binding protein 2 | 1488 | Signaling | Signaling |
| CTSH | Cathepsin H | 1512 | Neuropeptide | Biosynthesis |
| CTSV | Cathepsin V | 1515 | Neuropeptide | Biosynthesis |
| CXCR4 | C-X-C motif chemokine | 7852 | Other | Miscelaneous |
| | receptor 4 | | | |
| CYFIP1 | Cytoplasmic FMR1 interacting | 23191 | Signaling | Signaling |
| | protein 1 | | | |
| CYP19A1 | Cytochrome P450 family 19 | 1588 | Other | Miscelaneous |
| | subfamily A member 1 | | | |
| CYSLTR1 | Cysteinyl leukotriene receptor | 10800 | Neuropeptide | Receptor |
| | 1 | | | |
| CYSLTR2 | Cysteinyl leukotriene receptor | 57105 | Neuropeptide | Receptor |
| | 2 | | | |
| DAGLA | Diacylglycerol lipase alpha | 747 | Neurotransmitter | Biosynthesis |
| DAGLB | Diacylglycerol lipase beta | 221955 | Neurotransmitter | Biosynthesis |
| DBH | Dopamine beta-hydroxylase | 1621 | Neurotransmitter | Biosynthesis |
| DBI | Diazepam binding inhibitor, | 1622 | Neuropeptide | Ligand |
| | acyl-coa binding protein | | | |
| DCLK1 | Doublecortin like kinase 1 | 9201 | Neurotrophic | Signaling |
| DDC | Dopa decarboxylase | 1644 | Neurotransmitter | Biosynthesis |
| DDR1 | Discoidin domain receptor | 780 | Other | Miscelaneous |
| | tyrosine kinase 1 | | | |
| DDR2 | Discoidin domain receptor | 4921 | Other | Miscelaneous |
| | tyrosine kinase 2 | | | |
| DGKI | Diacylglycerol kinase iota | 9162 | Neurotransmitter | Biosynthesis |
| DIRC2 | Disrupted in renal carcinoma 2 | 84925 | Channel or | Transporter |
| | | | transporter | |
| DISC1 | Disrupted in schizophrenia 1 | 27185 | Neurotrophic | Signaling |
| DKK1 | Dickkopf WNT signaling | 22943 | Other | Miscelaneous |
| | pathway inhibitor 1 | | | |
| DLGAP2 | DLG associated protein 2 | 9228 | Vesicular | Vesicles |
| DNAJC5 | Dnaj heat shock protein family | 80331 | Neurotrophic | Signaling |
| | (Hsp40) member C5 | | | |
| DNM1 | Dynamin 1 | 1759 | Vesicular | Vesicles |
| DNM2 | Dynamin 2 | 1785 | Vesicular | Vesicles |
| DOC2A | Double C2 domain alpha | 8448 | Signaling | Signaling |
| DOC2B | Double C2 domain beta | 8447 | Signaling | Signaling |
| DPP4 | Dipeptidyl peptidase 4 | 1803 | Neurotransmitter | Biosynthesis |
| DPYSL2 | Dihydropyrimidinase like 2 | 1808 | Neurotrophic | Signaling |
| DRD1 | Dopamine receptor D1 | 1812 | Neurotransmitter | Receptor |
| DRD2 | Dopamine receptor D2 | 1813 | Neurotransmitter | Receptor |
| DRD3 | Dopamine receptor D3 | 1814 | Neurotransmitter | Receptor |
| DRD4 | Dopamine receptor D4 | 1815 | Neurotransmitter | Receptor |
| DRD5 | Dopamine receptor D5 | 1816 | Neurotransmitter | Receptor |
| DTNA | Dystrobrevin alpha | 1837 | Other | Miscelaneous |
| DTNBP1 | Dystrobrevin binding protein 1 | 84062 | Other | Miscelaneous |
| DVL1 | Dishevelled segment polarity | 1855 | Neurotrophic | Signaling |
| | protein 1 | | | |
| ECEL1 | Endothelin converting enzyme | 9427 | Neurotransmitter | Biosynthesis |
| | like 1 | | | |
| EDN1 | Endothelin 1 | 1906 | Neuropeptide | Ligand |
| EDN2 | Endothelin 2 | 1907 | Neuropeptide | Ligand |
| EDN3 | Endothelin 3 | 1908 | Neuropeptide | Ligand |
| EDNRA | Endothelin receptor type A | 1909 | Neuropeptide | Receptor |
| EDNRB | Endothelin receptor type B | 1910 | Neuropeptide | Receptor |
| EEF2 | Eukaryotic translation | 1938 | Other | Miscelaneous |
| | elongation factor 2 | | | |
| EEF2K | Eukaryotic elongation factor 2 | 29904 | Other | Miscelaneous |
| | kinase | | | |
| EFNA5 | Ephrin-A5 (AL-1) (EPH-related | 1946 | Neurotrophic | Ligand |
| | receptor tyrosine kinase ligand | | | |
| | 7) (LERK-7) | | | |
| EGF | Epidermal growth factor | 1950 | Other | Miscelaneous |
| EGFR | Epidermal growth factor | 1956 | Other | Miscelaneous |
| | receptor | | | |
| EGR3 | Early growth response 3 | 1960 | Neurotrophic | Signaling |
| EIF4A3 | Eukaryotic translation initiation | 9775 | Other | Miscelaneous |
| | factor 4A3 | | | |
| EIF4EBP2 | Eukaryotic translation initiation | 1979 | Other | Miscelaneous |
| | factor 4E binding protein 2 | | | |
| EN1 | Engrailed homeobox 1 | 2019 | Other | Miscelaneous |
| ENO2 | Enolase 2 | 2026 | Neurotrophic | Signaling |
| EphA1 | Ephrin type-A receptor 1 | 2041 | Neurotrophic | Receptor |
| EphA10 | Ephrin type-A receptor 10 | 284656 | Neurotrophic | Receptor |
| EphA2 | Ephrin type-A receptor 2 | 1969 | Neurotrophic | Receptor |
| EphA3 | Ephrin type-A receptor 3 | 2042 | Neurotrophic | Receptor |
| EphA4 | Ephrin type-A receptor 4 | 2043 | Neurotrophic | Receptor |
| EphA5 | Ephrin type-A receptor 5 | 2044 | Neurotrophic | Receptor |
| EphA6 | Ephrin type-A receptor 6 | 285220 | Neurotrophic | Receptor |
| EphA7 | Ephrin type-A receptor 7 | 2045 | Neurotrophic | Receptor |
| EphA8 | Ephrin type-A receptor 8 | 2046 | Neurotrophic | Receptor |
| EphB1 | Ephrin type-B receptor 1 | 2047 | Neurotrophic | Receptor |
| EphB2 | Ephrin type-B receptor 2 | 2048 | Neurotrophic | Receptor |
| EphB3 | Ephrin type-B receptor 3 | 2049 | Neurotrophic | Receptor |
| EphB4 | Ephrin type-B receptor 4 | 2050 | Neurotrophic | Receptor |
| EphB6 | Ephrin type-B receptor 6 | 2051 | Neurotrophic | Receptor |
| EPO | Erythropoietin | 2056 | Other | Miscelaneous |
| ERBB2 | Erb-b2 receptor tyrosine | 2064 | Other | Miscelaneous |
| | kinase 2 | | | |
| ERC1 | ELKS/RAB6-interacting/CAST | 23085 | Signaling | Signaling |
| | family member 1 | | | |
| ERC2 | ELKS/RAB6-interacting/CAST | 26059 | Signaling | Signaling |
| | family member 2 | | | |
| ERG | ERG, ETS transcription factor | 2078 | Other | Miscelaneous |
| ETBR2 | Receptor for prosaposin | 9283 | Neurotrophic | Receptor |
| ETV5 | ETS variant 5 | 2119 | Other | Miscelaneous |
| EXOC3L1 | Exocyst complex component 3 | 283849 | Other | Miscelaneous |
| | like 1 | | | |
| F2RL1 | F2R like trypsin receptor 1 | 2150 | Other | Miscelaneous |
| FAAH | Fatty acid amide hydrolase | 2166 | Neurotransmitter | Biosynthesis |
| FAP | Fibroblast activation protein | 2191 | Neuropeptide | Biosynthesis |
| | alpha | | | |
| FEV | FEV, ETS transcription factor | 54738 | Other | Miscelaneous |
| FFAR3 | Free fatty acid receptor 3 | 2865 | Other | Miscelaneous |
| FGF14 | Fibroblast growth factor 14 | 2259 | Other | Miscelaneous |
| FGF2 | Fibroblast growth factor 2 | 2247 | Other | Miscelaneous |
| FGF20 | Fibroblast growth factor 20 | 26281 | Other | Miscelaneous |
| FKBP5 | FK506 binding protein 5 | 2289 | Other | Miscelaneous |
| FLNA | Filamin A | 2316 | Other | Miscelaneous |
| FLVCR1 | Feline leukemia virus | 28982 | Channel or | Transporter |
| | subgroup C cellular receptor 1 | | transporter | |
| FLVCR2 | Feline leukemia virus | 55640 | Channel or | Transporter |
| | subgroup C cellular receptor | | transporter | |
| | family member 2 | | | |
| FMR1 | Fragile X mental retardation 1 | 2332 | Other | Miscelaneous |
| FNTA | Farnesyltransferase, CAAX | 2339 | Neurotransmitter | Signaling |
| | box, alpha | | | |
| FOLH1 | Folate hydrolase 1 | 2346 | Neuropeptide | Biosynthesis |
| FRS2 | Fibroblast growth factor | 10818 | Other | Miscelaneous |
| | receptor substrate 2 | | | |
| FRS3 | Fibroblast growth factor | 10817 | Other | Miscelaneous |
| | receptor substrate 3 | | | |
| FSHR | Follicle stimulating hormone | 2492 | Neuropeptide | Receptor |
| | receptor | | | |
| FSTL4 | Follistatin like 4 | 23105 | Neurotrophic | Receptor |
| FXYD2 | FXYD domain containing ion | 486 | Channel or | Transporter |
| | transport regulator 2 | | transporter | |
| GAB1 | GRB2 associated binding | 2549 | Signaling | Signaling |
| | protein 1 | | | |
| GABARAP | GABA type A receptor- | 11337 | Neurotransmitter | Receptor |
| | associated protein | | | |
| GABBR1 | Gamma-aminobutyric acid | 2550 | Neurotransmitter | Receptor |
| | type B receptor subunit 1 | | | |
| GABRA1 | Gamma-aminobutyric acid | 2554 | Neurotransmitter | Receptor |
| | type A receptor alpha1 subunit | | | |
| GABRA2 | Gamma-aminobutyric acid | 2555 | Neurotransmitter | Receptor |
| | type A receptor alpha2 subunit | | | |
| GABRA3 | Gamma-aminobutyric acid | 2556 | Neurotransmitter | Receptor |
| | type A receptor alpha3 subunit | | | |
| GABRA4 | Gamma-aminobutyric acid | 2557 | Neurotransmitter | Receptor |
| | type A receptor alpha4 subunit | | | |
| GABRA5 | Gamma-aminobutyric acid | 2558 | Neurotransmitter | Receptor |
| | type A receptor alpha5 subunit | | | |
| GABRA6 | Gamma-aminobutyric acid | 2559 | Neurotransmitter | Receptor |
| | type A receptor alpha6 subunit | | | |
| GABRB1 | Gamma-aminobutyric acid | 2560 | Neurotransmitter | Receptor |
| | type A receptor beta1 subunit | | | |
| GABRB2 | Gamma-aminobutyric acid | 2561 | Neurotransmitter | Receptor |
| | type A receptor beta2 subunit | | | |
| GABRB3 | Gamma-aminobutyric acid | 2562 | Neurotransmitter | Receptor |
| | type A receptor beta3 subunit | | | |
| GABRD | Gamma-aminobutyric acid | 2563 | Neurotransmitter | Receptor |
| | type A receptor delta subunit | | | |
| GABRE | Gamma-aminobutyric acid | 2564 | Neurotransmitter | Receptor |
| | type A receptor epsilon | | | |
| | subunit | | | |
| GABRG1 | Gamma-aminobutyric acid | 2565 | Neurotransmitter | Receptor |
| | type A receptor gamma1 | | | |
| | subunit | | | |
| GABRG2 | Gamma-aminobutyric acid | 2566 | Neurotransmitter | Receptor |
| | type A receptor gamma2 | | | |
| | subunit | | | |
| GABRG3 | Gamma-aminobutyric acid | 2567 | Neurotransmitter | Receptor |
| | type A receptor gamma3 | | | |
| | subunit | | | |
| GABRP | Gamma-aminobutyric acid | 2568 | Neurotransmitter | Receptor |
| | type A receptor pi subunit | | | |
| GABRQ | Gamma-aminobutyric acid | 55879 | Neurotransmitter | Receptor |
| | type A receptor theta subunit | | | |
| GABRR1 | Gamma-aminobutyric acid | 2569 | Neurotransmitter | Receptor |
| | type A receptor rho1 subunit | | | |
| GABRR2 | Gamma-aminobutyric acid | 2570 | Neurotransmitter | Receptor |
| | type A receptor rho2 subunit | | | |
| GABRR3 | Gamma-aminobutyric acid | 200959 | Neurotransmitter | Receptor |
| | type A receptor rho3 subunit | | | |
| | (gene/pseudogene) | | | |
| GAD1 | Glutamate decarboxylase 1 | 2571 | Neurotransmitter | Biosynthesis |
| GAD2 | Glutamate decarboxylase 2 | 2572 | Neurotransmitter | Biosynthesis |
| GAL | Galanin and GMAP | 51083 | Neuropeptide | Ligand |
| | prepropeptide | | | |
| GALP | Galanin like peptide | 85569 | Neuropeptide | Ligand |
| GALR1 | Galanin receptor 1 | 2587 | Neuropeptide | Receptor |
| GALR2 | Galanin receptor 2 | 8811 | Neuropeptide | Receptor |
| GALR3 | Galanin receptor 3 | 8484 | Neuropeptide | Receptor |
| GAST | Gastrin | 2520 | Neuropeptide | Ligand |
| GCGR | Glucagon receptor | 2642 | Other | Miscelaneous |
| GCHFR | GTP cyclohydrolase I | 2644 | Neurotransmitter | Biosynthesis |
| | feedback regulator | | | |
| GDNF | Glial cell derived neurotrophic | 2668 | Neurotrophic | Ligand |
| | factor | | | |
| GFAP | Glial fibrillary acidic protein | 2670 | Other | Miscelaneous |
| GFRA1 | GDNF family receptor alpha 1 | 2674 | Neurotrophic | Receptor |
| GFRA2 | GDNF family receptor alpha 2 | 2675 | Neurotrophic | Receptor |
| GFRA3 | GDNF family receptor alpha 3 | 2676 | Neurotrophic | Receptor |
| GFRA4 | GDNF family receptor alpha 4 | 64096 | Neurotrophic | Receptor |
| GH1 | Growth hormone 1 | 2688 | Neuropeptide | Ligand |
| GHRH | Growth hormone releasing | 2691 | Neuropeptide | Ligand |
| | hormone | | | |
| GHRHR | Growth hormone releasing | 2692 | Neuropeptide | Receptor |
| | hormone receptor | | | |
| GHRL | Ghrelin and obestatin | 51738 | Neuropeptide | Ligand |
| | prepropeptide | | | |
| GIP | Gastric inhibitory polypeptide | 2695 | Neuropeptide | Ligand |
| GJA1 | Gap junction protein alpha 1 | 2697 | Channel or | Channel |
| | | | transporter | |
| GJA10 | Gap junction protein alpha 10 | 84694 | Channel or | Channel |
| | | | transporter | |
| GJA3 | Gap junction protein alpha 3 | 2700 | Channel or | Channel |
| | | | transporter | |
| GJA4 | Gap junction protein alpha 4 | 2701 | Channel or | Channel |
| | | | transporter | |
| GJA5 | Gap junction protein alpha 5 | 2702 | Channel or | Channel |
| | | | transporter | |
| GJA8 | Gap junction protein alpha 8 | 2703 | Channel or | Channel |
| | | | transporter | |
| GJA9 | Gap junction protein alpha 9 | 81025 | Channel or | Channel |
| | | | transporter | |
| GJB1 | Gap junction protein beta 1 | 2705 | Channel or | Channel |
| | | | transporter | |
| GJB2 | Gap junction protein beta 2 | 2706 | Channel or | Channel |
| | | | transporter | |
| GJB3 | Gap junction protein beta 3 | 2707 | Channel or | Channel |
| | | | transporter | |
| GJB4 | Gap junction protein beta 4 | 127534 | Channel or | Channel |
| | | | transporter | |
| GJB5 | Gap junction protein beta 5 | 2709 | Channel or | Channel |
| | | | transporter | |
| GJB6 | Gap junction protein beta 6 | 10804 | Channel or | Channel |
| | | | transporter | |
| GJB7 | Gap junction protein beta 7 | 375519 | Channel or | Channel |
| | | | transporter | |
| GJC1 | Gap junction protein gamma 1 | 10052 | Channel or | Channel |
| | | | transporter | |
| GJC2 | Gap junction protein gamma 2 | 57165 | Channel or | Channel |
| | | | transporter | |
| GJC3 | Gap junction protein gamma 3 | 349149 | Channel or | Channel |
| | | | transporter | |
| GJD2 | Gap junction protein delta 2 | 57369 | Channel or | Channel |
| | | | transporter | |
| GJD3 | Gap junction protein delta 3 | 125111 | Channel or | Channel |
| | | | transporter | |
| GJD4 | Gap junction protein delta 4 | 219770 | Channel or | Channel |
| | | | transporter | |
| GJE1 | Gap junction protein epsilon 1 | 100126572 | Channel or | Channel |
| | | | transporter | |
| GLRA1 | Glycine receptor alpha 1 | 2741 | Neurotransmitter | Receptor |
| GLRA2 | Glycine receptor alpha 2 | 2742 | Neurotransmitter | Receptor |
| GLRA3 | Glycine receptor alpha 3 | 8001 | Neurotransmitter | Receptor |
| GLRA4 | Glycine receptor alpha 4 | 441509 | Neurotransmitter | Receptor |
| GLRB | Glycine receptor beta | 2743 | Neurotransmitter | Receptor |
| GLS | Glutaminase | 2744 | Neurotransmitter | Biosynthesis |
| GLS2 | Glutaminase 2 | 27165 | Neurotransmitter | Biosynthesis |
| GLUL | Glutamate-ammonia ligase | 2752 | Neurotransmitter | Biosynthesis |
| GNA11 | G protein subunit alpha 11 | 2767 | Signaling | Signaling |
| GNA13 | G protein subunit alpha 13 | 10672 | Signaling | Signaling |
| GNA14 | G protein subunit alpha 14 | 9630 | Signaling | Signaling |
| GNA15 | G protein subunit alpha 15 | 2769 | Signaling | Signaling |
| GNAI1 | G protein subunit alpha i1 | 2770 | Signaling | Signaling |
| GNAI2 | G protein subunit alpha i2 | 2771 | Signaling | Signaling |
| GNAI3 | G protein subunit alpha i3 | 2773 | Signaling | Signaling |
| GNAL | G protein subunit alpha L | 2774 | Signaling | Signaling |
| GNAO1 | G protein subunit alpha o1 | 2775 | Signaling | Signaling |
| GNAQ | G protein subunit alpha q | 2776 | Signaling | Signaling |
| GNAS | GNAS complex locus | 2778 | Signaling | Signaling |
| GNAZ | G protein subunit alpha z | 2781 | Signaling | Signaling |
| GNB1 | G protein subunit beta 1 | 2782 | Signaling | Signaling |
| GNB2 | G protein subunit beta 2 | 2783 | Signaling | Signaling |
| GNB3 | G protein subunit beta 3 | 2784 | Signaling | Signaling |
| GNB4 | G protein subunit beta 4 | 59345 | Signaling | Signaling |
| GNB5 | G protein subunit beta 5 | 10681 | Signaling | Signaling |
| GNG10 | G protein subunit gamma 10 | 2790 | Signaling | |
| GNG11 | G protein subunit gamma 11 | 2791 | Signaling | |
| GNG12 | G protein subunit gamma 12 | 55970 | Signaling | Signaling |
| GNG13 | G protein subunit gamma 13 | 51764 | Signaling | Signaling |
| GNG2 | G protein subunit gamma 2 | 54331 | Signaling | Signaling |
| GNG3 | G protein subunit gamma 3 | 2785 | Signaling | Signaling |
| GNG4 | G protein subunit gamma 4 | 2786 | Signaling | |
| GNG5 | G protein subunit gamma 5 | 2787 | Signaling | |
| GNG7 | G protein subunit gamma 7 | 2788 | Signaling | |
| GNG8 | G protein subunit gamma 8 | 94235 | Signaling | Signaling |
| GNGT2 | G protein subunit gamma | 2793 | Signaling | |
| | transducin 2 | | | |
| GNMT | Glycine N-methyltransferase | 27232 | Neurotransmitter | Biosynthesis |
| GNRH1 | Gonadotropin releasing | 2796 | Neuropeptide | Ligand |
| | hormone 1 | | | |
| GNRH2 | Gonadotropin releasing | 2797 | Neuropeptide | Ligand |
| | hormone 2 | | | |
| GPER1 | G protein-coupled estrogen | 2852 | Other | Receptor |
| | receptor 1 | | | |
| GPHN | Gephyrin | 10243 | Neuropeptide | Ligand |
| GPI | Glucose-6-phosphate | 2821 | Signaling | Signaling |
| | isomerase | | | |
| GPR1 | G protein-coupled receptor 1 | 2825 | Other | Receptor |
| GPR139 | G protein-coupled receptor | 124274 | Neurotransmitter | Receptor |
| | 139 | | | |
| GPR143 | G protein-coupled receptor | 4935 | Neurotransmitter | Receptor |
| | 143 | | | |
| GPR149 | G protein-coupled receptor | 344758 | Neurotransmitter | Receptor |
| | 149 | | | |
| GPR18 | G protein-coupled receptor 18 | 2841 | Other | Receptor |
| GPR21 | G protein-coupled receptor 21 | 2844 | Other | Receptor |
| GPR26 | G protein-coupled receptor 26 | 2849 | Other | Receptor |
| GPR35 | G protein-coupled receptor 35 | 2859 | Other | Receptor |
| GPR37 | Receptor for prosaposin | 2861 | Neurotrophic | Receptor |
| GPR52 | G protein-coupled receptor 52 | 9293 | Neurotransmitter | Receptor |
| GPR55 | G protein-coupled receptor 55 | 9290 | Neurotransmitter | Receptor |
| GPR78 | G protein-coupled receptor 78 | 27201 | Neurotransmitter | Receptor |
| GPR83 | G protein-coupled receptor 83 | 10888 | Neurotransmitter | Receptor |
| GPR84 | G protein-coupled receptor 84 | 53831 | Neurotransmitter | Receptor |
| GPRASP1 | G protein-coupled receptor | 9737 | Neurotransmitter | Receptor |
| | associated sorting protein 1 | | | |
| GPRC6A | G protein-coupled receptor | 222545 | Signaling | Signaling |
| | class C group 6 member A | | | |
| GPRIN1 | G protein regulated inducer of | 114787 | Neurotrophic | Signaling |
| | neurite outgrowth 1 | | | |
| GPRIN2 | G protein regulated inducer of | 9721 | Neurotrophic | Signaling |
| | neurite outgrowth 2 | | | |
| GPRIN3 | G protein regulated inducer of | 285513 | Neurotrophic | Signaling |
| | neurite outgrowth 3 | | | |
| GRB2 | Growth factor receptor bound | 2885 | Neurotrophic | Signaling |
| | protein 2 | | | |
| GRIA1 | Glutamate ionotropic receptor | 2890 | Neurotransmitter | Receptor |
| | AMPA type subunit 1 | | | |
| GRIA2 | Glutamate ionotropic receptor | 2891 | Neurotransmitter | Receptor |
| | AMPA type subunit 2 | | | |
| GRIA3 | Glutamate ionotropic receptor | 2892 | Neurotransmitter | Receptor |
| | AMPA type subunit 3 | | | |
| GRIA4 | Glutamate ionotropic receptor | 2893 | Neurotransmitter | Receptor |
| | AMPA type subunit 4 | | | |
| GRID1 | Glutamate ionotropic receptor | 2894 | Neurotransmitter | Receptor |
| | delta type subunit 1 | | | |
| GRID2 | Glutamate ionotropic receptor | 2895 | Neurotransmitter | Receptor |
| | delta type subunit 2 | | | |
| GRIK1 | Glutamate ionotropic receptor | 2897 | Neurotransmitter | Receptor |
| | kainate type subunit 1 | | | |
| GRIK2 | Glutamate ionotropic receptor | 2898 | Neurotransmitter | Receptor |
| | kainate type subunit 2 | | | |
| GRIK3 | Glutamate ionotropic receptor | 2899 | Neurotransmitter | Receptor |
| | kainate type subunit 3 | | | |
| GRIK4 | Glutamate ionotropic receptor | 2900 | Neurotransmitter | Receptor |
| | kainate type subunit 4 | | | |
| GRIK5 | Glutamate ionotropic receptor | 2901 | Neurotransmitter | Receptor |
| | kainate type subunit 5 | | | |
| GRIN1 | Glutamate ionotropic receptor | 2902 | Neurotransmitter | Receptor |
| | NMDA type subunit 1 | | | |
| GRIN2A | Glutamate ionotropic receptor | 2903 | Neurotransmitter | Receptor |
| | NMDA type subunit 2A | | | |
| GRIN2B | Glutamate ionotropic receptor | 2904 | Neurotransmitter | Receptor |
| | NMDA type subunit 2B | | | |
| GRIN2C | Glutamate ionotropic receptor | 2905 | Neurotransmitter | Receptor |
| | NMDA type subunit 2C | | | |
| GRIN2D | Glutamate ionotropic receptor | 2906 | Neurotransmitter | Receptor |
| | NMDA type subunit 2D | | | |
| GRIN3A | Glutamate ionotropic receptor | 116443 | Neurotransmitter | Receptor |
| | NMDA type subunit 3A | | | |
| GRIN3B | Glutamate ionotropic receptor | 116444 | Neurotransmitter | Receptor |
| | NMDA type subunit 3B | | | |
| GRK2 | G protein-coupled receptor | 156 | Neurotransmitter | Receptor |
| | kinase 2 | | | |
| GRK3 | G protein-coupled receptor | 157 | Neurotransmitter | Receptor |
| | kinase 3 | | | |
| GRK4 | G protein-coupled receptor | 2868 | Signaling | Signaling |
| | kinase 4 | | | |
| GRK5 | G protein-coupled receptor | 2869 | Signaling | Signaling |
| | kinase 5 | | | |
| GRM1 | Glutamate metabotropic | 2911 | Neurotransmitter | Receptor |
| | receptor 1 | | | |
| GRM2 | Glutamate metabotropic | 2912 | Neurotransmitter | Receptor |
| | receptor 2 | | | |
| GRM3 | Glutamate metabotropic | 2913 | Neurotransmitter | Receptor |
| | receptor 3 | | | |
| GRM4 | Glutamate metabotropic | 2914 | Neurotransmitter | Receptor |
| | receptor 4 | | | |
| GRM5 | Glutamate metabotropic | 2915 | Neurotransmitter | Receptor |
| | receptor 5 | | | |
| GRM6 | Glutamate metabotropic | 2916 | Neurotransmitter | Receptor |
| | receptor 6 | | | |
| GRM7 | Glutamate metabotropic | 2917 | Neurotransmitter | Receptor |
| | receptor 7 | | | |
| GRM8 | Glutamate metabotropic | 2918 | Neurotransmitter | Receptor |
| | receptor 8 | | | |
| GRP | Gastrin releasing peptide | 2922 | Neuropeptide | Ligand |
| GRPR | Gastrin releasing peptide | 2925 | Neuropeptide | Receptor |
| | receptor | | | |
| GSK3A | Glycogen synthase kinase 3 | 2931 | Signaling | Signaling |
| | alpha | | | |
| GSK3B | Glycogen synthase kinase 3 | 2932 | Signaling | Signaling |
| | beta | | | |
| GTF2H2 | General transcription factor | 2966 | Other | Miscelaneous |
| | IIH subunit 2 | | | |
| GZF1 | GDNF-inducible zinc finger | 64412 | Neurotrophic | Signaling |
| | protein 1 | | | |
| HAP1 | Huntingtin associated protein | 9001 | Other | Miscelaneous |
| | 1 | | | |
| HCN1 | Hyperpolarization activated | 348980 | Channel or | Channel |
| | cyclic nucleotide gated | | transporter | |
| | potassium channel 1 | | | |
| HCN2 | Hyperpolarization activated | 610 | Channel or | Channel |
| | cyclic nucleotide gated | | transporter | |
| | potassium channel 2 | | | |
| HCN3 | Hyperpolarization activated | 57657 | Channel or | Channel |
| | cyclic nucleotide gated | | transporter | |
| | potassium channel 3 | | | |
| HCN4 | Hyperpolarization activated | 10021 | Channel or | Channel |
| | cyclic nucleotide gated | | transporter | |
| | potassium channel 4 | | | |
| HCRT | Hypocretin neuropeptide | 3060 | Neuropeptide | Ligand |
| | precursor | | | |
| HCRTR1 | Hypocretin receptor 1 | 3061 | Neuropeptide | Receptor |
| HCRTR2 | Hypocretin receptor 2 | 3062 | Neuropeptide | Receptor |
| HIP1 | Huntingtin interacting protein 1 | 3092 | Other | Miscelaneous |
| HK2 | Hexokinase 2 | 3099 | Other | Miscelaneous |
| HMOX1 | Heme oxygenase 1 | 3162 | Other | Miscelaneous |
| HMOX2 | Heme oxygenase 2 | 3163 | Other | Miscelaneous |
| HNMT | Histamine N- | 3176 | Neurotransmitter | Biosynthesis |
| | methyltransferase | | | |
| HOMER1 | Homer scaffolding protein 1 | 9456 | Neurotransmitter | Receptor |
| HRAS | Hras proto-oncogene, gtpase | 3265 | Signaling | Signaling |
| HRH1 | Histamine receptor H1 | 3269 | Neurotransmitter | Receptor |
| HRH2 | Histamine receptor H2 | 3274 | Neurotransmitter | Receptor |
| HRH3 | Histamine receptor H3 | 11255 | Neurotransmitter | Receptor |
| HRH4 | Histamine receptor H4 | 59340 | Neurotransmitter | Receptor |
| HSPA8 | Heat shock protein family A | 3312 | Vesicular | Vesicles |
| | (Hsp70) member 8 | | | |
| HTL | High L-leucine transport | 3343 | Channel or | Transporter |
| | | | transporter | |
| HTR1A | 5-hydroxytryptamine receptor | 3350 | Neurotransmitter | Receptor |
| | 1A | | | |
| HTR1B | 5-hydroxytryptamine receptor | 3351 | Neurotransmitter | Receptor |
| | 1B | | | |
| HTR1D | 5-hydroxytryptamine receptor | 3352 | Neurotransmitter | Receptor |
| | 1D | | | |
| HTR1E | 5-hydroxytryptamine receptor | 3354 | Neurotransmitter | Receptor |
| | 1E | | | |
| HTR1F | 5-hydroxytryptamine receptor | 3355 | Neurotransmitter | Receptor |
| | 1F | | | |
| HTR2A | 5-hydroxytryptamine receptor | 3356 | Neurotransmitter | Receptor |
| | 2A | | | |
| HTR2B | 5-hydroxytryptamine receptor | 3357 | Neurotransmitter | Receptor |
| | 2B | | | |
| HTR2C | 5-hydroxytryptamine receptor | 3358 | Neurotransmitter | Receptor |
| | 2C | | | |
| HTR3A | 5-hydroxytryptamine receptor | 3359 | Neurotransmitter | Receptor |
| | 3A | | | |
| HTR3B | 5-hydroxytryptamine receptor | 9177 | Neurotransmitter | Receptor |
| | 3B | | | |
| HTR3C | 5-hydroxytryptamine receptor | 170572 | Neurotransmitter | Receptor |
| | 3C | | | |
| HTR3D | 5-hydroxytryptamine receptor | 200909 | Neurotransmitter | Receptor |
| | 3D | | | |
| HTR3E | 5-hydroxytryptamine receptor | 285242 | Neurotransmitter | Receptor |
| | 3E | | | |
| HTR4 | 5-hydroxytryptamine receptor | 3360 | Neurotransmitter | Receptor |
| | 4 | | | |
| HTR5A | 5-hydroxytryptamine receptor | 3361 | Neurotransmitter | Receptor |
| | 5A | | | |
| HTR5BP | 5-hydroxytryptamine receptor | 645694 | Neurotransmitter | Receptor |
| | 5B, pseudogene | | | |
| HTR6 | 5-hydroxytryptamine receptor | 3362 | Neurotransmitter | Receptor |
| | 6 | | | |
| HTR7 | 5-hydroxytryptamine receptor | 3363 | Neurotransmitter | Receptor |
| | 7 | | | |
| HTT | Huntingtin | 3064 | Other | Miscelaneous |
| HVCN1 | Hydrogen voltage gated | 84329 | Channel or | Channel |
| | channel 1 | | transporter | |
| IAPP | Islet amyloid polypeptide | 3375 | Neuropeptide | Ligand |
| ICA1 | Islet cell autoantigen 1 | 3382 | Other | Miscelaneous |
| IFNA1 | Interferon alpha 1 | 3439 | Neurotrophic | Ligand |
| IGF1 | Insulin like growth factor 1 | 3479 | Neurotrophic | Ligand |
| IGF2 | Insulin like growth factor 2 | 3481 | Neurotrophic | Ligand |
| IL11RA | Interleukin 11 receptor subunit | 3590 | Neurotrophic | Receptor |
| | alpha | | | |
| IL1B | Interleukin 1 beta | 3553 | Neurotrophic | Ligand |
| IL3 | Interleukin 3 | 3562 | Neurotrophic | Ligand |
| IL4 | Interleukin 4 | 3565 | Neurotrophic | Ligand |
| IL6 | Interleukin 6 | 3569 | Neurotrophic | Ligand |
| IL6R | Interleukin 6 receptor | 3570 | Neurotrophic | Receptor |
| IL6ST | Interleukin 6 signal transducer | 3572 | Neurotrophic | Signaling |
| INS | Insulin | 3630 | Neurotrophic | Ligand |
| ITPR1 | Inositol 1,4,5-trisphosphate | 3708 | Neurotransmitter | Signaling |
| | receptor type 1 | | | |
| ITPR2 | Inositol 1,4,5-trisphosphate | 3709 | Neurotransmitter | Signaling |
| | receptor type 2 | | | |
| ITPR3 | Inositol 1,4,5-trisphosphate | 3710 | Neurotransmitter | Signaling |
| | receptor type 3 | | | |
| KALRN | Kalirin, rhogef kinase | 8997 | Vesicular | Vesicles |
| KCNA1 | Potassium voltage-gated | 3736 | Channel or | Channel |
| | channel subfamily A member | | transporter | |
| | 1 | | | |
| KCNA10 | Potassium voltage-gated | 3744 | Channel or | Channel |
| | channel subfamily A member | | transporter | |
| | 10 | | | |
| KCNA2 | Potassium voltage-gated | 3737 | Channel or | Channel |
| | channel subfamily A member | | transporter | |
| | 2 | | | |
| KCNA3 | Potassium voltage-gated | 3738 | Channel or | Channel |
| | channel subfamily A member | | transporter | |
| | 3 | | | |
| KCNA4 | Potassium voltage-gated | 3739 | Channel or | Channel |
| | channel subfamily A member | | transporter | |
| | 4 | | | |
| KCNA5 | Potassium voltage-gated | 3741 | Channel or | Channel |
| | channel subfamily A member | | transporter | |
| | 5 | | | |
| KCNA6 | Potassium voltage-gated | 3742 | Channel or | Channel |
| | channel subfamily A member | | transporter | |
| | 6 | | | |
| KCNA7 | Potassium voltage-gated | 3743 | Channel or | Channel |
| | channel subfamily A member | | transporter | |
| | 7 | | | |
| KCNAB1 | Potassium voltage-gated | 7881 | Channel or | Channel |
| | channel subfamily A member | | transporter | |
| | regulatory beta subunit 1 | | | |
| KCNAB2 | Potassium voltage-gated | 8514 | Channel or | Channel |
| | channel subfamily A | | transporter | |
| | regulatory beta subunit 2 | | | |
| KCNB1 | Potassium voltage-gated | 3745 | Channel or | Channel |
| | channel subfamily B member | | transporter | |
| | 1 | | | |
| KCNB2 | Potassium voltage-gated | 9312 | Channel or | Channel |
| | channel subfamily B member | | transporter | |
| | 2 | | | |
| KCNC1 | Potassium voltage-gated | 3746 | Channel or | Channel |
| | channel subfamily C member | | transporter | |
| | 1 | | | |
| KCNC2 | Potassium voltage-gated | 3747 | Channel or | Channel |
| | channel subfamily C member | | transporter | |
| | 2 | | | |
| KCNC3 | Potassium voltage-gated | 3748 | Channel or | Channel |
| | channel subfamily C member | | transporter | |
| | 3 | | | |
| KCNC4 | Potassium voltage-gated | 3749 | Channel or | Channel |
| | channel subfamily C member | | transporter | |
| | 4 | | | |
| KCND1 | Potassium voltage-gated | 3750 | Channel or | Channel |
| | channel subfamily D member | | transporter | |
| | 1 | | | |
| KCND2 | Potassium voltage-gated | 3751 | Channel or | Channel |
| | channel subfamily D member | | transporter | |
| | 2 | | | |
| KCND3 | Potassium voltage-gated | 3752 | Channel or | Channel |
| | channel subfamily D member | | transporter | |
| | 3 | | | |
| KCNE2 | Potassium voltage-gated | 9992 | Channel or | Channel |
| | channel subfamily E | | transporter | |
| | regulatory subunit 2 | | | |
| KCNE3 | Potassium voltage-gated | 10008 | Channel or | Channel |
| | channel subfamily E | | transporter | |
| | regulatory subunit 3 | | | |
| KCNE4 | Potassium voltage-gated | 23704 | Channel or | Channel |
| | channel subfamily E | | transporter | |
| | regulatory subunit 4 | | | |
| KCNF1 | Potassium voltage-gated | 3754 | Channel or | Channel |
| | channel modifier subfamily F | | transporter | |
| | member 1 | | | |
| KCNG1 | Potassium voltage-gated | 3755 | Channel or | Channel |
| | channel modifier subfamily G | | transporter | |
| | member 1 | | | |
| KCNG2 | Potassium voltage-gated | 26251 | Channel or | Channel |
| | channel modifier subfamily G | | transporter | |
| | member 2 | | | |
| KCNG3 | Potassium voltage-gated | 170850 | Channel or | Channel |
| | channel modifier subfamily G | | transporter | |
| | member 3 | | | |
| KCNG4 | Potassium voltage-gated | 93107 | Channel or | Channel |
| | channel modifier subfamily G | | transporter | |
| | member 4 | | | |
| KCNH1 | Potassium voltage-gated | 3756 | Channel or | Channel |
| | channel subfamily H member | | transporter | |
| | 1 | | | |
| KCNH2 | Potassium voltage-gated | 3757 | Channel or | Channel |
| | channel subfamily H member | | transporter | |
| | 2 | | | |
| KCNH3 | Potassium voltage-gated | 23416 | Channel or | Channel |
| | channel subfamily H member | | transporter | |
| | 3 | | | |
| KCNH4 | Potassium voltage-gated | 23415 | Channel or | Channel |
| | channel subfamily H member | | transporter | |
| | 4 | | | |
| KCNH5 | Potassium voltage-gated | 27133 | Channel or | Channel |
| | channel subfamily H member | | transporter | |
| | 5 | | | |
| KCNH6 | Potassium voltage-gated | 81033 | Channel or | Channel |
| | channel subfamily H member | | transporter | |
| | 6 | | | |
| KCNH7 | Potassium voltage-gated | 90134 | Channel or | Channel |
| | channel subfamily H member | | transporter | |
| | 7 | | | |
| KCNH8 | Potassium voltage-gated | 131096 | Channel or | Channel |
| | channel subfamily H member | | transporter | |
| | 8 | | | |
| KCNJ1 | Potassium voltage-gated | 3758 | Channel or | Channel |
| | channel subfamily J member 1 | | transporter | |
| KCNJ10 | Potassium voltage-gated | 3766 | Channel or | Channel |
| | channel subfamily J member | | transporter | |
| | 10 | | | |
| KCNJ11 | Potassium voltage-gated | 3767 | Channel or | Channel |
| | channel subfamily J member | | transporter | |
| | 11 | | | |
| KCNJ12 | Potassium voltage-gated | 3768 | Channel or | Channel |
| | channel subfamily J member | | transporter | |
| | 12 | | | |
| KCNJ13 | Potassium voltage-gated | 3769 | Channel or | Channel |
| | channel subfamily J member | | transporter | |
| | 13 | | | |
| KCNJ14 | Potassium voltage-gated | 3770 | Channel or | Channel |
| | channel subfamily J member | | transporter | |
| | 14 | | | |
| KCNJ15 | Potassium voltage-gated | 3772 | Channel or | Channel |
| | channel subfamily J member | | transporter | |
| | 15 | | | |
| KCNJ16 | Potassium voltage-gated | 3773 | Channel or | Channel |
| | channel subfamily J member | | transporter | |
| | 16 | | | |
| KCNJ2 | Potassium voltage-gated | 3759 | Channel or | Channel |
| | channel subfamily J member 2 | | transporter | |
| KCNJ3 | Potassium voltage-gated | 3760 | Channel or | Channel |
| | channel subfamily J member 3 | | transporter | |
| KCNJ4 | Potassium voltage-gated | 3761 | Channel or | Channel |
| | channel subfamily J member 4 | | transporter | |
| KCNJ5 | Potassium voltage-gated | 3762 | Channel or | Channel |
| | channel subfamily J member 5 | | transporter | |
| KCNJ6 | Potassium voltage-gated | 3763 | Channel or | Channel |
| | channel subfamily J member 6 | | transporter | |
| KCNJ8 | Potassium voltage-gated | 3764 | Channel or | Channel |
| | channel subfamily J member 8 | | transporter | |
| KCNJ9 | Potassium voltage-gated | 3765 | Channel or | Channel |
| | channel subfamily J member 9 | | transporter | |
| KCNK1 | Potassium two pore domain | 3775 | Channel or | Channel |
| | channel subfamily K member | | transporter | |
| | 1 | | | |
| KCNK10 | Potassium two pore domain | 54207 | Channel or | Channel |
| | channel subfamily K member | | transporter | |
| | 10 | | | |
| KCNK12 | Potassium two pore domain | 56660 | Channel or | Channel |
| | channel subfamily K member | | transporter | |
| | 12 | | | |
| KCNK13 | Potassium two pore domain | 56659 | Channel or | Channel |
| | channel subfamily K member | | transporter | |
| | 13 | | | |
| KCNK15 | Potassium two pore domain | 60598 | Channel or | Channel |
| | channel subfamily K member | | transporter | |
| | 15 | | | |
| KCNK16 | Potassium two pore domain | 83795 | Channel or | Channel |
| | channel subfamily K member | | transporter | |
| | 16 | | | |
| KCNK17 | Potassium two pore domain | 89822 | Channel or | Channel |
| | channel subfamily K member | | transporter | |
| | 17 | | | |
| KCNK18 | Potassium two pore domain | 338567 | Channel or | Channel |
| | channel subfamily K member | | transporter | |
| | 18 | | | |
| KCNK2 | Potassium two pore domain | 3776 | Channel or | Channel |
| | channel subfamily K member | | transporter | |
| | 2 | | | |
| KCNK3 | Potassium two pore domain | 3777 | Channel or | Channel |
| | channel subfamily K member | | transporter | |
| | 3 | | | |
| KCNK4 | Potassium two pore domain | 50801 | Channel or | Channel |
| | channel subfamily K member | | transporter | |
| | 4 | | | |
| KCNK5 | Potassium two pore domain | 8645 | Channel or | Channel |
| | channel subfamily K member | | transporter | |
| | 5 | | | |
| KCNK6 | Potassium two pore domain | 9424 | Channel or | Channel |
| | channel subfamily K member | | transporter | |
| | 6 | | | |
| KCNK7 | Potassium two pore domain | 10089 | Channel or | Channel |
| | channel subfamily K member | | transporter | |
| | 7 | | | |
| KCNK9 | Potassium two pore domain | 51305 | Channel or | Channel |
| | channel subfamily K member | | transporter | |
| | 9 | | | |
| KCNMA1 | Potassium calcium-activated | 3778 | Channel or | Channel |
| | channel subfamily M alpha 1 | | transporter | |
| KCNMB4 | Potassium calcium-activated | 27345 | Channel or | Channel |
| | channel subfamily M | | transporter | |
| | regulatory beta subunit 4 | | | |
| KCNN1 | Potassium calcium-activated | 3780 | Channel or | Channel |
| | channel subfamily N member | | transporter | |
| | 1 | | | |
| KCNN2 | Potassium calcium-activated | 3781 | Channel or | Channel |
| | channel subfamily N member | | transporter | |
| | 2 | | | |
| KCNN3 | Potassium calcium-activated | 3782 | Channel or | Channel |
| | channel subfamily N member | | transporter | |
| | 3 | | | |
| KCNN4 | Potassium calcium-activated | 3783 | Channel or | Channel |
| | channel subfamily N member | | transporter | |
| | 4 | | | |
| KCNQ1 | Potassium voltage-gated | 3784 | Channel or | Channel |
| | channel subfamily Q member | | transporter | |
| | 1 | | | |
| KCNQ2 | Potassium voltage-gated | 3785 | Channel or | Channel |
| | channel subfamily Q member | | transporter | |
| | 2 | | | |
| KCNQ3 | Potassium voltage-gated | 3786 | Channel or | Channel |
| | channel subfamily Q member | | transporter | |
| | 3 | | | |
| KCNQ4 | Potassium voltage-gated | 9132 | Channel or | Channel |
| | channel subfamily Q member | | transporter | |
| | 4 | | | |
| KCNQ5 | Potassium voltage-gated | 56479 | Channel or | Channel |
| | channel subfamily Q member | | transporter | |
| | 5 | | | |
| KCNS1 | Potassium voltage-gated | 3787 | Channel or | Channel |
| | channel modifier subfamily S | | transporter | |
| | member 1 | | | |
| KCNS2 | Potassium voltage-gated | 3788 | Channel or | Channel |
| | channel modifier subfamily S | | transporter | |
| | member 2 | | | |
| KCNS3 | Potassium voltage-gated | 3790 | Channel or | Channel |
| | channel modifier subfamily S | | transporter | |
| | member 3 | | | |
| KCNT1 | Potassium sodium-activated | 57582 | Channel or | Channel |
| | channel subfamily T member | | transporter | |
| | 1 | | | |
| KCNT2 | Potassium sodium-activated | 343450 | Channel or | Channel |
| | channel subfamily T member | | transporter | |
| | 2 | | | |
| KCNU1 | Potassium calcium-activated | 157855 | Channel or | Channel |
| | channel subfamily U member | | transporter | |
| | 1 | | | |
| KCNV1 | Potassium voltage-gated | 27012 | Channel or | Channel |
| | channel modifier subfamily V | | transporter | |
| | member 1 | | | |
| KCNV2 | Potassium voltage-gated | 169522 | Channel or | Channel |
| | channel modifier subfamily V | | transporter | |
| | member 2 | | | |
| KIF1B | Kinesin family member 1B | 23095 | Vesicular | Vesicles |
| KISS1 | Kiss-1 metastasis-suppressor | 3814 | Neuropeptide | Ligand |
| KISS1R | KISS1 receptor | 84634 | Neuropeptide | Receptor |
| KLF16 | Kruppel like factor 16 | 83855 | Other | Miscelaneous |
| KRAS | KRAS proto-oncogene, gtpase | 3845 | Signaling | Signaling |
| L1CAM | L1 cell adhesion molecule | 3897 | Neurotrophic | Signaling |
| LAMTOR3 | Late endosomal/lysosomal | 8649 | Signaling | Signaling |
| | adaptor, MAPK and MTOR | | | |
| | activator 3 | | | |
| LEP | Leptin | 3952 | Neuropeptide | Ligand |
| LHCGR | Luteinizing | 3973 | Neuropeptide | Receptor |
| | hormone/choriogonadotropin | | | |
| | receptor | | | |
| LIF | Leukemia inhibitory factor | 3976 | Neuropeptide | Ligand |
| LIFR | Leukemia inhibitory factor | 3977 | Neurotrophic | Receptor |
| | receptor alpha | | | |
| LIN7A | Lin-7 homolog A, crumbs cell | 8825 | Vesicular | Vesicles |
| | polarity complex component | | | |
| LIN7B | Lin-7 homolog B, crumbs cell | 64130 | Vesicular | Vesicles |
| | polarity complex component | | | |
| LIN7C | Lin-7 homolog C, crumbs cell | 55327 | Vesicular | Vesicles |
| | polarity complex component | | | |
| LPAR3 | Lysophosphatidic acid | 23566 | Other | Miscelaneous |
| | receptor 3 | | | |
| LRP8 | LDL receptor related protein 8 | 7804 | Other | Miscelaneous |
| LRRK2 | Leucine rich repeat kinase 2 | 120892 | Other | Miscelaneous |
| LTB4R | Leukotriene B4 receptor | 1241 | Other | Miscelaneous |
| LTB4R2 | Leukotriene B4 receptor 2 | 56413 | Other | Miscelaneous |
| LYNX1 | Ly6/neurotoxin 1 | 66004 | Neurotransmitter | Receptor |
| MAGED1 | Melanoma-associated antigen | 9500 | Neurotrophic | Signaling |
| | D1 | | | |
| MANF | Mesencephalic astrocyte | 7873 | Neurotrophic | Ligand |
| | derived neurotrophic factor | | | |
| MAOA | Monoamine oxidase A | 4128 | Neurotransmitter | Biosynthesis |
| MAOB | Monoamine oxidase B | 4129 | Neurotransmitter | Biosynthesis |
| MAP2K1 | Mitogen-activated protein | 5604 | Signaling | Signaling |
| | kinase kinase 1 | | | |
| MAP2K2 | Mitogen-activated protein | 5605 | Signaling | Signaling |
| | kinase kinase 2 | | | |
| MAP3K1 | Mitogen-activated protein | 4214 | Signaling | Signaling |
| | kinase kinase kinase 1 | | | |
| MAPK1 | Mitogen-activated protein | 5594 | Signaling | Signaling |
| | kinase 1 | | | |
| MAPK14 | Mitogen-activated protein | 1432 | Signaling | Signaling |
| | kinase 14 | | | |
| MAPK3 | Mitogen-activated protein | 5595 | Signaling | Signaling |
| | kinase 3 | | | |
| MAPK8IP2 | Mitogen-activated protein | 23542 | Signaling | Signaling |
| | kinase 8 interacting protein 2 | | | |
| MC1R | Melanocortin 1 receptor | 4157 | Neuropeptide | Receptor |
| MC2R | Melanocortin 2 receptor | 4158 | Neuropeptide | Receptor |
| MC3R | Melanocortin 3 receptor | 4159 | Neuropeptide | Receptor |
| MC4R | Melanocortin 4 receptor | 4160 | Neuropeptide | Receptor |
| MC5R | Melanocortin 5 receptor | 4161 | Neuropeptide | Receptor |
| MCHR1 | Melanin concentrating | 2847 | Neuropeptide | Receptor |
| | hormone receptor 1 | | | |
| MCHR2 | Melanin concentrating | 84539 | Neuropeptide | Receptor |
| | hormone receptor 2 | | | |
| MCOLN1 | Mucolipin 1 | 57192 | Channel or | Channel |
| | | | transporter | |
| MCOLN2 | Mucolipin 2 | 255231 | Channel or | Channel |
| | | | transporter | |
| MCOLN3 | Mucolipin 3 | 55283 | Channel or | Channel |
| | | | transporter | |
| MEF2C | Myocyte enhancer factor 2C | 4208 | Other | Miscelaneous |
| MFSD7 | Major facilitator superfamily | 84179 | Channel or | Transporter |
| | domain containing 7 | | transporter | |
| MIR204 | Microrna 204 | 406987 | Other | Miscelaneous |
| MLN | Motilin | 4295 | Neuropeptide | Ligand |
| MME | Membrane | 4311 | Neuropeptide | Biosynthesis |
| | metalloendopeptidase | | | |
| MRAP | Melanocortin 2 receptor | 56246 | Neuropeptide | Receptor |
| | accessory protein | | | |
| MRAP2 | Melanocortin 2 receptor | 112609 | Neuropeptide | Receptor |
| | accessory protein 2 | | | |
| MRGPRF | MAS related GPR family | 116535 | Neurotransmitter | Receptor |
| | member F | | | |
| MRGPRX2 | MAS related GPR family | 117194 | Neurotransmitter | Receptor |
| | member X2 | | | |
| MT3 | Metallothionein 3 | 4504 | Other | Miscelaneous |
| MT-ATP6 | Mitochondrially encoded ATP | 4508 | Channel or | Transporter |
| | synthase 6 | | transporter | |
| MT-ATP8 | Mitochondrially encoded ATP | 4509 | Channel or | Transporter |
| | synthase 8 | | transporter | |
| MTCH1 | Mitochondrial carrier 1 | 23787 | Channel or | Transporter |
| | | | transporter | |
| MTCH2 | Mitochondrial carrier 2 | 23788 | Channel or | Transporter |
| | | | transporter | |
| MTNR1A | Melatonin receptor 1A | 4543 | Neuropeptide | Receptor |
| MTNR1B | Melatonin receptor 1B | 4544 | Neuropeptide | Receptor |
| NAAA | N-acylethanolamine acid | 27163 | Vesicular | Vesicles |
| | amidase | | | |
| NAALAD2 | N-acetylated alpha-linked | 10003 | Neuropeptide | Biosynthesis |
| | acidic dipeptidase 2 | | | |
| NALCN | Sodium leak channel, non- | 259232 | Channel or | Channel |
| | selective | | transporter | |
| NAMPT | Nicotinamide | 10135 | Neurotransmitter | Biosynthesis |
| | phosphoribosyltransferase | | | |
| NANOGNB | NANOG neighbor homeobox | 360030 | Vesicular | Vesicles |
| NDNF | Neuron derived neurotrophic | 79625 | Neurotrophic | Ligand |
| | factor | | | |
| NDP | NDP, norrin cystine knot | 4693 | Other | Miscelaneous |
| | growth factor | | | |
| NENF | Neudesin neurotrophic factor | 29937 | Neurotrophic | Ligand |
| NENFP1 | Neudesin neurotrophic factor | 106480294 | Neurotrophic | Ligand |
| | pseudogene 1 | | | |
| NENFP2 | Neudesin neurotrophic factor | 100129880 | Neurotrophic | Ligand |
| | pseudogene 2 | | | |
| NENFP3 | Neudesin neurotrophic factor | 106481703 | Neurotrophic | Ligand |
| | pseudogene 3 | | | |
| NF1 | Neurofibromin 1 | 4763 | Signaling | Signaling |
| NFKB1 | Nuclear factor kappa B | 4790 | Other | Miscelaneous |
| | subunit 1 | | | |
| NGF | Nerve growth factor | 4803 | Neurotrophic | Ligand |
| NGFR | Nerve growth factor receptor | 4804 | Neurotrophic | Receptor |
| NIPSNAP1 | Nipsnap homolog 1 | 8508 | Vesicular | Vesicles |
| | (C. Elegans) | | | |
| NISCH | Nischarin | 11188 | Neurotransmitter | Receptor |
| NLGN1 | Neuroligin 1 | 22871 | Synaptic | Synapse |
| NLGN2 | Neuroligin 2 | 57555 | Synaptic | Synapse |
| NLGN3 | Neuroligin 3 | 54413 | Synaptic | Synapse |
| NLGN4Y | Neuroligin 4, Y-linked | 22829 | Synaptic | Synapse |
| NMB | Neuromedin B | 4828 | Neuropeptide | Ligand |
| NMS | Neuromedin S | 129521 | Neuropeptide | Ligand |
| NMU | Neuromedin U | 10874 | Neuropeptide | Ligand |
| NMUR1 | Neuromedin U receptor 1 | 10316 | Neuropeptide | Receptor |
| NMUR2 | Neuromedin U receptor 2 | 56923 | Neuropeptide | Receptor |
| NOS1 | Nitric oxide synthase 1 | 4842 | Neurotransmitter | Biosynthesis |
| NPB | Neuropeptide B | 256933 | Neuropeptide | Ligand |
| NPBWR1 | Neuropeptides B/W receptor 1 | 2831 | Neuropeptide | Receptor |
| NPBWR2 | Neuropeptides B/W receptor 2 | 2832 | Neuropeptide | Receptor |
| NPC1L1 | NPC1 like intracellular | 29881 | Channel or | Transporter |
| | cholesterol transporter 1 | | transporter | |
| NPFF | Neuropeptide FF-amide | 8620 | Neuropeptide | Ligand |
| | peptide precursor | | | |
| NPFFR1 | Neuropeptide FF receptor 1 | 64106 | Neuropeptide | Receptor |
| NPFFR2 | Neuropeptide FF receptor 2 | 10886 | Neuropeptide | Receptor |
| NPPA | Natriuretic peptide A | 4878 | Neuropeptide | Ligand |
| NPPB | Natriuretic peptide B | 4879 | Neuropeptide | Ligand |
| NPPC | Natriuretic peptide C | 4880 | Neuropeptide | Ligand |
| NPS | Neuropeptide S | 594857 | Neuropeptide | Ligand |
| NPSR1 | Neuropeptide S receptor 1 | 387129 | Neuropeptide | Receptor |
| NPTN | Neuroplastin | 27020 | Neurotransmitter | Receptor |
| NPVF | Neuropeptide VF precursor | 64111 | Neuropeptide | Ligand |
| NPW | Neuropeptide W | 283869 | Neuropeptide | Ligand |
| NPY | Neuropeptide Y | 4852 | Neuropeptide | Ligand |
| NPY1R | Neuropeptide Y receptor Y1 | 4886 | Neuropeptide | Receptor |
| NPY2R | Neuropeptide Y receptor Y2 | 4887 | Neuropeptide | Receptor |
| NPY4R | Neuropeptide Y receptor Y4 | 5540 | Neuropeptide | Receptor |
| NPY5R | Neuropeptide Y receptor Y5 | 4889 | Neuropeptide | Receptor |
| NPY6R | Neuropeptide Y receptor Y6 | 4888 | Neuropeptide | Receptor |
| | (pseudogene) | | | |
| NQO1 | NAD(P)H quinone | 1728 | Other | Miscelaneous |
| | dehydrogenase 1 | | | |
| NR4A2 | Nuclear receptor subfamily 4 | 4929 | Other | Miscelaneous |
| | group A member 2 | | | |
| NRG1 | Neuregulin 1 | 3084 | Neurotrophic | Ligand |
| NRP1 | Neuropilin 1 | 8829 | Neurotrophic | Receptor |
| NRTN | Neurturin | 4902 | Neurotrophic | Ligand |
| NRXN1 | Neurexin 1 | 9378 | Synaptic | Receptor |
| NRXN2 | Neurexin 2 | 9379 | Synaptic | Receptor |
| NRXN3 | Neurexin 3 | 9369 | Synaptic | Receptor |
| NSF | N-ethylmaleimide sensitive | 4905 | Signaling | Signaling |
| | factor, vesicle fusing atpase | | | |
| NTF3 | Neurotrophin 3 | 4908 | Neurotrophic | Ligand |
| NTF4 | Neurotrophin 4 | 4909 | Neurotrophic | Ligand |
| NTRK1 | Neurotrophic receptor tyrosine | 4914 | Neurotrophic | Receptor |
| | kinase 1 | | | |
| NTRK2 | Neurotrophic receptor tyrosine | 4915 | Neurotrophic | Receptor |
| | kinase 2 | | | |
| NTRK3 | Neurotrophic receptor tyrosine | 4916 | Neurotrophic | Receptor |
| | kinase 3 | | | |
| NTS | Neurotensin | 4922 | Neuropeptide | Ligand |
| NTSR1 | Neurotensin receptor 1 | 4923 | Neuropeptide | Receptor |
| NTSR2 | Neurotensin receptor 2 | 23620 | Neuropeptide | Receptor |
| NUCB2 | Nucleobindin 2 | 4925 | Other | Miscelaneous |
| NXPH1 | Neurexophilin 1 | 30010 | Neuropeptide | Ligand |
| NXPH2 | Neurexophilin 2 | 11249 | Neuropeptide | Ligand |
| NXPH3 | Neurexophilin 3 | 11248 | Neuropeptide | Ligand |
| NXPH4 | Neurexophilin 4 | 11247 | Neuropeptide | Ligand |
| OGFR | Opioid growth factor receptor | 11054 | Neuropeptide | Receptor |
| OPHN1 | Oligophrenin 1 | 4983 | Other | Miscelaneous |
| OPRD1 | Opioid receptor delta 1 | 4985 | Neuropeptide | Receptor |
| OPRK1 | Opioid receptor kappa 1 | 4986 | Neuropeptide | Receptor |
| OPRL1 | Opioid related nociceptin | 4987 | Neuropeptide | Receptor |
| | receptor 1 | | | |
| OPRM1 | Opioid receptor mu 1 | 4988 | Neuropeptide | Receptor |
| OTOF | Otoferlin | 9381 | Other | Miscelaneous |
| OXT | Oxytocin/neurophysin I | 5020 | Neuropeptide | Ligand |
| | prepropeptide | | | |
| OXTR | Oxytocin receptor | 5021 | Neuropeptide | Receptor |
| P2RX1 | Purinergic receptor P2X 1 | 5023 | Neurotransmitter | Receptor |
| P2RX2 | Purinergic receptor P2X 2 | 22953 | Neurotransmitter | Receptor |
| P2RX3 | Purinergic receptor P2X 3 | 5024 | Neurotransmitter | Receptor |
| P2RX4 | Purinergic receptor P2X 4 | 5025 | Neurotransmitter | Receptor |
| P2RX5 | Purinergic receptor P2X 5 | 5026 | Neurotransmitter | Receptor |
| P2RX6 | Purinergic receptor P2X 6 | 9127 | Neurotransmitter | Receptor |
| P2RX7 | Purinergic receptor P2X 7 | 5027 | Neurotransmitter | Receptor |
| P2RY11 | Purinergic receptor P2Y11 | 5032 | Neurotransmitter | Receptor |
| PAH | Phenylalanine hydroxylase | 5053 | Neurotransmitter | Biosynthesis |
| PANX1 | Pannexin 1 | 24145 | Channel or | Channel |
| | | | transporter | |
| PANX2 | Pannexin 2 | 56666 | Channel or | Channel |
| | | | transporter | |
| PANX3 | Pannexin 3 | 116337 | Channel or | Channel |
| | | | transporter | |
| PARK2 | Parkin RBR E3 ubiquitin | 5071 | Other | Miscelaneous |
| | protein ligase | | | |
| PARK7 | Parkinsonism associated | 11315 | Other | Miscelaneous |
| | deglycase | | | |
| PATE4 | Prostate and testis expressed | 399968 | Other | Miscelaneous |
| | 4 | | | |
| PC | Pyruvate carboxylase | 5091 | Neurotransmitter | Biosynthesis |
| PCLO | Piccolo presynaptic cytomatrix | 27445 | Other | Miscelaneous |
| | protein | | | |
| PCSK1 | Proprotein convertase | 5122 | Neuropeptide | Biosynthesis |
| | subtilisin/kexin type 1 | | | |
| PCSK1N | Proprotein convertase | 27344 | Neuropeptide | Biosynthesis |
| | subtilisin/kexin type 1 inhibitor | | | |
| PDE1B | Phosphodiesterase 1B | 5153 | Neurotransmitter | Signaling |
| PDE4A | Phosphodiesterase 4A | 5141 | Neurotransmitter | Signaling |
| PDE4D | Phosphodiesterase 4D | 5144 | Neurotransmitter | Signaling |
| PDK1 | Pyruvate dehydrogenase | 5163 | Other | Miscelaneous |
| | kinase 1 | | | |
| PDPK1 | 3-phosphoinositide dependent | 5170 | Neurotrophic | Signaling |
| | protein kinase 1 | | | |
| PDYN | Prodynorphin | 5173 | Other | Miscelaneous |
| PDZD11 | PDZ domain containing 11 | 51248 | Vesicular | Vesicles |
| PENK | Proenkephalin | 5179 | Neuropeptide | Ligand |
| PHOX2A | Paired like homeobox 2a | 401 | Neurotransmitter | Biosynthesis |
| PHOX2B | Paired like homeobox 2b | 8929 | Neurotransmitter | Biosynthesis |
| PIK3CA | Phosphatidylinositol-4,5- | 5290 | Neurotransmitter | Signaling |
| | bisphosphate 3-kinase | | | |
| | catalytic subunit alpha | | | |
| PIK3CB | Phosphatidylinositol-4,5- | 5291 | Neurotransmitter | Signaling |
| | bisphosphate 3-kinase | | | |
| | catalytic subunit beta | | | |
| PIK3CG | Phosphatidylinositol-4,5- | 5294 | Neurotransmitter | Signaling |
| | bisphosphate 3-kinase | | | |
| | catalytic subunit gamma | | | |
| PINK1 | PTEN induced putative kinase | 65018 | Other | Miscelaneous |
| | 1 | | | |
| PITX3 | Paired like homeodomain 3 | 5309 | Other | Miscelaneous |
| PKD2 | Polycystin 2, transient | 5311 | Channel or | Channel |
| | receptor potential cation | | transporter | |
| | channel | | | |
| PKD2L1 | Polycystin 2 like 1, transient | 9033 | Channel or | Channel |
| | receptor potential cation | | transporter | |
| | channel | | | |
| PKD2L2 | Polycystin 2 like 2, transient | 27039 | Channel or | Channel |
| | receptor potential cation | | transporter | |
| | channel | | | |
| PLAT | Plasminogen activator, tissue | 5327 | Other | Miscelaneous |
| | type | | | |
| PLCB1 | Phospholipase C beta 1 | 23236 | Neurotransmitter | Signaling |
| PLCB2 | Phospholipase C beta 2 | 5330 | Neurotransmitter | Signaling |
| PLCB3 | Phospholipase C beta 3 | 5331 | Neurotransmitter | Signaling |
| PLCB4 | Phospholipase C beta 4 | 5332 | Neurotransmitter | Signaling |
| PLCD1 | Phospholipase C delta 1 | 5333 | Neurotransmitter | Signaling |
| PLCE1 | Phospholipase C epsilon 1 | 51196 | Neurotransmitter | Signaling |
| PLCG1 | Phospholipase C gamma 1 | 5335 | Neurotransmitter | Signaling |
| PLCL1 | Phospholipase C like 1 | 5334 | Neurotransmitter | Signaling |
| PLCL2 | Phospholipase C like 2 | 23228 | Neurotransmitter | Signaling |
| PLEKHH3 | Pleckstrin homology, myth4 | 79990 | Neurotrophic | Signaling |
| | and FERM domain containing | | | |
| | H3 | | | |
| PMCH | Pro-melanin concentrating | 5367 | Neuropeptide | Ligand |
| | hormone | | | |
| PNKD | Paroxysmal nonkinesigenic | 25953 | Vesicular | Vesicles |
| | dyskinesia | | | |
| PNOC | Prepronociceptin | 5368 | Neuropeptide | Ligand |
| POMC | Proopiomelanocortin | 5443 | Neuropeptide | Ligand |
| PPARG | Peroxisome proliferator | 5468 | Other | Miscelaneous |
| | activated receptor gamma | | | |
| PPFIA1 | PTPRF interacting protein | 8500 | Vesicular | Vesicles |
| | alpha 1 | | | |
| PPFIA2 | PTPRF interacting protein | 8499 | Vesicular | Vesicles |
| | alpha 2 | | | |
| PPFIA3 | PTPRF interacting protein | 8541 | Vesicular | Vesicles |
| | alpha 3 | | | |
| PPFIA4 | PTPRF interacting protein | 8497 | Vesicular | Vesicles |
| | alpha 4 | | | |
| PPP1CA | Protein phosphatase 1 | 5499 | Signaling | Signaling |
| | catalytic subunit alpha | | | |
| PPP1CB | Protein phosphatase 1 | 5500 | Neurotransmitter | Signaling |
| | catalytic subunit beta | | | |
| PPP1CC | Protein phosphatase 1 | 5501 | Neurotransmitter | Signaling |
| | catalytic subunit gamma | | | |
| PPP1R12A | Protein phosphatase 1 | 4659 | Signaling | Signaling |
| | regulatory subunit 12A | | | |
| PPP1R1B | Protein phosphatase 1 | 84152 | Signaling | Signaling |
| | regulatory inhibitor subunit 1B | | | |
| PPP1R9A | Protein phosphatase 1 | 55607 | Signaling | Signaling |
| | regulatory subunit 9A | | | |
| PPP2CA | Protein phosphatase 2 | 5515 | Signaling | Signaling |
| | catalytic subunit alpha | | | |
| PPP3CA | Protein phosphatase 3 | 5530 | Signaling | Signaling |
| | catalytic subunit alpha | | | |
| PPP3CB | Protein phosphatase 3 | 5532 | Signaling | Signaling |
| | catalytic subunit beta | | | |
| PPP3CC | Protein phosphatase 3 | 5533 | Signaling | Signaling |
| | catalytic subunit gamma | | | |
| PPP3R1 | Protein phosphatase 3 | 5534 | Signaling | Signaling |
| | regulatory subunit B, alpha | | | |
| PPP3R2 | Protein phosphatase 3 | 5535 | Signaling | Signaling |
| | regulatory subunit B, beta | | | |
| PPT1 | Palmitoyl-protein thioesterase | 5538 | Other | Miscelaneous |
| | 1 | | | |
| PPY | Pancreatic polypeptide | 5539 | Neuropeptide | Ligand |
| PPY2P | Pancreatic polypeptide 2, | 23614 | Neuropeptide | Ligand |
| | pseudogene | | | |
| PRIMA1 | Proline rich membrane anchor | 145270 | Neurotransmitter | Biosynthesis |
| | 1 | | | |
| PRKACA | Protein kinase camp-activated | 5566 | Signaling | Signaling |
| | catalytic subunit alpha | | | |
| PRKACB | Protein kinase camp-activated | 5567 | Signaling | Signaling |
| | catalytic subunit beta | | | |
| PRKACG | Protein kinase camp-activated | 5568 | Neurotransmitter | Signaling |
| | catalytic subunit gamma | | | |
| PRKAR1A | Protein kinase camp- | 5573 | Signaling | Signaling |
| | dependent type I regulatory | | | |
| | subunit alpha | | | |
| PRKAR2A | Protein kinase camp- | 5576 | Signaling | Signaling |
| | dependent type II regulatory | | | |
| | subunit alpha | | | |
| PRKAR2B | Protein kinase camp- | 5577 | Neurotransmitter | Signaling |
| | dependent type II regulatory | | | |
| | subunit beta | | | |
| PRKCA | Protein kinase C alpha | 5578 | Signaling | Signaling |
| PRKCD | Protein kinase C delta | 5580 | Signaling | Signaling |
| PRKCE | Protein kinase C epsilon | 5581 | Signaling | Signaling |
| PRKCG | Protein kinase C gamma | 5582 | Neurotransmitter | Signaling |
| PRKX | Protein kinase, X-linked | 5613 | Neurotransmitter | Signaling |
| PRL | Prolactin | 5617 | Neuropeptide | Ligand |
| PRLH | Prolactin releasing hormone | 51052 | Neuropeptide | Ligand |
| PRLHR | Prolactin releasing hormone | 2834 | Neuropeptide | Receptor |
| | receptor | | | |
| PRLR | Prolactin receptor | 5618 | Neuropeptide | Receptor |
| PROK1 | Prokineticin 1 | 84432 | Neuropeptide | Ligand |
| PROK2 | Prokineticin 2 | 60675 | Neuropeptide | Ligand |
| PROKR1 | Prokineticin receptor 1 | 10887 | Neuropeptide | Receptor |
| PROKR2 | Prokineticin receptor 2 | 128674 | Neuropeptide | Receptor |
| PROM1 | Prominin 1 | 8842 | Other | Miscelaneous |
| PSAP | Prosaposin | 5660 | Neurotrophic | Ligand |
| PSEN1 | Presenilin 1 | 5663 | Neurotrophic | Signaling |
| PSPN | Persephin | 5623 | Neurotrophic | Ligand |
| PTEN | Phosphatase and tensin | 5728 | Signaling | Signaling |
| | homolog | | | |
| PTGDR | Prostaglandin D2 receptor | 5729 | Neuropeptide | Receptor |
| PTGDR2 | Prostaglandin D2 receptor 2 | 11251 | Neuropeptide | Receptor |
| PTGER1 | Prostaglandin E receptor 1 | 5731 | Neuropeptide | Receptor |
| PTGER2 | Prostaglandin E receptor 2 | 5732 | Neuropeptide | Receptor |
| PTGER3 | Prostaglandin E receptor 3 | 5733 | Neuropeptide | Receptor |
| PTGER4 | Prostaglandin E receptor 4 | 5734 | Neuropeptide | Receptor |
| PTGFR | Prostaglandin F receptor | 5737 | Neuropeptide | Receptor |
| PTGIR | Prostaglandin I2 (prostacyclin) | 5739 | Neuropeptide | Receptor |
| | receptor (IP) | | | |
| PTGS2 | Prostaglandin-endoperoxide | 5743 | Neuropeptide | Biosynthesis |
| | synthase 2 | | | |
| PTH | Parathyroid hormone | 5741 | Neuropeptide | Ligand |
| PTH1R | Parathyroid hormone 1 | 5745 | Neuropeptide | Receptor |
| | receptor | | | |
| PTH2 | Parathyroid hormone 2 | 113091 | Neuropeptide | Ligand |
| PTH2R | Parathyroid hormone 2 | 5746 | Neuropeptide | Receptor |
| | receptor | | | |
| PTHLH | Parathyroid hormone like | 5744 | Neuropeptide | Ligand |
| | hormone | | | |
| PTK2 | Protein tyrosine kinase 2 | 5747 | Neuropeptide | Signaling |
| PTK2B | Protein tyrosine kinase 2 beta | 2185 | Neuropeptide | Signaling |
| PTN | Pleiotrophin | 5764 | Neurotrophic | Ligand |
| PTPA | Protein phosphatase 2 | 5524 | Signaling | Signaling |
| | phosphatase activator | | | |
| PTPRN2 | Protein tyrosine phosphatase, | 5799 | Other | Miscelaneous |
| | receptor type N2 | | | |
| PXK | PX domain containing | 54899 | Vesicular | Vesicles |
| | serine/threonine kinase like | | | |
| PYY | Peptide YY | 5697 | Neuropeptide | Ligand |
| PYY2 | Peptide YY 2 (pseudogene) | 23615 | Neuropeptide | Ligand |
| PYY3 | Peptide YY 3 (pseudogene) | 644059 | Neuropeptide | Ligand |
| QRFP | Pyroglutamylated rfamide | 347148 | Neuropeptide | Ligand |
| | peptide | | | |
| QRFPR | Pyroglutamylated rfamide | 84109 | Neuropeptide | Receptor |
| | peptide receptor | | | |
| RAB3A | RAB3A, member RAS | 5864 | Signaling | Signaling |
| | oncogene family | | | |
| RAB3GAP1 | RAB3 gtpase activating | 22930 | Vesicular | Vesicles |
| | protein catalytic subunit 1 | | | |
| RAF1 | Raf-1 proto-oncogene, | 5894 | Signaling | Signaling |
| | serine/threonine kinase | | | |
| RAI1 | Retinoic acid induced 1 | 10743 | Other | Miscelaneous |
| RAMP1 | Receptor activity modifying | 10267 | Neuropeptide | Receptor |
| | protein 1 | | | |
| RAMP2 | Receptor activity modifying | 10266 | Neuropeptide | Receptor |
| | protein 2 | | | |
| RAMP3 | Receptor activity modifying | 10268 | Neuropeptide | Receptor |
| | protein 3 | | | |
| RAP1A | RAP1A, member of RAS | 5906 | Signaling | Signaling |
| | oncogene family | | | |
| RAP1GAP | RAP1 gtpase activating | 5909 | Other | Miscelaneous |
| | protein | | | |
| RAPGEF2 | Rap guanine nucleotide | 9693 | Other | Miscelaneous |
| | exchange factor 2 | | | |
| RAPGEF3 | Rap guanine nucleotide | 10411 | Signaling | Signaling |
| | exchange factor 3 | | | |
| RAPSN | Receptor associated protein of | 5913 | Synaptic | Receptor |
| | the synapse | | | |
| RELN | Reelin | 5649 | Neurotrophic | Ligand |
| RET | Ret proto-oncogene | 5979 | Neurotrophic | Signaling |
| RETN | Resistin | 56729 | Other | Miscelaneous |
| RETNLB | Resistin like beta | 84666 | Other | Miscelaneous |
| RGN | Regucalcin | 9104 | Signaling | Signaling |
| RGS10 | Regulator of G-protein | 6001 | Signaling | Signaling |
| | signaling 10 | | | |
| RGS8 | Regulator of G-protein | 85397 | Signaling | Signaling |
| | signaling 8 | | | |
| RGS9 | Regulator of G-protein | 8787 | Signaling | Signaling |
| | signaling 9 | | | |
| RHAG | Rh-associated glycoprotein | 6005 | Channel or | Transporter |
| | | | transporter | |
| RHBG | Rh family B glycoprotein | 57127 | Channel or | Transporter |
| | (gene/pseudogene) | | transporter | |
| RHCG | Rh family C glycoprotein | 51458 | Channel or | Transporter |
| | | | transporter | |
| RHOA | Ras homolog family member | 387 | Signaling | Signaling |
| | A | | | |
| RIC3 | RIC3 acetylcholine receptor | 79608 | Neurotransmitter | Receptor |
| | chaperone | | | |
| RIC8A | RIC8 guanine nucleotide | 60626 | Signaling | Signaling |
| | exchange factor A | | | |
| RIMS1 | Regulating synaptic | 22999 | Vesicular | Vesicles |
| | membrane exocytosis 1 | | | |
| RIMS2 | Regulating synaptic | 9699 | Vesicular | Vesicles |
| | membrane exocytosis 2 | | | |
| RIMS3 | Regulating synaptic | 9783 | Vesicular | Vesicles |
| | membrane exocytosis 3 | | | |
| RIMS4 | Regulating synaptic | 140730 | Vesicular | Vesicles |
| | membrane exocytosis 4 | | | |
| RLN1 | Relaxin 1 | 6013 | Neuropeptide | Ligand |
| RLN2 | Relaxin 2 | 6019 | Neuropeptide | Ligand |
| RLN3 | Relaxin 3 | 117579 | Neuropeptide | Ligand |
| RNF40 | Ring finger protein 40 | 9810 | Other | Miscelaneous |
| ROR1 | Receptor tyrosine kinase like | 4919 | Neurotrophic | Receptor |
| | orphan receptor 1 | | | |
| ROR2 | Receptor tyrosine kinase like | 4920 | Neurotrophic | Receptor |
| | orphan receptor 2 | | | |
| RPH3A | Rabphilin 3A | 22895 | Vesicular | Vesicles |
| RPH3AL | Rabphilin 3A-like (without C2 | 9501 | Vesicular | Vesicles |
| | domains) | | | |
| RPS6KA3 | Ribosomal protein S6 kinase | 6197 | Neurotrophic | Signaling |
| | A3 | | | |
| RPSA | Ribosomal protein SA | 3921 | Other | Miscelaneous |
| RXFP1 | Relaxin/insulin like family | 59350 | Neuropeptide | Receptor |
| | peptide receptor 1 | | | |
| RXFP2 | Relaxin/insulin like family | 122042 | Neuropeptide | Receptor |
| | peptide receptor 2 | | | |
| RXFP3 | Relaxin/insulin like family | 51289 | Neuropeptide | Receptor |
| | peptide receptor 3 | | | |
| RXFP4 | Relaxin/insulin like family | 339403 | Neuropeptide | Receptor |
| | peptide receptor 4 | | | |
| RYR1 | Ryanodine receptor 1 | 6261 | Channel or | Channel |
| | | | transporter | |
| RYR2 | Ryanodine receptor 2 | 6262 | Channel or | Channel |
| | | | transporter | |
| RYR3 | Ryanodine receptor 3 | 6263 | Channel or | Channel |
| | | | transporter | |
| S100B | S100 calcium binding protein | 6285 | Signaling | Signaling |
| | B | | | |
| S1PR4 | Sphingosine-1-phosphate | 8698 | Neuropeptide | Receptor |
| | receptor 4 | | | |
| SCG2 | Secretogranin II | 7857 | Neuropeptide | Vesicles |
| SCG3 | Secretogranin III | 29106 | Neuropeptide | Vesicles |
| SCG5 | Secretogranin V | 6447 | Neuropeptide | Vesicles |
| SCN10A | Sodium voltage-gated channel | 6336 | Channel or | Channel |
| | alpha subunit 10 | | transporter | |
| SCN11A | Sodium voltage-gated channel | 11280 | Channel or | Channel |
| | alpha subunit 11 | | transporter | |
| SCN1A | Sodium voltage-gated channel | 6323 | Channel or | Channel |
| | alpha subunit 1 | | transporter | |
| SCN1B | Sodium voltage-gated channel | 6324 | Channel or | Channel |
| | beta subunit 1 | | transporter | |
| SCN2A | Sodium voltage-gated channel | 6326 | Channel or | Channel |
| | alpha subunit 2 | | transporter | |
| SCN2B | Sodium voltage-gated channel | 6327 | Channel or | Channel |
| | beta subunit 2 | | transporter | |
| SCN3A | Sodium voltage-gated channel | 6328 | Channel or | Channel |
| | alpha subunit 3 | | transporter | |
| SCN3B | Sodium voltage-gated channel | 55800 | Channel or | Channel |
| | beta subunit 3 | | transporter | |
| SCN4A | Sodium voltage-gated channel | 6329 | Channel or | Channel |
| | alpha subunit 4 | | transporter | |
| SCN4B | Sodium voltage-gated channel | 6330 | Channel or | Channel |
| | beta subunit 4 | | transporter | |
| SCN5A | Sodium voltage-gated channel | 6331 | Channel or | Channel |
| | alpha subunit 5 | | transporter | |
| SCN7A | Sodium voltage-gated channel | 6332 | Channel or | Channel |
| | alpha subunit 7 | | transporter | |
| SCN8A | Sodium voltage-gated channel | 6334 | Channel or | Channel |
| | alpha subunit 8 | | transporter | |
| SCN9A | Sodium voltage-gated channel | 6335 | Channel or | Channel |
| | alpha subunit 9 | | transporter | |
| SCNN1A | Sodium channel epithelial 1 | 6337 | Channel or | Channel |
| | alpha subunit | | transporter | |
| SCNN1B | Sodium channel epithelial 1 | 6338 | Channel or | Channel |
| | beta subunit | | transporter | |
| SCNN1D | Sodium channel epithelial 1 | 6339 | Channel or | Channel |
| | delta subunit | | transporter | |
| SCNN1G | Sodium channel epithelial 1 | 6340 | Channel or | Channel |
| | gamma subunit | | transporter | |
| SCT | Secretin | 6343 | Neuropeptide | Ligand |
| SDC3 | Syndecan 3 | 9672 | Neurotrophic | Receptor |
| SEC14L1 | SEC14 like lipid binding 1 | 6397 | Other | Miscelaneous |
| SEMA3E | Semaphorin 3E | 9723 | Neurotrophic | Ligand |
| SERPINE2 | Serpin family E member 2 | 5270 | Neurotrophic | Ligand |
| SERPINF1 | Serpin family F member 1 | 5176 | Neurotrophic | Ligand |
| SHANK3 | SH3 and multiple ankyrin | 85358 | Neurotransmitter | Signaling |
| | repeat domains 3 | | | |
| SHC1 | SHC adaptor protein 1 | 6464 | Neurotrophic | Signaling |
| SHROOM1 | Shroom family member 1 | 134549 | Channel or | Channel |
| | | | transporter | |
| SHROOM2 | Shroom family member 2 | 357 | Channel or | Channel |
| | | | transporter | |
| SHROOM3 | Shroom family member 3 | 57619 | Channel or | Channel |
| | | | transporter | |
| SHROOM4 | Shroom family member 4 | 57477 | Channel or | Channel |
| | | | transporter | |
| SLC10A1 | Solute carrier family 10 | 6554 | Channel or | Transporter |
| | member 1 | | transporter | |
| SLC10A2 | Solute carrier family 10 | 6555 | Channel or | Transporter |
| | member 2 | | transporter | |
| SLC10A3 | Solute carrier family 10 | 8273 | Channel or | Transporter |
| | member 3 | | transporter | |
| SLC10A4 | Solute carrier family 10 | 201780 | Channel or | Transporter |
| | member 4 | | transporter | |
| SLC10A5 | Solute carrier family 10 | 347051 | Channel or | Transporter |
| | member 5 | | transporter | |
| SLC10A6 | Solute carrier family 10 | 345274 | Channel or | Transporter |
| | member 6 | | transporter | |
| SLC10A7 | Solute carrier family 10 | 84068 | Channel or | Transporter |
| | member 7 | | transporter | |
| SLC11A1 | Solute carrier family 11 | 6556 | Channel or | Transporter |
| | member 1 | | transporter | |
| SLC11A2 | Solute carrier family 11 | 4891 | Channel or | Transporter |
| | member 2 | | transporter | |
| SLC12A1 | Solute carrier family 12 | 6557 | Channel or | Transporter |
| | member 1 | | transporter | |
| SLC12A2 | Solute carrier family 12 | 6558 | Channel or | Transporter |
| | member 2 | | transporter | |
| SLC12A3 | Solute carrier family 12 | 6559 | Channel or | Transporter |
| | member 3 | | transporter | |
| SLC12A4 | Solute carrier family 12 | 6560 | Channel or | Transporter |
| | member 4 | | transporter | |
| SLC12A5 | Solute carrier family 12 | 57468 | Channel or | Transporter |
| | member 5 | | transporter | |
| SLC12A6 | Solute carrier family 12 | 9990 | Channel or | Transporter |
| | member 6 | | transporter | |
| SLC12A7 | Solute carrier family 12 | 10723 | Channel or | Transporter |
| | member 7 | | transporter | |
| SLC12A8 | Solute carrier family 12 | 84561 | Channel or | Transporter |
| | member 8 | | transporter | |
| SLC12A9 | Solute carrier family 12 | 56996 | Channel or | Transporter |
| | member 9 | | transporter | |
| SLC13A1 | Solute carrier family 13 | 6561 | Channel or | Transporter |
| | member 1 | | transporter | |
| SLC13A2 | Solute carrier family 13 | 9058 | Channel or | Transporter |
| | member 2 | | transporter | |
| SLC13A3 | Solute carrier family 13 | 64849 | Channel or | Transporter |
| | member 3 | | transporter | |
| SLC13A4 | Solute carrier family 13 | 26266 | Channel or | Transporter |
| | member 4 | | transporter | |
| SLC13A5 | Solute carrier family 13 | 284111 | Channel or | Transporter |
| | member 5 | | transporter | |
| SLC14A1 | Solute carrier family 14 | 6563 | Channel or | Transporter |
| | member 1 (Kidd blood group) | | transporter | |
| SLC14A2 | Solute carrier family 14 | 8170 | Channel or | Transporter |
| | member 2 | | transporter | |
| SLC15A1 | Solute carrier family 15 | 6564 | Channel or | Transporter |
| | member 1 | | transporter | |
| SLC15A2 | Solute carrier family 15 | 6565 | Channel or | Transporter |
| | member 2 | | transporter | |
| SLC15A3 | Solute carrier family 15 | 51296 | Channel or | Transporter |
| | member 3 | | transporter | |
| SLC15A4 | Solute carrier family 15 | 121260 | Channel or | Transporter |
| | member 4 | | transporter | |
| SLC16A1 | Solute carrier family 16 | 6566 | Channel or | Transporter |
| | member 1 | | transporter | |
| SLC16A10 | Solute carrier family 16 | 117247 | Channel or | Transporter |
| | member 10 | | transporter | |
| SLC16A11 | Solute carrier family 16 | 162515 | Channel or | Transporter |
| | member 11 | | transporter | |
| SLC16A12 | Solute carrier family 16 | 387700 | Channel or | Transporter |
| | member 12 | | transporter | |
| SLC16A13 | Solute carrier family 16 | 201232 | Channel or | Transporter |
| | member 13 | | transporter | |
| SLC16A14 | Solute carrier family 16 | 151473 | Channel or | Transporter |
| | member 14 | | transporter | |
| SLC16A2 | Solute carrier family 16 | 6567 | Channel or | Transporter |
| | member 2 | | transporter | |
| SLC16A3 | Solute carrier family 16 | 9123 | Channel or | Transporter |
| | member 3 | | transporter | |
| SLC16A4 | Solute carrier family 16 | 9122 | Channel or | Transporter |
| | member 4 | | transporter | |
| SLC16A5 | Solute carrier family 16 | 9121 | Channel or | Transporter |
| | member 5 | | transporter | |
| SLC16A6 | Solute carrier family 16 | 9120 | Channel or | Transporter |
| | member 6 | | transporter | |
| SLC16A7 | Solute carrier family 16 | 9194 | Channel or | Transporter |
| | member 7 | | transporter | |
| SLC16A8 | Solute carrier family 16 | 23539 | Channel or | Transporter |
| | member 8 | | transporter | |
| SLC16A9 | Solute carrier family 16 | 220963 | Channel or | Transporter |
| | member 9 | | transporter | |
| SLC17A1 | Solute carrier family 17 | 6568 | Channel or | Transporter |
| | member 1 | | transporter | |
| SLC17A2 | Solute carrier family 17 | 10246 | Channel or | Transporter |
| | member 2 | | transporter | |
| SLC17A3 | Solute carrier family 17 | 10786 | Channel or | Transporter |
| | member 3 | | transporter | |
| SLC17A4 | Solute carrier family 17 | 10050 | Channel or | Transporter |
| | member 4 | | transporter | |
| SLC17A5 | Solute carrier family 17 | 26503 | Channel or | Transporter |
| | member 5 | | transporter | |
| SLC17A6 | Solute carrier family 17 | 57084 | Channel or | Transporter |
| | member 6 | | transporter | |
| SLC17A7 | Solute carrier family 17 | 57030 | Channel or | Transporter |
| | member 7 | | transporter | |
| SLC17A8 | Solute carrier family 17 | 246213 | Channel or | Transporter |
| | member 8 | | transporter | |
| SLC17A9 | Solute carrier family 17 | 63910 | Channel or | Transporter |
| | member 9 | | transporter | |
| SLC18A1 | Solute carrier family 18 | 6570 | Channel or | Transporter |
| | member A1 | | transporter | |
| SLC18A2 | Solute carrier family 18 | 6571 | Channel or | Transporter |
| | member A2 | | transporter | |
| SLC18A3 | Solute carrier family 18 | 6572 | Channel or | Transporter |
| | member A3 | | transporter | |
| SLC18B1 | Solute carrier family 18 | 116843 | Channel or | Transporter |
| | member B1 | | transporter | |
| SLC19A1 | Solute carrier family 19 | 6573 | Channel or | Transporter |
| | member 1 | | transporter | |
| SLC19A2 | Solute carrier family 19 | 10560 | Channel or | Transporter |
| | member 2 | | transporter | |
| SLC19A3 | Solute carrier family 19 | 80704 | Channel or | Transporter |
| | member 3 | | transporter | |
| SLC1A1 | Solute carrier family 1 | 6505 | Channel or | Transporter |
| | member 1 | | transporter | |
| SLC1A2 | Solute carrier family 1 | 6506 | Channel or | Transporter |
| | member 2 | | transporter | |
| SLC1A3 | Solute carrier family 1 | 6507 | Channel or | Transporter |
| | member 3 | | transporter | |
| SLC1A6 | Solute carrier family 1 | 6511 | Channel or | Transporter |
| | member 6 | | transporter | |
| SLC1A7 | Solute carrier family 1 | 6512 | Channel or | Transporter |
| | member 7 | | transporter | |
| SLC20A1 | Solute carrier family 20 | 6574 | Channel or | Transporter |
| | member 1 | | transporter | |
| SLC20A2 | Solute carrier family 20 | 6575 | Channel or | Transporter |
| | member 2 | | transporter | |
| SLC22A1 | Solute carrier family 22 | 6580 | Channel or | Transporter |
| | member 1 | | transporter | |
| SLC22A10 | Solute carrier family 22 | 387775 | Channel or | Transporter |
| | member 10 | | transporter | |
| SLC22A11 | Solute carrier family 22 | 55867 | Channel or | Transporter |
| | member 11 | | transporter | |
| SLC22A12 | Solute carrier family 22 | 116085 | Channel or | Transporter |
| | member 12 | | transporter | |
| SLC22A13 | Solute carrier family 22 | 9390 | Channel or | Transporter |
| | member 13 | | transporter | |
| SLC22A14 | Solute carrier family 22 | 9389 | Channel or | Transporter |
| | member 14 | | transporter | |
| SLC22A15 | Solute carrier family 22 | 55356 | Channel or | Transporter |
| | member 15 | | transporter | |
| SLC22A16 | Solute carrier family 22 | 85413 | Channel or | Transporter |
| | member 16 | | transporter | |
| SLC22A17 | Solute carrier family 22 | 51310 | Channel or | Transporter |
| | member 17 | | transporter | |
| SLC22A18 | Solute carrier family 22 | 5002 | Channel or | Transporter |
| | member 18 | | transporter | |
| SLC22A2 | Solute carrier family 22 | 6582 | Channel or | Transporter |
| | member 2 | | transporter | |
| SLC22A20 | Solute carrier family 22 | 440044 | Channel or | Transporter |
| | member 20 | | transporter | |
| SLC22A23 | Solute carrier family 22 | 63027 | Channel or | Transporter |
| | member 23 | | transporter | |
| SLC22A24 | Solute carrier family 22 | 283238 | Channel or | Transporter |
| | member 24 | | transporter | |
| SLC22A25 | Solute carrier family 22 | 387601 | Channel or | Transporter |
| | member 25 | | transporter | |
| SLC22A3 | Solute carrier family 22 | 6581 | Channel or | Transporter |
| | member 3 | | transporter | |
| SLC22A31 | Solute carrier family 22 | 146429 | Channel or | Transporter |
| | member 31 | | transporter | |
| SLC22A4 | Solute carrier family 22 | 6583 | Channel or | Transporter |
| | member 4 | | transporter | |
| SLC22A5 | Solute carrier family 22 | 6584 | Channel or | Transporter |
| | member 5 | | transporter | |
| SLC22A6 | Solute carrier family 22 | 9356 | Channel or | Transporter |
| | member 6 | | transporter | |
| SLC22A7 | Solute carrier family 22 | 10864 | Channel or | Transporter |
| | member 7 | | transporter | |
| SLC22A8 | Solute carrier family 22 | 9376 | Channel or | Transporter |
| | member 8 | | transporter | |
| SLC22A9 | Solute carrier family 22 | 114571 | Channel or | Transporter |
| | member 9 | | transporter | |
| SLC23A1 | Solute carrier family 23 | 9963 | Channel or | Transporter |
| | member 1 | | transporter | |
| SLC23A2 | Solute carrier family 23 | 9962 | Channel or | Transporter |
| | member 2 | | transporter | |
| SLC23A3 | Solute carrier family 23 | 151295 | Channel or | Transporter |
| | member 3 | | transporter | |
| SLC23A4P | Solute carrier family 23 | 641842 | Channel or | Transporter |
| | member 4, pseudogene | | transporter | |
| SLC24A1 | Solute carrier family 24 | 9187 | Channel or | Transporter |
| | member 1 | | transporter | |
| SLC24A2 | Solute carrier family 24 | 25769 | Channel or | Transporter |
| | member 2 | | transporter | |
| SLC24A3 | Solute carrier family 24 | 57419 | Channel or | Transporter |
| | member 3 | | transporter | |
| SLC24A4 | Solute carrier family 24 | 123041 | Channel or | Transporter |
| | member 4 | | transporter | |
| SLC24A5 | Solute carrier family 24 | 283652 | Channel or | Transporter |
| | member 5 | | transporter | |
| SLC25A1 | Solute carrier family 25 | 6576 | Channel or | Transporter |
| | member 1 | | transporter | |
| SLC25A10 | Solute carrier family 25 | 1468 | Channel or | Transporter |
| | member 10 | | transporter | |
| SLC25A11 | Solute carrier family 25 | 8402 | Channel or | Transporter |
| | member 11 | | transporter | |
| SLC25A12 | Solute carrier family 25 | 8604 | Channel or | Transporter |
| | member 12 | | transporter | |
| SLC25A13 | Solute carrier family 25 | 10165 | Channel or | Transporter |
| | member 13 | | transporter | |
| SLC25A14 | Solute carrier family 25 | 9016 | Channel or | Transporter |
| | member 14 | | transporter | |
| SLC25A15 | Solute carrier family 25 | 10166 | Channel or | Transporter |
| | member 15 | | transporter | |
| SLC25A16 | Solute carrier family 25 | 8034 | Channel or | Transporter |
| | member 16 | | transporter | |
| SLC25A17 | Solute carrier family 25 | 10478 | Channel or | Transporter |
| | member 17 | | transporter | |
| SLC25A18 | Solute carrier family 25 | 83733 | Channel or | Transporter |
| | member 18 | | transporter | |
| SLC25A19 | Solute carrier family 25 | 60386 | Channel or | Transporter |
| | member 19 | | transporter | |
| SLC25A2 | Solute carrier family 25 | 83884 | Channel or | Transporter |
| | member 2 | | transporter | |
| SLC25A20 | Solute carrier family 25 | 788 | Channel or | Transporter |
| | member 20 | | transporter | |
| SLC25A21 | Solute carrier family 25 | 89874 | Channel or | Transporter |
| | member 21 | | transporter | |
| SLC25A22 | Solute carrier family 25 | 79751 | Channel or | Transporter |
| | member 22 | | transporter | |
| SLC25A23 | Solute carrier family 25 | 79085 | Channel or | Transporter |
| | member 23 | | transporter | |
| SLC25A24 | Solute carrier family 25 | 29957 | Channel or | Transporter |
| | member 24 | | transporter | |
| SLC25A25 | Solute carrier family 25 | 114789 | Channel or | Transporter |
| | member 25 | | transporter | |
| SLC25A26 | Solute carrier family 25 | 115286 | Channel or | Transporter |
| | member 26 | | transporter | |
| SLC25A27 | Solute carrier family 25 | 9481 | Channel or | Transporter |
| | member 27 | | transporter | |
| SLC25A28 | Solute carrier family 25 | 81894 | Channel or | Transporter |
| | member 28 | | transporter | |
| SLC25A29 | Solute carrier family 25 | 123096 | Channel or | Transporter |
| | member 29 | | transporter | |
| SLC25A3 | Solute carrier family 25 | 5250 | Channel or | Transporter |
| | member 3 | | transporter | |
| SLC25A30 | Solute carrier family 25 | 253512 | Channel or | Transporter |
| | member 30 | | transporter | |
| SLC25A31 | Solute carrier family 25 | 83447 | Channel or | Transporter |
| | member 31 | | transporter | |
| SLC25A32 | Solute carrier family 25 | 81034 | Channel or | Transporter |
| | member 32 | | transporter | |
| SLC25A33 | Solute carrier family 25 | 84275 | Channel or | Transporter |
| | member 33 | | transporter | |
| SLC25A34 | Solute carrier family 25 | 284723 | Channel or | Transporter |
| | member 34 | | transporter | |
| SLC25A35 | Solute carrier family 25 | 399512 | Channel or | Transporter |
| | member 35 | | transporter | |
| SLC25A36 | Solute carrier family 25 | 55186 | Channel or | Transporter |
| | member 36 | | transporter | |
| SLC25A37 | Solute carrier family 25 | 51312 | Channel or | Transporter |
| | member 37 | | transporter | |
| SLC25A38 | Solute carrier family 25 | 54977 | Channel or | Transporter |
| | member 38 | | transporter | |
| SLC25A39 | Solute carrier family 25 | 51629 | Channel or | Transporter |
| | member 39 | | transporter | |
| SLC25A4 | Solute carrier family 25 | 291 | Channel or | Transporter |
| | member 4 | | transporter | |
| SLC25A40 | Solute carrier family 25 | 55972 | Channel or | Transporter |
| | member 40 | | transporter | |
| SLC25A41 | Solute carrier family 25 | 284427 | Channel or | Transporter |
| | member 41 | | transporter | |
| SLC25A42 | Solute carrier family 25 | 284439 | Channel or | Transporter |
| | member 42 | | transporter | |
| SLC25A43 | Solute carrier family 25 | 203427 | Channel or | Transporter |
| | member 43 | | transporter | |
| SLC25A44 | Solute carrier family 25 | 9673 | Channel or | Transporter |
| | member 44 | | transporter | |
| SLC25A45 | Solute carrier family 25 | 283130 | Channel or | Transporter |
| | member 45 | | transporter | |
| SLC25A46 | Solute carrier family 25 | 91137 | Channel or | Transporter |
| | member 46 | | transporter | |
| SLC25A47 | Solute carrier family 25 | 283600 | Channel or | Transporter |
| | member 47 | | transporter | |
| SLC25A48 | Solute carrier family 25 | 153328 | Channel or | Transporter |
| | member 48 | | transporter | |
| SLC25A5 | Solute carrier family 25 | 292 | Channel or | Transporter |
| | member 5 | | transporter | |
| SLC25A51 | Solute carrier family 25 | 92014 | Channel or | Transporter |
| | member 51 | | transporter | |
| SLC25A52 | Solute carrier family 25 | 147407 | Channel or | Transporter |
| | member 52 | | transporter | |
| SLC25A53 | Solute carrier family 25 | 401612 | Channel or | Transporter |
| | member 53 | | transporter | |
| SLC25A6 | Solute carrier family 25 | 293 | Channel or | Transporter |
| | member 6 | | transporter | |
| SLC26A1 | Solute carrier family 26 | 10861 | Channel or | Transporter |
| | member 1 | | transporter | |
| SLC26A10 | Solute carrier family 26 | 65012 | Channel or | Transporter |
| | member 10 | | transporter | |
| SLC26A11 | Solute carrier family 26 | 284129 | Channel or | Transporter |
| | member 11 | | transporter | |
| SLC26A2 | Solute carrier family 26 | 1836 | Channel or | Transporter |
| | member 2 | | transporter | |
| SLC26A3 | Solute carrier family 26 | 1811 | Channel or | Transporter |
| | member 3 | | transporter | |
| SLC26A4 | Solute carrier family 26 | 5172 | Channel or | Transporter |
| | member 4 | | transporter | |
| SLC26A5 | Solute carrier family 26 | 375611 | Channel or | Transporter |
| | member 5 | | transporter | |
| SLC26A6 | Solute carrier family 26 | 65010 | Channel or | Transporter |
| | member 6 | | transporter | |
| SLC26A7 | Solute carrier family 26 | 115111 | Channel or | Transporter |
| | member 7 | | transporter | |
| SLC26A8 | Solute carrier family 26 | 116369 | Channel or | Transporter |
| | member 8 | | transporter | |
| SLC26A9 | Solute carrier family 26 | 115019 | Channel or | Transporter |
| | member 9 | | transporter | |
| SLC27A1 | Solute carrier family 27 | 376497 | Channel or | Transporter |
| | member 1 | | transporter | |
| SLC27A2 | Solute carrier family 27 | 11001 | Channel or | Transporter |
| | member 2 | | transporter | |
| SLC27A3 | Solute carrier family 27 | 11000 | Channel or | Transporter |
| | member 3 | | transporter | |
| SLC27A4 | Solute carrier family 27 | 10999 | Channel or | Transporter |
| | member 4 | | transporter | |
| SLC27A5 | Solute carrier family 27 | 10998 | Channel or | Transporter |
| | member 5 | | transporter | |
| SLC27A6 | Solute carrier family 27 | 28965 | Channel or | Transporter |
| | member 6 | | transporter | |
| SLC28A1 | Solute carrier family 28 | 9154 | Channel or | Transporter |
| | member 1 | | transporter | |
| SLC28A2 | Solute carrier family 28 | 9153 | Channel or | Transporter |
| | member 2 | | transporter | |
| SLC28A3 | Solute carrier family 28 | 64078 | Channel or | Transporter |
| | member 3 | | transporter | |
| SLC29A1 | Solute carrier family 29 | 2030 | Channel or | Transporter |
| | member 1 (Augustine blood | | transporter | |
| | group) | | | |
| SLC29A2 | Solute carrier family 29 | 3177 | Channel or | Transporter |
| | member 2 | | transporter | |
| SLC29A3 | Solute carrier family 29 | 55315 | Channel or | Transporter |
| | member 3 | | transporter | |
| SLC29A4 | Solute carrier family 29 | 222962 | Channel or | Transporter |
| | member 4 | | transporter | |
| SLC2A1 | Solute carrier family 2 | 6513 | Channel or | Transporter |
| | member 1 | | transporter | |
| SLC2A10 | Solute carrier family 2 | 81031 | Channel or | Transporter |
| | member 10 | | transporter | |
| SLC2A11 | Solute carrier family 2 | 66035 | Channel or | Transporter |
| | member 11 | | transporter | |
| SLC2A12 | Solute carrier family 2 | 154091 | Channel or | Transporter |
| | member 12 | | transporter | |
| SLC2A13 | Solute carrier family 2 | 114134 | Channel or | Transporter |
| | member 13 | | transporter | |
| SLC2A14 | Solute carrier family 2 | 144195 | Channel or | Transporter |
| | member 14 | | transporter | |
| SLC2A2 | Solute carrier family 2 | 6514 | Channel or | Transporter |
| | member 2 | | transporter | |
| SLC2A3 | Solute carrier family 2 | 6515 | Channel or | Transporter |
| | member 3 | | transporter | |
| SLC2A4 | Solute carrier family 2 | 6517 | Channel or | Transporter |
| | member 4 | | transporter | |
| SLC2A5 | Solute carrier family 2 | 6518 | Channel or | Transporter |
| | member 5 | | transporter | |
| SLC2A6 | Solute carrier family 2 | 11182 | Channel or | Transporter |
| | member 6 | | transporter | |
| SLC2A7 | Solute carrier family 2 | 155184 | Channel or | Transporter |
| | member 7 | | transporter | |
| SLC2A8 | Solute carrier family 2 | 29988 | Channel or | Transporter |
| | member 8 | | transporter | |
| SLC2A9 | Solute carrier family 2 | 56606 | Channel or | Transporter |
| | member 9 | | transporter | |
| SLC30A1 | Solute carrier family 30 | 7779 | Channel or | Transporter |
| | member 1 | | transporter | |
| SLC30A10 | Solute carrier family 30 | 55532 | Channel or | Transporter |
| | member 10 | | transporter | |
| SLC30A2 | Solute carrier family 30 | 7780 | Channel or | Transporter |
| | member 2 | | transporter | |
| SLC30A3 | Solute carrier family 30 | 7781 | Channel or | Transporter |
| | member 3 | | transporter | |
| SLC30A4 | Solute carrier family 30 | 7782 | Channel or | Transporter |
| | member 4 | | transporter | |
| SLC30A5 | Solute carrier family 30 | 64924 | Channel or | Transporter |
| | member 5 | | transporter | |
| SLC30A6 | Solute carrier family 30 | 55676 | Channel or | Transporter |
| | member 6 | | transporter | |
| SLC30A7 | Solute carrier family 30 | 148867 | Channel or | Transporter |
| | member 7 | | transporter | |
| SLC30A8 | Solute carrier family 30 | 169026 | Channel or | Transporter |
| | member 8 | | transporter | |
| SLC30A9 | Solute carrier family 30 | 10463 | Channel or | Transporter |
| | member 9 | | transporter | |
| SLC31A1 | Solute carrier family 31 | 1317 | Channel or | Transporter |
| | member 1 | | transporter | |
| SLC31A2 | Solute carrier family 31 | 1318 | Channel or | Transporter |
| | member 2 | | transporter | |
| SLC32A1 | Solute carrier family 32 | 140679 | Channel or | Transporter |
| | member 1 | | transporter | |
| SLC33A1 | Solute carrier family 33 | 9197 | Channel or | Transporter |
| | member 1 | | transporter | |
| SLC34A1 | Solute carrier family 34 | 6569 | Channel or | Transporter |
| | member 1 | | transporter | |
| SLC34A2 | Solute carrier family 34 | 10568 | Channel or | Transporter |
| | member 2 | | transporter | |
| SLC34A3 | Solute carrier family 34 | 142680 | Channel or | Transporter |
| | member 3 | | transporter | |
| SLC35A1 | Solute carrier family 35 | 10559 | Channel or | Transporter |
| | member A1 | | transporter | |
| SLC35A2 | Solute carrier family 35 | 7355 | Channel or | Transporter |
| | member A2 | | transporter | |
| SLC35A3 | Solute carrier family 35 | 23443 | Channel or | Transporter |
| | member A3 | | transporter | |
| SLC35A4 | Solute carrier family 35 | 113829 | Channel or | Transporter |
| | member A4 | | transporter | |
| SLC35A5 | Solute carrier family 35 | 55032 | Channel or | Transporter |
| | member A5 | | transporter | |
| SLC35B1 | Solute carrier family 35 | 10237 | Channel or | Transporter |
| | member B1 | | transporter | |
| SLC35B2 | Solute carrier family 35 | 347734 | Channel or | Transporter |
| | member B2 | | transporter | |
| SLC35B3 | Solute carrier family 35 | 51000 | Channel or | Transporter |
| | member B3 | | transporter | |
| SLC35B4 | Solute carrier family 35 | 84912 | Channel or | Transporter |
| | member B4 | | transporter | |
| SLC35C1 | Solute carrier family 35 | 55343 | Channel or | Transporter |
| | member C1 | | transporter | |
| SLC35C2 | Solute carrier family 35 | 51006 | Channel or | Transporter |
| | member C2 | | transporter | |
| SLC35D1 | Solute carrier family 35 | 23169 | Channel or | Transporter |
| | member D1 | | transporter | |
| SLC35D2 | Solute carrier family 35 | 11046 | Channel or | Transporter |
| | member D2 | | transporter | |
| SLC35D3 | Solute carrier family 35 | 340146 | Channel or | Transporter |
| | member D3 | | transporter | |
| SLC35E1 | Solute carrier family 35 | 79939 | Channel or | Transporter |
| | member E1 | | transporter | |
| SLC35E2 | Solute carrier family 35 | 9906 | Channel or | Transporter |
| | member E2 | | transporter | |
| SLC35E2B | Solute carrier family 35 | 728661 | Channel or | Transporter |
| | member E2B | | transporter | |
| SLC35E3 | Solute carrier family 35 | 55508 | Channel or | Transporter |
| | member E3 | | transporter | |
| SLC35E4 | Solute carrier family 35 | 339665 | Channel or | Transporter |
| | member E4 | | transporter | |
| SLC35F1 | Solute carrier family 35 | 222553 | Channel or | Transporter |
| | member F1 | | transporter | |
| SLC35F2 | Solute carrier family 35 | 54733 | Channel or | Transporter |
| | member F2 | | transporter | |
| SLC35F3 | Solute carrier family 35 | 148641 | Channel or | Transporter |
| | member F3 | | transporter | |
| SLC35F4 | Solute carrier family 35 | 341880 | Channel or | Transporter |
| | member F4 | | transporter | |
| SLC35F5 | Solute carrier family 35 | 80255 | Channel or | Transporter |
| | member F5 | | transporter | |
| SLC35F6 | Solute carrier family 35 | 54978 | Channel or | Transporter |
| | member F6 | | transporter | |
| SLC35G1 | Solute carrier family 35 | 159371 | Channel or | Transporter |
| | member G1 | | transporter | |
| SLC35G2 | Solute carrier family 35 | 80723 | Channel or | Transporter |
| | member G2 | | transporter | |
| SLC35G3 | Solute carrier family 35 | 146861 | Channel or | Transporter |
| | member G3 | | transporter | |
| SLC35G4 | Solute carrier family 35 | 646000 | Channel or | Transporter |
| | member G4 | | transporter | |
| SLC35G5 | Solute carrier family 35 | 83650 | Channel or | Transporter |
| | member G5 | | transporter | |
| SLC35G6 | Solute carrier family 35 | 643664 | Channel or | Transporter |
| | member G6 | | transporter | |
| SLC36A1 | Solute carrier family 36 | 206358 | Channel or | Transporter |
| | member 1 | | transporter | |
| SLC36A2 | Solute carrier family 36 | 153201 | Channel or | Transporter |
| | member 2 | | transporter | |
| SLC36A3 | Solute carrier family 36 | 285641 | Channel or | Transporter |
| | member 3 | | transporter | |
| SLC36A4 | Solute carrier family 36 | 120103 | Channel or | Transporter |
| | member 4 | | transporter | |
| SLC37A1 | Solute carrier family 37 | 54020 | Channel or | Transporter |
| | member 1 | | transporter | |
| SLC37A2 | Solute carrier family 37 | 219855 | Channel or | Transporter |
| | member 2 | | transporter | |
| SLC37A3 | Solute carrier family 37 | 84255 | Channel or | Transporter |
| | member 3 | | transporter | |
| SLC37A4 | Solute carrier family 37 | 2542 | Channel or | Transporter |
| | member 4 | | transporter | |
| SLC38A1 | Solute carrier family 38 | 81539 | Channel or | Transporter |
| | member 1 | | transporter | |
| SLC38A10 | Solute carrier family 38 | 124565 | Channel or | Transporter |
| | member 10 | | transporter | |
| SLC38A11 | Solute carrier family 38 | 151258 | Channel or | Transporter |
| | member 11 | | transporter | |
| SLC38A2 | Solute carrier family 38 | 54407 | Channel or | Transporter |
| | member 2 | | transporter | |
| SLC38A3 | Solute carrier family 38 | 10991 | Channel or | Transporter |
| | member 3 | | transporter | |
| SLC38A4 | Solute carrier family 38 | 55089 | Channel or | Transporter |
| | member 4 | | transporter | |
| SLC38A5 | Solute carrier family 38 | 92745 | Channel or | Transporter |
| | member 5 | | transporter | |
| SLC38A6 | Solute carrier family 38 | 145389 | Channel or | Transporter |
| | member 6 | | transporter | |
| SLC38A7 | Solute carrier family 38 | 55238 | Channel or | Transporter |
| | member 7 | | transporter | |
| SLC38A8 | Solute carrier family 38 | 146167 | Channel or | Transporter |
| | member 8 | | transporter | |
| SLC38A9 | Solute carrier family 38 | 153129 | Channel or | Transporter |
| | member 9 | | transporter | |
| SLC39A1 | Solute carrier family 39 | 27173 | Channel or | Transporter |
| | member 1 | | transporter | |
| SLC39A10 | Solute carrier family 39 | 57181 | Channel or | Transporter |
| | member 10 | | transporter | |
| SLC39A11 | Solute carrier family 39 | 201266 | Channel or | Transporter |
| | member 11 | | transporter | |
| SLC39A12 | Solute carrier family 39 | 221074 | Channel or | Transporter |
| | member 12 | | transporter | |
| SLC39A13 | Solute carrier family 39 | 91252 | Channel or | Transporter |
| | member 13 | | transporter | |
| SLC39A14 | Solute carrier family 39 | 23516 | Channel or | Transporter |
| | member 14 | | transporter | |
| SLC39A2 | Solute carrier family 39 | 29986 | Channel or | Transporter |
| | member 2 | | transporter | |
| SLC39A3 | Solute carrier family 39 | 29985 | Channel or | Transporter |
| | member 3 | | transporter | |
| SLC39A4 | Solute carrier family 39 | 55630 | Channel or | Transporter |
| | member 4 | | transporter | |
| SLC39A5 | Solute carrier family 39 | 283375 | Channel or | Transporter |
| | member 5 | | transporter | |
| SLC39A6 | Solute carrier family 39 | 25800 | Channel or | Transporter |
| | member 6 | | transporter | |
| SLC39A7 | Solute carrier family 39 | 7922 | Channel or | Transporter |
| | member 7 | | transporter | |
| SLC39A8 | Solute carrier family 39 | 64116 | Channel or | Transporter |
| | member 8 | | transporter | |
| SLC39A9 | Solute carrier family 39 | 55334 | Channel or | Transporter |
| | member 9 | | transporter | |
| SLC3A1 | Solute carrier family 3 | 6519 | Channel or | Transporter |
| | member 1 | | transporter | |
| SLC3A2 | Solute carrier family 3 | 6520 | Channel or | Transporter |
| | member 2 | | transporter | |
| SLC40A1 | Solute carrier family 40 | 30061 | Channel or | Transporter |
| | member 1 | | transporter | |
| SLC41A1 | Solute carrier family 41 | 254428 | Channel or | Transporter |
| | member 1 | | transporter | |
| SLC41A2 | Solute carrier family 41 | 84102 | Channel or | Transporter |
| | member 2 | | transporter | |
| SLC41A3 | Solute carrier family 41 | 54946 | Channel or | Transporter |
| | member 3 | | transporter | |
| SLC43A1 | Solute carrier family 43 | 8501 | Channel or | Transporter |
| | member 1 | | transporter | |
| SLC43A2 | Solute carrier family 43 | 124935 | Channel or | Transporter |
| | member 2 | | transporter | |
| SLC43A3 | Solute carrier family 43 | 29015 | Channel or | Transporter |
| | member 3 | | transporter | |
| SLC44A1 | Solute carrier family 44 | 23446 | Channel or | Transporter |
| | member 1 | | transporter | |
| SLC44A2 | Solute carrier family 44 | 57153 | Channel or | Transporter |
| | member 2 | | transporter | |
| SLC44A3 | Solute carrier family 44 | 126969 | Channel or | Transporter |
| | member 3 | | transporter | |
| SLC44A4 | Solute carrier family 44 | 80736 | Channel or | Transporter |
| | member 4 | | transporter | |
| SLC44A5 | Solute carrier family 44 | 204962 | Channel or | Transporter |
| | member 5 | | transporter | |
| SLC45A1 | Solute carrier family 45 | 50651 | Channel or | Transporter |
| | member 1 | | transporter | |
| SLC45A2 | Solute carrier family 45 | 51151 | Channel or | Transporter |
| | member 2 | | transporter | |
| SLC45A3 | Solute carrier family 45 | 85414 | Channel or | Transporter |
| | member 3 | | transporter | |
| SLC45A4 | Solute carrier family 45 | 57210 | Channel or | Transporter |
| | member 4 | | transporter | |
| SLC46A1 | Solute carrier family 46 | 113235 | Channel or | Transporter |
| | member 1 | | transporter | |
| SLC46A2 | Solute carrier family 46 | 57864 | Channel or | Transporter |
| | member 2 | | transporter | |
| SLC46A3 | Solute carrier family 46 | 283537 | Channel or | Transporter |
| | member 3 | | transporter | |
| SLC47A1 | Solute carrier family 47 | 55244 | Channel or | Transporter |
| | member 1 | | transporter | |
| SLC47A2 | Solute carrier family 47 | 146802 | Channel or | Transporter |
| | member 2 | | transporter | |
| SLC48A1 | Solute carrier family 48 | 55652 | Channel or | Transporter |
| | member 1 | | transporter | |
| SLC4A1 | Solute carrier family 4 | 6521 | Channel or | Transporter |
| | member 1 | | transporter | |
| SLC4A10 | Solute carrier family 4 | 57282 | Channel or | Transporter |
| | member 10 | | transporter | |
| SLC4A11 | Solute carrier family 4 | 83959 | Channel or | Transporter |
| | member 11 | | transporter | |
| SLC4A2 | Solute carrier family 4 | 6522 | Channel or | Transporter |
| | member 2 | | transporter | |
| SLC4A3 | Solute carrier family 4 | 6508 | Channel or | Transporter |
| | member 3 | | transporter | |
| SLC4A4 | Solute carrier family 4 | 8671 | Channel or | Transporter |
| | member 4 | | transporter | |
| SLC4A5 | Solute carrier family 4 | 57835 | Channel or | Transporter |
| | member 5 | | transporter | |
| SLC4A7 | Solute carrier family 4 | 9497 | Channel or | Transporter |
| | member 7 | | transporter | |
| SLC4A8 | Solute carrier family 4 | 9498 | Channel or | Transporter |
| | member 8 | | transporter | |
| SLC4A9 | Solute carrier family 4 | 83697 | Channel or | Transporter |
| | member 9 | | transporter | |
| SLC50A1 | Solute carrier family 50 | 55974 | Channel or | Transporter |
| | member 1 | | transporter | |
| SLC51A | Solute carrier family 51 alpha | 200931 | Channel or | Transporter |
| | subunit | | transporter | |
| SLC51B | Solute carrier family 51 beta | 123264 | Channel or | Transporter |
| | subunit | | transporter | |
| SLC52A1 | Solute carrier family 52 | 55065 | Channel or | Transporter |
| | member 1 | | transporter | |
| SLC52A2 | Solute carrier family 52 | 79581 | Channel or | Transporter |
| | member 2 | | transporter | |
| SLC52A3 | Solute carrier family 52 | 113278 | Channel or | Transporter |
| | member 3 | | transporter | |
| SLC5A1 | Solute carrier family 5 | 6523 | Channel or | Transporter |
| | member 1 | | transporter | |
| SLC5A10 | Solute carrier family 5 | 125206 | Channel or | Transporter |
| | member 10 | | transporter | |
| SLC5A11 | Solute carrier family 5 | 115584 | Channel or | Transporter |
| | member 11 | | transporter | |
| SLC5A12 | Solute carrier family 5 | 159963 | Channel or | Transporter |
| | member 12 | | transporter | |
| SLC5A2 | Solute carrier family 5 | 6524 | Channel or | Transporter |
| | member 2 | | transporter | |
| SLC5A3 | Solute carrier family 5 | 6526 | Channel or | Transporter |
| | member 3 | | transporter | |
| SLC5A4 | Solute carrier family 5 | 6527 | Channel or | Transporter |
| | member 4 | | transporter | |
| SLC5A5 | Solute carrier family 5 | 6528 | Channel or | Transporter |
| | member 5 | | transporter | |
| SLC5A6 | Solute carrier family 5 | 8884 | Channel or | Transporter |
| | member 6 | | transporter | |
| SLC5A7 | Solute carrier family 5 | 60482 | Channel or | Transporter |
| | member 7 | | transporter | |
| SLC5A8 | Solute carrier family 5 | 160728 | Channel or | Transporter |
| | member 8 | | transporter | |
| SLC5A9 | Solute carrier family 5 | 200010 | Channel or | Transporter |
| | member 9 | | transporter | |
| SLC6A1 | Solute carrier family 6 | 6529 | Channel or | Transporter |
| | member 1 | | transporter | |
| SLC6A10P | Solute carrier family 6 | 386757 | Channel or | Transporter |
| | member 10, pseudogene | | transporter | |
| SLC6A10PB | Solute carrier family 6 | 653562 | Channel or | Transporter |
| | member 8 pseudogene | | transporter | |
| SLC6A11 | Solute carrier family 6 | 6538 | Channel or | Transporter |
| | member 11 | | transporter | |
| SLC6A12 | Solute carrier family 6 | 6539 | Channel or | Transporter |
| | member 12 | | transporter | |
| SLC6A13 | Solute carrier family 6 | 6540 | Channel or | Transporter |
| | member 13 | | transporter | |
| SLC6A14 | Solute carrier family 6 | 11254 | Channel or | Transporter |
| | member 14 | | transporter | |
| SLC6A15 | Solute carrier family 6 | 55117 | Channel or | Transporter |
| | member 15 | | transporter | |
| SLC6A16 | Solute carrier family 6 | 28968 | Channel or | Transporter |
| | member 16 | | transporter | |
| SLC6A17 | Solute carrier family 6 | 388662 | Channel or | Transporter |
| | member 17 | | transporter | |
| SLC6A18 | Solute carrier family 6 | 348932 | Channel or | Transporter |
| | member 18 | | transporter | |
| SLC6A19 | Solute carrier family 6 | 340024 | Channel or | Transporter |
| | member 19 | | transporter | |
| SLC6A2 | Solute carrier family 6 | 6530 | Channel or | Transporter |
| | member 2 | | transporter | |
| SLC6A20 | Solute carrier family 6 | 54716 | Channel or | Transporter |
| | member 20 | | transporter | |
| SLC6A21P | Solute carrier family 6 | 652969 | Channel or | Transporter |
| | member 21, pseudogene | | transporter | |
| SLC6A3 | Solute carrier family 6 | 6531 | Channel or | Transporter |
| | member 3 | | transporter | |
| SLC6A4 | Solute carrier family 6 | 6532 | Channel or | Transporter |
| | member 4 | | transporter | |
| SLC6A5 | Solute carrier family 6 | 9152 | Channel or | Transporter |
| | member 5 | | transporter | |
| SLC6A6 | Solute carrier family 6 | 6533 | Channel or | Transporter |
| | member 6 | | transporter | |
| SLC6A7 | Solute carrier family 6 | 6534 | Channel or | Transporter |
| | member 7 | | transporter | |
| SLC6A8 | Solute carrier family 6 | 6535 | Channel or | Transporter |
| | member 8 | | transporter | |
| SLC6A9 | Solute carrier family 6 | 6536 | Channel or | Transporter |
| | member 9 | | transporter | |
| SLC7A1 | Solute carrier family 7 | 6541 | Channel or | Transporter |
| | member 1 | | transporter | |
| SLC7A10 | Solute carrier family 7 | 56301 | Channel or | Transporter |
| | member 10 | | transporter | |
| SLC7A11 | Solute carrier family 7 | 23657 | Channel or | Transporter |
| | member 11 | | transporter | |
| SLC7A13 | Solute carrier family 7 | 157724 | Channel or | Transporter |
| | member 13 | | transporter | |
| SLC7A14 | Solute carrier family 7 | 57709 | Channel or | Transporter |
| | member 14 | | transporter | |
| SLC7A2 | Solute carrier family 7 | 6542 | Channel or | Transporter |
| | member 2 | | transporter | |
| SLC7A3 | Solute carrier family 7 | 84889 | Channel or | Transporter |
| | member 3 | | transporter | |
| SLC7A4 | Solute carrier family 7 | 6545 | Channel or | Transporter |
| | member 4 | | transporter | |
| SLC7A5 | Solute carrier family 7 | 8140 | Channel or | Transporter |
| | member 5 | | transporter | |
| SLC7A6 | Solute carrier family 7 | 9057 | Channel or | Transporter |
| | member 6 | | transporter | |
| SLC7A7 | Solute carrier family 7 | 9056 | Channel or | Transporter |
| | member 7 | | transporter | |
| SLC7A8 | Solute carrier family 7 | 23428 | Channel or | Transporter |
| | member 8 | | transporter | |
| SLC7A9 | Solute carrier family 7 | 11136 | Channel or | Transporter |
| | member 9 | | transporter | |
| SLC8A1 | Solute carrier family 8 | 6546 | Channel or | Transporter |
| | member A1 | | transporter | |
| SLC8A2 | Solute carrier family 8 | 6543 | Channel or | Transporter |
| | member A2 | | transporter | |
| SLC8A3 | Solute carrier family 8 | 6547 | Channel or | Transporter |
| | member A3 | | transporter | |
| SLC8B1 | Solute carrier family 8 | 80024 | Channel or | Transporter |
| | member B1 | | transporter | |
| SLC9A1 | Solute carrier family 9 | 6548 | Channel or | Transporter |
| | member A1 | | transporter | |
| SLC9A2 | Solute carrier family 9 | 6549 | Channel or | Transporter |
| | member A2 | | transporter | |
| SLC9A3 | Solute carrier family 9 | 6550 | Channel or | Transporter |
| | member A3 | | transporter | |
| SLC9A4 | Solute carrier family 9 | 389015 | Channel or | Transporter |
| | member A4 | | transporter | |
| SLC9A5 | Solute carrier family 9 | 6553 | Channel or | Transporter |
| | member A5 | | transporter | |
| SLC9A6 | Solute carrier family 9 | 10479 | Channel or | Transporter |
| | member A6 | | transporter | |
| SLC9A7 | Solute carrier family 9 | 84679 | Channel or | Transporter |
| | member A7 | | transporter | |
| SLC9A8 | Solute carrier family 9 | 23315 | Channel or | Transporter |
| | member A8 | | transporter | |
| SLC9A9 | Solute carrier family 9 | 285195 | Channel or | Transporter |
| | member A9 | | transporter | |
| SLC9B1 | Solute carrier family 9 | 150159 | Channel or | Transporter |
| | member B1 | | transporter | |
| SLC9B2 | Solute carrier family 9 | 133308 | Channel or | Transporter |
| | member B2 | | transporter | |
| SLC9C1 | Solute carrier family 9 | 285335 | Channel or | Transporter |
| | member C1 | | transporter | |
| SLC9C2 | Solute carrier family 9 | 284525 | Channel or | Transporter |
| | member C2 (putative) | | transporter | |
| SLCO1A2 | Solute carrier organic anion | 6579 | Channel or | Transporter |
| | transporter family member | | transporter | |
| | 1A2 | | | |
| SLCO1B1 | Solute carrier organic anion | 10599 | Channel or | Transporter |
| | transporter family member | | transporter | |
| | 1B1 | | | |
| SLCO1B3 | Solute carrier organic anion | 28234 | Channel or | Transporter |
| | transporter family member | | transporter | |
| | 1B3 | | | |
| SLCO1C1 | Solute carrier organic anion | 53919 | Channel or | Transporter |
| | transporter family member | | transporter | |
| | 1C1 | | | |
| SLCO2A1 | Solute carrier organic anion | 6578 | Channel or | Transporter |
| | transporter family member | | transporter | |
| | 2A1 | | | |
| SLCO2B1 | Solute carrier organic anion | 11309 | Channel or | Transporter |
| | transporter family member | | transporter | |
| | 2B1 | | | |
| SLCO3A1 | Solute carrier organic anion | 28232 | Channel or | Transporter |
| | transporter family member | | transporter | |
| | 3A1 | | | |
| SLCO4A1 | Solute carrier organic anion | 28231 | Channel or | Transporter |
| | transporter family member | | transporter | |
| | 4A1 | | | |
| SLCO4C1 | Solute carrier organic anion | 353189 | Channel or | Transporter |
| | transporter family member | | transporter | |
| | 4C1 | | | |
| SLCO5A1 | Solute carrier organic anion | 81796 | Channel or | Transporter |
| | transporter family member | | transporter | |
| | 5A1 | | | |
| SLCO6A1 | Solute carrier organic anion | 133482 | Channel or | Transporter |
| | transporter family member | | transporter | |
| | 6A1 | | | |
| SLURP1 | Secreted LY6/PLAUR domain | 57152 | Other | Miscelaneous |
| | containing 1 | | | |
| SMPD3 | Sphingomyelin | 55512 | Other | Miscelaneous |
| | phosphodiesterase 3 | | | |
| SNAP23 | Synaptosome associated | 8773 | Vesicular | Vesicles |
| | protein 23 | | | |
| SNAP25 | Synaptosome associated | 6616 | Vesicular | Vesicles |
| | protein 25 | | | |
| SNAP29 | Synaptosome associated | 9342 | Vesicular | Vesicles |
| | protein 29 | | | |
| SNAPIN | SNAP associated protein | 23557 | Signaling | Signaling |
| SNCA | Synuclein alpha | 6622 | Vesicular | Vesicles |
| SNCAIP | Synuclein alpha interacting | 9627 | Vesicular | Vesicles |
| | protein | | | |
| SNCB | Synuclein beta | 6620 | Vesicular | Vesicles |
| SNCG | Synuclein gamma | 6623 | Vesicular | Vesicles |
| SNPH | Syntaphilin | 9751 | Vesicular | Vesicles |
| SNTG1 | Syntrophin gamma 1 | 54212 | Neurotrophic | Biosynthesis |
| SNX13 | Sorting nexin 13 | 23161 | Neurotransmitter | Signaling |
| SOD2 | Superoxide dismutase 2, | 6648 | Other | Miscelaneous |
| | mitochondrial | | | |
| SORCS1 | Sortilin related VPS10 domain | 114815 | Neurotrophic | Receptor |
| | containing receptor 1 | | | |
| SORCS2 | Sortilin related VPS10 domain | 57537 | Neurotrophic | Receptor |
| | containing receptor 2 | | | |
| SORCS3 | Sortilin related VPS10 domain | 22986 | Neurotrophic | Receptor |
| | containing receptor 3 | | | |
| SORT1 | Sortilin 1 | 6272 | Neurotrophic | Receptor |
| SOS1 | SOS Ras/Rac guanine | 6654 | Signaling | Signaling |
| | nucleotide exchange factor 1 | | | |
| SPX | Spexin hormone | 80763 | Neuropeptide | Ligand |
| SRC | SRC proto-oncogene, non- | 6714 | Signaling | Signaling |
| | receptor tyrosine kinase | | | |
| SST | Somatostatin | 6750 | Neuropeptide | Ligand |
| SSTR1 | Somatostatin receptor 1 | 6751 | Neuropeptide | Receptor |
| SSTR2 | Somatostatin receptor 2 | 6752 | Neuropeptide | Receptor |
| SSTR3 | Somatostatin receptor 3 | 6753 | Neuropeptide | Receptor |
| SSTR4 | Somatostatin receptor 4 | 6754 | Neuropeptide | Receptor |
| SSTR5 | Somatostatin receptor 5 | 6755 | Neuropeptide | Receptor |
| STAC3 | SH3 and cysteine rich domain | 246329 | Other | Miscelaneous |
| | 3 | | | |
| STRN | Striatin | 6801 | Signaling | Signaling |
| STX10 | Syntaxin 10 | 8677 | Vesicular | Vesicles |
| STX11 | Syntaxin 11 | 8676 | Vesicular | Vesicles |
| STX16 | Syntaxin 16 | 8675 | Vesicular | Vesicles |
| STX19 | Syntaxin 19 | 415117 | Vesicular | Vesicles |
| STX1A | Syntaxin 1A | 6804 | Vesicular | Vesicles |
| STX1B | Syntaxin 1B | 112755 | Vesicular | Vesicles |
| STX2 | Syntaxin 2 | 2054 | Vesicular | Vesicles |
| STX3 | Syntaxin 3 | 6809 | Vesicular | Vesicles |
| STX4 | Syntaxin 4 | 6810 | Vesicular | Vesicles |
| STX6 | Syntaxin 6 | 10228 | Vesicular | Vesicles |
| STXBP1 | Syntaxin binding protein 1 | 6812 | Vesicular | Vesicles |
| STXBP5 | Syntaxin binding protein 5 | 134957 | Vesicular | Vesicles |
| SULF1 | Sulfatase 1 | 23213 | Neurotrophic | Signaling |
| SULF2 | Sulfatase 2 | 55959 | Neurotrophic | Signaling |
| SV2A | Synaptic vesicle glycoprotein | 9900 | Vesicular | Vesicles |
| | 2A | | | |
| SV2B | Synaptic vesicle glycoprotein | 9899 | Vesicular | Vesicles |
| | 2B | | | |
| SV2C | Synaptic vesicle glycoprotein | 22987 | Vesicular | Vesicles |
| | 2C | | | |
| SYN1 | Synapsin I | 6853 | Vesicular | Vesicles |
| SYN2 | Synapsin II | 6854 | Vesicular | Vesicles |
| SYN3 | Synapsin III | 8224 | Vesicular | Vesicles |
| SYNJ1 | Synaptojanin 1 | 8867 | Vesicular | Vesicles |
| SYT1 | Synaptotagmin 1 | 6857 | Vesicular | Vesicles |
| SYT10 | Synaptotagmin 10 | 341359 | Vesicular | Vesicles |
| SYT11 | Synaptotagmin 11 | 23208 | Vesicular | Vesicles |
| SYT12 | Synaptotagmin 12 | 91683 | Vesicular | Vesicles |
| SYT13 | Synaptotagmin 13 | 57586 | Vesicular | Vesicles |
| SYT14 | Synaptotagmin 14 | 255928 | Vesicular | Vesicles |
| SYT14P1 | Synaptotagmin 14 | 401135 | Vesicular | Vesicles |
| | pseudogene 1 | | | |
| SYT15 | Synaptotagmin 15 | 83849 | Vesicular | Vesicles |
| SYT16 | Synaptotagmin 16 | 83851 | Vesicular | Vesicles |
| SYT17 | Synaptotagmin 17 | 51760 | Vesicular | Vesicles |
| SYT2 | Synaptotagmin 2 | 127833 | Vesicular | Vesicles |
| SYT3 | Synaptotagmin 3 | 84258 | Vesicular | Vesicles |
| SYT4 | Synaptotagmin 4 | 6860 | Vesicular | Vesicles |
| SYT5 | Synaptotagmin 5 | 6861 | Vesicular | Vesicles |
| SYT6 | Synaptotagmin 6 | 148281 | Vesicular | Vesicles |
| SYT7 | Synaptotagmin 7 | 9066 | Vesicular | Vesicles |
| SYT8 | Synaptotagmin 8 | 90019 | Vesicular | Vesicles |
| SYT9 | Synaptotagmin 9 | 143425 | Vesicular | Vesicles |
| SYTL1 | Synaptotagmin like 1 | 84958 | Vesicular | Vesicles |
| SYTL2 | Synaptotagmin like 2 | 54843 | Vesicular | Vesicles |
| SYTL3 | Synaptotagmin like 3 | 94120 | Vesicular | Vesicles |
| SYTL4 | Synaptotagmin like 4 | 94121 | Vesicular | Vesicles |
| SYTL5 | Synaptotagmin like 5 | 94122 | Vesicular | Vesicles |
| TAAR5 | Trace amine associated | 9038 | Neurotransmitter | Receptor |
| | receptor 5 | | | |
| TAC1 | Tachykinin precursor 1 | 6863 | Neuropeptide | Ligand |
| TAC3 | Tachykinin 3 | 6866 | Neuropeptide | Ligand |
| TAC4 | Tachykinin 4 (hemokinin) | 255061 | Neuropeptide | Ligand |
| TACR1 | Tachykinin receptor 1 | 6869 | Neuropeptide | Receptor |
| TACR2 | Tachykinin receptor 2 | 6865 | Neuropeptide | Receptor |
| TACR3 | Tachykinin receptor 3 | 6870 | Neuropeptide | Receptor |
| TBXA2R | Thromboxane A2 receptor | 6915 | Neuropeptide | Receptor |
| TCIRG1 | T-cell immune regulator 1, | 10312 | Channel or | Transporter |
| | atpase H+ transporting V0 | | transporter | |
| | subunit a3 | | | |
| TENM1 | Teneurin transmembrane | 10178 | Other | Miscelaneous |
| | protein 1 | | | |
| TGM2 | Transglutaminase 2 | 7052 | Other | Miscelaneous |
| TH | Tyrosine hydroxylase | 7054 | Neurotransmitter | Biosynthesis |
| TMEM158 | Transmembrane protein 158 | 25907 | Neurotrophic | Receptor |
| | (gene/pseudogene) | | | |
| TMOD2 | Tropomodulin 2 | 29767 | Vesicular | Vesicles |
| TNF | Tumor necrosis factor | 7124 | Neurotrophic | Ligand |
| TNR | Tenascin R | 7143 | Other | Miscelaneous |
| TP73 | Tumor protein p73 | 7161 | Other | Miscelaneous |
| TPCN1 | Two pore segment channel 1 | 53373 | Channel or | Channel |
| | | | transporter | |
| TPCN2 | Two pore segment channel 2 | 219931 | Channel or | Channel |
| | | | transporter | |
| TPH1 | Tryptophan hydroxylase 1 | 7166 | Neurotransmitter | Biosynthesis |
| TPH2 | Tryptophan hydroxylase 2 | 121278 | Neurotransmitter | Biosynthesis |
| TPM3 | Tropomyosin 3 | 7170 | Neurotrophic | Receptor |
| TPR | Translocated promoter region, | 7175 | Other | Miscelaneous |
| | nuclear basket protein | | | |
| TRH | Thyrotropin releasing | 7200 | Neuropeptide | Ligand |
| | hormone | | | |
| TRHDE | Thyrotropin releasing | 29953 | Neurotransmitter | Biosynthesis |
| | hormone degrading enzyme | | | |
| TRHR | Thyrotropin releasing | 7201 | Neuropeptide | Receptor |
| | hormone receptor | | | |
| TRPA1 | Transient receptor potential | 8989 | Channel or | Channel |
| | cation channel subfamily A | | transporter | |
| | member 1 | | | |
| TRPC1 | Transient receptor potential | 7220 | Channel or | Channel |
| | cation channel subfamily C | | transporter | |
| | member 1 | | | |
| TRPC2 | Transient receptor potential | 7221 | Channel or | Channel |
| | cation channel subfamily C | | transporter | |
| | member 2, pseudogene | | | |
| TRPC3 | Transient receptor potential | 7222 | Channel or | Channel |
| | cation channel subfamily C | | transporter | |
| | member 3 | | | |
| TRPC4 | Transient receptor potential | 7223 | Channel or | Channel |
| | cation channel subfamily C | | transporter | |
| | member 4 | | | |
| TRPC5 | Transient receptor potential | 7224 | Channel or | Channel |
| | cation channel subfamily C | | transporter | |
| | member 5 | | | |
| TRPC6 | Transient receptor potential | 7225 | Channel or | Channel |
| | cation channel subfamily C | | transporter | |
| | member 6 | | | |
| TRPC7 | Transient receptor potential | 57113 | Channel or | Channel |
| | cation channel subfamily C | | transporter | |
| | member 7 | | | |
| TRPM1 | Transient receptor potential | 4308 | Channel or | Channel |
| | cation channel subfamily M | | transporter | |
| | member 1 | | | |
| TRPM2 | Transient receptor potential | 7226 | Channel or | Channel |
| | cation channel subfamily M | | transporter | |
| | member 2 | | | |
| TRPM3 | Transient receptor potential | 80036 | Channel or | Channel |
| | cation channel subfamily M | | transporter | |
| | member 3 | | | |
| TRPM4 | Transient receptor potential | 54795 | Channel or | Channel |
| | cation channel subfamily M | | transporter | |
| | member 4 | | | |
| TRPM5 | Transient receptor potential | 29850 | Channel or | Channel |
| | cation channel subfamily M | | transporter | |
| | member 5 | | | |
| TRPM6 | Transient receptor potential | 140803 | Channel or | Channel |
| | cation channel subfamily M | | transporter | |
| | member 6 | | | |
| TRPM7 | Transient receptor potential | 54822 | Channel or | Channel |
| | cation channel subfamily M | | transporter | |
| | member 7 | | | |
| TRPM8 | Transient receptor potential | 79054 | Channel or | Channel |
| | cation channel subfamily M | | transporter | |
| | member 8 | | | |
| TRPV1 | Transient receptor potential | 7442 | Channel or | Channel |
| | cation channel subfamily V | | transporter | |
| | member 1 | | | |
| TRPV2 | Transient receptor potential | 51393 | Channel or | Channel |
| | cation channel subfamily V | | transporter | |
| | member 2 | | | |
| TRPV3 | Transient receptor potential | 162514 | Channel or | Channel |
| | cation channel subfamily V | | transporter | |
| | member 3 | | | |
| TRPV4 | Transient receptor potential | 59341 | Channel or | Channel |
| | cation channel subfamily V | | transporter | |
| | member 4 | | | |
| TRPV5 | Transient receptor potential | 56302 | Channel or | Channel |
| | cation channel subfamily V | | transporter | |
| | member 5 | | | |
| TRPV6 | Transient receptor potential | 55503 | Channel or | Channel |
| | cation channel subfamily V | | transporter | |
| | member 6 | | | |
| TSHR | Thyroid stimulating hormone | 7253 | Neuropeptide | Receptor |
| | receptor | | | |
| TSPOAP1 | TSPO associated protein 1 | 9256 | Vesicular | Vesicles |
| UBL5 | Ubiquitin like 5 | 59286 | Other | Miscelaneous |
| UCN | Urocortin | 7349 | Neuropeptide | Ligand |
| UCN2 | Urocortin 2 | 90226 | Neuropeptide | Ligand |
| UCN3 | Urocortin 3 | 114131 | Neuropeptide | Ligand |
| UCP1 | Uncoupling protein 1 | 7350 | Channel or | Transporter |
| | | | transporter | |
| UCP2 | Uncoupling protein 2 | 7351 | Channel or | Transporter |
| | | | transporter | |
| UCP3 | Uncoupling protein 3 | 7352 | Channel or | Transporter |
| | | | transporter | |
| UNC119 | Unc-119 lipid binding | 9094 | Vesicular | Vesicles |
| | chaperone | | | |
| UNC13A | Unc-13 homolog A | 23025 | Vesicular | Vesicles |
| UNC13B | Unc-13 homolog B | 10497 | Vesicular | Vesicles |
| USP46 | Ubiquitin specific peptidase 46 | 64854 | Other | Miscelaneous |
| UTS2 | Urotensin 2 | 10911 | Neuropeptide | Ligand |
| UTS2B | Urotensin 2B | 257313 | Neuropeptide | Ligand |
| UTS2R | Urotensin 2 receptor | 2837 | Neuropeptide | Receptor |
| VAMP1 | Vesicle associated membrane | 6843 | Vesicular | Vesicles |
| | protein 1 | | | |
| VAMP2 | Vesicle associated membrane | 6844 | Vesicular | Vesicles |
| | protein 2 | | | |
| VAMP3 | Vesicle associated membrane | 9341 | Vesicular | Vesicles |
| | protein 3 | | | |
| VAMP8 | Vesicle associated membrane | 8673 | Vesicular | Vesicles |
| | protein 8 | | | |
| VDAC1 | Voltage dependent anion | 7416 | Channel or | Channel |
| | channel 1 | | transporter | |
| VEGFA | Vascular endothelial growth | 7422 | Neurotrophic | Ligand |
| | factor A | | | |
| VGF | VGF nerve growth factor | 7425 | Neurotrophic | Ligand |
| | inducible | | | |
| VIP | Vasoactive intestinal peptide | 7432 | Neuropeptide | Ligand |
| VIPR1 | Vasoactive intestinal peptide | 7433 | Neuropeptide | Receptor |
| | receptor 1 | | | |
| VIPR2 | Vasoactive intestinal peptide | 7434 | Neuropeptide | Receptor |
| | receptor 2 | | | |
| XCR1 | X-C motif chemokine receptor | 2829 | Neurotrophic | Receptor |
| | 1 | | | |
| ZACN | Zinc activated ion channel | 353174 | Channel or | Channel |
| | | | transporter | |
| ZN274 | Neurotrophin receptor- | 10782 | Neurotrophic | Signaling |
| | interacting factor homolog | | | |

TABLE 8
ION CHANNEL AND TRANSPORTER GENES

| | | | | | Ion |
|---|---|---|---|---|---|
| Approved | | Entrez | Gene type/ | | channel |
| Symbol | Approved name | Gene ID | family | Category | type |
| ASIC1 | Acid sensing ion channel | 41 | Channel or | Channel | Lgic |
| | subunit 1 | | transporter | | |
| ASIC2 | Acid sensing ion channel | 40 | Channel or | Channel | Lgic |
| | subunit 2 | | transporter | | |
| ASIC3 | Acid sensing ion channel | 9311 | Channel or | Channel | Lgic |
| | subunit 3 | | transporter | | |
| GABRA1 | Gamma-aminobutyric acid | 2554 | Neurotransmitter | Receptor | Lgic |
| | type A receptor alpha1 | | | | |
| | subunit | | | | |
| GABRA2 | Gamma-aminobutyric acid | 2555 | Neurotransmitter | Receptor | Lgic |
| | type A receptor alpha2 | | | | |
| | subunit | | | | |
| GABRA3 | Gamma-aminobutyric acid | 2556 | Neurotransmitter | Receptor | Lgic |
| | type A receptor alpha3 | | | | |
| | subunit | | | | |
| GABRA4 | Gamma-aminobutyric acid | 2557 | Neurotransmitter | Receptor | Lgic |
| | type A receptor alpha4 | | | | |
| | subunit | | | | |
| GABRA5 | Gamma-aminobutyric acid | 2558 | Neurotransmitter | Receptor | Lgic |
| | type A receptor alpha5 | | | | |
| | subunit | | | | |
| GABRA6 | Gamma-aminobutyric acid | 2559 | Neurotransmitter | Receptor | Lgic |
| | type A receptor alpha6 | | | | |
| | subunit | | | | |
| GABRB1 | Gamma-aminobutyric acid | 2560 | Neurotransmitter | Receptor | Lgic |
| | type A receptor beta1 | | | | |
| | subunit | | | | |
| GABRB2 | Gamma-aminobutyric acid | 2561 | Neurotransmitter | Receptor | Lgic |
| | type A receptor beta2 | | | | |
| | subunit | | | | |
| GABRB3 | Gamma-aminobutyric acid | 2562 | Neurotransmitter | Receptor | Lgic |
| | type A receptor beta3 | | | | |
| | subunit | | | | |
| GABRD | Gamma-aminobutyric acid | 2563 | Neurotransmitter | Receptor | Lgic |
| | type A receptor delta | | | | |
| | subunit | | | | |
| GABRE | Gamma-aminobutyric acid | 2564 | Neurotransmitter | Receptor | Lgic |
| | type A receptor epsilon | | | | |
| | subunit | | | | |
| GABRG1 | Gamma-aminobutyric acid | 2565 | Neurotransmitter | Receptor | Lgic |
| | type A receptor gamma1 | | | | |
| | subunit | | | | |
| GABRG2 | Gamma-aminobutyric acid | 2566 | Neurotransmitter | Receptor | Lgic |
| | type A receptor gamma2 | | | | |
| | subunit | | | | |
| GABRG3 | Gamma-aminobutyric acid | 2567 | Neurotransmitter | Receptor | Lgic |
| | type A receptor gamma3 | | | | |
| | subunit | | | | |
| GABRP | Gamma-aminobutyric acid | 2568 | Neurotransmitter | Receptor | Lgic |
| | type A receptor pi subunit | | | | |
| GABRQ | Gamma-aminobutyric acid | 55879 | Neurotransmitter | Receptor | Lgic |
| | type A receptor theta | | | | |
| | subunit | | | | |
| GABRR1 | Gamma-aminobutyric acid | 2569 | Neurotransmitter | Receptor | Lgic |
| | type A receptor rho1 subunit | | | | |
| GABRR2 | Gamma-aminobutyric acid | 2570 | Neurotransmitter | Receptor | Lgic |
| | type A receptor rho2 subunit | | | | |
| GABRR3 | Gamma-aminobutyric acid | 200959 | Neurotransmitter | Receptor | Lgic |
| | type A receptor rho3 subunit | | | | |
| | (gene/pseudogene) | | | | |
| GLRA1 | Glycine receptor alpha 1 | 2741 | Neurotransmitter | Receptor | Lgic |
| GLRA2 | Glycine receptor alpha 2 | 2742 | Neurotransmitter | Receptor | Lgic |
| GLRA3 | Glycine receptor alpha 3 | 8001 | Neurotransmitter | Receptor | Lgic |
| GLRA4 | Glycine receptor alpha 4 | 441509 | Neurotransmitter | Receptor | Lgic |
| GLRB | Glycine receptor beta | 2743 | Neurotransmitter | Receptor | Lgic |
| GRIA1 | Glutamate ionotropic | 2890 | Neurotransmitter | Receptor | Lgic |
| | receptor AMPA type subunit 1 | | | | |
| GRIA2 | Glutamate ionotropic | 2891 | Neurotransmitter | Receptor | Lgic |
| | receptor AMPA type subunit 2 | | | | |
| GRIA3 | Glutamate ionotropic | 2892 | Neurotransmitter | Receptor | Lgic |
| | receptor AMPA type subunit 3 | | | | |
| GRIA4 | Glutamate ionotropic | 2893 | Neurotransmitter | Receptor | Lgic |
| | receptor AMPA type subunit 4 | | | | |
| GRID1 | Glutamate ionotropic | 2894 | Neurotransmitter | Receptor | Lgic |
| | receptor delta type subunit 1 | | | | |
| GRID2 | Glutamate ionotropic | 2895 | Neurotransmitter | Receptor | Lgic |
| | receptor delta type subunit 2 | | | | |
| GRIK1 | Glutamate ionotropic | 2897 | Neurotransmitter | Receptor | Lgic |
| | receptor kainate type | | | | |
| | subunit 1 | | | | |
| GRIK2 | Glutamate ionotropic | 2898 | Neurotransmitter | Receptor | Lgic |
| | receptor kainate type | | | | |
| | subunit 2 | | | | |
| GRIK3 | Glutamate ionotropic | 2899 | Neurotransmitter | Receptor | Lgic |
| | receptor kainate type | | | | |
| | subunit 3 | | | | |
| GRIK4 | Glutamate ionotropic | 2900 | Neurotransmitter | Receptor | Lgic |
| | receptor kainate type | | | | |
| | subunit 4 | | | | |
| GRIK5 | Glutamate ionotropic | 2901 | Neurotransmitter | Receptor | Lgic |
| | receptor kainate type | | | | |
| | subunit 5 | | | | |
| GRIN1 | Glutamate ionotropic | 2902 | Neurotransmitter | Receptor | Lgic |
| | receptor NMDA type subunit 1 | | | | |
| GRIN2A | Glutamate ionotropic | 2903 | Neurotransmitter | Receptor | Lgic |
| | receptor NMDA type subunit | | | | |
| | 2A | | | | |
| GRIN2B | Glutamate ionotropic | 2904 | Neurotransmitter | Receptor | Lgic |
| | receptor NMDA type subunit | | | | |
| | 2B | | | | |
| GRIN2C | Glutamate ionotropic | 2905 | Neurotransmitter | Receptor | Lgic |
| | receptor NMDA type subunit | | | | |
| | 2C | | | | |
| GRIN2D | Glutamate ionotropic | 2906 | Neurotransmitter | Receptor | Lgic |
| | receptor NMDA type subunit | | | | |
| | 2D | | | | |
| GRIN3A | Glutamate ionotropic | 116443 | Neurotransmitter | Receptor | Lgic |
| | receptor NMDA type subunit | | | | |
| | 3A | | | | |
| GRIN3B | Glutamate ionotropic | 116444 | Neurotransmitter | Receptor | Lgic |
| | receptor NMDA type subunit | | | | |
| | 3B | | | | |
| HTR3A | 5-hydroxytryptamine | 3359 | Neurotransmitter | Receptor | Lgic |
| | receptor 3A | | | | |
| HTR3B | 5-hydroxytryptamine | 9177 | Neurotransmitter | Receptor | Lgic |
| | receptor 3B | | | | |
| HTR3C | 5-hydroxytryptamine | 170572 | Neurotransmitter | Receptor | Lgic |
| | receptor 3C | | | | |
| HTR3D | 5-hydroxytryptamine | 200909 | Neurotransmitter | Receptor | Lgic |
| | receptor 3D | | | | |
| HTR3E | 5-hydroxytryptamine | 285242 | Neurotransmitter | Receptor | Lgic |
| | receptor 3E | | | | |
| ITPR1 | Inositol 1,4,5-trisphosphate | 3708 | Neurotransmitter | Signaling | Lgic |
| | receptor type 1 | | | | |
| ITPR2 | Inositol 1,4,5-trisphosphate | 3709 | Neurotransmitter | Signaling | Lgic |
| | receptor type 2 | | | | |
| ITPR3 | Inositol 1,4,5-trisphosphate | 3710 | Neurotransmitter | Signaling | Lgic |
| | receptor type 3 | | | | |
| SCNN1A | Sodium channel epithelial 1 | 6337 | Channel or | Channel | Lgic |
| | alpha subunit | | transporter | | |
| SCNN1B | Sodium channel epithelial 1 | 6338 | Channel or | Channel | Lgic |
| | beta subunit | | transporter | | |
| SCNN1D | Sodium channel epithelial 1 | 6339 | Channel or | Channel | Lgic |
| | delta subunit | | transporter | | |
| SCNN1G | Sodium channel epithelial 1 | 6340 | Channel or | Channel | Lgic |
| | gamma subunit | | transporter | | |
| ZACN | Zinc activated ion channel | 353174 | Channel or | Channel | Lgic |
| | | | transporter | | |
| CLCN1 | Chloride voltage-gated | 1180 | Channel or | Channel | Other_ic |
| | channel 1 | | transporter | | |
| CLCN2 | Chloride voltage-gated | 1181 | Channel or | Channel | Other_ic |
| | channel 2 | | transporter | | |
| CLCN3 | Chloride voltage-gated | 1182 | Channel or | Channel | Other_ic |
| | channel 3 | | transporter | | |
| CLCN4 | Chloride voltage-gated | 1183 | Channel or | Channel | Other_ic |
| | channel 4 | | transporter | | |
| CLCN5 | Chloride voltage-gated | 1184 | Channel or | Channel | Other_ic |
| | channel 5 | | transporter | | |
| CLCN6 | Chloride voltage-gated | 1185 | Channel or | Channel | Other_ic |
| | channel 6 | | transporter | | |
| CLCN7 | Chloride voltage-gated | 1186 | Channel or | Channel | Other_ic |
| | channel 7 | | transporter | | |
| CLCNKA | Chloride voltage-gated | 1187 | Channel or | Channel | Other_ic |
| | channel Ka | | transporter | | |
| CLCNKB | Chloride voltage-gated | 1188 | Channel or | Channel | Other_ic |
| | channel Kb | | transporter | | |
| CLIC6 | Chloride intracellular | 54102 | Channel or | Channel | Other_ic |
| | channel 6 | | transporter | | |
| GJA1 | Gap junction protein alpha 1 | 2697 | Channel or | Channel | Other_ic |
| | | | transporter | | |
| GJA10 | Gap junction protein alpha | 84694 | Channel or | Channel | Other_ic |
| | 10 | | transporter | | |
| GJA3 | Gap junction protein alpha 3 | 2700 | Channel or | Channel | Other_ic |
| | | | transporter | | |
| GJA4 | Gap junction protein alpha 4 | 2701 | Channel or | Channel | Other_ic |
| | | | transporter | | |
| GJA5 | Gap junction protein alpha 5 | 2702 | Channel or | Channel | Other_ic |
| | | | transporter | | |
| GJA8 | Gap junction protein alpha 8 | 2703 | Channel or | Channel | Other_ic |
| | | | transporter | | |
| GJA9 | Gap junction protein alpha 9 | 81025 | Channel or | Channel | Other_ic |
| | | | transporter | | |
| GJB1 | Gap junction protein beta 1 | 2705 | Channel or | Channel | Other_ic |
| | | | transporter | | |
| GJB2 | Gap junction protein beta 2 | 2706 | Channel or | Channel | Other_ic |
| | | | transporter | | |
| GJB3 | Gap junction protein beta 3 | 2707 | Channel or | Channel | Other_ic |
| | | | transporter | | |
| GJB4 | Gap junction protein beta 4 | 127534 | Channel or | Channel | Other_ic |
| | | | transporter | | |
| GJB5 | Gap junction protein beta 5 | 2709 | Channel or | Channel | Other_ic |
| | | | transporter | | |
| GJB6 | Gap junction protein beta 6 | 10804 | Channel or | Channel | Other_ic |
| | | | transporter | | |
| GJB7 | Gap junction protein beta 7 | 375519 | Channel or | Channel | Other_ic |
| | | | transporter | | |
| GJC1 | Gap junction protein gamma 1 | 10052 | Channel or | Channel | Other_ic |
| | | | transporter | | |
| GJC2 | Gap junction protein gamma 2 | 57165 | Channel or | Channel | Other_ic |
| | | | transporter | | |
| GJC3 | Gap junction protein gamma 3 | 349149 | Channel or | Channel | Other_ic |
| | | | transporter | | |
| GJD2 | Gap junction protein delta 2 | 57369 | Channel or | Channel | Other_ic |
| | | | transporter | | |
| GJD3 | Gap junction protein delta 3 | 125111 | Channel or | Channel | Other_ic |
| | | | transporter | | |
| GJD4 | Gap junction protein delta 4 | 219770 | Channel or | Channel | Other_ic |
| | | | transporter | | |
| GJE1 | Gap junction protein epsilon 1 | 100126572 | Channel or | Channel | Other_ic |
| | | | transporter | | |
| KCNMB4 | Potassium calcium- | 27345 | Channel or | Channel | Other_ic |
| | activated channel subfamily | | transporter | | |
| | M regulatory beta subunit 4 | | | | |
| NALCN | Sodium leak channel, non- | 259232 | Channel or | Channel | Other_ic |
| | selective | | transporter | | |
| PANX1 | Pannexin 1 | 24145 | Channel or | Channel | Other_ic |
| | | | transporter | | |
| PANX2 | Pannexin 2 | 56666 | Channel or | Channel | Other_ic |
| | | | transporter | | |
| PANX3 | Pannexin 3 | 116337 | Channel or | Channel | Other_ic |
| | | | transporter | | |
| SHROOM1 | Shroom family member 1 | 134549 | Channel or | Channel | Other_ic |
| | | | transporter | | |
| SHROOM2 | Shroom family member 2 | 357 | Channel or | Channel | Other_ic |
| | | | transporter | | |
| SHROOM3 | Shroom family member 3 | 57619 | Channel or | Channel | Other_ic |
| | | | transporter | | |
| SHROOM4 | Shroom family member 4 | 57477 | Channel or | Channel | Other_ic |
| | | | transporter | | |
| ATP10A | Atpase phospholipid | 57194 | Channel or | Transporter | Transporter |
| | transporting 10A (putative) | | transporter | | |
| ATP10B | Atpase phospholipid | 23120 | Channel or | Transporter | Transporter |
| | transporting 10B (putative) | | transporter | | |
| ATP10D | Atpase phospholipid | 57205 | Channel or | Transporter | Transporter |
| | transporting 10D (putative) | | transporter | | |
| ATP11A | Atpase phospholipid | 23250 | Channel or | Transporter | Transporter |
| | transporting 11A | | transporter | | |
| ATP11B | Atpase phospholipid | 23200 | Channel or | Transporter | Transporter |
| | transporting 11B (putative) | | transporter | | |
| ATP11C | Atpase phospholipid | 286410 | Channel or | Transporter | Transporter |
| | transporting 11C | | transporter | | |
| ATP12A | Atpase H+/K+ transporting | 479 | Channel or | Transporter | Transporter |
| | non-gastric alpha2 subunit | | transporter | | |
| ATP1A1 | Atpase Na+/K+ transporting | 476 | Channel or | Transporter | Transporter |
| | subunit alpha 1 | | transporter | | |
| ATP1A2 | Atpase Na+/K+ transporting | 477 | Channel or | Transporter | Transporter |
| | subunit alpha 2 | | transporter | | |
| ATP1A3 | Atpase Na+/K+ transporting | 478 | Channel or | Transporter | Transporter |
| | subunit alpha 3 | | transporter | | |
| ATP1A4 | Atpase Na+/K+ transporting | 480 | Channel or | Transporter | Transporter |
| | subunit alpha 4 | | transporter | | |
| ATP1B1 | Atpase Na+/K+ transporting | 481 | Channel or | Transporter | Transporter |
| | subunit beta 1 | | transporter | | |
| ATP1B2 | Atpase Na+/K+ transporting | 482 | Channel or | Transporter | Transporter |
| | subunit beta 2 | | transporter | | |
| ATP1B3 | Atpase Na+/K+ transporting | 483 | Channel or | Transporter | Transporter |
| | subunit beta 3 | | transporter | | |
| ATP2A1 | Atpase | 487 | Channel or | Transporter | Transporter |
| | sarcoplasmic/endoplasmic | | transporter | | |
| | reticulum Ca2+ transporting 1 | | | | |
| ATP2A2 | Atpase | 488 | Channel or | Transporter | Transporter |
| | sarcoplasmic/endoplasmic | | transporter | | |
| | reticulum Ca2+ transporting 2 | | | | |
| ATP2A3 | Atpase | 489 | Channel or | Transporter | Transporter |
| | sarcoplasmic/endoplasmic | | transporter | | |
| | reticulum Ca2+ transporting 3 | | | | |
| ATP2B1 | Atpase plasma membrane | 490 | Channel or | Transporter | Transporter |
| | Ca2+ transporting 1 | | transporter | | |
| ATP2B2 | Atpase plasma membrane | 491 | Channel or | Transporter | Transporter |
| | Ca2+ transporting 2 | | transporter | | |
| ATP2B3 | Atpase plasma membrane | 492 | Channel or | Transporter | Transporter |
| | Ca2+ transporting 3 | | transporter | | |
| ATP2B4 | Atpase plasma membrane | 493 | Channel or | Transporter | Transporter |
| | Ca2+ transporting 4 | | transporter | | |
| ATP2C1 | Atpase secretory pathway | 27032 | Channel or | Transporter | Transporter |
| | Ca2+ transporting 1 | | transporter | | |
| ATP2C2 | Atpase secretory pathway | 9914 | Channel or | Transporter | Transporter |
| | Ca2+ transporting 2 | | transporter | | |
| ATP4A | Atpase H+/K+ transporting | 495 | Channel or | Transporter | Transporter |
| | alpha subunit | | transporter | | |
| ATP4B | Atpase H+/K+ transporting | 496 | Channel or | Transporter | Transporter |
| | beta subunit | | transporter | | |
| ATP5A1 | ATP synthase, H+ | 498 | Channel or | Transporter | Transporter |
| | transporting, mitochondrial | | transporter | | |
| | F1 complex, alpha subunit | | | | |
| | 1, cardiac muscle | | | | |
| ATP5B | ATP synthase, H+ | 506 | Channel or | Transporter | Transporter |
| | transporting, mitochondrial | | transporter | | |
| | F1 complex, beta | | | | |
| | polypeptide | | | | |
| ATP5C1 | ATP synthase, H+ | 509 | Channel or | Transporter | Transporter |
| | transporting, mitochondrial | | transporter | | |
| | F1 complex, gamma | | | | |
| | polypeptide 1 | | | | |
| ATP5D | ATP synthase, H+ | 513 | Channel or | Transporter | Transporter |
| | transporting, mitochondrial | | transporter | | |
| | F1 complex, delta subunit | | | | |
| ATP5E | ATP synthase, H+ | 514 | Channel or | Transporter | Transporter |
| | transporting, mitochondrial | | transporter | | |
| | F1 complex, epsilon subunit | | | | |
| ATP5F1 | ATP synthase, H+ | 515 | Channel or | Transporter | Transporter |
| | transporting, mitochondrial | | transporter | | |
| | Fo complex subunit B1 | | | | |
| ATP5H | ATP synthase, H+ | 10476 | Channel or | Transporter | Transporter |
| | transporting, mitochondrial | | transporter | | |
| | Fo complex subunit D | | | | |
| ATP5I | ATP synthase, H+ | 521 | Channel or | Transporter | Transporter |
| | transporting, mitochondrial | | transporter | | |
| | Fo complex subunit E | | | | |
| ATP5J | ATP synthase, H+ | 522 | Channel or | Transporter | Transporter |
| | transporting, mitochondrial | | transporter | | |
| | Fo complex subunit F6 | | | | |
| ATP5J2 | ATP synthase, H+ | 9551 | Channel or | Transporter | Transporter |
| | transporting, mitochondrial | | transporter | | |
| | Fo complex subunit F2 | | | | |
| ATP5L2 | ATP synthase, H+ | 267020 | Channel or | Transporter | Transporter |
| | transporting, mitochondrial | | transporter | | |
| | Fo complex subunit G2 | | | | |
| ATP6V0A1 | Atpase H+ transporting V0 | 535 | Channel or | Transporter | Transporter |
| | subunit a1 | | transporter | | |
| ATP6V0A2 | Atpase H+ transporting V0 | 23545 | Channel or | Transporter | Transporter |
| | subunit a2 | | transporter | | |
| ATP6V0A4 | Atpase H+ transporting V0 | 50617 | Channel or | Transporter | Transporter |
| | subunit a4 | | transporter | | |
| ATP6V0B | Atpase H+ transporting V0 | 533 | Channel or | Transporter | Transporter |
| | subunit b | | transporter | | |
| ATP6V0C | Atpase H+ transporting V0 | 527 | Channel or | Transporter | Transporter |
| | subunit c | | transporter | | |
| ATP6V0D1 | Atpase H+ transporting V0 | 9114 | Channel or | Transporter | Transporter |
| | subunit d1 | | transporter | | |
| ATP6V0D2 | Atpase H+ transporting V0 | 245972 | Channel or | Transporter | Transporter |
| | subunit d2 | | transporter | | |
| ATP6V0E1 | Atpase H+ transporting V0 | 8992 | Channel or | Transporter | Transporter |
| | subunit e1 | | transporter | | |
| ATP6V0E2 | Atpase H+ transporting V0 | 155066 | Channel or | Transporter | Transporter |
| | subunit e2 | | transporter | | |
| ATP6V1A | Atpase H+ transporting V1 | 523 | Channel or | Transporter | Transporter |
| | subunit A | | transporter | | |
| ATP6V1B1 | Atpase H+ transporting V1 | 525 | Channel or | Transporter | Transporter |
| | subunit B1 | | transporter | | |
| ATP6V1B2 | Atpase H+ transporting V1 | 526 | Channel or | Transporter | Transporter |
| | subunit B2 | | transporter | | |
| ATP6V1C1 | Atpase H+ transporting V1 | 528 | Channel or | Transporter | Transporter |
| | subunit C1 | | transporter | | |
| ATP6V1C2 | Atpase H+ transporting V1 | 245973 | Channel or | Transporter | Transporter |
| | subunit C2 | | transporter | | |
| ATP6V1D | Atpase H+ transporting V1 | 51382 | Channel or | Transporter | Transporter |
| | subunit D | | transporter | | |
| ATP6V1E1 | Atpase H+ transporting V1 | 529 | Channel or | Transporter | Transporter |
| | subunit E1 | | transporter | | |
| ATP6V1E2 | Atpase H+ transporting V1 | 90423 | Channel or | Transporter | Transporter |
| | subunit E2 | | transporter | | |
| ATP6V1F | Atpase H+ transporting V1 | 9296 | Channel or | Transporter | Transporter |
| | subunit F | | transporter | | |
| ATP6V1G1 | Atpase H+ transporting V1 | 9550 | Channel or | Transporter | Transporter |
| | subunit G1 | | transporter | | |
| ATP6V1G2 | Atpase H+ transporting V1 | 534 | Channel or | Transporter | Transporter |
| | subunit G2 | | transporter | | |
| ATP6V1G3 | Atpase H+ transporting V1 | 127124 | Channel or | Transporter | Transporter |
| | subunit G3 | | transporter | | |
| ATP6V1H | Atpase H+ transporting V1 | 51606 | Channel or | Transporter | Transporter |
| | subunit H | | transporter | | |
| ATP7A | Atpase copper transporting | 538 | Channel or | Transporter | Transporter |
| | alpha | | transporter | | |
| ATP7B | Atpase copper transporting | 540 | Channel or | Transporter | Transporter |
| | beta | | transporter | | |
| ATP8A1 | Atpase phospholipid | 10396 | Channel or | Transporter | Transporter |
| | transporting 8A1 | | transporter | | |
| ATP8A2 | Atpase phospholipid | 51761 | Channel or | Transporter | Transporter |
| | transporting 8A2 | | transporter | | |
| ATP8B1 | Atpase phospholipid | 5205 | Channel or | Transporter | Transporter |
| | transporting 8B1 | | transporter | | |
| ATP8B2 | Atpase phospholipid | 57198 | Channel or | Transporter | Transporter |
| | transporting 8B2 | | transporter | | |
| ATP8B3 | Atpase phospholipid | 148229 | Channel or | Transporter | Transporter |
| | transporting 8B3 | | transporter | | |
| ATP8B4 | Atpase phospholipid | 79895 | Channel or | Transporter | Transporter |
| | transporting 8B4 (putative) | | transporter | | |
| ATP9A | Atpase phospholipid | 10079 | Channel or | Transporter | Transporter |
| | transporting 9A (putative) | | transporter | | |
| ATP9B | Atpase phospholipid | 374868 | Channel or | Transporter | Transporter |
| | transporting 9B (putative) | | transporter | | |
| DIRC2 | Disrupted in renal | 84925 | Channel or | Transporter | Transporter |
| | carcinoma 2 | | transporter | | |
| FLVCR1 | Feline leukemia virus | 28982 | Channel or | Transporter | Transporter |
| | subgroup C cellular receptor 1 | | transporter | | |
| FLVCR2 | Feline leukemia virus | 55640 | Channel or | Transporter | Transporter |
| | subgroup C cellular receptor | | transporter | | |
| | family member 2 | | | | |
| FXYD2 | FXYD domain containing | 486 | Channel or | Transporter | Transporter |
| | ion transport regulator 2 | | transporter | | |
| HTL | High L-leucine transport | 3343 | Channel or | Transporter | Transporter |
| | | | transporter | | |
| MFSD7 | Major facilitator superfamily | 84179 | Channel or | Transporter | Transporter |
| | domain containing 7 | | transporter | | |
| MT-ATP6 | Mitochondrially encoded | 4508 | Channel or | Transporter | Transporter |
| | ATP synthase 6 | | transporter | | |
| MT-ATP8 | Mitochondrially encoded | 4509 | Channel or | Transporter | Transporter |
| | ATP synthase 8 | | transporter | | |
| MTCH1 | Mitochondrial carrier 1 | 23787 | Channel or | Transporter | Transporter |
| | | | transporter | | |
| MTCH2 | Mitochondrial carrier 2 | 23788 | Channel or | Transporter | Transporter |
| | | | transporter | | |
| NPC1L1 | NPC1 like intracellular | 29881 | Channel or | Transporter | Transporter |
| | cholesterol transporter 1 | | transporter | | |
| RHAG | Rh-associated glycoprotein | 6005 | Channel or | Transporter | Transporter |
| | | | transporter | | |
| RHBG | Rh family B glycoprotein | 57127 | Channel or | Transporter | Transporter |
| | (gene/pseudogene) | | transporter | | |
| RHCG | Rh family C glycoprotein | 51458 | Channel or | Transporter | Transporter |
| | | | transporter | | |
| SLC10A1 | Solute carrier family 10 | 6554 | Channel or | Transporter | Transporter |
| | member 1 | | transporter | | |
| SLC10A2 | Solute carrier family 10 | 6555 | Channel or | Transporter | Transporter |
| | member 2 | | transporter | | |
| SLC10A3 | Solute carrier family 10 | 8273 | Channel or | Transporter | Transporter |
| | member 3 | | transporter | | |
| SLC10A4 | Solute carrier family 10 | 201780 | Channel or | Transporter | Transporter |
| | member 4 | | transporter | | |
| SLC10A5 | Solute carrier family 10 | 347051 | Channel or | Transporter | Transporter |
| | member 5 | | transporter | | |
| SLC10A6 | Solute carrier family 10 | 345274 | Channel or | Transporter | Transporter |
| | member 6 | | transporter | | |
| SLC10A7 | Solute carrier family 10 | 84068 | Channel or | Transporter | Transporter |
| | member 7 | | transporter | | |
| SLC11A1 | Solute carrier family 11 | 6556 | Channel or | Transporter | Transporter |
| | member 1 | | transporter | | |
| SLC11A2 | Solute carrier family 11 | 4891 | Channel or | Transporter | Transporter |
| | member 2 | | transporter | | |
| SLC12A1 | Solute carrier family 12 | 6557 | Channel or | Transporter | Transporter |
| | member 1 | | transporter | | |
| SLC12A2 | Solute carrier family 12 | 6558 | Channel or | Transporter | Transporter |
| | member 2 | | transporter | | |
| SLC12A3 | Solute carrier family 12 | 6559 | Channel or | Transporter | Transporter |
| | member 3 | | transporter | | |
| SLC12A4 | Solute carrier family 12 | 6560 | Channel or | Transporter | Transporter |
| | member 4 | | transporter | | |
| SLC12A5 | Solute carrier family 12 | 57468 | Channel or | Transporter | Transporter |
| | member 5 | | transporter | | |
| SLC12A6 | Solute carrier family 12 | 9990 | Channel or | Transporter | Transporter |
| | member 6 | | transporter | | |
| SLC12A7 | Solute carrier family 12 | 10723 | Channel or | Transporter | Transporter |
| | member 7 | | transporter | | |
| SLC12A8 | Solute carrier family 12 | 84561 | Channel or | Transporter | Transporter |
| | member 8 | | transporter | | |
| SLC12A9 | Solute carrier family 12 | 56996 | Channel or | Transporter | Transporter |
| | member 9 | | transporter | | |
| SLC13A1 | Solute carrier family 13 | 6561 | Channel or | Transporter | Transporter |
| | member 1 | | transporter | | |
| SLC13A2 | Solute carrier family 13 | 9058 | Channel or | Transporter | Transporter |
| | member 2 | | transporter | | |
| SLC13A3 | Solute carrier family 13 | 64849 | Channel or | Transporter | Transporter |
| | member 3 | | transporter | | |
| SLC13A4 | Solute carrier family 13 | 26266 | Channel or | Transporter | Transporter |
| | member 4 | | transporter | | |
| SLC13A5 | Solute carrier family 13 | 284111 | Channel or | Transporter | Transporter |
| | member 5 | | transporter | | |
| SLC14A1 | Solute carrier family 14 | 6563 | Channel or | Transporter | Transporter |
| | member 1 (Kidd blood | | transporter | | |
| | group) | | | | |
| SLC14A2 | Solute carrier family 14 | 8170 | Channel or | Transporter | Transporter |
| | member 2 | | transporter | | |
| SLC15A1 | Solute carrier family 15 | 6564 | Channel or | Transporter | Transporter |
| | member 1 | | transporter | | |
| SLC15A2 | Solute carrier family 15 | 6565 | Channel or | Transporter | Transporter |
| | member 2 | | transporter | | |
| SLC15A3 | Solute carrier family 15 | 51296 | Channel or | Transporter | Transporter |
| | member 3 | | transporter | | |
| SLC15A4 | Solute carrier family 15 | 121260 | Channel or | Transporter | Transporter |
| | member 4 | | transporter | | |
| SLC16A1 | Solute carrier family 16 | 6566 | Channel or | Transporter | Transporter |
| | member 1 | | transporter | | |
| SLC16A10 | Solute carrier family 16 | 117247 | Channel or | Transporter | Transporter |
| | member 10 | | transporter | | |
| SLC16A11 | Solute carrier family 16 | 162515 | Channel or | Transporter | Transporter |
| | member 11 | | transporter | | |
| SLC16A12 | Solute carrier family 16 | 387700 | Channel or | Transporter | Transporter |
| | member 12 | | transporter | | |
| SLC16A13 | Solute carrier family 16 | 201232 | Channel or | Transporter | Transporter |
| | member 13 | | transporter | | |
| SLC16A14 | Solute carrier family 16 | 151473 | Channel or | Transporter | Transporter |
| | member 14 | | transporter | | |
| SLC16A2 | Solute carrier family 16 | 6567 | Channel or | Transporter | Transporter |
| | member 2 | | transporter | | |
| SLC16A3 | Solute carrier family 16 | 9123 | Channel or | Transporter | Transporter |
| | member 3 | | transporter | | |
| SLC16A4 | Solute carrier family 16 | 9122 | Channel or | Transporter | Transporter |
| | member 4 | | transporter | | |
| SLC16A5 | Solute carrier family 16 | 9121 | Channel or | Transporter | Transporter |
| | member 5 | | transporter | | |
| SLC16A6 | Solute carrier family 16 | 9120 | Channel or | Transporter | Transporter |
| | member 6 | | transporter | | |
| SLC16A7 | Solute carrier family 16 | 9194 | Channel or | Transporter | Transporter |
| | member 7 | | transporter | | |
| SLC16A8 | Solute carrier family 16 | 23539 | Channel or | Transporter | Transporter |
| | member 8 | | transporter | | |
| SLC16A9 | Solute carrier family 16 | 220963 | Channel or | Transporter | Transporter |
| | member 9 | | transporter | | |
| SLC17A1 | Solute carrier family 17 | 6568 | Channel or | Transporter | Transporter |
| | member 1 | | transporter | | |
| SLC17A2 | Solute carrier family 17 | 10246 | Channel or | Transporter | Transporter |
| | member 2 | | transporter | | |
| SLC17A3 | Solute carrier family 17 | 10786 | Channel or | Transporter | Transporter |
| | member 3 | | transporter | | |
| SLC17A4 | Solute carrier family 17 | 10050 | Channel or | Transporter | Transporter |
| | member 4 | | transporter | | |
| SLC17A5 | Solute carrier family 17 | 26503 | Channel or | Transporter | Transporter |
| | member 5 | | transporter | | |
| SLC17A6 | Solute carrier family 17 | 57084 | Channel or | Transporter | Transporter |
| | member 6 | | transporter | | |
| SLC17A7 | Solute carrier family 17 | 57030 | Channel or | Transporter | Transporter |
| | member 7 | | transporter | | |
| SLC17A8 | Solute carrier family 17 | 246213 | Channel or | Transporter | Transporter |
| | member 8 | | transporter | | |
| SLC17A9 | Solute carrier family 17 | 63910 | Channel or | Transporter | Transporter |
| | member 9 | | transporter | | |
| SLC18A1 | Solute carrier family 18 | 6570 | Channel or | Transporter | Transporter |
| | member A1 | | transporter | | |
| SLC18A2 | Solute carrier family 18 | 6571 | Channel or | Transporter | Transporter |
| | member A2 | | transporter | | |
| SLC18A3 | Solute carrier family 18 | 6572 | Channel or | Transporter | Transporter |
| | member A3 | | transporter | | |
| SLC18B1 | Solute carrier family 18 | 116843 | Channel or | Transporter | Transporter |
| | member B1 | | transporter | | |
| SLC19A1 | Solute carrier family 19 | 6573 | Channel or | Transporter | Transporter |
| | member 1 | | transporter | | |
| SLC19A2 | Solute carrier family 19 | 10560 | Channel or | Transporter | Transporter |
| | member 2 | | transporter | | |
| SLC19A3 | Solute carrier family 19 | 80704 | Channel or | Transporter | Transporter |
| | member 3 | | transporter | | |
| SLC1A1 | Solute carrier family 1 | 6505 | Channel or | Transporter | Transporter |
| | member 1 | | transporter | | |
| SLC1A2 | Solute carrier family 1 | 6506 | Channel or | Transporter | Transporter |
| | member 2 | | transporter | | |
| SLC1A3 | Solute carrier family 1 | 6507 | Channel or | Transporter | Transporter |
| | member 3 | | transporter | | |
| SLC1A6 | Solute carrier family 1 | 6511 | Channel or | Transporter | Transporter |
| | member 6 | | transporter | | |
| SLC1A7 | Solute carrier family 1 | 6512 | Channel or | Transporter | Transporter |
| | member 7 | | transporter | | |
| SLC20A1 | Solute carrier family 20 | 6574 | Channel or | Transporter | Transporter |
| | member 1 | | transporter | | |
| SLC20A2 | Solute carrier family 20 | 6575 | Channel or | Transporter | Transporter |
| | member 2 | | transporter | | |
| SLC22A1 | Solute carrier family 22 | 6580 | Channel or | Transporter | Transporter |
| | member 1 | | transporter | | |
| SLC22A10 | Solute carrier family 22 | 387775 | Channel or | Transporter | Transporter |
| | member 10 | | transporter | | |
| SLC22A11 | Solute carrier family 22 | 55867 | Channel or | Transporter | Transporter |
| | member 11 | | transporter | | |
| SLC22A12 | Solute carrier family 22 | 116085 | Channel or | Transporter | Transporter |
| | member 12 | | transporter | | |
| SLC22A13 | Solute carrier family 22 | 9390 | Channel or | Transporter | Transporter |
| | member 13 | | transporter | | |
| SLC22A14 | Solute carrier family 22 | 9389 | Channel or | Transporter | Transporter |
| | member 14 | | transporter | | |
| SLC22A15 | Solute carrier family 22 | 55356 | Channel or | Transporter | Transporter |
| | member 15 | | transporter | | |
| SLC22A16 | Solute carrier family 22 | 85413 | Channel or | Transporter | Transporter |
| | member 16 | | transporter | | |
| SLC22A17 | Solute carrier family 22 | 51310 | Channel or | Transporter | Transporter |
| | member 17 | | transporter | | |
| SLC22A18 | Solute carrier family 22 | 5002 | Channel or | Transporter | Transporter |
| | member 18 | | transporter | | |
| SLC22A2 | Solute carrier family 22 | 6582 | Channel or | Transporter | Transporter |
| | member 2 | | transporter | | |
| SLC22A20 | Solute carrier family 22 | 440044 | Channel or | Transporter | Transporter |
| | member 20 | | transporter | | |
| SLC22A23 | Solute carrier family 22 | 63027 | Channel or | Transporter | Transporter |
| | member 23 | | transporter | | |
| SLC22A24 | Solute carrier family 22 | 283238 | Channel or | Transporter | Transporter |
| | member 24 | | transporter | | |
| SLC22A25 | Solute carrier family 22 | 387601 | Channel or | Transporter | Transporter |
| | member 25 | | transporter | | |
| SLC22A3 | Solute carrier family 22 | 6581 | Channel or | Transporter | Transporter |
| | member 3 | | transporter | | |
| SLC22A31 | Solute carrier family 22 | 146429 | Channel or | Transporter | Transporter |
| | member 31 | | transporter | | |
| SLC22A4 | Solute carrier family 22 | 6583 | Channel or | Transporter | Transporter |
| | member 4 | | transporter | | |
| SLC22A5 | Solute carrier family 22 | 6584 | Channel or | Transporter | Transporter |
| | member 5 | | transporter | | |
| SLC22A6 | Solute carrier family 22 | 9356 | Channel or | Transporter | Transporter |
| | member 6 | | transporter | | |
| SLC22A7 | Solute carrier family 22 | 10864 | Channel or | Transporter | Transporter |
| | member 7 | | transporter | | |
| SLC22A8 | Solute carrier family 22 | 9376 | Channel or | Transporter | Transporter |
| | member 8 | | transporter | | |
| SLC22A9 | Solute carrier family 22 | 114571 | Channel or | Transporter | Transporter |
| | member 9 | | transporter | | |
| SLC23A1 | Solute carrier family 23 | 9963 | Channel or | Transporter | Transporter |
| | member 1 | | transporter | | |
| SLC23A2 | Solute carrier family 23 | 9962 | Channel or | Transporter | Transporter |
| | member 2 | | transporter | | |
| SLC23A3 | Solute carrier family 23 | 151295 | Channel or | Transporter | Transporter |
| | member 3 | | transporter | | |
| SLC23A4P | Solute carrier family 23 | 641842 | Channel or | Transporter | Transporter |
| | member 4, pseudogene | | transporter | | |
| SLC24A1 | Solute carrier family 24 | 9187 | Channel or | Transporter | Transporter |
| | member 1 | | transporter | | |
| SLC24A2 | Solute carrier family 24 | 25769 | Channel or | Transporter | Transporter |
| | member 2 | | transporter | | |
| SLC24A3 | Solute carrier family 24 | 57419 | Channel or | Transporter | Transporter |
| | member 3 | | transporter | | |
| SLC24A4 | Solute carrier family 24 | 123041 | Channel or | Transporter | Transporter |
| | member 4 | | transporter | | |
| SLC24A5 | Solute carrier family 24 | 283652 | Channel or | Transporter | Transporter |
| | member 5 | | transporter | | |
| SLC25A1 | Solute carrier family 25 | 6576 | Channel or | Transporter | Transporter |
| | member 1 | | transporter | | |
| SLC25A10 | Solute carrier family 25 | 1468 | Channel or | Transporter | Transporter |
| | member 10 | | transporter | | |
| SLC25A11 | Solute carrier family 25 | 8402 | Channel or | Transporter | Transporter |
| | member 11 | | transporter | | |
| SLC25A12 | Solute carrier family 25 | 8604 | Channel or | Transporter | Transporter |
| | member 12 | | transporter | | |
| SLC25A13 | Solute carrier family 25 | 10165 | Channel or | Transporter | Transporter |
| | member 13 | | transporter | | |
| SLC25A14 | Solute carrier family 25 | 9016 | Channel or | Transporter | Transporter |
| | member 14 | | transporter | | |
| SLC25A15 | Solute carrier family 25 | 10166 | Channel or | Transporter | Transporter |
| | member 15 | | transporter | | |
| SLC25A16 | Solute carrier family 25 | 8034 | Channel or | Transporter | Transporter |
| | member 16 | | transporter | | |
| SLC25A17 | Solute carrier family 25 | 10478 | Channel or | Transporter | Transporter |
| | member 17 | | transporter | | |
| SLC25A18 | Solute carrier family 25 | 83733 | Channel or | Transporter | Transporter |
| | member 18 | | transporter | | |
| SLC25A19 | Solute carrier family 25 | 60386 | Channel or | Transporter | Transporter |
| | member 19 | | transporter | | |
| SLC25A2 | Solute carrier family 25 | 83884 | Channel or | Transporter | Transporter |
| | member 2 | | transporter | | |
| SLC25A20 | Solute carrier family 25 | 788 | Channel or | Transporter | Transporter |
| | member 20 | | transporter | | |
| SLC25A21 | Solute carrier family 25 | 89874 | Channel or | Transporter | Transporter |
| | member 21 | | transporter | | |
| SLC25A22 | Solute carrier family 25 | 79751 | Channel or | Transporter | Transporter |
| | member 22 | | transporter | | |
| SLC25A23 | Solute carrier family 25 | 79085 | Channel or | Transporter | Transporter |
| | member 23 | | transporter | | |
| SLC25A24 | Solute carrier family 25 | 29957 | Channel or | Transporter | Transporter |
| | member 24 | | transporter | | |
| SLC25A25 | Solute carrier family 25 | 114789 | Channel or | Transporter | Transporter |
| | member 25 | | transporter | | |
| SLC25A26 | Solute carrier family 25 | 115286 | Channel or | Transporter | Transporter |
| | member 26 | | transporter | | |
| SLC25A27 | Solute carrier family 25 | 9481 | Channel or | Transporter | Transporter |
| | member 27 | | transporter | | |
| SLC25A28 | Solute carrier family 25 | 81894 | Channel or | Transporter | Transporter |
| | member 28 | | transporter | | |
| SLC25A29 | Solute carrier family 25 | 123096 | Channel or | Transporter | Transporter |
| | member 29 | | transporter | | |
| SLC25A3 | Solute carrier family 25 | 5250 | Channel or | Transporter | Transporter |
| | member 3 | | transporter | | |
| SLC25A30 | Solute carrier family 25 | 253512 | Channel or | Transporter | Transporter |
| | member 30 | | transporter | | |
| SLC25A31 | Solute carrier family 25 | 83447 | Channel or | Transporter | Transporter |
| | member 31 | | transporter | | |
| SLC25A32 | Solute carrier family 25 | 81034 | Channel or | Transporter | Transporter |
| | member 32 | | transporter | | |
| SLC25A33 | Solute carrier family 25 | 84275 | Channel or | Transporter | Transporter |
| | member 33 | | transporter | | |
| SLC25A34 | Solute carrier family 25 | 284723 | Channel or | Transporter | Transporter |
| | member 34 | | transporter | | |
| SLC25A35 | Solute carrier family 25 | 399512 | Channel or | Transporter | Transporter |
| | member 35 | | transporter | | |
| SLC25A36 | Solute carrier family 25 | 55186 | Channel or | Transporter | Transporter |
| | member 36 | | transporter | | |
| SLC25A37 | Solute carrier family 25 | 51312 | Channel or | Transporter | Transporter |
| | member 37 | | transporter | | |
| SLC25A38 | Solute carrier family 25 | 54977 | Channel or | Transporter | Transporter |
| | member 38 | | transporter | | |
| SLC25A39 | Solute carrier family 25 | 51629 | Channel or | Transporter | Transporter |
| | member 39 | | transporter | | |
| SLC25A4 | Solute carrier family 25 | 291 | Channel or | Transporter | Transporter |
| | member 4 | | transporter | | |
| SLC25A40 | Solute carrier family 25 | 55972 | Channel or | Transporter | Transporter |
| | member 40 | | transporter | | |
| SLC25A41 | Solute carrier family 25 | 284427 | Channel or | Transporter | Transporter |
| | member 41 | | transporter | | |
| SLC25A42 | Solute carrier family 25 | 284439 | Channel or | Transporter | Transporter |
| | member 42 | | transporter | | |
| SLC25A43 | Solute carrier family 25 | 203427 | Channel or | Transporter | Transporter |
| | member 43 | | transporter | | |
| SLC25A44 | Solute carrier family 25 | 9673 | Channel or | Transporter | Transporter |
| | member 44 | | transporter | | |
| SLC25A45 | Solute carrier family 25 | 283130 | Channel or | Transporter | Transporter |
| | member 45 | | transporter | | |
| SLC25A46 | Solute carrier family 25 | 91137 | Channel or | Transporter | Transporter |
| | member 46 | | transporter | | |
| SLC25A47 | Solute carrier family 25 | 283600 | Channel or | Transporter | Transporter |
| | member 47 | | transporter | | |
| SLC25A48 | Solute carrier family 25 | 153328 | Channel or | Transporter | Transporter |
| | member 48 | | transporter | | |
| SLC25A5 | Solute carrier family 25 | 292 | Channel or | Transporter | Transporter |
| | member 5 | | transporter | | |
| SLC25A51 | Solute carrier family 25 | 92014 | Channel or | Transporter | Transporter |
| | member 51 | | transporter | | |
| SLC25A52 | Solute carrier family 25 | 147407 | Channel or | Transporter | Transporter |
| | member 52 | | transporter | | |
| SLC25A53 | Solute carrier family 25 | 401612 | Channel or | Transporter | Transporter |
| | member 53 | | transporter | | |
| SLC25A6 | Solute carrier family 25 | 293 | Channel or | Transporter | Transporter |
| | member 6 | | transporter | | |
| SLC26A1 | Solute carrier family 26 | 10861 | Channel or | Transporter | Transporter |
| | member 1 | | transporter | | |
| SLC26A10 | Solute carrier family 26 | 65012 | Channel or | Transporter | Transporter |
| | member 10 | | transporter | | |
| SLC26A11 | Solute carrier family 26 | 284129 | Channel or | Transporter | Transporter |
| | member 11 | | transporter | | |
| SLC26A2 | Solute carrier family 26 | 1836 | Channel or | Transporter | Transporter |
| | member 2 | | transporter | | |
| SLC26A3 | Solute carrier family 26 | 1811 | Channel or | Transporter | Transporter |
| | member 3 | | transporter | | |
| SLC26A4 | Solute carrier family 26 | 5172 | Channel or | Transporter | Transporter |
| | member 4 | | transporter | | |
| SLC26A5 | Solute carrier family 26 | 375611 | Channel or | Transporter | Transporter |
| | member 5 | | transporter | | |
| SLC26A6 | Solute carrier family 26 | 65010 | Channel or | Transporter | Transporter |
| | member 6 | | transporter | | |
| SLC26A7 | Solute carrier family 26 | 115111 | Channel or | Transporter | Transporter |
| | member 7 | | transporter | | |
| SLC26A8 | Solute carrier family 26 | 116369 | Channel or | Transporter | Transporter |
| | member 8 | | transporter | | |
| SLC26A9 | Solute carrier family 26 | 115019 | Channel or | Transporter | Transporter |
| | member 9 | | transporter | | |
| SLC27A1 | Solute carrier family 27 | 376497 | Channel or | Transporter | Transporter |
| | member 1 | | transporter | | |
| SLC27A2 | Solute carrier family 27 | 11001 | Channel or | Transporter | Transporter |
| | member 2 | | transporter | | |
| SLC27A3 | Solute carrier family 27 | 11000 | Channel or | Transporter | Transporter |
| | member 3 | | transporter | | |
| SLC27A4 | Solute carrier family 27 | 10999 | Channel or | Transporter | Transporter |
| | member 4 | | transporter | | |
| SLC27A5 | Solute carrier family 27 | 10998 | Channel or | Transporter | Transporter |
| | member 5 | | transporter | | |
| SLC27A6 | Solute carrier family 27 | 28965 | Channel or | Transporter | Transporter |
| | member 6 | | transporter | | |
| SLC28A1 | Solute carrier family 28 | 9154 | Channel or | Transporter | Transporter |
| | member 1 | | transporter | | |
| SLC28A2 | Solute carrier family 28 | 9153 | Channel or | Transporter | Transporter |
| | member 2 | | transporter | | |
| SLC28A3 | Solute carrier family 28 | 64078 | Channel or | Transporter | Transporter |
| | member 3 | | transporter | | |
| SLC29A1 | Solute carrier family 29 | 2030 | Channel or | Transporter | Transporter |
| | member 1 (Augustine blood | | transporter | | |
| | group) | | | | |
| SLC29A2 | Solute carrier family 29 | 3177 | Channel or | Transporter | Transporter |
| | member 2 | | transporter | | |
| SLC29A3 | Solute carrier family 29 | 55315 | Channel or | Transporter | Transporter |
| | member 3 | | transporter | | |
| SLC29A4 | Solute carrier family 29 | 222962 | Channel or | Transporter | Transporter |
| | member 4 | | transporter | | |
| SLC2A1 | Solute carrier family 2 | 6513 | Channel or | Transporter | Transporter |
| | member 1 | | transporter | | |
| SLC2A10 | Solute carrier family 2 | 81031 | Channel or | Transporter | Transporter |
| | member 10 | | transporter | | |
| SLC2A11 | Solute carrier family 2 | 66035 | Channel or | Transporter | Transporter |
| | member 11 | | transporter | | |
| SLC2A12 | Solute carrier family 2 | 154091 | Channel or | Transporter | Transporter |
| | member 12 | | transporter | | |
| SLC2A13 | Solute carrier family 2 | 114134 | Channel or | Transporter | Transporter |
| | member 13 | | transporter | | |
| SLC2A14 | Solute carrier family 2 | 144195 | Channel or | Transporter | Transporter |
| | member 14 | | transporter | | |
| SLC2A2 | Solute carrier family 2 | 6514 | Channel or | Transporter | Transporter |
| | member 2 | | transporter | | |
| SLC2A3 | Solute carrier family 2 | 6515 | Channel or | Transporter | Transporter |
| | member 3 | | transporter | | |
| SLC2A4 | Solute carrier family 2 | 6517 | Channel or | Transporter | Transporter |
| | member 4 | | transporter | | |
| SLC2A5 | Solute carrier family 2 | 6518 | Channel or | Transporter | Transporter |
| | member 5 | | transporter | | |
| SLC2A6 | Solute carrier family 2 | 11182 | Channel or | Transporter | Transporter |
| | member 6 | | transporter | | |
| SLC2A7 | Solute carrier family 2 | 155184 | Channel or | Transporter | Transporter |
| | member 7 | | transporter | | |
| SLC2A8 | Solute carrier family 2 | 29988 | Channel or | Transporter | Transporter |
| | member 8 | | transporter | | |
| SLC2A9 | Solute carrier family 2 | 56606 | Channel or | Transporter | Transporter |
| | member 9 | | transporter | | |
| SLC30A1 | Solute carrier family 30 | 7779 | Channel or | Transporter | Transporter |
| | member 1 | | transporter | | |
| SLC30A10 | Solute carrier family 30 | 55532 | Channel or | Transporter | Transporter |
| | member 10 | | transporter | | |
| SLC30A2 | Solute carrier family 30 | 7780 | Channel or | Transporter | Transporter |
| | member 2 | | transporter | | |
| SLC30A3 | Solute carrier family 30 | 7781 | Channel or | Transporter | Transporter |
| | member 3 | | transporter | | |
| SLC30A4 | Solute carrier family 30 | 7782 | Channel or | Transporter | Transporter |
| | member 4 | | transporter | | |
| SLC30A5 | Solute carrier family 30 | 64924 | Channel or | Transporter | Transporter |
| | member 5 | | transporter | | |
| SLC30A6 | Solute carrier family 30 | 55676 | Channel or | Transporter | Transporter |
| | member 6 | | transporter | | |
| SLC30A7 | Solute carrier family 30 | 148867 | Channel or | Transporter | Transporter |
| | member 7 | | transporter | | |
| SLC30A8 | Solute carrier family 30 | 169026 | Channel or | Transporter | Transporter |
| | member 8 | | transporter | | |
| SLC30A9 | Solute carrier family 30 | 10463 | Channel or | Transporter | Transporter |
| | member 9 | | transporter | | |
| SLC31A1 | Solute carrier family 31 | 1317 | Channel or | Transporter | Transporter |
| | member 1 | | transporter | | |
| SLC31A2 | Solute carrier family 31 | 1318 | Channel or | Transporter | Transporter |
| | member 2 | | transporter | | |
| SLC32A1 | Solute carrier family 32 | 140679 | Channel or | Transporter | Transporter |
| | member 1 | | transporter | | |
| SLC33A1 | Solute carrier family 33 | 9197 | Channel or | Transporter | Transporter |
| | member 1 | | transporter | | |
| SLC34A1 | Solute carrier family 34 | 6569 | Channel or | Transporter | Transporter |
| | member 1 | | transporter | | |
| SLC34A2 | Solute carrier family 34 | 10568 | Channel or | Transporter | Transporter |
| | member 2 | | transporter | | |
| SLC34A3 | Solute carrier family 34 | 142680 | Channel or | Transporter | Transporter |
| | member 3 | | transporter | | |
| SLC35A1 | Solute carrier family 35 | 10559 | Channel or | Transporter | Transporter |
| | member A1 | | transporter | | |
| SLC35A2 | Solute carrier family 35 | 7355 | Channel or | Transporter | Transporter |
| | member A2 | | transporter | | |
| SLC35A3 | Solute carrier family 35 | 23443 | Channel or | Transporter | Transporter |
| | member A3 | | transporter | | |
| SLC35A4 | Solute carrier family 35 | 113829 | Channel or | Transporter | Transporter |
| | member A4 | | transporter | | |
| SLC35A5 | Solute carrier family 35 | 55032 | Channel or | Transporter | Transporter |
| | member A5 | | transporter | | |
| SLC35B1 | Solute carrier family 35 | 10237 | Channel or | Transporter | Transporter |
| | member B1 | | transporter | | |
| SLC35B2 | Solute carrier family 35 | 347734 | Channel or | Transporter | Transporter |
| | member B2 | | transporter | | |
| SLC35B3 | Solute carrier family 35 | 51000 | Channel or | Transporter | Transporter |
| | member B3 | | transporter | | |
| SLC35B4 | Solute carrier family 35 | 84912 | Channel or | Transporter | Transporter |
| | member B4 | | transporter | | |
| SLC35C1 | Solute carrier family 35 | 55343 | Channel or | Transporter | Transporter |
| | member C1 | | transporter | | |
| SLC35C2 | Solute carrier family 35 | 51006 | Channel or | Transporter | Transporter |
| | member C2 | | transporter | | |
| SLC35D1 | Solute carrier family 35 | 23169 | Channel or | Transporter | Transporter |
| | member D1 | | transporter | | |
| SLC35D2 | Solute carrier family 35 | 11046 | Channel or | Transporter | Transporter |
| | member D2 | | transporter | | |
| SLC35D3 | Solute carrier family 35 | 340146 | Channel or | Transporter | Transporter |
| | member D3 | | transporter | | |
| SLC35E1 | Solute carrier family 35 | 79939 | Channel or | Transporter | Transporter |
| | member E1 | | transporter | | |
| SLC35E2 | Solute carrier family 35 | 9906 | Channel or | Transporter | Transporter |
| | member E2 | | transporter | | |
| SLC35E2B | Solute carrier family 35 | 728661 | Channel or | Transporter | Transporter |
| | member E2B | | transporter | | |
| SLC35E3 | Solute carrier family 35 | 55508 | Channel or | Transporter | Transporter |
| | member E3 | | transporter | | |
| SLC35E4 | Solute carrier family 35 | 339665 | Channel or | Transporter | Transporter |
| | member E4 | | transporter | | |
| SLC35F1 | Solute carrier family 35 | 222553 | Channel or | Transporter | Transporter |
| | member F1 | | transporter | | |
| SLC35F2 | Solute carrier family 35 | 54733 | Channel or | Transporter | Transporter |
| | member F2 | | transporter | | |
| SLC35F3 | Solute carrier family 35 | 148641 | Channel or | Transporter | Transporter |
| | member F3 | | transporter | | |
| SLC35F4 | Solute carrier family 35 | 341880 | Channel or | Transporter | Transporter |
| | member F4 | | transporter | | |
| SLC35F5 | Solute carrier family 35 | 80255 | Channel or | Transporter | Transporter |
| | member F5 | | transporter | | |
| SLC35F6 | Solute carrier family 35 | 54978 | Channel or | Transporter | Transporter |
| | member F6 | | transporter | | |
| SLC35G1 | Solute carrier family 35 | 159371 | Channel or | Transporter | Transporter |
| | member G1 | | transporter | | |
| SLC35G2 | Solute carrier family 35 | 80723 | Channel or | Transporter | Transporter |
| | member G2 | | transporter | | |
| SLC35G3 | Solute carrier family 35 | 146861 | Channel or | Transporter | Transporter |
| | member G3 | | transporter | | |
| SLC35G4 | Solute carrier family 35 | 646000 | Channel or | Transporter | Transporter |
| | member G4 | | transporter | | |
| SLC35G5 | Solute carrier family 35 | 83650 | Channel or | Transporter | Transporter |
| | member G5 | | transporter | | |
| SLC35G6 | Solute carrier family 35 | 643664 | Channel or | Transporter | Transporter |
| | member G6 | | transporter | | |
| SLC36A1 | Solute carrier family 36 | 206358 | Channel or | Transporter | Transporter |
| | member 1 | | transporter | | |
| SLC36A2 | Solute carrier family 36 | 153201 | Channel or | Transporter | Transporter |
| | member 2 | | transporter | | |
| SLC36A3 | Solute carrier family 36 | 285641 | Channel or | Transporter | Transporter |
| | member 3 | | transporter | | |
| SLC36A4 | Solute carrier family 36 | 120103 | Channel or | Transporter | Transporter |
| | member 4 | | transporter | | |
| SLC37A1 | Solute carrier family 37 | 54020 | Channel or | Transporter | Transporter |
| | member 1 | | transporter | | |
| SLC37A2 | Solute carrier family 37 | 219855 | Channel or | Transporter | Transporter |
| | member 2 | | transporter | | |
| SLC37A3 | Solute carrier family 37 | 84255 | Channel or | Transporter | Transporter |
| | member 3 | | transporter | | |
| SLC37A4 | Solute carrier family 37 | 2542 | Channel or | Transporter | Transporter |
| | member 4 | | transporter | | |
| SLC38A1 | Solute carrier family 38 | 81539 | Channel or | Transporter | Transporter |
| | member 1 | | transporter | | |
| SLC38A10 | Solute carrier family 38 | 124565 | Channel or | Transporter | Transporter |
| | member 10 | | transporter | | |
| SLC38A11 | Solute carrier family 38 | 151258 | Channel or | Transporter | Transporter |
| | member 11 | | transporter | | |
| SLC38A2 | Solute carrier family 38 | 54407 | Channel or | Transporter | Transporter |
| | member 2 | | transporter | | |
| SLC38A3 | Solute carrier family 38 | 10991 | Channel or | Transporter | Transporter |
| | member 3 | | transporter | | |
| SLC38A4 | Solute carrier family 38 | 55089 | Channel or | Transporter | Transporter |
| | member 4 | | transporter | | |
| SLC38A5 | Solute carrier family 38 | 92745 | Channel or | Transporter | Transporter |
| | member 5 | | transporter | | |
| SLC38A6 | Solute carrier family 38 | 145389 | Channel or | Transporter | Transporter |
| | member 6 | | transporter | | |
| SLC38A7 | Solute carrier family 38 | 55238 | Channel or | Transporter | Transporter |
| | member 7 | | transporter | | |
| SLC38A8 | Solute carrier family 38 | 146167 | Channel or | Transporter | Transporter |
| | member 8 | | transporter | | |
| SLC38A9 | Solute carrier family 38 | 153129 | Channel or | Transporter | Transporter |
| | member 9 | | transporter | | |
| SLC39A1 | Solute carrier family 39 | 27173 | Channel or | Transporter | Transporter |
| | member 1 | | transporter | | |
| SLC39A10 | Solute carrier family 39 | 57181 | Channel or | Transporter | Transporter |
| | member 10 | | transporter | | |
| SLC39A11 | Solute carrier family 39 | 201266 | Channel or | Transporter | Transporter |
| | member 11 | | transporter | | |
| SLC39A12 | Solute carrier family 39 | 221074 | Channel or | Transporter | Transporter |
| | member 12 | | transporter | | |
| SLC39A13 | Solute carrier family 39 | 91252 | Channel or | Transporter | Transporter |
| | member 13 | | transporter | | |
| SLC39A14 | Solute carrier family 39 | 23516 | Channel or | Transporter | Transporter |
| | member 14 | | transporter | | |
| SLC39A2 | Solute carrier family 39 | 29986 | Channel or | Transporter | Transporter |
| | member 2 | | transporter | | |
| SLC39A3 | Solute carrier family 39 | 29985 | Channel or | Transporter | Transporter |
| | member 3 | | transporter | | |
| SLC39A4 | Solute carrier family 39 | 55630 | Channel or | Transporter | Transporter |
| | member 4 | | transporter | | |
| SLC39A5 | Solute carrier family 39 | 283375 | Channel or | Transporter | Transporter |
| | member 5 | | transporter | | |
| SLC39A6 | Solute carrier family 39 | 25800 | Channel or | Transporter | Transporter |
| | member 6 | | transporter | | |
| SLC39A7 | Solute carrier family 39 | 7922 | Channel or | Transporter | Transporter |
| | member 7 | | transporter | | |
| SLC39A8 | Solute carrier family 39 | 64116 | Channel or | Transporter | Transporter |
| | member 8 | | transporter | | |
| SLC39A9 | Solute carrier family 39 | 55334 | Channel or | Transporter | Transporter |
| | member 9 | | transporter | | |
| SLC3A1 | Solute carrier family 3 | 6519 | Channel or | Transporter | Transporter |
| | member 1 | | transporter | | |
| SLC3A2 | Solute carrier family 3 | 6520 | Channel or | Transporter | Transporter |
| | member 2 | | transporter | | |
| SLC40A1 | Solute carrier family 40 | 30061 | Channel or | Transporter | Transporter |
| | member 1 | | transporter | | |
| SLC41A1 | Solute carrier family 41 | 254428 | Channel or | Transporter | Transporter |
| | member 1 | | transporter | | |
| SLC41A2 | Solute carrier family 41 | 84102 | Channel or | Transporter | Transporter |
| | member 2 | | transporter | | |
| SLC41A3 | Solute carrier family 41 | 54946 | Channel or | Transporter | Transporter |
| | member 3 | | transporter | | |
| SLC43A1 | Solute carrier family 43 | 8501 | Channel or | Transporter | Transporter |
| | member 1 | | transporter | | |
| SLC43A2 | Solute carrier family 43 | 124935 | Channel or | Transporter | Transporter |
| | member 2 | | transporter | | |
| SLC43A3 | Solute carrier family 43 | 29015 | Channel or | Transporter | Transporter |
| | member 3 | | transporter | | |
| SLC44A1 | Solute carrier family 44 | 23446 | Channel or | Transporter | Transporter |
| | member 1 | | transporter | | |
| SLC44A2 | Solute carrier family 44 | 57153 | Channel or | Transporter | Transporter |
| | member 2 | | transporter | | |
| SLC44A3 | Solute carrier family 44 | 126969 | Channel or | Transporter | Transporter |
| | member 3 | | transporter | | |
| SLC44A4 | Solute carrier family 44 | 80736 | Channel or | Transporter | Transporter |
| | member 4 | | transporter | | |
| SLC44A5 | Solute carrier family 44 | 204962 | Channel or | Transporter | Transporter |
| | member 5 | | transporter | | |
| SLC45A1 | Solute carrier family 45 | 50651 | Channel or | Transporter | Transporter |
| | member 1 | | transporter | | |
| SLC45A2 | Solute carrier family 45 | 51151 | Channel or | Transporter | Transporter |
| | member 2 | | transporter | | |
| SLC45A3 | Solute carrier family 45 | 85414 | Channel or | Transporter | Transporter |
| | member 3 | | transporter | | |
| SLC45A4 | Solute carrier family 45 | 57210 | Channel or | Transporter | Transporter |
| | member 4 | | transporter | | |
| SLC46A1 | Solute carrier family 46 | 113235 | Channel or | Transporter | Transporter |
| | member 1 | | transporter | | |
| SLC46A2 | Solute carrier family 46 | 57864 | Channel or | Transporter | Transporter |
| | member 2 | | transporter | | |
| SLC46A3 | Solute carrier family 46 | 283537 | Channel or | Transporter | Transporter |
| | member 3 | | transporter | | |
| SLC47A1 | Solute carrier family 47 | 55244 | Channel or | Transporter | Transporter |
| | member 1 | | transporter | | |
| SLC47A2 | Solute carrier family 47 | 146802 | Channel or | Transporter | Transporter |
| | member 2 | | transporter | | |
| SLC48A1 | Solute carrier family 48 | 55652 | Channel or | Transporter | Transporter |
| | member 1 | | transporter | | |
| SLC4A1 | Solute carrier family 4 | 6521 | Channel or | Transporter | Transporter |
| | member 1 | | transporter | | |
| SLC4A10 | Solute carrier family 4 | 57282 | Channel or | Transporter | Transporter |
| | member 10 | | transporter | | |
| SLC4A11 | Solute carrier family 4 | 83959 | Channel or | Transporter | Transporter |
| | member 11 | | transporter | | |
| SLC4A2 | Solute carrier family 4 | 6522 | Channel or | Transporter | Transporter |
| | member 2 | | transporter | | |
| SLC4A3 | Solute carrier family 4 | 6508 | Channel or | Transporter | Transporter |
| | member 3 | | transporter | | |
| SLC4A4 | Solute carrier family 4 | 8671 | Channel or | Transporter | Transporter |
| | member 4 | | transporter | | |
| SLC4A5 | Solute carrier family 4 | 57835 | Channel or | Transporter | Transporter |
| | member 5 | | transporter | | |
| SLC4A7 | Solute carrier family 4 | 9497 | Channel or | Transporter | Transporter |
| | member 7 | | transporter | | |
| SLC4A8 | Solute carrier family 4 | 9498 | Channel or | Transporter | Transporter |
| | member 8 | | transporter | | |
| SLC4A9 | Solute carrier family 4 | 83697 | Channel or | Transporter | Transporter |
| | member 9 | | transporter | | |
| SLC50A1 | Solute carrier family 50 | 55974 | Channel or | Transporter | Transporter |
| | member 1 | | transporter | | |
| SLC51A | Solute carrier family 51 | 200931 | Channel or | Transporter | Transporter |
| | alpha subunit | | transporter | | |
| SLC51B | Solute carrier family 51 beta | 123264 | Channel or | Transporter | Transporter |
| | subunit | | transporter | | |
| SLC52A1 | Solute carrier family 52 | 55065 | Channel or | Transporter | Transporter |
| | member 1 | | transporter | | |
| SLC52A2 | Solute carrier family 52 | 79581 | Channel or | Transporter | Transporter |
| | member 2 | | transporter | | |
| SLC52A3 | Solute carrier family 52 | 113278 | Channel or | Transporter | Transporter |
| | member 3 | | transporter | | |
| SLC5A1 | Solute carrier family 5 | 6523 | Channel or | Transporter | Transporter |
| | member 1 | | transporter | | |
| SLC5A10 | Solute carrier family 5 | 125206 | Channel or | Transporter | Transporter |
| | member 10 | | transporter | | |
| SLC5A11 | Solute carrier family 5 | 115584 | Channel or | Transporter | Transporter |
| | member 11 | | transporter | | |
| SLC5A12 | Solute carrier family 5 | 159963 | Channel or | Transporter | Transporter |
| | member 12 | | transporter | | |
| SLC5A2 | Solute carrier family 5 | 6524 | Channel or | Transporter | Transporter |
| | member 2 | | transporter | | |
| SLC5A3 | Solute carrier family 5 | 6526 | Channel or | Transporter | Transporter |
| | member 3 | | transporter | | |
| SLC5A4 | Solute carrier family 5 | 6527 | Channel or | Transporter | Transporter |
| | member 4 | | transporter | | |
| SLC5A5 | Solute carrier family 5 | 6528 | Channel or | Transporter | Transporter |
| | member 5 | | transporter | | |
| SLC5A6 | Solute carrier family 5 | 8884 | Channel or | Transporter | Transporter |
| | member 6 | | transporter | | |
| SLC5A7 | Solute carrier family 5 | 60482 | Channel or | Transporter | Transporter |
| | member 7 | | transporter | | |
| SLC5A8 | Solute carrier family 5 | 160728 | Channel or | Transporter | Transporter |
| | member 8 | | transporter | | |
| SLC5A9 | Solute carrier family 5 | 200010 | Channel or | Transporter | Transporter |
| | member 9 | | transporter | | |
| SLC6A1 | Solute carrier family 6 | 6529 | Channel or | Transporter | Transporter |
| | member 1 | | transporter | | |
| SLC6A10P | Solute carrier family 6 | 386757 | Channel or | Transporter | Transporter |
| | member 10, pseudogene | | transporter | | |
| SLC6A10PB | Solute carrier family 6 | 653562 | Channel or | Transporter | Transporter |
| | member 8 pseudogene | | transporter | | |
| SLC6A11 | Solute carrier family 6 | 6538 | Channel or | Transporter | Transporter |
| | member 11 | | transporter | | |
| SLC6A12 | Solute carrier family 6 | 6539 | Channel or | Transporter | Transporter |
| | member 12 | | transporter | | |
| SLC6A13 | Solute carrier family 6 | 6540 | Channel or | Transporter | Transporter |
| | member 13 | | transporter | | |
| SLC6A14 | Solute carrier family 6 | 11254 | Channel or | Transporter | Transporter |
| | member 14 | | transporter | | |
| SLC6A15 | Solute carrier family 6 | 55117 | Channel or | Transporter | Transporter |
| | member 15 | | transporter | | |
| SLC6A16 | Solute carrier family 6 | 28968 | Channel or | Transporter | Transporter |
| | member 16 | | transporter | | |
| SLC6A17 | Solute carrier family 6 | 388662 | Channel or | Transporter | Transporter |
| | member 17 | | transporter | | |
| SLC6A18 | Solute carrier family 6 | 348932 | Channel or | Transporter | Transporter |
| | member 18 | | transporter | | |
| SLC6A19 | Solute carrier family 6 | 340024 | Channel or | Transporter | Transporter |
| | member 19 | | transporter | | |
| SLC6A2 | Solute carrier family 6 | 6530 | Channel or | Transporter | Transporter |
| | member 2 | | transporter | | |
| SLC6A20 | Solute carrier family 6 | 54716 | Channel or | Transporter | Transporter |
| | member 20 | | transporter | | |
| SLC6A21P | Solute carrier family 6 | 652969 | Channel or | Transporter | Transporter |
| | member 21, pseudogene | | transporter | | |
| SLC6A3 | Solute carrier family 6 | 6531 | Channel or | Transporter | Transporter |
| | member 3 | | transporter | | |
| SLC6A4 | Solute carrier family 6 | 6532 | Channel or | Transporter | Transporter |
| | member 4 | | transporter | | |
| SLC6A5 | Solute carrier family 6 | 9152 | Channel or | Transporter | Transporter |
| | member 5 | | transporter | | |
| SLC6A6 | Solute carrier family 6 | 6533 | Channel or | Transporter | Transporter |
| | member 6 | | transporter | | |
| SLC6A7 | Solute carrier family 6 | 6534 | Channel or | Transporter | Transporter |
| | member 7 | | transporter | | |
| SLC6A8 | Solute carrier family 6 | 6535 | Channel or | Transporter | Transporter |
| | member 8 | | transporter | | |
| SLC6A9 | Solute carrier family 6 | 6536 | Channel or | Transporter | Transporter |
| | member 9 | | transporter | | |
| SLC7A1 | Solute carrier family 7 | 6541 | Channel or | Transporter | Transporter |
| | member 1 | | transporter | | |
| SLC7A10 | Solute carrier family 7 | 56301 | Channel or | Transporter | Transporter |
| | member 10 | | transporter | | |
| SLC7A11 | Solute carrier family 7 | 23657 | Channel or | Transporter | Transporter |
| | member 11 | | transporter | | |
| SLC7A13 | Solute carrier family 7 | 157724 | Channel or | Transporter | Transporter |
| | member 13 | | transporter | | |
| SLC7A14 | Solute carrier family 7 | 57709 | Channel or | Transporter | Transporter |
| | member 14 | | transporter | | |
| SLC7A2 | Solute carrier family 7 | 6542 | Channel or | Transporter | Transporter |
| | member 2 | | transporter | | |
| SLC7A3 | Solute carrier family 7 | 84889 | Channel or | Transporter | Transporter |
| | member 3 | | transporter | | |
| SLC7A4 | Solute carrier family 7 | 6545 | Channel or | Transporter | Transporter |
| | member 4 | | transporter | | |
| SLC7A5 | Solute carrier family 7 | 8140 | Channel or | Transporter | Transporter |
| | member 5 | | transporter | | |
| SLC7A6 | Solute carrier family 7 | 9057 | Channel or | Transporter | Transporter |
| | member 6 | | transporter | | |
| SLC7A7 | Solute carrier family 7 | 9056 | Channel or | Transporter | Transporter |
| | member 7 | | transporter | | |
| SLC7A8 | Solute carrier family 7 | 23428 | Channel or | Transporter | Transporter |
| | member 8 | | transporter | | |
| SLC7A9 | Solute carrier family 7 | 11136 | Channel or | Transporter | Transporter |
| | member 9 | | transporter | | |
| SLC8A1 | Solute carrier family 8 | 6546 | Channel or | Transporter | Transporter |
| | member A1 | | transporter | | |
| SLC8A2 | Solute carrier family 8 | 6543 | Channel or | Transporter | Transporter |
| | member A2 | | transporter | | |
| SLC8A3 | Solute carrier family 8 | 6547 | Channel or | Transporter | Transporter |
| | member A3 | | transporter | | |
| SLC8B1 | Solute carrier family 8 | 80024 | Channel or | Transporter | Transporter |
| | member B1 | | transporter | | |
| SLC9A1 | Solute carrier family 9 | 6548 | Channel or | Transporter | Transporter |
| | member A1 | | transporter | | |
| SLC9A2 | Solute carrier family 9 | 6549 | Channel or | Transporter | Transporter |
| | member A2 | | transporter | | |
| SLC9A3 | Solute carrier family 9 | 6550 | Channel or | Transporter | Transporter |
| | member A3 | | transporter | | |
| SLC9A4 | Solute carrier family 9 | 389015 | Channel or | Transporter | Transporter |
| | member A4 | | transporter | | |
| SLC9A5 | Solute carrier family 9 | 6553 | Channel or | Transporter | Transporter |
| | member A5 | | transporter | | |
| SLC9A6 | Solute carrier family 9 | 10479 | Channel or | Transporter | Transporter |
| | member A6 | | transporter | | |
| SLC9A7 | Solute carrier family 9 | 84679 | Channel or | Transporter | Transporter |
| | member A7 | | transporter | | |
| SLC9A8 | Solute carrier family 9 | 23315 | Channel or | Transporter | Transporter |
| | member A8 | | transporter | | |
| SLC9A9 | Solute carrier family 9 | 285195 | Channel or | Transporter | Transporter |
| | member A9 | | transporter | | |
| SLC9B1 | Solute carrier family 9 | 150159 | Channel or | Transporter | Transporter |
| | member B1 | | transporter | | |
| SLC9B2 | Solute carrier family 9 | 133308 | Channel or | Transporter | Transporter |
| | member B2 | | transporter | | |
| SLC9C1 | Solute carrier family 9 | 285335 | Channel or | Transporter | Transporter |
| | member C1 | | transporter | | |
| SLC9C2 | Solute carrier family 9 | 284525 | Channel or | Transporter | Transporter |
| | member C2 (putative) | | transporter | | |
| SLCO1A2 | Solute carrier organic anion | 6579 | Channel or | Transporter | Transporter |
| | transporter family member | | transporter | | |
| | 1A2 | | | | |
| SLCO1B1 | Solute carrier organic anion | 10599 | Channel or | Transporter | Transporter |
| | transporter family member | | transporter | | |
| | 1B1 | | | | |
| SLCO1B3 | Solute carrier organic anion | 28234 | Channel or | Transporter | Transporter |
| | transporter family member | | transporter | | |
| | 1B3 | | | | |
| SLCO1C1 | Solute carrier organic anion | 53919 | Channel or | Transporter | Transporter |
| | transporter family member | | transporter | | |
| | 1C1 | | | | |
| SLCO2A1 | Solute carrier organic anion | 6578 | Channel or | Transporter | Transporter |
| | transporter family member | | transporter | | |
| | 2A1 | | | | |
| SLCO2B1 | Solute carrier organic anion | 11309 | Channel or | Transporter | Transporter |
| | transporter family member | | transporter | | |
| | 2B1 | | | | |
| SLCO3A1 | Solute carrier organic anion | 28232 | Channel or | Transporter | Transporter |
| | transporter family member | | transporter | | |
| | 3A1 | | | | |
| SLCO4A1 | Solute carrier organic anion | 28231 | Channel or | Transporter | Transporter |
| | transporter family member | | transporter | | |
| | 4A1 | | | | |
| SLCO4C1 | Solute carrier organic anion | 353189 | Channel or | Transporter | Transporter |
| | transporter family member | | transporter | | |
| | 4C1 | | | | |
| SLCO5A1 | Solute carrier organic anion | 81796 | Channel or | Transporter | Transporter |
| | transporter family member | | transporter | | |
| | 5A1 | | | | |
| SLCO6A1 | Solute carrier organic anion | 133482 | Channel or | Transporter | Transporter |
| | transporter family member | | transporter | | |
| | 6A1 | | | | |
| TCIRG1 | T-cell immune regulator 1, | 10312 | Channel or | Transporter | Transporter |
| | atpase H+ transporting V0 | | transporter | | |
| | subunit a3 | | | | |
| UCP1 | Uncoupling protein 1 | 7350 | Channel or | Transporter | Transporter |
| | | | transporter | | |
| UCP2 | Uncoupling protein 2 | 7351 | Channel or | Transporter | Transporter |
| | | | transporter | | |
| UCP3 | Uncoupling protein 3 | 7352 | Channel or | Transporter | Transporter |
| | | | transporter | | |
| CACNA1A | Calcium voltage-gated | 773 | Channel or | Channel | Vgic |
| | channel subunit alpha1 A | | transporter | | |
| CACNA1B | Calcium voltage-gated | 774 | Channel or | Channel | Vgic |
| | channel subunit alpha1 B | | transporter | | |
| CACNA1C | Calcium voltage-gated | 775 | Channel or | Channel | Vgic |
| | channel subunit alpha1 C | | transporter | | |
| CACNA1D | Calcium voltage-gated | 776 | Channel or | Channel | Vgic |
| | channel subunit alpha1 D | | transporter | | |
| CACNA1E | Calcium voltage-gated | 777 | Channel or | Channel | Vgic |
| | channel subunit alpha1 E | | transporter | | |
| CACNA1F | Calcium voltage-gated | 778 | Channel or | Channel | Vgic |
| | channel subunit alpha1 F | | transporter | | |
| CACNA1G | Calcium voltage-gated | 8913 | Channel or | Channel | Vgic |
| | channel subunit alpha1 G | | transporter | | |
| CACNA1H | Calcium voltage-gated | 8912 | Channel or | Channel | Vgic |
| | channel subunit alpha1 H | | transporter | | |
| CACNA1I | Calcium voltage-gated | 8911 | Channel or | Channel | Vgic |
| | channel subunit alpha1 I | | transporter | | |
| CACNA1S | Calcium voltage-gated | 779 | Channel or | Channel | Vgic |
| | channel subunit alpha1 S | | transporter | | |
| CACNB1 | Calcium voltage-gated | 782 | Channel or | Channel | Vgic |
| | channel auxiliary subunit | | transporter | | |
| | beta 1 | | | | |
| CACNB2 | Calcium voltage-gated | 783 | Channel or | Channel | Vgic |
| | channel auxiliary subunit | | transporter | | |
| | beta 2 | | | | |
| CACNB4 | Calcium voltage-gated | 785 | Channel or | Channel | Vgic |
| | channel auxiliary subunit | | transporter | | |
| | beta 4 | | | | |
| CATSPER1 | Cation channel sperm | 117144 | Channel or | Channel | Vgic |
| | associated 1 | | transporter | | |
| CATSPER2 | Cation channel sperm | 117155 | Channel or | Channel | Vgic |
| | associated 2 | | transporter | | |
| CATSPER3 | Cation channel sperm | 347732 | Channel or | Channel | Vgic |
| | associated 3 | | transporter | | |
| CATSPER4 | Cation channel sperm | 378807 | Channel or | Channel | Vgic |
| | associated 4 | | transporter | | |
| CNGA1 | Cyclic nucleotide gated | 1259 | Channel or | Channel | Vgic |
| | channel alpha 1 | | transporter | | |
| CNGA2 | Cyclic nucleotide gated | 1260 | Channel or | Channel | Vgic |
| | channel alpha 2 | | transporter | | |
| CNGA3 | Cyclic nucleotide gated | 1261 | Channel or | Channel | Vgic |
| | channel alpha 3 | | transporter | | |
| CNGA4 | Cyclic nucleotide gated | 1262 | Channel or | Channel | Vgic |
| | channel alpha 4 | | transporter | | |
| CNGB1 | Cyclic nucleotide gated | 1258 | Channel or | Channel | Vgic |
| | channel beta 1 | | transporter | | |
| CNGB3 | Cyclic nucleotide gated | 54714 | Channel or | Channel | Vgic |
| | channel beta 3 | | transporter | | |
| HCN1 | Hyperpolarization activated | 348980 | Channel or | Channel | Vgic |
| | cyclic nucleotide gated | | transporter | | |
| | potassium channel 1 | | | | |
| HCN2 | Hyperpolarization activated | 610 | Channel or | Channel | Vgic |
| | cyclic nucleotide gated | | transporter | | |
| | potassium channel 2 | | | | |
| HCN3 | Hyperpolarization activated | 57657 | Channel or | Channel | Vgic |
| | cyclic nucleotide gated | | transporter | | |
| | potassium channel 3 | | | | |
| HCN4 | Hyperpolarization activated | 10021 | Channel or | Channel | Vgic |
| | cyclic nucleotide gated | | transporter | | |
| | potassium channel 4 | | | | |
| HVCN1 | Hydrogen voltage gated | 84329 | Channel or | Channel | Vgic |
| | channel 1 | | transporter | | |
| KCNA1 | Potassium voltage-gated | 3736 | Channel or | Channel | Vgic |
| | channel subfamily A | | transporter | | |
| | member 1 | | | | |
| KCNA10 | Potassium voltage-gated | 3744 | Channel or | Channel | Vgic |
| | channel subfamily A | | transporter | | |
| | member 10 | | | | |
| KCNA2 | Potassium voltage-gated | 3737 | Channel or | Channel | Vgic |
| | channel subfamily A | | transporter | | |
| | member 2 | | | | |
| KCNA3 | Potassium voltage-gated | 3738 | Channel or | Channel | Vgic |
| | channel subfamily A | | transporter | | |
| | member 3 | | | | |
| KCNA4 | Potassium voltage-gated | 3739 | Channel or | Channel | Vgic |
| | channel subfamily A | | transporter | | |
| | member 4 | | | | |
| KCNA5 | Potassium voltage-gated | 3741 | Channel or | Channel | Vgic |
| | channel subfamily A | | transporter | | |
| | member 5 | | | | |
| KCNA6 | Potassium voltage-gated | 3742 | Channel or | Channel | Vgic |
| | channel subfamily A | | transporter | | |
| | member 6 | | | | |
| KCNA7 | Potassium voltage-gated | 3743 | Channel or | Channel | Vgic |
| | channel subfamily A | | transporter | | |
| | member 7 | | | | |
| KCNAB1 | Potassium voltage-gated | 7881 | Channel or | Channel | Vgic |
| | channel subfamily A | | transporter | | |
| | member regulatory beta | | | | |
| | subunit 1 | | | | |
| KCNAB2 | Potassium voltage-gated | 8514 | Channel or | Channel | Vgic |
| | channel subfamily A | | transporter | | |
| | regulatory beta subunit 2 | | | | |
| KCNB1 | Potassium voltage-gated | 3745 | Channel or | Channel | Vgic |
| | channel subfamily B | | transporter | | |
| | member 1 | | | | |
| KCNB2 | Potassium voltage-gated | 9312 | Channel or | Channel | Vgic |
| | channel subfamily B | | transporter | | |
| | member 2 | | | | |
| KCNC1 | Potassium voltage-gated | 3746 | Channel or | Channel | Vgic |
| | channel subfamily C | | transporter | | |
| | member 1 | | | | |
| KCNC2 | Potassium voltage-gated | 3747 | Channel or | Channel | Vgic |
| | channel subfamily C | | transporter | | |
| | member 2 | | | | |
| KCNC3 | Potassium voltage-gated | 3748 | Channel or | Channel | Vgic |
| | channel subfamily C | | transporter | | |
| | member 3 | | | | |
| KCNC4 | Potassium voltage-gated | 3749 | Channel or | Channel | Vgic |
| | channel subfamily C | | transporter | | |
| | member 4 | | | | |
| KCND1 | Potassium voltage-gated | 3750 | Channel or | Channel | Vgic |
| | channel subfamily D | | transporter | | |
| | member 1 | | | | |
| KCND2 | Potassium voltage-gated | 3751 | Channel or | Channel | Vgic |
| | channel subfamily D | | transporter | | |
| | member 2 | | | | |
| KCND3 | Potassium voltage-gated | 3752 | Channel or | Channel | Vgic |
| | channel subfamily D | | transporter | | |
| | member 3 | | | | |
| KCNE2 | Potassium voltage-gated | 9992 | Channel or | Channel | Vgic |
| | channel subfamily E | | transporter | | |
| | regulatory subunit 2 | | | | |
| KCNE3 | Potassium voltage-gated | 10008 | Channel or | Channel | Vgic |
| | channel subfamily E | | transporter | | |
| | regulatory subunit 3 | | | | |
| KCNE4 | Potassium voltage-gated | 23704 | Channel or | Channel | Vgic |
| | channel subfamily E | | transporter | | |
| | regulatory subunit 4 | | | | |
| KCNF1 | Potassium voltage-gated | 3754 | Channel or | Channel | Vgic |
| | channel modifier subfamily | | transporter | | |
| | F member 1 | | | | |
| KCNG1 | Potassium voltage-gated | 3755 | Channel or | Channel | Vgic |
| | channel modifier subfamily | | transporter | | |
| | G member 1 | | | | |
| KCNG2 | Potassium voltage-gated | 26251 | Channel or | Channel | Vgic |
| | channel modifier subfamily | | transporter | | |
| | G member 2 | | | | |
| KCNG3 | Potassium voltage-gated | 170850 | Channel or | Channel | Vgic |
| | channel modifier subfamily | | transporter | | |
| | G member 3 | | | | |
| KCNG4 | Potassium voltage-gated | 93107 | Channel or | Channel | Vgic |
| | channel modifier subfamily | | transporter | | |
| | G member 4 | | | | |
| KCNH1 | Potassium voltage-gated | 3756 | Channel or | Channel | Vgic |
| | channel subfamily H | | transporter | | |
| | member 1 | | | | |
| KCNH2 | Potassium voltage-gated | 3757 | Channel or | Channel | Vgic |
| | channel subfamily H | | transporter | | |
| | member 2 | | | | |
| KCNH3 | Potassium voltage-gated | 23416 | Channel or | Channel | Vgic |
| | channel subfamily H | | transporter | | |
| | member 3 | | | | |
| KCNH4 | Potassium voltage-gated | 23415 | Channel or | Channel | Vgic |
| | channel subfamily H | | transporter | | |
| | member 4 | | | | |
| KCNH5 | Potassium voltage-gated | 27133 | Channel or | Channel | Vgic |
| | channel subfamily H | | transporter | | |
| | member 5 | | | | |
| KCNH6 | Potassium voltage-gated | 81033 | Channel or | Channel | Vgic |
| | channel subfamily H | | transporter | | |
| | member 6 | | | | |
| KCNH7 | Potassium voltage-gated | 90134 | Channel or | Channel | Vgic |
| | channel subfamily H | | transporter | | |
| | member 7 | | | | |
| KCNH8 | Potassium voltage-gated | 131096 | Channel or | Channel | Vgic |
| | channel subfamily H | | transporter | | |
| | member 8 | | | | |
| KCNJ1 | Potassium voltage-gated | 3758 | Channel or | Channel | Vgic |
| | channel subfamily J | | transporter | | |
| | member 1 | | | | |
| KCNJ10 | Potassium voltage-gated | 3766 | Channel or | Channel | Vgic |
| | channel subfamily J | | transporter | | |
| | member 10 | | | | |
| KCNJ11 | Potassium voltage-gated | 3767 | Channel or | Channel | Vgic |
| | channel subfamily J | | transporter | | |
| | member 11 | | | | |
| KCNJ12 | Potassium voltage-gated | 3768 | Channel or | Channel | Vgic |
| | channel subfamily J | | transporter | | |
| | member 12 | | | | |
| KCNJ13 | Potassium voltage-gated | 3769 | Channel or | Channel | Vgic |
| | channel subfamily J | | transporter | | |
| | member 13 | | | | |
| KCNJ14 | Potassium voltage-gated | 3770 | Channel or | Channel | Vgic |
| | channel subfamily J | | transporter | | |
| | member 14 | | | | |
| KCNJ15 | Potassium voltage-gated | 3772 | Channel or | Channel | Vgic |
| | channel subfamily J | | transporter | | |
| | member 15 | | | | |
| KCNJ16 | Potassium voltage-gated | 3773 | Channel or | Channel | Vgic |
| | channel subfamily J | | transporter | | |
| | member 16 | | | | |
| KCNJ2 | Potassium voltage-gated | 3759 | Channel or | Channel | Vgic |
| | channel subfamily J | | transporter | | |
| | member 2 | | | | |
| KCNJ3 | Potassium voltage-gated | 3760 | Channel or | Channel | Vgic |
| | channel subfamily J | | transporter | | |
| | member 3 | | | | |
| KCNJ4 | Potassium voltage-gated | 3761 | Channel or | Channel | Vgic |
| | channel subfamily J | | transporter | | |
| | member 4 | | | | |
| KCNJ5 | Potassium voltage-gated | 3762 | Channel or | Channel | Vgic |
| | channel subfamily J | | transporter | | |
| | member 5 | | | | |
| KCNJ6 | Potassium voltage-gated | 3763 | Channel or | Channel | Vgic |
| | channel subfamily J | | transporter | | |
| | member 6 | | | | |
| KCNJ8 | Potassium voltage-gated | 3764 | Channel or | Channel | Vgic |
| | channel subfamily J | | transporter | | |
| | member 8 | | | | |
| KCNJ9 | Potassium voltage-gated | 3765 | Channel or | Channel | Vgic |
| | channel subfamily J | | transporter | | |
| | member 9 | | | | |
| KCNK1 | Potassium two pore domain | 3775 | Channel or | Channel | Vgic |
| | channel subfamily K | | transporter | | |
| | member 1 | | | | |
| KCNK10 | Potassium two pore domain | 54207 | Channel or | Channel | Vgic |
| | channel subfamily K | | transporter | | |
| | member 10 | | | | |
| KCNK12 | Potassium two pore domain | 56660 | Channel or | Channel | Vgic |
| | channel subfamily K | | transporter | | |
| | member 12 | | | | |
| KCNK13 | Potassium two pore domain | 56659 | Channel or | Channel | Vgic |
| | channel subfamily K | | transporter | | |
| | member 13 | | | | |
| KCNK15 | Potassium two pore domain | 60598 | Channel or | Channel | Vgic |
| | channel subfamily K | | transporter | | |
| | member 15 | | | | |
| KCNK16 | Potassium two pore domain | 83795 | Channel or | Channel | Vgic |
| | channel subfamily K | | transporter | | |
| | member 16 | | | | |
| KCNK17 | Potassium two pore domain | 89822 | Channel or | Channel | Vgic |
| | channel subfamily K | | transporter | | |
| | member 17 | | | | |
| KCNK18 | Potassium two pore domain | 338567 | Channel or | Channel | Vgic |
| | channel subfamily K | | transporter | | |
| | member 18 | | | | |
| KCNK2 | Potassium two pore domain | 3776 | Channel or | Channel | Vgic |
| | channel subfamily K | | transporter | | |
| | member 2 | | | | |
| KCNK3 | Potassium two pore domain | 3777 | Channel or | Channel | Vgic |
| | channel subfamily K | | transporter | | |
| | member 3 | | | | |
| KCNK4 | Potassium two pore domain | 50801 | Channel or | Channel | Vgic |
| | channel subfamily K | | transporter | | |
| | member 4 | | | | |
| KCNK5 | Potassium two pore domain | 8645 | Channel or | Channel | Vgic |
| | channel subfamily K | | transporter | | |
| | member 5 | | | | |
| KCNK6 | Potassium two pore domain | 9424 | Channel or | Channel | Vgic |
| | channel subfamily K | | transporter | | |
| | member 6 | | | | |
| KCNK7 | Potassium two pore domain | 10089 | Channel or | Channel | Vgic |
| | channel subfamily K | | transporter | | |
| | member 7 | | | | |
| KCNK9 | Potassium two pore domain | 51305 | Channel or | Channel | Vgic |
| | channel subfamily K | | transporter | | |
| | member 9 | | | | |
| KCNMA1 | Potassium calcium- | 3778 | Channel or | Channel | Vgic |
| | activated channel subfamily | | transporter | | |
| | M alpha 1 | | | | |
| KCNN1 | Potassium calcium- | 3780 | Channel or | Channel | Vgic |
| | activated channel subfamily | | transporter | | |
| | N member 1 | | | | |
| KCNN2 | Potassium calcium- | 3781 | Channel or | Channel | Vgic |
| | activated channel subfamily | | transporter | | |
| | N member 2 | | | | |
| KCNN3 | Potassium calcium- | 3782 | Channel or | Channel | Vgic |
| | activated channel subfamily | | transporter | | |
| | N member 3 | | | | |
| KCNN4 | Potassium calcium- | 3783 | Channel or | Channel | Vgic |
| | activated channel subfamily | | transporter | | |
| | N member 4 | | | | |
| KCNQ1 | Potassium voltage-gated | 3784 | Channel or | Channel | Vgic |
| | channel subfamily Q | | transporter | | |
| | member 1 | | | | |
| KCNQ2 | Potassium voltage-gated | 3785 | Channel or | Channel | Vgic |
| | channel subfamily Q | | transporter | | |
| | member 2 | | | | |
| KCNQ3 | Potassium voltage-gated | 3786 | Channel or | Channel | Vgic |
| | channel subfamily Q | | transporter | | |
| | member 3 | | | | |
| KCNQ4 | Potassium voltage-gated | 9132 | Channel or | Channel | Vgic |
| | channel subfamily Q | | transporter | | |
| | member 4 | | | | |
| KCNQ5 | Potassium voltage-gated | 56479 | Channel or | Channel | Vgic |
| | channel subfamily Q | | transporter | | |
| | member 5 | | | | |
| KCNS1 | Potassium voltage-gated | 3787 | Channel or | Channel | Vgic |
| | channel modifier subfamily | | transporter | | |
| | S member 1 | | | | |
| KCNS2 | Potassium voltage-gated | 3788 | Channel or | Channel | Vgic |
| | channel modifier subfamily | | transporter | | |
| | S member 2 | | | | |
| KCNS3 | Potassium voltage-gated | 3790 | Channel or | Channel | Vgic |
| | channel modifier subfamily | | transporter | | |
| | S member 3 | | | | |
| KCNT1 | Potassium sodium-activated | 57582 | Channel or | Channel | Vgic |
| | channel subfamily T | | transporter | | |
| | member 1 | | | | |
| KCNT2 | Potassium sodium-activated | 343450 | Channel or | Channel | Vgic |
| | channel subfamily T | | transporter | | |
| | member 2 | | | | |
| KCNU1 | Potassium calcium- | 157855 | Channel or | Channel | Vgic |
| | activated channel subfamily | | transporter | | |
| | U member 1 | | | | |
| KCNV1 | Potassium voltage-gated | 27012 | Channel or | Channel | Vgic |
| | channel modifier subfamily | | transporter | | |
| | V member 1 | | | | |
| KCNV2 | Potassium voltage-gated | 169522 | Channel or | Channel | Vgic |
| | channel modifier subfamily | | transporter | | |
| | V member 2 | | | | |
| MCOLN1 | Mucolipin 1 | 57192 | Channel or | Channel | Vgic |
| | | | transporter | | |
| MCOLN2 | Mucolipin 2 | 255231 | Channel or | Channel | Vgic |
| | | | transporter | | |
| MCOLN3 | Mucolipin 3 | 55283 | Channel or | Channel | Vgic |
| | | | transporter | | |
| PKD2 | Polycystin 2, transient | 5311 | Channel or | Channel | Vgic |
| | receptor potential cation | | transporter | | |
| | channel | | | | |
| PKD2L1 | Polycystin 2 like 1, transient | 9033 | Channel or | Channel | Vgic |
| | receptor potential cation | | transporter | | |
| | channel | | | | |
| PKD2L2 | Polycystin 2 like 2, transient | 27039 | Channel or | Channel | Vgic |
| | receptor potential cation | | transporter | | |
| | channel | | | | |
| RYR1 | Ryanodine receptor 1 | 6261 | Channel or | Channel | Vgic |
| | | | transporter | | |
| RYR2 | Ryanodine receptor 2 | 6262 | Channel or | Channel | Vgic |
| | | | transporter | | |
| RYR3 | Ryanodine receptor 3 | 6263 | Channel or | Channel | Vgic |
| | | | transporter | | |
| SCN10A | Sodium voltage-gated | 6336 | Channel or | Channel | Vgic |
| | channel alpha subunit 10 | | transporter | | |
| SCN11A | Sodium voltage-gated | 11280 | Channel or | Channel | Vgic |
| | channel alpha subunit 11 | | transporter | | |
| SCN1A | Sodium voltage-gated | 6323 | Channel or | Channel | Vgic |
| | channel alpha subunit 1 | | transporter | | |
| SCN1B | Sodium voltage-gated | 6324 | Channel or | Channel | Vgic |
| | channel beta subunit 1 | | transporter | | |
| SCN2A | Sodium voltage-gated | 6326 | Channel or | Channel | Vgic |
| | channel alpha subunit 2 | | transporter | | |
| SCN2B | Sodium voltage-gated | 6327 | Channel or | Channel | Vgic |
| | channel beta subunit 2 | | transporter | | |
| SCN3A | Sodium voltage-gated | 6328 | Channel or | Channel | Vgic |
| | channel alpha subunit 3 | | transporter | | |
| SCN3B | Sodium voltage-gated | 55800 | Channel or | Channel | Vgic |
| | channel beta subunit 3 | | transporter | | |
| SCN4A | Sodium voltage-gated | 6329 | Channel or | Channel | Vgic |
| | channel alpha subunit 4 | | transporter | | |
| SCN4B | Sodium voltage-gated | 6330 | Channel or | Channel | Vgic |
| | channel beta subunit 4 | | transporter | | |
| SCN5A | Sodium voltage-gated | 6331 | Channel or | Channel | Vgic |
| | channel alpha subunit 5 | | transporter | | |
| SCN7A | Sodium voltage-gated | 6332 | Channel or | Channel | Vgic |
| | channel alpha subunit 7 | | transporter | | |
| SCN8A | Sodium voltage-gated | 6334 | Channel or | Channel | Vgic |
| | channel alpha subunit 8 | | transporter | | |
| SCN9A | Sodium voltage-gated | 6335 | Channel or | Channel | Vgic |
| | channel alpha subunit 9 | | transporter | | |
| TPCN1 | Two pore segment channel 1 | 53373 | Channel or | Channel | Vgic |
| | | | transporter | | |
| TPCN2 | Two pore segment channel 2 | 219931 | Channel or | Channel | Vgic |
| | | | transporter | | |
| TRPA1 | Transient receptor potential | 8989 | Channel or | Channel | Vgic |
| | cation channel subfamily A | | transporter | | |
| | member 1 | | | | |
| TRPC1 | Transient receptor potential | 7220 | Channel or | Channel | Vgic |
| | cation channel subfamily C | | transporter | | |
| | member 1 | | | | |
| TRPC2 | Transient receptor potential | 7221 | Channel or | Channel | Vgic |
| | cation channel subfamily C | | transporter | | |
| | member 2, pseudogene | | | | |
| TRPC3 | Transient receptor potential | 7222 | Channel or | Channel | Vgic |
| | cation channel subfamily C | | transporter | | |
| | member 3 | | | | |
| TRPC4 | Transient receptor potential | 7223 | Channel or | Channel | Vgic |
| | cation channel subfamily C | | transporter | | |
| | member 4 | | | | |
| TRPC5 | Transient receptor potential | 7224 | Channel or | Channel | Vgic |
| | cation channel subfamily C | | transporter | | |
| | member 5 | | | | |
| TRPC6 | Transient receptor potential | 7225 | Channel or | Channel | Vgic |
| | cation channel subfamily C | | transporter | | |
| | member 6 | | | | |
| TRPC7 | Transient receptor potential | 57113 | Channel or | Channel | Vgic |
| | cation channel subfamily C | | transporter | | |
| | member 7 | | | | |
| TRPM1 | Transient receptor potential | 4308 | Channel or | Channel | Vgic |
| | cation channel subfamily M | | transporter | | |
| | member 1 | | | | |
| TRPM2 | Transient receptor potential | 7226 | Channel or | Channel | Vgic |
| | cation channel subfamily M | | transporter | | |
| | member 2 | | | | |
| TRPM3 | Transient receptor potential | 80036 | Channel or | Channel | Vgic |
| | cation channel subfamily M | | transporter | | |
| | member 3 | | | | |
| TRPM4 | Transient receptor potential | 54795 | Channel or | Channel | Vgic |
| | cation channel subfamily M | | transporter | | |
| | member 4 | | | | |
| TRPM5 | Transient receptor potential | 29850 | Channel or | Channel | Vgic |
| | cation channel subfamily M | | transporter | | |
| | member 5 | | | | |
| TRPM6 | Transient receptor potential | 140803 | Channel or | Channel | Vgic |
| | cation channel subfamily M | | transporter | | |
| | member 6 | | | | |
| TRPM7 | Transient receptor potential | 54822 | Channel or | Channel | Vgic |
| | cation channel subfamily M | | transporter | | |
| | member 7 | | | | |
| TRPM8 | Transient receptor potential | 79054 | Channel or | Channel | Vgic |
| | cation channel subfamily M | | transporter | | |
| | member 8 | | | | |
| TRPV1 | Transient receptor potential | 7442 | Channel or | Channel | Vgic |
| | cation channel subfamily V | | transporter | | |
| | member 1 | | | | |
| TRPV2 | Transient receptor potential | 51393 | Channel or | Channel | Vgic |
| | cation channel subfamily V | | transporter | | |
| | member 2 | | | | |
| TRPV3 | Transient receptor potential | 162514 | Channel or | Channel | Vgic |
| | cation channel subfamily V | | transporter | | |
| | member 3 | | | | |
| TRPV4 | Transient receptor potential | 59341 | Channel or | Channel | Vgic |
| | cation channel subfamily V | | transporter | | |
| | member 4 | | | | |
| TRPV5 | Transient receptor potential | 56302 | Channel or | Channel | Vgic |
| | cation channel subfamily V | | transporter | | |
| | member 5 | | | | |
| TRPV6 | Transient receptor potential | 55503 | Channel or | Channel | Vgic |
| | cation channel subfamily V | | transporter | | |
| | member 6 | | | | |

Lgic = ligand-gated ion channel,
Vgic = voltage-gated ion channel,
Other_ic = other ion channel

Agent Modalities

A neuromodulating agent can be a number of different modalities. A neuromodulating agent can be a nucleic acid molecule (e.g., DNA molecule or RNA molecule, e.g., mRNA, guide RNA (gRNA), or inhibitory RNA molecule (e.g., siRNA, shRNA, or miRNA), or a hybrid DNA-RNA molecule), a small molecule (e.g., a neurotransmitter, an agonist, antagonist, or an epigenetic modifier), a peptide, or a polypeptide (e.g., an antibody molecule, e.g., an antibody or antigen binding fragment thereof, or a neuropeptide). A neuromodulating agent can also be a viral vector expressing a neurome gene or a cell infected with a viral vector. Any of these modalities can be a neuromodulating agent directed to target (e.g., to agonize or to inhibit) a gene or protein in a neurotransmitter, neuropeptide, neuronal growth factor, or neurome gene (e.g., biosynthesis, channel, transporter, ligand, receptor, signaling, synaptic, structural, or vesicular) pathway described herein (e.g., a gene or protein listed in Tables 1A-1C, Table 7, or Table 8).

The nucleic acid molecule, small molecule, peptide, polypeptide, or antibody molecule can be modified. For example, the modification can be a chemical modification, e.g., conjugation to a marker, e.g., fluorescent marker or a radioactive marker. In other examples, the modification can include conjugation to a molecule that enhances the stability or half-life of the neuromodulating agent. The modification can also include conjugation to an antibody to target the agent to a particular cell or tissue. Additionally, the modification can be a chemical modification, packaging modification (e.g., packaging within a nanoparticle or microparticle), or targeting modification to prevent the agent from crossing the blood brain barrier.

Small Molecules

Numerous small molecule neuromodulating agents useful in the methods of the invention are described herein and additional small molecule neuromodulating agents useful as therapies for cancer can also be screened based on their ability to modulate sympathetic and parasympathetic neural pathways. Small molecules include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds.

In some embodiments, the neuromodulating agent is an agonist or antagonist listed in column 2 or column 3 of Table 2A or column 2 of Tables 2B-2L, which is directed to the corresponding neurotransmitter pathway member listed in column 1 of Tables 2A-2L. In some embodiments, the neuromodulating agent is a neurotransmitter or neuropeptide listed in Table 1A, 1B, or encoded by a gene in Table 7, or a neuronal growth factor listed in Table 10 or encoded by a gene in Table 7. Agonists and antagonists can be used to treat a disorder or condition described herein. A pharmaceutical composition comprising the agonist, antagonist, neurotransmitter, neuropeptide, or neuronal growth factor can be formulated for treatment of a cancer described herein. In some embodiments, a pharmaceutical composition that includes the agonist or antagonist is formulated for local administration, e.g., to the affected site in a subject.

Polypeptides

In embodiments, a neuromodulating agent described herein comprises a neuromodulating agent polypeptide or an analog thereof. For example, a neuromodulating agent described herein is a neuropeptide or an analog thereof.

The neuromodulating agent can be a neuropeptide listed in Table 1A or 1B, a neuronal growth factor listed in Table 10, or a protein encoded by a neurome gene listed in Table 7 or Table 8 (e.g., a biosynthesis, channel, transporter, ligand, receptor, signaling, synaptic, structural, or vesicular protein), wherein the primary sequence of the neuromodulating agent is provided by reference to accession number or Entrez Gene ID. The agent can be a polypeptide having the sequence referenced by accession number or Entrez Gene ID of a neuropeptide listed in Table 1A or 1B, a neuronal growth factor listed in Table 10, or a protein encoded by a neurome gene listed in Table 7, or an analog thereof, e.g., a sequence having at least 75%, 80%, 85%, 90%, 90%, 98%, 99% or 100% identity to the sequence referenced by accession number or Entrez Gene ID.

Percent identity in the context of two or more polypeptide sequences or nucleic acids, refers to two or more sequences that are the same. Two sequences are “substantially identical” if two sequences have a specified percentage of amino acid residues or nucleotides that are the same (e.g., at least 60% identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region, or, when not specified, over the entire sequence), when compared and aligned for maximum correspondence over a comparison window, or designated region as measured using a sequence comparison algorithms or by manual alignment and visual inspection. In some cases, the identity (or substantial identity) exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, Adv. Appl. Math. 2:482c, 1970, by the homology alignment algorithm of Needleman and Wunsch, J. Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, 2003).

Two examples of algorithms that are suitable for determining percent sequence identity and sequence similarity are the BLAST and BLAST 2.0 algorithms, which are described in Altschul et al., Nuc. Acids Res. 25:3389, 1977; and Altschul et al., J. Mol. Biol. 215:403, 1990, respectively. Software for performing BLAST analyses is publicly available through the National Center for Biotechnology Information.

The percent identity between two amino acid sequences can also be determined using the algorithm of E. Meyers and W. Miller, Comput. Appl. Biosci. 4:11, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444, 1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Methods of making a therapeutic polypeptide are routine in the art. See, in general, Smales & James (Eds.), Therapeutic Proteins: Methods and Protocols (Methods in Molecular Biology), Humana Press 2005; and Crommelin, Sindelar & Meibohm (Eds.), Pharmaceutical Biotechnology: Fundamentals and Applications, Springer 2013.

Some methods for producing a neuromodulating agent polypeptide involve expression in mammalian cells, although recombinant proteins can also be produced using insect cells, yeast, bacteria, or other cells under the control of appropriate promoters. Mammalian expression vectors may comprise nontranscribed elements such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press 2012.

Various mammalian cell culture systems can be employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer 2014.

Purification of protein therapeutics is known and is described, e.g., in Franks, Protein Biotechnology: Isolation, Characterization, and Stabilization, Humana Press 2013; and in Cutler, Protein Purification Protocols (Methods in Molecular Biology), Humana Press 2010.

Formulation of protein therapeutics is known and is described, e.g., in Meyer (Ed.), Therapeutic Protein Drug Products: Practical Approaches to formulation in the Laboratory, Manufacturing, and the Clinic, Woodhead Publishing Series 2012.

Antibodies

The neuromodulating agent can be an antibody or antigen binding fragment thereof. For example, a neuromodulating agent described herein is an antibody that blocks or potentiates activity and/or function of a receptor, neuropeptide, neurotransmitter or transporter listed in Table 1A, a ligand listed in Table 1B, a neuronal growth factor listed in Table 10, or a neurome gene listed in Table 7 or Table 8 (e.g., a biosynthesis, channel, transporter, ligand, receptor, signaling, synaptic, structural, or vesicular gene).

The making and use of therapeutic antibodies against a target antigen (e.g., against a protein in a neurotransmitter pathway described herein (e.g., a protein product of a gene listed in Table 1)) is known in the art. See, for example, the references cited herein above, as well as Zhiqiang An (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

Synthetic mRNA

In some embodiments, the neuromodulating agent is an mRNA molecule, e.g., a synthetic mRNA molecule encoding a protein listed in Tables 1A-1C, or a protein encoded by a gene in Table 7 or Table 8. The mRNA molecule may increase the level (e.g., protein and/or mRNA level) and/or activity or function of a neurotransmitter, neurotransmitter receptor, neuropeptide, neuropeptide receptor, neuronal growth factor, or neurome gene in Table 7 or Table 8 (e.g., a biosynthesis, channel, transporter, ligand, receptor, signaling, synaptic, structural, or vesicular gene), e.g., a positive regulator of function. The mRNA molecule can encode a neuromodulating agent or a fragment thereof. For example, the mRNA molecule encodes a polypeptide having at least 50% (e.g., at least 50%, 60%, 70%, 80%, 90%, 95%, 97%, 99%, or greater) identity to the amino acid sequence of a neuromodulating agent listed in Table 1A, a ligand listed in Table 1B, a neuronal growth factor listed in Table 10, or neurome gene in Table 7 or Table 8 (e.g., a biosynthesis, channel, transporter, ligand, receptor, signaling, synaptic, structural, or vesicular gene), all with reference to accession number or Entrez Gene ID provided. In other examples, the mRNA molecule has at least 50% (e.g., at least 50%, 60%, 70%, 80%, 90%, 95%, 97%, 99%, or greater) identity to the nucleic acid sequence of a neuromodulating agent listed in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene). The mRNA molecule can encode an amino acid sequence differing by no more than 30 (e.g., no more than 30, 20, 10, 5, 4, 3, 2, or 1) amino acids to the amino acid sequence of a neuromodulating agent listed in Table 1A, a ligand listed in Table 1B, a neuronal growth factor listed in Table 10, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided. The mRNA molecule can have a sequence encoding a fragment of a neuromodulating agent listed in Table 1A, a ligand listed in Table 1B, a neuronal growth factor listed in Table 10, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided. For example, the fragment comprises 10-20, 20-40, 40-60, 60-80, 80-100, 100-120, 120-140, 140-160, 160-180, 180-200, 200-250, 250-300, 300-400, 400-500, 500-600, or more amino acids in length. In embodiments, the fragment is a functional fragment, e.g., having at least 20%, e.g., at least 20%, 30%, 40%, 50%, 60%, 70%, 80%, 90%, or greater, of an activity of a full length neuromodulating agent listed in Table 1A, a ligand listed in Table 1B, a neuronal growth factor listed in Table 10, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided. In embodiments, the mRNA molecule increases the level and/or activity or function of or encodes a neuromodulating agent (or fragment thereof).

The synthetic mRNA molecule can be modified, e.g., chemically. The mRNA molecule can be chemically synthesized or transcribed in vitro. The mRNA molecule can be disposed on a plasmid, e.g., a viral vector, bacterial vector, or eukaryotic expression vector. In some examples, the mRNA molecule can be delivered to cells by transfection, electroporation, or transduction (e.g., adenoviral or lentiviral transduction).

In some embodiments, the modified RNA encoding a neuromodulating agent of interest described herein has modified nucleosides or nucleotides. Such modifications are known and are described, e.g., in WO2012019168. Additional modifications are described, e.g., in WO2015038892; WO2015038892; WO2015089511; WO2015196130; WO2015196118 and WO2015196128A2.

In some embodiments, the modified RNA encoding a polypeptide of interest described herein has one or more terminal modifications, e.g., a 5′Cap structure and/or a poly-A tail (e.g., of between 100-200 nucleotides in length). The 5′ cap structure may be selected from the group consisting of CapO, CapI, ARCA, inosine, NI-methyl-guanosine, 2′fluoro-guanosine, 7-deaza-guanosine, 8-oxo-guanosine, 2-amino-guanosine, LNA-guanosine, and 2-azido-guanosine. In some cases, the modified RNAs also contain a 5′ UTR comprising at least one Kozak sequence, and a 3′ UTR. Such modifications are known and are described, e.g., in WO2012135805 and WO2013052523. Additional terminal modifications are described, e.g., in WO2014164253 and WO2016011306. WO2012045075 and WO2014093924

Chimeric enzymes for synthesizing capped RNA molecules (e.g., modified mRNA) which may include at least one chemical modification are described in WO2014028429.

In some embodiments, a modified mRNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO2013151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO2013151666, WO2013151668, WO2013151663, WO2013151669, WO2013151670, WO2013151664, WO2013151665, WO2013151671, WO2013151672, WO2013151667 and WO2013151736.S Methods of purification include purifying an RNA transcript comprising a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO2014152031); using ion (e.g., anion) exchange chromatography that allows for separation of longer RNAs up to 10,000 nucleotides in length via a scalable method (WO2014144767); and subjecting a modified mRNA sample to DNAse treatment (WO2014152030).

Formulations of modified RNAs are known and are described, e.g., in WO2013090648. For example, the formulation may be, but is not limited to, nanoparticles, poly(lactic-co-glycolic acid)(PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof.

Modified RNAs encoding polypeptides in the fields of human disease, antibodies, viruses, and a variety of in vivo settings are known and are disclosed in for example, Table 6 of International Publication Nos. WO2013151666, WO2013151668, WO2013151663, WO2013151669, WO2013151670, WO2013151664, WO2013151665, and WO2013151736; Tables 6 and 7 of International Publication No. WO2013151672; Tables 6, 178 and 179 of International Publication No. WO2013151671; Tables 6, 185 and 186 of International Publication No. WO2013151667. Any of the foregoing may be synthesized as an IVT polynucleotide, chimeric polynucleotide or a circular polynucleotide, and each may comprise one or more modified nucleotides or terminal modifications.

Inhibitory RNA

In some embodiments, the neuromodulating agent is an inhibitory RNA molecule, e.g., that acts by way of the RNA interference (RNAi) pathway. An inhibitory RNA molecule can decrease the expression level (e.g., protein level or mRNA level) of a neurotransmitter, neuropeptide, receptor, neuronal growth factor, or neurome gene listed herein. For example, an inhibitory RNA molecule includes a short interfering RNA, short hairpin RNA, and/or a microRNA that targets a full length neuromodulating agent listed in Table 1A, a ligand listed in Table 1B, a neuronal growth factor listed in Table 10, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided. A siRNA is a double-stranded RNA molecule that typically has a length of about 19-25 base pairs. A shRNA is a RNA molecule comprising a hairpin turn that decreases expression of target genes via RNAi. shRNAs can be delivered to cells in the form of plasmids, e.g., viral or bacterial vectors, e.g., by transfection, electroporation, or transduction). A microRNA is a non-coding RNA molecule that typically has a length of about 22 nucleotides. MiRNAs bind to target sites on mRNA molecules and silence the mRNA, e.g., by causing cleavage of the mRNA, destabilization of the mRNA, or inhibition of translation of the mRNA. In embodiments, the inhibitory RNA molecule decreases the level and/or activity of a negative regulator of function or a positive regulator of function. In other embodiments, the inhibitor RNA molecule decreases the level and/or activity of an inhibitor of a positive regulator of function.

An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity.

In some embodiments, the inhibitory RNA molecule decreases the level and/or activity or function of a neuromodulating agent. In embodiments, the inhibitory RNA molecule inhibits expression of a neuromodulating agent (e.g., inhibits translation to protein). In other embodiments, the inhibitor RNA molecule increases degradation of a neuromodulating agent and/or decreases the stability (i.e., half-life) of a neuromodulating agent. The inhibitory RNA molecule can be chemically synthesized or transcribed in vitro.

The making and use of inhibitory therapeutic agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press 2010.

Gene Editing

In some embodiments, the neuromodulating agent is a component of a gene editing system. For example, the neuromodulating agent introduces an alteration (e.g., insertion, deletion (e.g., knockout), translocation, inversion, single point mutation, or other mutation) in a gene related to a neurotransmitter pathway, e.g., a neuropeptide or receptor gene described in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided. Exemplary gene editing systems include the zinc finger nucleases (ZFNs), Transcription Activator-Like Effector-based Nucleases (TALEN), and the clustered regulatory interspaced short palindromic repeat (CRISPR) system. ZFNs, TALENs, and CRISPR-based methods are described, e.g., in Gaj et al. Trends Biotechnol. 31.7(2013):397-405.

CRISPR refers to a set of (or system comprising a set of) clustered regularly interspaced short palindromic repeats. A CRISPR system refers to a system derived from CRISPR and Cas (a CRISPR-associated protein) or other nuclease that can be used to silence or mutate a gene described herein. The CRISPR system is a naturally occurring system found in bacterial and archeal genomes. The CRISPR locus is made up of alternating repeat and spacer sequences. In naturally-occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature 482: 331, 2012. For example, such modification of the system includes introducing into a eukaryotic cell a plasmid containing a specifically-designed CRISPR and one or more appropriate Cas proteins. The CRISPR locus is transcribed into RNA and processed by Cas proteins into small RNAs that comprise a repeat sequence flanked by a spacer. The RNAs serve as guides to direct Cas proteins to silence specific DNA/RNA sequences, depending on the spacer sequence. See, e.g., Horvath et al., Science 327: 167, 2010; Makarova et al., Biology Direct 1:7, 2006; Pennisi, Science 341: 833, 2013. In some examples, the CRISPR system includes the Cas9 protein, a nuclease that cuts on both strands of the DNA. See, e.g., i.d.

In some embodiments, in a CRISPR system for use described herein, e.g., in accordance with one or more methods described herein, the spacers of the CRISPR are derived from a target gene sequence, e.g., from a sequence (with reference to the accession number) of a neurotransmitter pathway gene, e.g., a neuropeptide or receptor gene listed in Table 1A, a ligand listed in Table 1B, a neuronal growth factor listed in Table 10, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided.

In some embodiments, the neuromodulating agent includes a guide RNA (gRNA) for use in a clustered regulatory interspaced short palindromic repeat (CRISPR) system for gene editing. In embodiments, the neuromodulating agent comprises a zinc finger nuclease (ZFN), or an mRNA encoding a ZFN, that targets (e.g., cleaves) a nucleic acid sequence (e.g., DNA sequence) of a gene related to a neurotransmitter pathway, e.g., a neuropeptide or receptor gene described in Table 1. In embodiments, the neuromodulating agent comprises a TALEN, or an mRNA encoding a TALEN, that targets (e.g., cleaves) a nucleic acid sequence (e.g., DNA sequence) in a gene related to a neurotransmitter pathway, e.g., a neuropeptide or receptor gene described in Table 1A, a ligand listed in Table 1B, a neuronal growth factor listed in Table 10, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided.

For example, the gRNA can be used in a CRISPR system to engineer an alteration in a gene (e.g., a gene related to a neurotransmitter pathway, e.g., a neuropeptide, neurotransmitter, neuronal growth factor or receptor gene described in Tables 1A, 1B, or 10, or a neurome gene listed in Table 7 or Table 8 (e.g., a biosynthesis, channel, transporter, ligand, receptor, signaling, synaptic, structural, or vesicular gene)). In other examples, the ZFN and/or TALEN can be used to engineer an alteration in a gene (e.g., a gene related to a neurotransmitter pathway, e.g., a neuropeptide, neurotransmitter, neuronal growth factor, or receptor gene described in Tables 1A, 1B, or 10, or a neurome gene listed in Table 7 or Table 8 (e.g., a biosynthesis, channel, transporter, ligand, receptor, signaling, synaptic, structural, or vesicular gene)). Exemplary alterations include insertions, deletions (e.g., knockouts), translocations, inversions, single point mutations, or other mutations. The alteration can be introduced in the gene in a cell, e.g., in vitro, ex vivo, or in vivo. In some examples, the alteration increases the level and/or activity of a neuromodulator, e.g., the alteration is a positive regulator of function. In other examples, the alteration decreases the level and/or activity of (e.g., knocks down or knocks out) a neuromodulator, e.g., the alteration is a negative regulator of function. In yet another example, the alteration corrects a defect (e.g., a mutation causing a defect), in a gene related to a neurotransmitter pathway, e.g., a neuropeptide or receptor gene described in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided.

In certain embodiments, the CRISPR system is used to edit (e.g., to add or delete a base pair) a target gene, e.g., a neuromodulating agent, e.g., described herein. In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In embodiments, the CRISPR system is used to direct Cas to a promoter of a neuromodulator, e.g., described herein, for example, thereby blocking an RNA polymerase sterically.

In some embodiments, a CRISPR system can be generated to edit a neuromodulator (e.g., a gene related to a neurotransmitter pathway, e.g., a neuropeptide or receptor gene described in Table 1A-1C), using technology described in, e.g., U.S. Publication No. 20140068797; Cong, Science 339: 819, 2013; Tsai, Nature Biotechnol., 32:569, 2014; and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., a gene encoding a neuromodulating agent (e.g., a neuropeptide, neurotransmitter, neuronal growth factor, neurome gene, or receptor described herein). In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-g RNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression. In some embodiments, CRISPR-mediated gene activation (CRISPRa) can be used for transcriptional activation, e.g., of one or more genes described herein, e.g., a neuromodulating agent (e.g., a neuropeptide, neurotransmitter, neuronal growth factor, neurome gene, or receptor described herein). In the CRISPRa technique, dCas9 fusion proteins recruit transcriptional activators. For example, dCas9 can be used to recruit polypeptides (e.g., activation domains) such as VP64 or the p65 activation domain (p65D) and used with sgRNA (e.g., a single sgRNA or multiple sgRNAs), to activate a gene or genes, e.g., endogenous gene(s). Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1).

The CRISPRi and CRISPRa techniques are described in greater detail, e.g., in Dominguez et al., Nat. Rev. Mol. Cell Biol. 17:5, 2016, incorporated herein by reference. In addition, dCas9-mediated epigenetic modifications and simultaneous activation and repression using CRISPR systems, as described in Dominguez et al., can be used to modulate a thymic function modulator or thymic function factor described herein.

Viral Vectors

The neuromodulating agent can be a viral vector (e.g., a viral vector expressing a neurome gene). Viral vectors can be used to express a transgene encoding a neurotransmitter, neuropeptide, receptor, or neuronal growth factor from Tables 1A-1C or a neurome gene in Table 7 or Table 8 (e.g., a biosynthesis, channel, transporter, ligand, receptor, signaling, synaptic, structural, or vesicular gene), all with reference to accession number or Entrez Gene ID provided. A viral vector may be administered to a cell or to a subject (e.g., a human subject or animal model) to increase expression of a neurotransmitter, neuropeptide, receptor, or neuronal growth factor from Tables 1A-1C or a neurome gene in Table 7 or Table 8 (e.g., a biosynthesis, channel, transporter, ligand, receptor, signaling, synaptic, structural, or vesicular gene). Viral vectors can also be used to express a neurotoxin from Table 3. A viral vector expressing a neurotoxin from Table 3 can be administered to a cell or to a subject (e.g., a human subject or animal model) to decrease neurotransmission. Viral vectors can be directly administered (e.g., injected) to a lymph node, site of inflammation, or tumor to treat cancer.

Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference.

Cell-Based Therapies

A neuromodulating agent described herein can be administered to a cell in vitro (e.g., an immune cell), which can subsequently be administered to a subject (e.g., a human subject or animal model). The neuromodulating agent can be administered to the cell to effect an immune response (e.g., activation, polarization, antigen presentation, cytokine production, migration, proliferation, or differentiation) as described herein. Once the immune response is elicited, the cell can be administered to a subject (e.g., injected) to treat cancer. The immune cell can be locally administered (e.g., injected into a tumor, lymph node or secondary lymphoid organ, or a site of inflammation).

A neuromodulating agent can also be administered to a cell in vitro (e.g., an immune cell) to alter gene expression in the cell. The neuromodulating agent can increase or decrease the expression of a gene in Table 12 in a corresponding immune cell, or the neuromodulating agent can increase or decrease the expression of a neurotransmitter, neuropeptide, receptor, or neuronal growth factor from Tables 1A-10 or a neurome gene in Table 7 or Table 8 (e.g., a biosynthesis, channel, transporter, ligand, receptor, signaling, synaptic, structural, or vesicular gene). The neuromodulating agent can be a polypeptide or nucleic acid (e.g., mRNA or inhibitory RNA) described above. The neuromodulating agent can be an exogenous gene encoded by a plasmid that is introduced into the cell using standard methods (e.g., calcium phosphate precipitation, electroporation, rnicroinjection, infection, lipofection, impaiefection, laserfection, or rnagnetofection). The neuromodulating agent can be a viral vector (e.g., a viral vector expressing a neurome gene) that is introduced to the cell using standard transduction methods. The plasmid or vector can also contain a reporter construct (e.g., a fluorescent reporter) that can be used to confirm expression of the transgene by the immune cell. After the immune cell has been contacted with a neuromodulating agent to increase or decrease gene expression, the cell can be administered to a subject (e.g., injected) to treat cancer. The immune cell can be locally administered (e.g., injected into a tumor, lymph node or secondary lymphoid organ, or a site of inflammation).

The cell can be administered to a subject immediately after being contacted with a neuromodulating agent (e.g., within 5, 10, 15, 30, 45, or 60 minutes of being contacted with a neuromodulating agent), or 6 hours, 12 hours, 24 hours, 2 days, 3, days, 4 days, 5, days, 6 days, 7 days or more after being contacted with a neuromodulating agent. The method can include an additional step of evaluating the immune cell for an immune cell activity (e.g., activation, polarization, antigen presentation, cytokine production, migration, proliferation, or differentiation) or modulation of gene expression after contact with a neuromodulating agent and before administration to a subject.

Screening for New Agents

The invention also features a method of screening for an agent for the treatment of cancer. The method includes (a) providing a plurality of test agents, (b) evaluating the plurality of test agents for neuromodulating activity, and (c) selecting a test agent of the plurality as an anti-cancer agent if the test agent exhibits neuromodulating activity. The evaluation method can include introducing one or more test agents into a co-culture system containing at least one neuronal cell and at least one non-neuronal cell.

In certain embodiments, evaluating an agent for neuromodulating activity includes one or more of evaluating the agent for: ability to inhibit or potentiate a beta adrenergic pathway, ability to inhibit or potentiate a cholinergic pathway, ability to inhibit or potentiate a dopaminergic pathway, ability to inhibit or potentiate a serotonin pathway, ability of the agent to increase or decrease neurogenesis; ability to potentiate or inhibit the transmission of a nerve impulse; ability of the agent to increase or decrease neurome gene expression; ability of the agent to increase neurite (e.g., axon or dendrite) outgrowth; ability to increase or decrease synapse formation or maintenance; ability to increase or decrease neuropeptide signaling; or ability to increase or decrease innervation of a tissue or tumor. The method can include correlating the neuromodulating effect of an agent with a predicted effect of the agent on a mammal, e.g., a human, e.g., by providing (e.g., to the government, a health care provider, insurance company or patient) informational, marketing or instructional material, e.g., print material or computer readable material (e.g., a label, patient record or email), related to the agent or its use, identifying the agent as a possible or predicted treatment in a mammal, e.g., a human. The method can include identifying the agent as a treatment for, or lead compound for treatment of cancer, e.g., a condition described herein. The identification can be in the form of informational, marketing or instructional material. In one embodiment, the methods include correlating a value for neuromodulation activity with ability to treat cancer described herein, e.g., generating a dataset of the correlation.

Evaluating the effect of the agent on neuromodulation can include administering the agent in-vivo to an experimental mammal, or in-vitro or ex-vivo to a nerve or nervous tissue of an animal and evaluating the effect of the agent on the mammal, nerve or nervous tissue. In some embodiments, the evaluation includes entering a value for the evaluation, e.g., into a database or other record. In some embodiments, the subject is an experimental animal, e.g., a wild-type or a transgenic experimental animal.

In some embodiments, the identifying step includes: (a) contacting the agent with a cell or tissue or non-human animal whose genome includes an exogenous nucleic acid that includes a regulatory region of a neuroactive protein, operably linked to a nucleotide sequence encoding a reporter polypeptide (e.g., a light based, e.g., a colorimeteric (e.g., LacZ) or flourescently detectable label, e.g., a fluorescent reporter polypeptide, e.g., GFP, EGFP, BFP, RFP); (b) evaluating the ability of a test agent to modulate the expression of the reporter polypeptide in the cell, tissue or non-human animal; and (c) selecting a test agent that modulates the expression of the reporter polypeptide as an agent that is useful in the treatment of cancer described herein. In one embodiment, the cell or tissue is a nerve cell or tissue. In another embodiment, the non-human animal is a transgenic animal, e.g., a transgenic rodent, e.g., a mouse, rat or guinea pig, harboring the nucleic acid.

The test agents can be, e.g., nucleic acids (e.g., antisense RNA, ribozymes, modified mRNAs encoding an agent protein), polypeptides (antibodies or antigen-binding fragment thereof), peptide fragments, peptidomimetics, or small molecules (e.g., a small organic molecule with a molecular weight of less than 2000 daltons). In another embodiment, the test agent is a member of a combinatorial library, e.g., a peptide, antibody or organic combinatorial library, or a natural product library. In some embodiments, a plurality of test agents, e.g., library members, is tested. The test agents of the plurality, e.g., library, may share structural or functional characteristics. The test agent can also be a crude or semi-purified extract, e.g., a botanical extract such as a plant extract, or algal extract.

In one embodiment, the method includes two evaluating steps, e.g., the method includes a first step of evaluating the test agent in a first system, e.g., an in-vitro or cell-based or tissue system, and a second step of evaluating the test agent in a second system, e.g., a second cell or tissue system or in a non-human experimental animal (e.g., a rodent, a pig, a dog, a non-human primate). In other embodiments, the methods include two evaluating steps in the same type of system, e.g., the agent is re-evaluated in a non-human animal after a first evaluation in the same or a different non-human animal. The two evaluations can be separated by any length of time, e.g., days, weeks, months or years.

In some embodiments, the plurality of test agents are agents that do not cross the blood brain barrier. In some embodiments, the plurality of test agents is evaluated for ability to cross the blood brain barrier.

II. Blood Brain Barrier Permeability

In some embodiments, the neuromodulating agents for use in the present invention are agents that are not capable of crossing, or that do not cross, the blood brain barrier (BBB) of a mammalian subject. The BBB is a highly selective semipermeable membrane barrier that separates the circulating blood from the brain extracellular fluid (e.g., cerebrospinal fluid) in the central nervous system (CNS). The BBB is made up of high-density endothelial cells, which are connected by tight junctions. These cells prevent most molecular compounds in the bloodstream (e.g., large molecules and hydrophilic molecules) from entering the brain. Water, some gases (e.g., oxygen and carbon dioxide), and lipid-soluble molecules (e.g., hydrophobic molecules, such as steroid hormones) can cross the BBB by passive diffusion. Molecules that are needed for neural function, such as glucose and amino acids, are actively transported across the BBB.

A number of approaches can be used to render an agent BBB impermeable. These methods include modifications to increase an agent's size, polarity, or flexibility or reduce its lipophilicity, targeting approaches to direct an agent to another part of the body and away from the brain, and packaging approaches to deliver an agent in a form that does not freely diffuse across the BBB. These approaches can be used to render a BBB permeable neuromodulating agent impermeable, and they can also be used to improve the properties (e.g., cell-specific targeting) of a neuromodulating agent that does not cross the BBB. The methods that can be used to render an agent BBB impermeable are discussed in greater detail herein below.

Formulation of BBB-Permeable Agents for Enhanced Cell Targeting

One approach that can be used to render a neuromodulating agent BBB impermeable is to conjugate the agent to a targeting moiety that directs it somewhere other than the brain. The targeting moiety can be an antibody for a receptor expressed by the target cell (e.g., N-Acetylgalactosamine for liver transport; DGCR2, GBF1, GPR44 or SerpinB10 for pancreas transport; Secretoglobin, family 1A, member 1 for lung transport). The targeting moiety can also be a ligand of any receptor or other molecular identifier expressed on the target cell in the periphery. These targeting moieties can direct the neuromodulating agent of interest to its corresponding target cell, and can also prevent BBB crossing by directing the agent away from the BBB and increasing the size of the neuromodulating agent via conjugation of the targeting moiety.

Neuromodulating agents can also be rendered BBB impermeable through formulation in a particulate delivery system (e.g., a nanoparticle, liposome, or microparticle), such that the agent is not freely diffusible in blood and cannot cross the BBB. The particulate formulation used can be chosen based on the desired localization of the neuromodulating agent (e.g., a tumor, lymph node, lymphoid organ, or site of inflammation), as particles of different sizes accumulate in different locations. For example, nanoparticles with a diameter of 45 nm or less enter the lymph node, while 100 nm nanoparticles exhibit poor lymph node trafficking. Some examples of the link between particle size and localization in vivo are described in Reddy et al., J Controlled Release 112:26 2006, and Reddy et al., Nature Biotechnology 25:1159 2007.

Neuromodulating agents can be tested after the addition of a targeting moiety or after formulation in a particulate delivery system to determine whether or not they cross the BBB. Models for assessing BBB permeability include in vitro models (e.g., monolayer models, co-culture models, dynamic models, multi-fluidic models, isolated brain microvessels), in vivo models, and computational models as described in He et al., Stroke 45:2514 2014; Bickel, NeuroRx 2:15 2005; and Wang et al., Int J Pharm 288:349 2005. A neuromodulating agent that exhibits BBB impermeability can be used in the methods described herein.

Modification of Existing Compounds to Render them BBB Impermeable

There are multiple parameters that have been empirically derived in the field of medicinal chemistry to predict whether a compound will cross the BBB. The most common numeric value for describing permeability across the BBB is the log BB, defined as the logarithmic ratio of the concentration of a compound in the brain and in the blood. Empirical rules of thumb have been developed to predict BBB permeability, including rules regarding molecular size, polar surface area, sum of oxygen and nitrogen atoms, lipophilicity (e.g., partition coefficient between apolar solvent and water), “lipoaffinity”, molecular flexibility, and number of rotable bonds (summarized in Muehlbacher et al., J Comput Aided Mol Des. 25: 1095 2011; and Geldenhuys et al., Ther Deliv. 6: 961 2015). Some preferred limits on various parameters for BBB permeability are listed in Table 1 of Ghose et al., ACS Chem Neurosci. 3: 50 2012, which is incorporated herein by reference. Based on the parameters shown in the table, one of skill in the art could modify an existing neuromodulating agent to render it BBB impermeable.

One method of modifying a neuromodulating agent to prevent BBB crossing is to add a molecular adduct that does not affect the target binding specificity, kinetics, or theromodynamics of the agent. Molecular adducts that can be used to render an agent BBB impermeable include polyethylene glycol (PEG), a carbohydrate monomer or polymer, a dendrimer, a polypeptide, a charged ion, a hydrophilic group, deuterium, and fluorine. Neuromodulating agents can be tested after the addition of one or more molecular adducts or after any other properties are altered to determine whether or not they cross the BBB. Models for assessing BBB permeability include in vitro models (e.g., monolayer models, co-culture models, dynamic models, multi-fluidic models, isolated brain microvessels), in vivo models, and computational models as described in He et al., Stroke 45:2514 2014; Bickel, NeuroRx 2:15 2005; and Wang et al., Int J Pharm 288:349 2005. A neuromodulating agent that exhibits BBB impermeability can be used in the methods described herein.

Screening for or Development of BBB Impermeable Agents

Another option for developing BBB impermeable agents is to find or develop new agents that do not cross the BBB. One method for finding new BBB impermeable agents is to screen for compounds that are BBB impermeable. Compound screening can be performed using in vitro models (e.g., monolayer models, co-culture models, dynamic models, multi-fluidic models, isolated brain microvessels), in vivo models, and computational models, as described in He et al., Stroke 45:2514 2014; Bickel, NeuroRx 2:15 2005; Wang et al., Int J Pharm 288:349 2005, and Czupalla et al., Methods Mol Biol 1135:415 2014. For example, the ability of a molecule to cross the blood brain barrier can be determined in vitro using a transwell BBB assay in which microvascular endothelial cells and pericytes are co-cultured separated by a thin macroporous membrane, see e.g., Naik et al., J Pharm Sci 101:1337 2012 and Hanada et al., Int J Mol Sci 15:1812 2014; or in vivo by tracking the brain uptake of the target molecule by histology or radio-detection. Compounds would be deemed appropriate for use as neuromodulating agents in the methods described herein if they do not display BBB permeability in the aforementioned models.

III. Modulation of Immune Cells

The methods described herein can be used to modulate an immune response in a subject or cell by administering to a subject or cell a neuromodulating agent in a dose (e.g., an effective amount) and for a time sufficient to modulate the immune response. These methods can be used to treat a subject in need of modulating an immune response, e.g., a subject with cancer. One way to modulate an immune response is to modulate an immune cell activity. This modulation can occur in vivo (e.g., in a human subject or animal model) or in vitro (e.g., in acutely isolated or cultured cells, such as human cells from a patient, repository, or cell line, or rodent cells). The types of cells that can be modulated include T cells (e.g., peripheral T cells, cytotoxic T cells/CD8+ T cells, T helper cells/CD4+ T cells, memory T cells, regulatory T cells/Tregs, natural killer T cells/NKTs, mucosal associated invariant T cells, and gamma delta T cells), B cells (e.g., memory B cells, plasmablasts, plasma cells, follicular B cells/B-2 cells, marginal zone B cells, B-1 cells, regulatory B cells/Bregs), dendritic cells (e.g., myeloid DCs/conventional DCs, plasmacytoid DCs, or follicular DCs), granulocytes (e.g., eosinophils, mast cells, neutrophils, and basophils), monocytes, macrophages (e.g., peripheral macrophages or tissue resident macrophages or tumor-resident macrophages), myeloid-derived suppressor cells, natural killer (NK) cells, innate lymphoid cells, thymocytes, and megakaryocytes.

The immune cell activities that can be modulated by administering to a subject or contacting a cell with an effective amount of a neuromodulating agent described herein include activation (e.g., macrophage, T cell, NK cell, B cell, dendritic cell, neutrophil, eosinophil, or basophil activation), phagocytosis (e.g., macrophage, neutrophil, monocyte, mast cell, B cell, eosinophil, or dendritic cell phagocytosis), antibody-dependent cellular phagocytosis (e.g., ADCP by monocytes, macrophages, neutrophils, or dendritic cells), antibody-dependent cellular cytotoxicity (e.g., ADCC by NK cells, monocytes, macrophages, neutrophils, eosinophils, dendritic cells, or T cells), polarization (e.g., macrophage polarization toward an M1 or M2 phenotype or T cell polarization), proliferation (e.g., proliferation of B cells, T cells, monocytes, macrophages, dendritic cells, NK cells, mast cells, neutrophils, eosinophils, or basophils), lymph node homing (e.g., lymph node homing of T cells, B cells, dendritic cells, or macrophages), lymph node egress (e.g., lymph node egress of T cells, B cells, dendritic cells, or macrophages), recruitment (e.g., recruitment of B cells, T cells, monocytes, macrophages, dendritic cells, NK cells, mast cells, neutrophils, eosinophils, or basophils), migration (e.g., migration of B cells, T cells, monocytes, macrophages, dendritic cells, NK cells, mast cells, neutrophils, eosinophils, or basophils), differentiation (e.g., regulatory T cell differentiation), immune cell cytokine production, antigen presentation (e.g., dendritic cell, macrophage, and B cell antigen presentation), maturation (e.g., dendritic cell maturation), and degranulation (e.g., mast cell, NK cell, cytotoxic T cell, neutrophil, eosinophil, or basophil degranulation). Innervation of lymph nodes or lymphoid organs, development of high endothelial venules (HEVs), and development of ectopic or tertiary lymphoid organs (TLOs) can also be modulated using the methods described herein. Modulation can increase or decrease these activities, depending on the neuromodulating agent used to contact the cell or treat a subject.

In some embodiments, an effective amount of a neuromodulating agent is an amount sufficient to modulate (e.g., increase or decrease) one or more (e.g., 2 or more, 3 or more, 4 or more) of the following immune cell activities in the subject or cell: T cell polarization; T cell activation; dendritic cell activation; neutrophil activation; eosinophil activation; basophil activation; T cell proliferation; B cell proliferation; T cell proliferation; monocyte proliferation; macrophage proliferation; dendritic cell proliferation; NK cell proliferation; mast cell proliferation; neutrophil proliferation; eosinophil proliferation; basophil proliferation; cytotoxic T cell activation; circulating monocytes; peripheral blood hematopoietic stem cells; macrophage polarization; macrophage phagocytosis; macrophage ADCP, neutrophil phagocytosis; monocyte phagocytosis; mast cell phagocytosis; B cell phagocytosis; eosinophil phagocytosis; dendritic cell phagocytosis; macrophage activation; antigen presentation (e.g., dendritic cell, macrophage, and B cell antigen presentation); antigen presenting cell migration (e.g., dendritic cell, macrophage, and B cell migration); lymph node immune cell homing and cell egress (e.g., lymph node homing and egress of T cells, B cells, dendritic cells, or macrophages); NK cell activation; NK cell ADCC, mast cell degranulation; NK cell degranulation; cytotoxic T cell degranulation; neutrophil degranulation; eosinophil degranulation; basophil degranulation; neutrophil recruitment; eosinophil recruitment; NKT cell activation; B cell activation; regulatory T cell differentiation; dendritic cell maturation; development of high endothelial venules (HEVs); development of ectopic or tertiary lymphoid organs (TLOs); or lymph node or secondary lymphoid organ innervation. In certain embodiments, the immune response (e.g., an immune cell activity listed herein) is increased or decreased in the subject or cell at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the immune response is increased or decreased in the subject or cell between 5-20%, between 5-50%, between 10-50%, between 20-80%, between 20-70%, between 50-200%, between 100%-500%.

After a neuromodulating agent is administered to treat a patient or contact a cell, a readout can be used to assess the effect on immune cell activity. Immune cell activity can be assessed by measuring a cytokine or marker associated with a particular immune cell type, as listed in Table 9 (e.g., performing an assay listed in Table 9 for the cytokine or marker). In certain embodiments, the parameter is increased or decreased in the subject at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the parameter is increased or decreased in the subject between 5-20%, between 5-50%, between 10-50%, between 20-80%, between 20-70%, between 50-200%, between 100%-500%. A neuromodulating agent can be administered at a dose (e.g., an effective amount) and for a time sufficient to modulate an immune cell activity described herein below.

After a neuromodulating agent is administered to treat a patient or contact a cell, a readout can be used to assess the effect on immune cell migration. Immune cell migration can be assessed by measuring the number of immune cells in a location of interest (e.g., a lymph node or secondary lymphoid organ, site of inflammation, or a tumor). Immune cell migration can also be assessed by measuring a chemokine, receptor, or marker associated with immune cell migration, as listed in Tables 10 and 11. In certain embodiments, the parameter is increased or decreased in the subject at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the parameter is increased or decreased in the subject between 5-20%, between 5-50%, between 10-50%, between 20-80%, between 20-70%, between 50-200%, between 100%-500%. A neuromodulating agent can be administered at a dose (e.g., an effective amount) and for a time sufficient to modulate an immune cell migration as described herein below.

A neuromodulating agent described herein can affect immune cell migration. Immune cell migration between peripheral tissues, the blood, and the lymphatic system as well as lymphoid organs is essential for the orchestration of productive innate and adaptive immune responses. Immune cell migration is largely regulated by trafficking molecules including integrins, immunoglobulin cell-adhesion molecules (IgSF CAMs), cadherins, selectins, and a family of small cytokines called chemokines (Table 10). Cell adhesion molecules and chemokines regulate immune cell migration by both inducing extravasation from the circulation into peripheral tissues and acting as guidance cues within peripheral tissues themselves. For extravasation to occur, chemokines must act in concert with multiple trafficking molecules including C-type lectins (L-, P-, and E-selectin), multiple integrins, and cell adhesion molecules (ICAM-1, VCAM-1 and MAdCAM-1) to enable a multi-step cascade of immune cell capturing, rolling, arrest, and transmigration via the blood endothelial barrier (Table 11). Some trafficking molecules are constitutively expressed and manage the migration of immune cells during homeostasis, while others are specifically upregulated by inflammatory processes such as cancer.

The expression of trafficking molecules important for extravasation is mainly regulated on specialized blood vessels called high endothelial venules (HEVs), which are the entry portals from the circulation into the periphery and are usually present in secondary lymphoid organs (SLOs) and chronically inflamed tissue. Chronically inflamed tissues often develop lymphoid-like structures called ectopic or tertiary lymphoid organs (TLOs) that contain structures resembling SLOs including HEVs, lymphoid stromal cells, and confined compartments of T and B lymphocytes. As they can act as major gateways for immune cell migration into peripheral tissues, TLOs have been shown to be important in the pathogenesis of cancer.

Once within peripheral tissues, four modes of immune cell migration have been observed: 1) chemokinesis: migration driven by soluble chemokines, without concentration gradients to provide directional bias, 2) haptokinesis: migration along surfaces presenting immobilized ligands such as chemokines or integrins, without concentration gradients to provide directional bias, 3) chemotaxis: directional migration driven by concentration gradients of soluble chemokines, and 4) haptotaxis: directional migration along surfaces presenting gradients of immobilized ligands such as chemokines or integrins. The response of immune cells to trafficking molecules present on the endothelium depends on the composition, expression, and/or functional activity of their cognate receptors, which in turn depends on activation state and immune cell subtype.

Innate immune cells generally migrate toward inflammation-induced trafficking molecules in the periphery. In contrast, naïve T and B cells constantly re-circulate between the blood and secondary lymphoid organs to screen for their cognate antigen presented by activated dendritic cells (DCs) or fibroblastic reticular cells (FRCs), respectively. If activated by recognition of their cognate antigen and appropriate co-stimulation within SLOs, both cell types undergo a series of complex maturation steps, including differentiation and proliferation, ultimately leading to effector and memory immune cell phenotypes. To reach their peripheral target sites, certain effector and memory T and B cell subsets egress from SLOs to the blood circulation via efferent lymphatics. In order to do so, they migrate toward a Sphingosine-1-phosphate (S1P) gradient sensed using their Sphingosine-1-phosphate receptor 1 (S1P1 or S1PR1). For successful egress into efferent lymphatics, immune cells need to overcome SLO retention signals through the CCR7/CCL21 axis or through CD69-mediated downregulation of S1P₁.

Finally, certain immune cell subsets, for example mature dendritic cells (DCs) and memory T cells, migrate from peripheral tissues into SLOs via afferent lymphatics. To exit from peripheral tissues and enter afferent lymphatics, immune cells again largely depend on the CCR7/CCL21 and S1P₁/S1P axis. Specifically, immune cells need to overcome retention signals delivered via the CCR7/CCL21 axis, and migrate toward an S1P gradient established by the lymphatic endothelial cells using S1P₁. The selective action of trafficking molecules on distinct immune cell subsets as well as the distinct spatial and temporal expression patterns of both the ligands and receptors are crucial for the fine-tuning of immune responses during homeostasis and disease.

Aberrant immune cell migration is observed in multiple immune-related pathologies. Immune cell adhesion deficiencies, caused by molecular defects in integrin expression, fucosylation of selectin ligands, or inside-out activation of integrins on leukocytes and platelets, lead to impaired immune cell migration into peripheral tissues. This results in leukocytosis and in increased susceptibility to recurrent bacterial and fungal infections, which can be difficult to treat and potentially life-threatening. Alternatively, exaggerated migration of specific immune cell subsets into specific peripheral tissues is associated with a multitude of pathologies. For example, excessive neutrophil accumulation in peripheral tissues contributes to the development of ischemia-reperfusion injury, such as that observed during acute myocardial infarction, stroke, shock and acute respiratory distress syndrome. Excessive Th1 inflammation characterized by tissue infiltration of interferon-gamma secreting effector T cells and activated macrophages is associated with atherosclerosis, allograft rejection, hepatitis, and multiple autoimmune diseases including multiple sclerosis, rheumatoid arthritis, psoriasis, Crohn's disease, type 1 diabetes and lupus erythematodes. Excessive Th2 inflammation characterized by tissue infiltration of IL-4, IL-5, and IL-13 secreting Th2 cells, eosinophils and mast cells is associated with asthma, food allergies and atopic dermatitis.

In the context of tumor biology, the balance between effector immune cell infiltrates eliminating tumor cells and suppressive immune cell infiltrates protecting tumor cells is critical in determining the net outcome of tumor development, namely elimination, equilibrium, or escape. The main anti-tumor immune cell subsets are natural killer (NK) cells, γδ T cells, Th1 CD4+ and cytotoxic CD8+ T cells (CTLs), mature dendritic cells (mDCs), and inflammatory macrophages (often referred to as M1 macrophages). The main pro-tumor immune cell subsets are suppressive tumor-associated macrophages (TAM, often referred to as M2 macrophages), myeloid-derived suppressor cells (MDSC), regulatory T cells (Treg), and immature dendritic cells (iDCs). While effector immune cells subsets are generally attracted to migrate into the tumor microenvironment via CXCR3 and its ligands CXCL9, CXCL10 and CXCL11, suppressive immune cell subsets depend on multiple sets of chemokine and chemokine receptors, including CCR2/CCL2, CCR5/CCL5, CXCR1/CXCL8 (IL8), CXCR2/CXCL5, and CXCR4/CXCL12. Accordingly, the upregulation of CXCL9 and CXCL10 within the tumor generally correlates with good prognosis, and upregulation of suppressive chemokines correlates with bad prognosis of cancer patients.

Specific chemokine pathways not only increase the infiltration of immunosuppressive immune cell subsets, but also promote tumor angiogenesis and metastasis and are thus interesting targets for the development of anti-cancer therapies. Inducing T cell migration into tumors might be especially beneficial in the context of cancer immunotherapy, as a T-cell inflamed microenvironment correlates with good response to these types of interventions.

Finally, tumor-draining lymph nodes (tdLNs) are essential gateways for the induction of adaptive immune responses against tumor cells. However, even though tdLNs are exposed to antigens shed by the upstream tumor cells, they often contain more immunosuppressive cytokines and cells than a non-involved lymph node. This is because a multitude of immunosuppressive molecules are secreted by the upstream tumor microenvironment, thus influencing the immune status of the downstream lymph node. Therefore, strategies that could alter immune cell migration into the tumor-draining lymph node could shift the balance between suppressive and effector immune cells in favor of the latter, thus unleashing potent anti-tumor immune responses.

Immune Effects

A variety of in vitro and in vivo assays can be used to determine how a neuromodulating agent affects an immune cell activity. The effect of a neuromodulating agent on T cell polarization in a subject can be assessed by evaluation of cell surface markers on T cells obtained from the subject. A blood sample, lymph node biopsy, or tissue sample can be collected from a subject and T cells from the sample evaluated for one or more (e.g., 2, 3, or 4 or more) Th1-specific markers: T-bet, IL-12R, STAT4, or chemokine receptors CCR5, CXCR6, and CXCR3; or Th2-specific markers: CCR3, CXCR4, or IL-4Rα. T cell polarization can also be assessed using the same methods in an in vivo animal model. This assay can also be performed by adding a neuromodulating agent to T cells in vitro (e.g., T cells obtained from a subject, animal model, repository, or commercial source) and measuring the aforementioned markers to evaluate T cell polarization. These markers can be assessed using flow cytometry, immunohistochemistry, in situ hybridization, and other assays that allow for measurement of cellular markers. Comparing results from before and after administration of a neuromodulating agent can be used to determine its effect.

The effect of a neuromodulating agent on T cell activation in a subject can be assessed by evaluation of cellular markers on T cells obtained from the subject. A blood sample, lymph node biopsy, or tissue sample can be collected from a subject and T cells from the sample evaluated for one or more (e.g., 2, 3, 4 or more) activation markers: CD25, CD71, CD26, CD27, CD28, CD30, CD154, CD40L, CD134, CD69, CD62L or CD44. T cell activation can also be assessed using the same methods in an in vivo animal model. This assay can also be performed by adding a neuromodulating agent to T cells in vitro (e.g., T cells obtained from a subject, animal model, repository, or commercial source) and measuring the aforementioned markers to evaluate T cell activation. Similar approaches can be used to assess the effect of a neuromodulating agent on activation of other immune cells, such as eosinophils (markers: CD35, CD11b, CD66, CD69 and CD81), dendritic cells (makers: IL-8, MHC class II, CD40, CD80, CD83, and CD86), basophils (CD63, CD13, CD4, and CD203c), and neutrophils (CD11 b, CD35, CD66b and CD63). These markers can be assessed using flow cytometry, immunohistochemistry, in situ hybridization, and other assays that allow for measurement of cellular markers. Comparing results from before and after administration of a neuromodulating agent can be used to determine its effect.

The effect of a neuromodulating agent on immune cell activation can also be assessed through measurement of secreted cytokines and chemokines. An activated immune cell (e.g., T cell, B cell, macrophage, monocyte, dendritic cell, eosinophil, basophil, mast cell, NK cell, or neutrophil) can produce pro-inflammatory cytokines and chemokines (e.g., IL-1β, IL-5, IL-6, IL-8, IL-10, IL-12, IL-13, IL-18, TNFα, and IFN-γ). Activation can be assessed by measuring cytokine levels in a blood sample, lymph node biopsy, or tissue sample from a human subject or animal model, with higher levels of pro-inflammatory cytokines following treatment with a neuromodulating agent indicating increased activation, and lower levels indicating decreased activation. Activation can also be assessed in vitro by measuring cytokines secreted into the media by cultured cells. Cytokines can be measured using ELISA, western blot analysis, and other approaches for quantifying secreted proteins. Comparing results from before and after administration of a neuromodulating agent can be used to determine its effect.

The effect of a neuromodulating agent on T cell proliferation in a subject can be assessed by evaluation of markers of proliferation in T cells obtained from the subject. A blood sample, lymph node biopsy, or tissue sample can be collected from a subject and T cells from the sample evaluated for Ki67 marker expression. T cell proliferation can also be assessed using the same methods in an in vivo animal model. This assay can also be performed by adding a neuromodulating agent to T cells in vitro (e.g., T cells obtained from a subject, animal model, repository, or commercial source) and measuring Ki67 to evaluate T cell proliferation. Assessing whether a neuromodulating agent induces T cell proliferation can also be performed by in vivo (e.g., in a human subject or animal model) by collecting blood samples before and after neuromodulating agent administration and comparing T cell numbers, and in vitro by quantifying T cell numbers before and after contacting T cells with a neuromodulating agent. These approaches can also be used to measure the effect of a neuromodulating agent on proliferation of any immune cell (e.g., B cells, T cells, macrophages, monocytes, dendritic cells, NK cells, mast cells, eosinophils, basophils, and neutrophils). Ki67 can be assessed using flow cytometry, immunohistochemistry, in situ hybridization, and other assays that allow for measurement of nuclear markers. Comparing results from before and after administration of a neuromodulating agent can be used to determine its effect.

The effect of a neuromodulating agent on cytotoxic T cell activation in a subject can be assessed by evaluation of T cell granule markers in T cells obtained from the subject. A blood sample, lymph node biopsy, or tissue sample can be collected from a subject and T cells from the sample evaluated for granzyme or perforin expression. Cytotoxic T cell activation can also be assessed using the same methods in an in vivo animal model. This assay can also be performed by adding a neuromodulating agent to cytotoxic T cells in vitro (e.g., cytotoxic T cells obtained from a subject, animal model, repository, or commercial source) and measuring the aforementioned markers to evaluate T cell proliferation. These markers can be detected in the media from cytotoxic T cell cultures. Techniques including ELISA, western blot analysis can be used to detect granzyme and perforin in conditioned media, flow cytometry, immunohistochemistry, in situ hybridization, and other assays can detect intracellular granzyme and perforin and their synthesis. Comparing results from before and after administration of a neuromodulating agent can be used to determine its effect.

The effect of a neuromodulating agent on circulating monocytes in a subject can be assessed by evaluation of cell surface markers on primary blood mononuclear cells obtained from the subject. A blood sample, lymph node biopsy, or tissue sample can be collected from a subject and monocytes from the sample evaluated for CD14 and/or CD16 expression. Circulating monocytes can also be assessed using the same methods in an in vivo animal model. This assay can be performed by taking a blood sample before treatment with a neuromodulating agent and comparing it to a blood sample taken after treatment. CD14 and CD16 can be detected using flow cytometry, immunohistochemistry, western blot analysis, or any other technique that can measure cell surface protein levels. Comparing results from before and after administration of a neuromodulating agent can be used to determine its effect. This assay can be used to detect the number of monocytes in the bloodstream or to determine whether monocytes have adopted a CD14+/CD16+ phenotype, which indicates a pro-inflammatory function.

The effect of a neuromodulating agent on peripheral blood hematopoietic stem cells in a subject can be assessed by evaluation of cell surface markers on primary blood mononuclear cells obtained from the subject. A blood sample, lymph node biopsy, or tissue sample can be collected from a subject and stem cells from the sample evaluated for one or more (2, 3 or 4 or more) specific markers: CD34, c-kit, Sca-1, or Thy1.1. Peripheral blood hematopoietic stem cells can also be assessed using the same methods in an in vivo animal model. This assay can be performed by taking a blood sample before treatment with a neuromodulating agent and comparing it to a blood sample taken after treatment. The aforementioned markers can be detected using flow cytometry, immunohistochemistry, western blot analysis, or any other technique that can measure cell surface protein levels. Comparing results from before and after administration of a neuromodulating agent can be used to determine its effect. This assay can be used to detect the number of stem cells mobilized into the bloodstream or to determine whether treatment induces differentiation into a particular hematopoietic lineage (e.g., decreased CD34 and increased GPA indicates differentiation into red blood cells, decreased CD34 and increased CD14 indicates differentiation into monocytes, decreased CD34 and increased CD11 b or CD68 indicates differentiation into macrophages, decreased CD34 and increased CD42b indicates differentiation into platelets, decreased CD34 and increased CD3 indicates differentiation into T cells, decreased CD34 and increased CD19 indicates differentiation into B cells, decreased CD34 and increased CD25 or CD69 indicates differentiation into activated T cells, decreased CD34 and increased CD1c, CD83, CD141, CD209, or MHC II indicates differentiation into dendritic cells, decreased CD34 and increased CD56 indicates differentiation into NK cells, decreased CD34 and increased CD15 indicates differentiation into neutrophils, decreased CD34 and increased 2D7 antigen, CD123, or CD203c indicates differentiation into basophils, and decreased CD34 and increased CD193, EMR1, or Siglec-8 indicates differentiation into eosinophils.

The effect of a neuromodulating agent on macrophage polarization in a subject can be assessed by evaluation of cellular markers in macrophages cells obtained from the subject. A blood sample, lymph node biopsy, or tissue sample can be collected from a subject and macrophages from the sample evaluated for one of more (2, 3 or 4 or more) specific markers. Markers for M1 polarization include IL-12, TNF, IL-1β, IL-6, IL-23, MARCO, MHC-II, CD86, iNOS, CXCL9, and CXCL10. Markers for M2 polarized macrophages include IL-10, IL1-RA, TGFβ, MR, CD163, DC-SIGN, Dectin-1, HO-1, arginase (Arg-1), CCL17, CCL22 and CCL24. Macrophage polarization can also be assessed using the same methods in an in vivo animal model. This assay can also be performed on cultured macrophages obtained from a subject, an animal model, repository, or commercial source to determine how contacting a macrophage with a neuromodulating agent affects polarization. The aforementioned markers can be evaluated by comparing measurements obtained before and after administration of a neuromodulating agent to a subject, animal model, or cultured cell. Surface markers or intracellular proteins (e.g., MHC-11, CD86, iNOS, CD163, Dectin-1, HO-1, Arg-1, etc.) can be measured using flow cytometry, immunohistochemistry, in situ hybridization, or western blot analysis, and secreted proteins (e.g., IL-12, TNF, IL-1β, IL-10, TGFβ, IL1-RA, chemokines CXC8, CXC9, CCL17, CCL22, and CCL24, etc.) can be measured using the same methods or by ELISA or western blot analysis of culture media or blood samples. Comparing results from before and after administration of a neuromodulating agent can be used to determine its effect.

The effect of a neuromodulating agent on macrophage phagocytosis in a subject can be assessed by culturing macrophages obtained from the subject with fluorescent beads. A blood sample, lymph node biopsy, or tissue sample can be collected from a subject and macrophages from the sample evaluated for engulfment of fluorescent beads. This assay can also be performed on cultured macrophages obtained from an animal model, repository, or commercial source to determine how contacting a macrophage with a neuromodulating agent affects phagocytosis. The same phagocytosis assay can be used to evaluate the effect of a neuromodulating agent on phagocytosis in other immune cells (e.g., neutrophils, monocytes, mast cells, B cells, eosinophils, or dendritic cells). Comparing results from before and after administration of a neuromodulating agent can be used to determine its effect on phagocytosis.

In some embodiments, phagocytosis is ADCP. ADCP can be assessed using similar methods to those described above by incubating immune cells (e.g., macrophages, neutrophils, monocytes, mast cells, B cells, eosinophils, or dendritic cells) isolated from a blood sample, lymph node biopsy, or tissue sample with fluorescent beads coated with IgG antibodies. In some embodiments, immune cells are incubated with a target cell line that has been pre-coated with antibodies to a surface antigen expressed by the target cell line. ADCP can be evaluated by measuring fluorescence inside the immune cell or quantifying the number of beads or cells engulfed. This assay can also be performed on cultured immune cells obtained from an animal model, repository, or commercial source to determine how contacting an immune cell with a neuromodulating agent affects ADCP. The ability of an immune cell to perform ADCP can also be evaluated by assessing expression of certain Fc receptors (e.g., FcγRIIa, FcγRIIIa, and FcγRI). Fc receptor expression can be assessed using flow cytometry, immunohistochemistry, in situ hybridization, or other assays that allow for measurement of cell surface markers. Comparing phagocytosis or Fc receptor expression before and after administration of a neuromodulating agent can be used to determine its effect on ACDP. In some embodiments, the neuromodulating agent increases macrophage ADCP of antibody-coated tumor cells.

The effect of a neuromodulating agent on macrophage activation in a subject can be assessed by evaluation of cell surface markers on macrophages cells obtained from the subject. A blood sample, lymph node biopsy, or tissue sample can be collected from a subject and macrophages from the sample evaluated for one or more (e.g., 1, 2, 3 or 4 or more) specific markers: F4/80, HLA molecules (e.g., MHC-II), CD80, CD68, CD11b, or CD86. Macrophage activation can also be assessed using the same methods in an in vivo animal model. This assay can also be performed by adding a neuromodulating agent to macrophages in vitro (e.g., macrophages obtained from a subject, animal model, repository, or commercial source) and measuring the aforementioned markers to evaluate macrophage activation. These markers can be assessed using flow cytometry, immunohistochemistry, in situ hybridization, and other assays that allow for measurement of cell surface markers. As mentioned above, macrophage activation can also be evaluated based on cytokine production (e.g., pro-inflammatory cytokine production) as measured by ELISA and western blot analysis. Comparing results from before and after administration of a neuromodulating agent can be used to determine its effect.

The effect of a neuromodulating agent on antigen presentation in a subject can be assessed by evaluation of cell surface markers on antigen presenting cells (e.g., dendritic cells, macrophages, and B cells) obtained from the subject. A blood sample, lymph node biopsy, or tissue sample can be collected from a subject and antigen presenting cells (e.g., dendritic cells, macrophages, and B cells) from the sample evaluated for one or more (e.g., 2, 3 or 4 or more) specific markers: CD11c, CD11b, HLA molecules (e.g., MHC-II), CD40, B7, IL-2, CD80 or CD86. Antigen presentation can also be assessed using the same methods in an in vivo animal model. This assay can also be performed by adding a neuromodulating agent to antigen presenting cells (e.g., dendritic cells) in vitro (e.g., antigen presenting cells obtained from a subject, animal model, repository, or commercial source) and measuring the aforementioned markers to evaluate antigen presentation. These markers can be assessed using flow cytometry, immunohistochemistry, in situ hybridization, and other assays that allow for measurement of cell surface markers. Comparing results from before and after administration of a neuromodulating agent can be used to determine its effect.

The effect of a neuromodulating agent on antigen presenting cell migration in a subject can be assessed by evaluation of cell surface markers on antigen presenting cells (e.g., dendritic cells, B cells, and macrophages) obtained from the subject. A blood sample, lymph node biopsy, or tissue sample can be collected from a subject and antigen presenting cells (e.g., dendritic cells, B cells, and macrophages) from the sample evaluated for CCR7 expression. Antigen presenting cell migration can also be assessed using the same methods in an in vivo animal model. This assay can also be performed by adding a neuromodulating agent to antigen presenting cells (e.g., dendritic cells, B cells, and macrophages) in vitro (e.g., antigen presenting cells obtained from a subject, animal model, repository, or commercial source) and measuring CCR7 to evaluate antigen presenting cell migration. CCR7 can be assessed using flow cytometry, immunohistochemistry, in situ hybridization, and other assays that allow for measurement of cell surface markers. Comparing results from before and after administration of a neuromodulating agent can be used to determine its effect.

The effect of a neuromodulating agent on lymph node immune cell homing and cell egress in a subject can be assessed by evaluation of cell surface markers on T or B cells obtained from the subject. A blood sample, lymph node biopsy, or tissue sample can be collected from a subject and T or B cells from the sample evaluated for one or more specific markers: CCR7 or S1PR1. Lymph node immune cell homing and cell egress can also be assessed using the same methods in an in vivo animal model. This assay can also be performed by adding a neuromodulating agent to T or B cells in vitro (e.g., T or B cells obtained from a subject, animal model, repository, or commercial source) and measuring the aforementioned markers to evaluate T or B cell lymph node homing. These markers can also be used to assess lymph node homing and cell egress of dendritic cells and macrophages. CCR7 and S1PR1 can be assessed using flow cytometry, immunohistochemistry, in situ hybridization, and other assays that allow for measurement of cell surface markers. If using an animal model, lymph nodes or sites of inflammation can be imaged in vivo (e.g., using a mouse that expresses fluorescently labeled T or B cells) or after biopsy to determine whether T or B cell numbers change as a result of administration of a neuromodulating agent. Comparing results from before and after administration of a neuromodulating agent can be used to determine its effect.

In some embodiments, a neuromodulating agent increases homing or decreases egress of naïve T cells into or out of secondary lymphoid organs prior to antigen challenge (e.g., prior to administration of a vaccine) to generate a better antigen-specific response. In some embodiments, a neuromodulating agent decreases homing or increases egress of inflammatory immune cells (e.g., neutrophils) into or out of peripheral tissues during acute infection or injury to prevent conditions such as ischemia-reperfusion disorders. In some embodiments, a neuromodulating agent decreases homing or increases egress of effector immune subsets into or out of peripheral tissues to avoid inflammation-induced tissue damage.

The effect of a neuromodulating agent on NK cell activation in a subject can be assessed by evaluation of cell surface markers on NK cells obtained from the subject. A blood sample, lymph node biopsy, or tissue sample can be collected from a subject and NK cells from the sample evaluated for one or more (e.g., 2, 3 or 4 or more) specific markers: CD117, NKp46, CD94, CD56, CD16, KIR, CD69, HLA-DR, CD38, KLRG1, and TIA-1. NK cell activation can also be assessed using the same methods in an in vivo animal model. This assay can also be performed by adding a neuromodulating agent to NK cells in vitro (e.g., NK cells obtained from a subject, animal model, repository, or commercial source) and measuring the aforementioned markers to evaluate NK cell activation. The effect of a neuromodulating agent can be determined by comparing results from before and after neuromodulating agent administration.

In some embodiments, activated NK cells have increased lytic function or are cytotoxic (e.g., capable of performing ADCC). The effect of a neuromodulating agent on ADCC can be assessed by incubating immune cells capable of ADCC (e.g., NK cells, monocytes, macrophages, neutrophils, eosinophils, dendritic cells, or T cells) with a target cell line that has been pre-coated with antibodies to a surface antigen expressed by the target cell line. ADCC can be assessed by measuring the number of surviving target cells with a fluorescent viability stain or by measuring the secretion of cytolytic granules (e.g., perforin, granzymes, or other cytolytic proteins released from immune cells). Immune cells can be collected from a blood sample, lymph node biopsy, or tissue sample from a human subject or animal model treated with a neuromodulating agent. This assay can also be performed by adding a neuromodulating agent to immune cells in vitro (e.g., immune cells obtained from a subject, animal model, repository, or commercial source). The effect of a neuromodulating agent on ADCC can be determined by comparing results from before and after neuromodulating agent administration. In some embodiments, the neuromodulating agent increases NK cell ADCC of antibody-targeted tumors.

The effect of a neuromodulating agent on mast cell degranulation in a subject can be assessed by evaluation of markers in mast cells obtained from the subject. A blood sample, lymph node biopsy, or tissue sample can be collected from a subject and mast cells from the sample evaluated for one or more (e.g., 1, 2, 3 or 4 or more) specific markers: IgE, histamine, IL-4, TNFα, CD300a, tryptase, or MMP9. Mast cell degranulation can also be assessed using the same methods in an in vivo animal model. This assay can also be performed by adding a neuromodulating agent to mast cells in vitro (e.g., mast cells obtained from a subject, animal model, repository, or commercial source) and measuring the aforementioned markers to evaluate mast cell degranulation. Some of these markers (e.g., histamine, TNFα, and IL-4) can be detected by measuring levels in the mast cell culture medium after mast cells are contacted with a neuromodulating agent. The effect of a neuromodulating agent can be determined by comparing results from before and after neuromodulating agent administration. This approach can also be used to evaluate the effect of a neuromodulating agent on degranulation by other cells, such as neutrophils (markers: CD11 b, CD13, CD18, CD45, CD15, CD66b IL-1β, IL-8, and IL-6), eosinophils (markers: major basic protein (MBP), eosinophil cationic protein (ECP), eosinophil peroxidase (EPX), eosinophil-derived neurotoxin (EDN)), basophils (markers: histamine, heparin, chondroitin, elastase, lysophospholipase, and LTD-4), NK cells (markers: LAMP-1, perforin, and granzymes), and cytotoxic T cells (markers: LAMP-1, perforin, and granzymes). Markers can be detected using flow cytometry, immunohistochemistry, ELISA, western blot analysis, or in situ hybridization.

The effect of a neuromodulating agent on neutrophil recruitment in a subject can be assessed by evaluation of cell surface markers on neutrophils obtained from the subject. A blood sample, lymph node biopsy, or tissue sample can be collected from a subject and neutrophils from the sample evaluated for one or more (e.g., 1, 2, 3 or 4 or more) specific markers: CD11b, CD14, CD114, CD177, CD354, or CD66. To determine whether neutrophils are being recruited to a specific site (e.g., a site of inflammation or a tumor), the same markers can be measured at the site of inflammation or in a tumor biopsy. Neutrophil recruitment can also be assessed using the same methods in an in vivo animal model. This assay can also be performed by adding a neuromodulating agent to neutrophils in vitro (e.g., neutrophils obtained from a subject, animal model, repository, or commercial source) and measuring the aforementioned markers to evaluate neutrophil recruitment. These markers can be assessed using flow cytometry, immunohistochemistry, in situ hybridization, and other assays that allow for measurement of cell surface markers. The effect of a neuromodulating agent can be determined by comparing results from before and after neuromodulating agent administration.

The effect of a neuromodulating agent on eosinophil recruitment in a subject can be assessed by evaluation of cell surface markers on eosinophil obtained from the subject. A blood sample, lymph node biopsy, or tissue sample can be collected from a subject and eosinophils from the sample evaluated for one or more (e.g., 1, 2, 3 or 4 or more) specific markers: CD15, IL-3R, CD38, CD106, CD294 or CD85G. To determine whether eosinophils are being recruited to a specific site (e.g., a site of inflammation or a tumor), the same markers can be measured at the site of inflammation or in a tumor biopsy. Eosinophil recruitment can also be assessed using the same methods in an in vivo animal model. This assay can also be performed by adding a neuromodulating agent to eosinophils in vitro (e.g., eosinophils obtained from a subject, animal model, repository, or commercial source) and measuring the aforementioned markers to evaluate eosinophil recruitment. These markers can be assessed using flow cytometry, immunohistochemistry, in situ hybridization, and other assays that allow for measurement of cell surface markers. The effect of a neuromodulating agent can be determined by comparing results from before and after neuromodulating agent administration.

The effect of a neuromodulating agent on NKT cell activation in a subject can be assessed by evaluation of cell surface markers on NKT cells obtained from the subject. A blood sample, lymph node biopsy, or tissue sample can be collected from a subject and NKT cells from the sample evaluated for one or more specific markers: CD272 or CD352. Activated NKT cells produce IFN-γ, IL-4, GM-CSF, IL-2, IL-13, IL-17, IL-21 and TNFα. NKT cell activation can also be assessed using the same methods in an in vivo animal model. This assay can also be performed by adding a neuromodulating agent to NKT cells in vitro (e.g., NKT cells obtained from a subject, animal model, repository, or commercial source) and measuring the aforementioned markers to evaluate NKT cell activation. Cell surface markers CD272 and CD352 can be assessed using flow cytometry, immunohistochemistry, in situ hybridization, and other assays that allow for measurement of cell surface markers. The secreted proteins can be detected in blood samples or cell culture media using ELISA, western blot analysis, or other methods for detecting proteins in solution. The effect of a neuromodulating agent can be determined by comparing results from before and after neuromodulating agent administration.

The effects of a neuromodulating agent on B cell activation in a subject can be assessed by evaluation of cell surface markers on B cells obtained from the subject. A blood sample, lymph node biopsy, or tissue sample can be collected from a subject and B cells from the sample evaluated for one or more (e.g., 2, 3 or 4 or more) specific markers: CD19, CD20, CD40, CD80, CD86, CD69, IgM, IgD, IgG, IgE, or IgA. B cell activation can also be assessed using the same methods in an in vivo animal model. This assay can also be performed by adding a neuromodulating agent to B cells in vitro (e.g., B cells obtained from a subject, animal model, repository, or commercial source) and measuring the aforementioned markers to evaluate B cell activation. These markers can be assessed using flow cytometry, immunohistochemistry, in situ hybridization, and other assays that allow for measurement of cell surface markers. The effect of a neuromodulating agent can be determined by comparing results from before and after neuromodulating agent administration.

The effect of a neuromodulating agent on regulatory T cell differentiation in a subject can be assessed by evaluation of markers in regulatory T cells obtained from the subject. A blood sample, lymph node biopsy, or tissue sample can be collected from a subject and regulatory T cells from the sample evaluated for one or more (e.g., 1, 2, 3, 4 or more) specific markers: CD4, CD25, or FoxP3. Regulatory T cell differentiation can also be assessed using the same methods in an in vivo animal model. This assay can also be performed by adding a neuromodulating agent to regulatory T cells in vitro (e.g., regulatory T cells obtained from a subject, animal model, repository, or commercial source) and measuring the aforementioned markers to evaluate regulatory T cell differentiation. These markers can be assessed using flow cytometry, immunohistochemistry, in situ hybridization, and other assays that allow for measurement of cellular markers. The effect of a neuromodulating agent can be determined by comparing results from before and after neuromodulating agent administration.

The effect of a neuromodulating agent on innervation of a lymph node or secondary lymphoid organ can be assessed by evaluation of neuronal markers in a lymph node or secondary lymphoid organ biopsy sample obtained from a human subject or animal model. A biopsy can be collected from the subject and evaluated for one or more (e.g., 1, 2, 3, 4, or 4 or more) neuronal markers selected from: Neurofilament, synapsin, synaptotagmin, or neuron specific enolase. Lymph node innervation can also be assessed using electrophysiological approaches (e.g., recording neuronal activity in a lymph node or secondary lymphoid organ in a human subject or animal model). The effect of a neuromodulating agent can be determined by comparing results from before and after neuromodulating agent administration.

The neuromodulating agent can also reduce the number of nerve fibers in the affected tissue or reduce the activity of peripheral nerve fibers in the affected tissue. For example, the method includes administering to the subject (e.g., a human subject or animal model) a neuromodulating agent in an amount and for a time sufficient to reduce the number of nerve fibers in the affected tissue or reduce the activity of peripheral nerve fibers in the affected tissue. The affected tissue can be a lymph node, a lymphoid organ, a tumor, a tumor micro-environment, or the bone marrow niche. The number of nerve fibers in the affected tissue or the activity of peripheral nerve fibers in the affected tissue can be decreased in the subject at least 1%, 2%, 5%, 10%, 15%, 20%, 25%, 30%, 35%, 40%, 50%, 60%, 70%, 80%, 90%, 95% or more, compared to before the administration. The number of nerve fibers in the affected tissue or the activity of peripheral nerve fibers in the affected tissue can be decreased in the subject between 5-20%, between 5-50%, between 10-50%, between 20-80%, between 20-70%.

The neuromodulating agent can also increase the number of nerve fibers in the affected tissue or increase the activity of peripheral nerve fibers in the affected tissue. For example, the method includes administering to the subject (e.g., a human subject or animal model) a neuromodulating agent in an amount and for a time sufficient to increase the number of nerve fibers in the affected tissue or increase the activity of peripheral nerve fibers in the affected tissue. The affected tissue can be a lymph node, a lymphoid organ, a tumor, a tumor micro-environment, or the bone marrow niche. The number of nerve fibers in the affected tissue or the activity of peripheral nerve fibers in the affected tissue can be increased in the subject at least 1%, 2%, 5%, 10%, 15%, 20%, 25%, 30%, 35%, 40%, 50%, 60%, 70%, 80% or more, compared to before the administration. The number of nerve fibers in the affected tissue or the activity of peripheral nerve fibers in the affected tissue can be increased in the subject between 5-20%, between 5-50%, between 10-50%, between 20-80%, between 20-70%.

The nerve fibers that are modulated can be part of the peripheral nervous system, e.g., a somatic nerve, an autonomic nerve, a sensory nerve, a cranial nerve, an optic nerve, an olfactory nerve, a sympathetic nerve, a parasympathetic nerve, a chemoreceptor, a photoreceptor, a mechanoreceptor, a thermoreceptor, a nociceptor, an efferent nerve fiber, or an afferent nerve fiber.

The effect of a neuromodulating agent on immune cell cytokine production can be assessed by evaluation of cellular markers in an immune cell sample obtained from a human subject or animal model. A blood sample, lymph node biopsy, or tissue sample can be collected for the subject and evaluated for one or more (e.g., 1, 2, 3, 4, or 4 or more) cytokine markers selected from: pro-inflammatory cytokines (e.g., IL-1β, IL-5, IL-6, IL-8, IL-10, IL-12, IL-13, IL-18, TNFα, IFNγ, GMCSF), pro-survival cytokines (e.g., IL-2, IL-4, IL-6, IL-7, and IL-15) and anti-inflammatory cytokines (e.g., IL-4, IL-10, IL-11, IL-13, IFNα, and TGFβ). Some cytokines can function as both pro- and anti-inflammatory cytokines depending on context or indication (e.g., IL-4 is often categorized as an anti-inflammatory cytokine, but plays a pro-inflammatory role in mounting an allergic or anti-parasitic immune response). Cytokines can be also detected in the culture media of immune cells contacted with a neuromodulating agent. Cytokines can be detected using ELISA, western blot analysis, or other methods for detecting protein levels in solution. The effect of a neuromodulating agent can be determined by comparing results from before and after neuromodulating agent administration.

In some embodiments, a neuromodulating agent decreases or prevents the development of ectopic or tertiary lymphoid organs (TLOs) to decrease local inflammation. TLOs are highly similar to SLOs and exhibit T and B cell compartmentalization, APCs such as DCs and follicular DCs, stromal cells, and a highly organized vascular system of high endothelial venules. In some embodiments, a neuromodulating agent decreases or prevents the development of high endothelial venules (HEVs) within tertiary lymphoid organs to decrease local inflammation. HEVs can be detected using the monoclonal antibody MECA-79.

In some embodiments, a neuromodulating agent modulates dendritic cell maturation (e.g., activation). Dendritic cell maturation can be increased to promote their migration from peripheral tissues into secondary lymphoid organs to improve T cell activation in the draining lymph node (e.g., to increase vaccine efficacy or to increase priming of an anti-tumor immune response). Dendritic cell maturation can be decreased to decrease their migration from peripheral tissues into secondary lymphoid organs to inhibit T cell activation in the draining lymph node.

The effect of a neuromodulating agent on immune cell recruitment or migration to a tumor can be assessed by evaluation of cellular markers on immune cells obtained from a human subject or animal model. A blood sample or tumor biopsy can be collected from a human subject or animal model and T cells, B cells, dendritic cells, or macrophages can be evaluated for marker CCR7. Immune cell recruitment to a tumor can also be assessed by taking a tumor biopsy before and after administration of a neuromodulating agent to a human subject or animal model and quantifying the number of immune cells in the tumor. Immune cells can be identified based on the markers described above and others listed in Table 9. A bulk gene expression signature can also be deconvolved into signatures indicative of specific immune cell types using published algorithms, such as the CIBERSORT algorithm described in Gentles et al, Nature Medicine 21:938 2015. Mouse models of cancer that express fluorescent reporters in immune cells can also be used for live imaging-based approaches to evaluate the effect of a neuromodulating agent on immune cell migration or recruitment to a tumor. Immune cell recruitment or migration to a tumor can also be assessed by adding a neuromodulating agent to immune cells in vitro (e.g., immune cells obtained from a subject, animal model, repository, or commercial source) and measuring CCR7 to evaluate immune cell migration or recruitment. The effect of a neuromodulating agent can be determined by comparing results from before and after neuromodulating agent administration.

In some embodiments, a neuromodulating agent increases homing or decreases egress of naïve T cells into or out of secondary lymphoid organs prior to inducing immunogenic tumor cell death to generate a better anti-tumor response (e.g., prior to radio- or chemotherapy). In some embodiments, a neuromodulating agent increases homing or decreases egress of immune cells into or out of the tumor microenvironment to turn a “cold tumor” into a “hot tumor” prior to immunotherapy. In some embodiments, a neuromodulating agent increases homing or decreases egress of effector immune cell subsets into or out of the tumor microenvironment to promote anti-tumor immunity. In some embodiments, a neuromodulating agent decreases homing or increases egress of immunosuppressive immune subsets into or out of the tumor microenvironment to promote anti-tumor immunity. In some embodiments, a neuromodulating agent induces or increases the development of high endothelial venules (HEVs) within the tumor microenvironment to increase TIL recruitment. HEVs can be detected using the monoclonal antibody MECA-79. In some embodiments, the neuromodulating agent induces or increases the development of ectopic or tertiary lymphoid organs (TLOs) within the tumor microenvironment to increase TIL recruitment. TLOs can be recognized by their similarity to SLOs, as they exhibit T and B cell compartmentalization, APCs such as DCs and follicular DCs, stromal cells, and a highly organized vascular system of HEVs.

The effect of a neuromodulating agent on NK cell lytic function can be assessed by evaluation of cellular markers on NK cells obtained from a human subject or animal model. A blood sample or tumor biopsy can be collected from a human subject or animal model and NK cells can be evaluated for one or more (e.g., 1, 2, 3 or more) of the markers: CD95L, CSD154, and CD253. NK cell lytic function can also be assessed using the same methods in an in vivo animal model. This assay can also be performed by adding a neuromodulating agent to NK cells in vitro (e.g., NK cells obtained from a subject, animal model, repository, or commercial source) and measuring the aforementioned markers to evaluate NK cell activation. These markers can be assessed using flow cytometry, immunohistochemistry, in situ hybridization, and other assays that allow for measurement of cell surface markers. The effect of a neuromodulating agent can be determined by comparing results from before and after neuromodulating agent administration.

Table 9 lists additional markers and relevant assays that may be used to assess the level, function and/or activity of immune cells in the methods described herein.

TABLE 9
ASSESSMENT OF IMMUNE CELL PHENOTYPES

| | ASSOCIATED | | |
|---|---|---|---|
| IMMUNE CELL | CYTOKINES | MARKER | ASSAYS |
| Th1 helper | IFN-γ | CD4 | ELISPOT |
| | IL-2 | CD94 | In situ hybridization |
| | IL-12 | CD119 | Immunohistochemistry |
| | IL-18 | (IFNγ R1) | Limiting dilution Analysis |
| | IL-27 | CD183 | Single-cell PCR |
| | TNFα | (CXCR3) | In vivo capture assay |
| | TNFβ/LTα | CD186 | ELISA |
| | | (CXCR6) | Flow cytometry |
| | | CD191 | |
| | | (CCR1) | |
| | | CD195 | |
| | | (CCR5) | |
| | | CD212 (IL- | |
| | | 12Rβ1&2) | |
| | | CD254 | |
| | | (RANKL) | |
| | | CD278 | |
| | | (ICOS) | |
| | | IL-18R | |
| | | MRP1 | |
| | | NOTCH3 | |
| | | TCR | |
| | | TIM3 | |
| Th2 helper | IL-4 | CD4 | ELISPOT |
| | IL-2 | CD30 | In situ hybridization |
| | IL-6 | CD119 | Immunohistochemistry |
| | IL-33 | (IFNγ R1) | Limiting dilution |
| | IL-17E (IL-25) | CD184 | Analysis |
| | IL-31 | (CXCR4) | Single-cell PCR |
| | IL-3 | CD185 | In vivo capture |
| | IL-10 | (CXCR5) | assay |
| | IL-13 | CD193 | ELISA |
| | | (CCR3) | Flow cytometry |
| | | CD194 | |
| | | (CCR4) | |
| | | CD197 | |
| | | (CCR7) | |
| | | CD278 | |
| | | (ICOS) | |
| | | CD294 | |
| | | (CRTh2) | |
| | | CDw198 | |
| | | (CCR8) | |
| | | IL-17RB | |
| | | IL-33Rα | |
| | | (ST2) | |
| | | NOTCH1 | |
| | | NOTCH2 | |
| | | TCR | |
| | | TIM1 | |
| Th17 helper | TGFβ1 | CD4 | ELISPOT |
| | IL-1β | CD27 | In situ hybridization |
| | IL-6 | CD62L | Immunohistochemistry |
| | IL-21 | CD127 (IL- | Limiting dilution |
| | IL-23 | 7R) | Analysis |
| | IL-17A | CD161 | Single-cell PCR |
| | IL-17F | CD184 | In vivo capture |
| | IL-22 | (CXCR4) | assay |
| | IL-26 | CD194 | ELISA |
| | GM-CSF | (CCR4) | Flow cytometry |
| | MIP-3α | CD196 | |
| | TNFα | (CCR6) | |
| | | CD197 | |
| | | (CCR7) | |
| | | CD212b1 | |
| | | (IL-12Rβ1) | |
| | | CD213a1 | |
| | | (IL-13Rα1) | |
| | | CD278 | |
| | | (ICOS) | |
| | | IL-1R1 | |
| | | IL-21R | |
| | | IL-23R | |
| Treg | TGFβ1 | CD4 | ELISPOT |
| | IL-2 | CD25 | In situ hybridization |
| | IL-10 | CD39 | Immunohistochemistry |
| | IL-35 | CD73 | Limiting dilution |
| | | CD45RO | Analysis |
| | | CD121a (IL- | Single-cell PCR |
| | | 1R1) | In vivo capture |
| | | CD121b (IL- | assay |
| | | 1R2) | ELISA |
| | | CD127low | Flow cytometry |
| | | CD134 | |
| | | (OX40) | |
| | | CD137 (4- | |
| | | 1BB) | |
| | | CD152 | |
| | | (CTLA-4) | |
| | | CD357 | |
| | | (GITR/AITR) | |
| | | Foxp3 | |
| | | FR4 (m) | |
| | | GARP | |
| | | (activated) | |
| | | Helios | |
| | | LAP/TGFβ | |
| | | (activated) | |
| | | TIGIT | |
| Dendritic cell | GM-CSF | CD1a | ELISPOT |
| | IFNγ | CD8 | In situ hybridization |
| | IL-4 | CD11c | Immunohistochemistry |
| | GM-CSF | CD80 | Limiting dilution |
| | IFNα | CD83 | Analysis |
| | IL-1α | CD85 (ILT) family | Single-cell PCR |
| | IL-1β | CD86 | In vivo capture |
| | IL-6 | CD141 (h) | assay |
| | IL-8 | CD169 | ELISA |
| | IL-10 | CD172 | Flow cytometry |
| | IL-12 | CD184 (CXCR4) | |
| | IL-15 | CD197 (CCR7) | |
| | IL-18 | CD205 | |
| | IL-23 | CD206 | |
| | IL-27 | CD207 | |
| | IP-10 | CD209 | |
| | M-CSF | CD215 (IL-15R) | |
| | RANTES (CCL5) | CD282 (TLR2) | |
| | TGFβ | CD284 (TLR4) | |
| | TNFα | CD286 (TLR6) | |
| | | Clec Family | |
| Macrophages/Monocytes | FLT3 Ligand | CD11b | ELISPOT |
| | GM-CSF | CD14 (mono) | In situ hybridization |
| | M-CSF | CD16 | Immunohistochemistry |
| | CXCL9 | CD32 | Limiting dilution |
| | CXCL10 | CD68 | Analysis |
| | CXCL11 | CD85a (ILT5) | Single-cell PCR |
| | G-CSF | CD163 | In vivo capture |
| | GM-CSF | CD169 | assay |
| | IFNβ | CD195 (CCR5) | ELISA |
| | IL-1α | CD204 | Flow cytometry |
| | IL-1β | CD206 | |
| | IL-6 | CD282 (TLR2) | |
| | IL-8 | CD284 (TLR4) | |
| | IL-10 | CD286 (TLR6) | |
| | IL-12p40 & p70 | CD354 (Trem-1) | |
| | IL-18 | Clec Family | |
| | IL-23 | F4/80 (m) | |
| | IL-27 | HLA-DR | |
| | M-CSF | | |
| | MIP-2α (CXCL2) | | |
| | RANTES (CCL5) | | |
| | TNFα | | |
| Natural Killer Cell | IL-2 | CD16 | ELISPOT |
| | IL-12 | CD25 | In situ hybridization |
| | IL-15/IL-15R | CD49b | Immunohistochemistry |
| | IL-18 | CD56 (h) | Limiting dilution |
| | Granzyme B | CD94 | Analysis |
| | IL-17A | CD158 family (KIR) | Single-cell PCR |
| | IL-22 | (h) | In vivo capture |
| | MIP-1α (CCL3) | CD181 (CXCR1) | assay |
| | MIP-1β (CCL4) | CD183 (CXCR3) | ELISA |
| | Perforin | CD184 (CXCR4) | Flow cytometry |
| | RANTES (CCL5) | CD186 (CXCR6) | |
| | TNFα | CD192 (activated) | |
| | | CD195 (CCR5) | |
| | | CD197 (CCR7) | |
| | | CD212 (IL-12R) | |
| | | CD244 | |
| | | CD314 (NKG2D) | |
| | | CX3CR1 | |
| | | Eomes | |
| | | KLRG1 | |
| | | Ly49 family (m) | |
| | | NK1.1 | |
| | | NKG2A | |
| | | NKp30 | |
| | | NKp42 | |
| | | NKp44 (h) | |
| | | NKp46 | |
| | | T-bet | |
| Activated B | Antibodies | CD19 | Flow cytometry |
| cell/Plasma cells | IgM | CD25 | |
| | IgG | CD30 | |
| | IgD | IgM | |
| | IgE | CD19 | |
| | IgA | IgG | |
| | | CD27 | |
| | | CD38 | |
| | | CD78 | |
| | | CD138 | |
| | | CD319 | |

TABLE 10
EXAMPLES OF HUMAN CHEMOKINES

| | | | | | |
|---|---|---|---|---|---|
| | | Alternate | | | |
| Systematic | Human | human | | Human receptor(s) and | Known |
| name | gene | Names | Expression | their expression | functions |

C family

| | | | | | |
|---|---|---|---|---|---|
| XCL1 | XCL1 | Lymphotactin, | Activated CD8+ T | XCR1: cross-presenting | Migration and |
| | | SCM-1 alpha, | cells and other | drendritic cells | activation of |
| | | ATAC | MHCI restricted T | | lymphocytes, |
| | | | cells | | NK cells |
| XCL2 | XCL2 | SCM-1 beta | Expressed in | XCR1: cross-presenting | Migration and |
| | | | activated T cells | drendritic cells | activation of |
| | | | | | lymphocytes, |
| | | | | | NK cells |

Cx3c family

| | | | | | |
|---|---|---|---|---|---|
| CX3CL1 | CX3CL1 | Fractalkine, | Brain, heart, lung, | CX3CR1: lymphocytes, | Migration and |
| | | Neurotactin, | kidney, skeletal | monocytes | adhesion of |
| | | ABCD-3 | muscle and testis. | | lymphocytes |
| | | | Up-regulated in | | and monocytes |
| | | | endothelial cells and | | |
| | | | microglia by | | |
| | | | inflammation | | |

Cc family

| | | | | | |
|---|---|---|---|---|---|
| CCL1 | CCL1 | I-309 | Activated T cells | CCR8: natural killer | Migration of |
| | | | | cells, monocytes and | monocytes, NK |
| | | | | lymphocytes | cells, immature |
| | | | | DARC: erytrocytes, | B cells and dcs |
| | | | | endothelial and epithelial | |
| | | | | cells | |
| CCL2 | CCL2 | MCP-1, | Monocytes, | CCR2: monocytes | Migration of |
| | | MCAF, HC11 | macrophages and | CCR4: lymphocytes | monocytes and |
| | | | dendritic cells, | CCR11: unkown | basophils |
| | | | activated NK cells | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL3 | CCL3 | MIP-1 alpha, | T cells, B cells, and | CCR1: lymphocytes, | Adhesion of |
| | | LD78 alpha, | monocytes after | monocytes, airway | lymphocytes |
| | | GOS19, | antigen or mitogen | smooth muscle cells | |
| | | Pat464 | stimulation | CCR4: lymphocytes | |
| | | | | CCR5: T cells, | |
| | | | | macrophages, dendritic | |
| | | | | cells, eosinophils and | |
| | | | | microglia | |
| | | | | D6: lymphocytes, | |
| | | | | lymphatic endothelial | |
| | | | | cells, macrophages | |
| CCL3L1 | CCL3L1 | LD78 beta | Unknown | CCR1: lymphocytes, | Migration of |
| | | | | monocytes, airway | lymphocytes |
| | | | | smooth muscle cells | and monocytes |
| | | | | CCR3: eosinophils, | |
| | | | | basophils, Th2 cells, | |
| | | | | CD34+ hematopoetic | |
| | | | | progenitors, | |
| | | | | keratinocytes, mast cells | |
| | | | | CCR5: T cells, | |
| | | | | macrophages, dendritic | |
| | | | | cells, eosinophils and | |
| | | | | microglia | |
| | | | | D6: lymphocytes, | |
| | | | | lymphatic endothelial | |
| | | | | cells, macrophages | |
| CCL3L3 | CCL3L3 | LD78 beta | Unknown | CCR1: lymphocytes, | Migration of |
| | | | | monocytes, airway | lymphocytes |
| | | | | smooth muscle cells | and monocytes |
| | | | | CCR3: eosinophils, | |
| | | | | basophils, Th2 cells, | |
| | | | | CD34+ hematopoetic | |
| | | | | progenitors, | |
| | | | | keratinocytes, mast cells | |
| | | | | CCR5: T cells, | |
| | | | | macrophages, dendritic | |
| | | | | cells, eosinophils and | |
| | | | | microglia | |
| CCL4 | CCL4 | MIP-1 beta, | Macrophages, | CCR1: lymphocytes, | Migration and |
| | | AT744.1, | dendritic cells | monocytes, airway | adhesion of |
| | | ACT-2, G-26, | | smooth muscle cells | lymphocytes, |
| | | HC21, H400, | | CCR5: T cells, | regulatory T |
| | | MAD-5, LAG-1 | | macrophages, dendritic | cells, NK cells, |
| | | | | cells, eosinophils and | monocyrtes |
| | | | | microglia | |
| | | | | CCR8: natural killer | |
| | | | | cells, monocytes and | |
| | | | | lymphocytes | |
| | | | | D6: lymphocytes, | |
| | | | | lymphatic endothelial | |
| | | | | cells, macrophages | |
| CCL4L1 | CCL4L1 | AT744.2 | Macrophages, | CCR1: lymphocytes, | CCR1 and |
| | | | dendritic cells | monocytes, airway | CCR5 |
| | | | | smooth muscle cells | expressing |
| | | | | CCR5: T cells, | cells |
| | | | | macrophages, dendritic | |
| | | | | cells, eosinophils and | |
| | | | | microglia | |
| CCL4L2 | CCL4L2 | | Macrophages, | CCR1: lymphocytes, | CCR1 and |
| | | | dendritic cells | monocytes, airway | CCR5 |
| | | | | smooth muscle cells | expressing |
| | | | | CCR5: T cells, | cells |
| | | | | macrophages, dendritic | |
| | | | | cells, eosinophils and | |
| | | | | microglia | |
| CCL5 | CCL5 | RANTES | T cells, | CCR1: lymphocytes, | Migration of |
| | | | macrophages, | monocytes, airway | monocytes, |
| | | | platelets, synovial | smooth muscle cells | memory T |
| | | | fibroblasts, tubular | CCR3: eosinophils, | helper cells and |
| | | | epithelium, certain | basophils, Th2 cells, | eosinophils, |
| | | | types of tumor cells | CD34+ hematopoetic | causes the |
| | | | | progenitors, | release of |
| | | | | keratinocytes, mast cells | histamine from |
| | | | | CCR4: lymphocytes | basophils and |
| | | | | CCR5: T cells, | activates |
| | | | | macrophages, dendritic | eosinophils |
| | | | | cells, eosinophils and | |
| | | | | microglia | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL7 | CCL7 | MCP-3 | Macrophages, | CCR1: lymphocytes, | Migration of |
| | | | certain types of | monocytes, airway | monocytes, |
| | | | tumor cells | smooth muscle cells | activation of |
| | | | | CCR2: monocytes | macrophages |
| | | | | CCR3: eosinophils, | |
| | | | | basophils, Th2 cells, | |
| | | | | CD34+ hematopoetic | |
| | | | | progenitors, | |
| | | | | keratinocytes, mast cells | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL8 | CCL8 | MCP-2, HC14 | Fibroblasts, | CCR1: lymphocytes, | Migration of |
| | | | endothelial cells | monocytes, airway | monocytes, |
| | | | | smooth muscle cells | lymphocytes, |
| | | | | CCR2: monocytes | basophils and |
| | | | | CCR3: eosinophils, | eosinophils |
| | | | | basophils, Th2 cells, | |
| | | | | CD34+ hematopoetic | |
| | | | | progenitors, | |
| | | | | keratinocytes, mast cells | |
| | | | | CCR5: T cells, | |
| | | | | macrophages, dendritic | |
| | | | | cells, eosinophils and | |
| | | | | microglia | |
| | | | | CCR11: unkown | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL11 | CCL11 | Eotaxin | Lung epithelial | CCR3: eosinophils, | Migration and |
| | | | cells, pleural | basophils, Th2 cells, | activation of |
| | | | mesothelial cells, | CD34+ hematopoetic | inflammatory |
| | | | bronchial airway | progenitors, | leukocytes, |
| | | | epithelial cells, | keratinocytes, mast cells | particularly |
| | | | smooth muscle cells | CCR5: T cells, | eosinophils |
| | | | | macrophages, dendritic | |
| | | | | cells, eosinophils and | |
| | | | | microglia | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL12 | | | Stromal cells in lung | CCR2: monocytes | Migration and |
| | | | and secondary | | activation of |
| | | | lymphoid organs | | monocytes |
| CCL13 | CCL13 | MCP-4, CK | Synovial fibroblasts, | CCR1: lymphocytes, | Migration of |
| | | beta 10, | chondrocytes | monocytes, airway | eosinophils, |
| | | NCC-1 | | smooth muscle cells | monocytes and |
| | | | | CCR2: monocytes | T lymphocytes |
| | | | | CCR3: eosinophils, | |
| | | | | basophils, Th2 cells, | |
| | | | | CD34+ hematopoetic | |
| | | | | progenitors, | |
| | | | | keratinocytes, mast cells | |
| | | | | CCR5: T cells, | |
| | | | | macrophages, dendritic | |
| | | | | cells, eosinophils and | |
| | | | | microglia | |
| | | | | CCR11: unkown | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL14 | CCL14 | HCC-1, | Spleen, bone | CCR1: lymphocytes, | Activation of |
| | | MCIF, CK | marrow, liver, | monocytes, airway | monocytes |
| | | beta 1, NCC-2 | muscle and gut | smooth muscle cells | |
| | | | | CCR3: eosinophils, | |
| | | | | basophils, Th2 cells, | |
| | | | | CD34+ hematopoetic | |
| | | | | progenitors, | |
| | | | | keratinocytes, mast cells | |
| | | | | CCR5: T cells, | |
| | | | | macrophages, dendritic | |
| | | | | cells, eosinophils and | |
| | | | | microglia | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL15 | CCL15 | MIP-1 delta, | Airway smooth | CCR1: lymphocytes, | Migration of |
| | | LKN-1, HCC- | muscle cells, lung | monocytes, airway | monocytes and |
| | | 2, MIP-5, | leukocytes, alveolar | smooth muscle cells | eosinophils, |
| | | NCC-3 | macrophages, | CCR3: eosinophils, | proliferation of |
| | | | basophils | basophils, Th2 cells, | CD34 myeloid |
| | | | | CD34+ hematopoetic | progenitor cells |
| | | | | progenitors, | |
| | | | | keratinocytes, mast cells | |
| CCL16 | CCL16 | HCC-4, LEC, | Liver, thymus, and | CCR1: lymphocytes, | Migration of |
| | | ILINCK, | spleen | monocytes, airway | lymphocytes |
| | | NCC-4, LMC, | | smooth muscle cells | and monocytes |
| | | CK beta 12 | | CCR2: monocytes | |
| | | | | CCR5: T cells, | |
| | | | | macrophages, dendritic | |
| | | | | cells, eosinophils and | |
| | | | | microglia | |
| | | | | CCR8: natural killer | |
| | | | | cells, monocytes and | |
| | | | | lymphocytes | |
| | | | | DARC: erytrocytes, | |
| | | | | endothelial and epithelial | |
| | | | | cells | |
| | | | | H4: bone marrow, | |
| | | | | eosinophils, T-cells, | |
| | | | | dendritic cells, | |
| | | | | monocytes, mast cells, | |
| | | | | neutrophil | |
| CCL17 | CCL17 | TARC, | Constitutively | CCR4: lymphocytes | Migration and |
| | | ABCD-2 | expressed in | CCR8: natural killer | activation of T |
| | | | thymus, dendritic | cells, monocytes and | cells |
| | | | cells, keratinocytes | lymphocytes | |
| | | | | D6: lymphocytes, | |
| | | | | lymphatic endothelial | |
| | | | | cells, macrophages | |
| | | | | DARC: erytrocytes, | |
| | | | | endothelial and epithelial | |
| | | | | cells | |
| CCL18 | CCL18 | PARC, DC- | Dendritic cells, | CCR8: natural killer | Migration of |
| | | CK1, AMAC- | monocytes, and | cells, monocytes and | naive and |
| | | 1, CK beta 7, | macrophages | lymphocytes | regulatory |
| | | MIP-4 | | PITPNM3: breast cancer | lymphocytes, |
| | | | | cells | dendritic cells |
| | | | | DARC: erytrocytes, | |
| | | | | endothelial and epithelial | |
| | | | | cells | |
| CCL19 | CCL19 | MIP-3 beta, | Fibroblastic reticular | CCR7: lymphocytes | Migration of |
| | | ELC, Exodus- | cells, dendritic cells | (mainly naive and | naive and |
| | | 3, CK beta 11 | | memory), mature | memory |
| | | | | dendritic cells | lymphocytes |
| | | | | CCR11: unkown | and mature |
| | | | | CCRL2: neutrophils, | dendritic cells |
| | | | | monocytes | |
| CCL20 | CCL20 | MIP-3 alpha, | Epidermis | CCR6: immature | Migration of |
| | | LARC, | (keratinocytes), | dendritic cells and | lymphocytes, |
| | | Exodus-1, | lymphocytes | memory T cells | dcs and |
| | | ST38, CK | | | neutrophils |
| | | beta 4 | | | |
| CCL21 | CCL21 | 6Ckine, | Stromal cells, | CCR7: lymphocytes | Migration of |
| | | Exodus-2, | lymphatic | (mainly naive and | lymphocytes |
| | | SLC, TCA-4, | endothelial cells, | memory), mature | homing to |
| | | CK beta 9 | fibroblastic reticular | dendritic cells | secondary |
| | | | cells, dendritic cells | CCR11: unkown | lymphoid |
| | | | | | organs, |
| | | | | | induces |
| | | | | | integrin- |
| | | | | | mediated |
| | | | | | lymphocyte |
| | | | | | adhesion |
| CCL22 | CCL22 | MDC | Macrophages | CCR4: lymphocytes | Migration of NK |
| | | | | D6: lymphocytes, | cells, |
| | | | | lymphatic endothelial | chronically |
| | | | | cells, macrophages | activated T |
| | | | | | cells, |
| | | | | | monocytes and |
| | | | | | dcs |
| CCL23 | CCL23 | MPIF-1, CK | Monocytes | CCR1: lymphocytes, | Migration of |
| | | beta 8, CK | | monocytes | monocytes, |
| | | beta 8-1, | | FPRL-1: monocytes, | resting T cells |
| | | MIP-3 | | mast cells | and neutrophils |
| CCL24 | CCL24 | Eotaxin-2, | Lung tissue | CCR3: eosinophils, | Migration of |
| | | MPIF-2, CK | | basophils, Th2 cells, | basophils |
| | | beta 6 | | CD34+ hematopoetic | |
| | | | | progenitors, | |
| | | | | keratinocytes, mast cells | |
| CCL25 | CCL25 | TECK, CK | Thymic dendritic | CCR9: T lymphocytes of | Migration of |
| | | beta 15 | cells and mucosal | small intestine | dendritic cells, |
| | | | epithelial cells | | thymocytes and |
| | | | | | activated |
| | | | | | macrophages |
| CCL26 | CCL26 | Eotaxin-3, | Heart, lung and | CCR3: eosinophils, | Migration of |
| | | MIP-4 alpha, | ovary and in | basophils, Th2 cells, | eosinophils and |
| | | IMAC, TSC-1 | endothelial cells | CD34+ hematopoetic | basophils |
| | | | stimulated with IL4 | progenitors, | |
| | | | | keratinocytes, mast cells | |
| | | | | CX3CR1: lymphocytes, | |
| | | | | monocytes | |
| CCL27 | CCL27 | CTACK, ILC, | Keratinocytes | CCR10: melanocytes, | Migration of |
| | | PESKY, | | plasma cells and skin- | memory T cells |
| | | ESKINE | | homing T cells | |
| CCL28 | CCL28 | MEC | Columnar epithelial | CCR3: eosinophils, | Migration of |
| | | | cells in the gut, lung, | basophils, Th2 T cells, | lymphocytes |
| | | | breast and the | CD34+ hematopoetic | and eosinophils |
| | | | salivary glands | progenitors, | |
| | | | | keratinocytes, mast cells | |
| | | | | CCR10: melanocytes, | |
| | | | | plasma cells and skin- | |
| | | | | homing T cells | |

Cxc family

| | | | | | |
|---|---|---|---|---|---|
| CXCL1 | CXCL1 | GRO alpha, | Mammary, | CXCR2 (IL8RB): | Migration of |
| | | MGSA, | fibroblasts, | neutrophils | neutrophils |
| | | GRO1, NAP-3 | mammary epithelial | DARC: erytrocytes, | |
| | | | cells, endothelial | endothelial and epithelial | |
| | | | cells, activated, | cells | |
| | | | monocytes, | | |
| | | | macrophages and | | |
| | | | neutrophils | | |
| CXCL2 | CXCL2 | GRO beta, | Monocytes, | CXCR2 (IL8RB): | Migration and |
| | | MIP-2 alpha, | macrophages | neutrophils | activation of |
| | | GRO2 | | DARC: erytrocytes, | neutrophils, |
| | | | | endothelial and epithelial | basophils, |
| | | | | cells | hematopoietic |
| | | | | | stem cells |
| CXCL3 | CXCL3 | GRO gamma, | Smooth muscle | CXCR2 (IL8RB): | Migration and |
| | | MIP-2 beta, | cells, epithelial cells | neutrophils | activation of |
| | | GRO3 | | DARC: erytrocytes, | neutrophils |
| | | | | endothelial and epithelial | |
| | | | | cells | |
| CXCL4 | PF4 | PF4 | Activated platelets, | CXCR3 (CD183b): T | Migration of |
| | | | megakaryocytes, | cells, NK cells | neutrophils and |
| | | | leukocytes, | CXCR3-B: T cells, NK | fibroblasts, |
| | | | endothelial cells | cells | inhibiting |
| | | | | DARC: erytrocytes, | endothelial cell |
| | | | | endothelial and epithelial | proliferation |
| | | | | cells | and chemotaxis |
| CXCL4L1 | PF4V1 | PF4V1 | Smooth muscle | CXCR3 (CD183b): T | Inhibiting |
| | | | cells, T cells, and | cells, NK cells | endothelial cell |
| | | | platelets | CXCR3-B: T cells, NK | proliferation |
| | | | | cells | and chemotaxis |
| CXCL5 | CXCL5 | ENA-78 | Fibroblasts, | CXCR2 (IL8RB): | Migration and |
| | | | epithelial cells, | neutrophils | activation of |
| | | | eosinophils | DARC: erytrocytes, | neutrophils |
| | | | | endothelial and epithelial | |
| | | | | cells | |
| CXCL6 | CXCL6 | GCP-2 | Fibroblasts, | CXCR1 (IL8RA): | Migration of |
| | | | epithelial cells | neutrophils | neutrophils |
| | | | | CXCR2 (IL8RB): | |
| | | | | neutrophils | |
| | | | | DARC: erytrocytes, | |
| | | | | endothelial and epithelial | |
| | | | | cells | |
| CXCL7 | PPBP | NAP-2, | Activated platelets | CXCR1 (IL8RA): | Migration of |
| | | CTAPIII, | | neutrophils | neutrophils |
| | | beta-TG | | CXCR2 (IL8RB): | |
| | | | | neutrophils | |
| CXCL8 | IL8 | IL-8, NAP-1, | Macrophages, | CXCR1 (IL8RA): | Migration of |
| | | MDNCF, | epithelial cells, | neutrophils | neutrophils, |
| | | GCP-1 | airway smooth | CXCR2 (IL8RB): | basophils, and |
| | | | muscle cells, | neutrophils | T-cells, and |
| | | | endothelial cells | DARC: erytrocytes, | angiogenic |
| | | | | endothelial and epithelial | factor |
| | | | | cells | |
| CXCL9 | CXCL9 | MIG, CRG-10 | Monocytes, | CXCR3 (CD183b): T | Migration of |
| | | | macrophages and | cells, NK cells | Th1 |
| | | | endothelial cells | CXCR3-B: T cells, NK | lymphocytes, |
| | | | | cells | angiogenic |
| | | | | DARC: erytrocytes, | factor |
| | | | | endothelial and epithelial | |
| | | | | cells | |
| CXCL10 | CXCL10 | IP-10 | Neutrophils, | CXCR3 (CD183b): T | Migration of |
| | | | hepatocytes, | cells, NK cells | CD4+ T cells |
| | | | endothelial cells and | CXCR3-B: T cells, NK | |
| | | | keratinocytes | cells | |
| | | | | DARC: erytrocytes, | |
| | | | | endothelial and epithelial | |
| | | | | cells | |
| CXCL11 | CXCL11 | I-TAC, beta- | Peripheral blood | CXCR3 (CD183b): T | Migration of |
| | | R1, H174, IP-9 | leukocytes, | cells, NK cells | interleukin- |
| | | | pancreas and liver | CXCR7 (ACKR3): tumor | activated T |
| | | | astrocytes and at | cells and tumor- | cells but not |
| | | | moderate levels in | associated blood | unstimulated T |
| | | | thymus, spleen and | endothelium | cells, |
| | | | lung | DARC: erytrocytes, | neutrophils or |
| | | | | endothelial and epithelial | monocytes. |
| | | | | cells | |
| CXCL12 | CXCL12 | SDF-1, PBSF | Ubiquitously | CXCR4: brain, heart, | Migration of |
| | | | expressed in many | lymphocytes, HSCs, | lymphocytes |
| | | | tissues and cell | blood endothelial cells | and |
| | | | types | and umbilical cord | hepatopoietic |
| | | | | endothelial cell | stem cells, |
| | | | | CXCR7 (ACKR3): tumor | angiogenic |
| | | | | cells and tumor- | factor |
| | | | | associated blood | |
| | | | | endothelium | |
| CXCL13 | CXCL13 | BCA-1, BLC | Follicles of the | CXCR3 (CD183b): T | Migration of B |
| | | | spleen, lymph | cells, NK cells | cells |
| | | | nodes, and Peyer's | CXCR5: Burkitt's | |
| | | | patches | lymphoma, lymph node | |
| | | | | follicules, spleen | |
| | | | | DARC: erytrocytes, | |
| | | | | endothelial and epithelial | |
| | | | | cells | |
| CXCL14 | CXCL14 | BRAK, BMAC | Fibroblasts | unknown | Migration of |
| | | | | | monocytes, NK |
| | | | | | cells, dcs |
| CXCL16 | CXCL16 | SR-PSOX | Dcs | CXCR6: T cells | Migration of |
| | | | | | several subsets |
| | | | | | of T cells and |
| | | | | | NKT cells |
| CXCL17 | CXCL17 | DMC, VCC-1 | Lung and tumor | unknown | Migration of |
| | | | tissue | | dcs and |
| | | | | | monocytes |

TABLE 11
EXAMPLES OF HUMAN IMMUNE CELL TRAFFICKING MOLECULES

| | Trafficking | | |
|---|---|---|---|
| | molecule | | |
| Trafficking | expressing or | | Function in the extravasation |
| molecule | presenting cells | Leukocyte ligand | cascade |
| P-selectin | Blood endothelial cell | PSGL-1, L-selectin, | Tethering/Rolling during |
| | | CD44 | extravasation cascade |
| E-selectin | Blood endothelial cell | Glycoprotein, | Tethering/Rolling during |
| | | glycolipid, PSGL-1 | extravasation cascade |
| PNAd | Blood endothelial cell | L-selectin | Tethering/Rolling during |
| | | | extravasation cascade |
| MAdCAM | Blood endothelial cell | L-selectin, integrins | Tethering/Rolling, arrest during |
| | | | extravasation cascade |
| VCAM-1 | Blood endothelial cell | Integrins (e.g. VLA- | Tethering/Rolling, arrest during |
| | | 4) | extravasation cascade |
| Chemokines | Blood endothelial cell | GPCRs | Integrin activation, allowing binding of |
| | | | cell adhesion molecules and arrest |
| ICAM-1 | Blood endothelial cell | Integrins (e.g. LFA- | Arrest during extravasation cascade |
| | | 1, Mac-1) | |
| ICAM-2 | Blood endothelial cell | Integrins (e.g. LFA- | Arrest during extravasation cascade |
| | | 1, Mac-1) | |
| PECAM1 | Blood endothelial cell | Integrins (e.g. alpha | Transmigration |
| (CD31) | | v beta 3), PECAM1 | |
| JAM-A/-B/-C | Blood endothelial cell | Integrins (e.g. LFA- | Transmigration |
| | | 1, Mac-1, VLA-4) | |
| ESAM | Blood endothelial cell | unknown | Transmigration |
| CD99 | Blood endothelial cell | CD99 | Transmigration |
| CD99L2 | Blood endothelial cell | possibly CD99L | Transmigration |
| VE-cadherin | Blood endothelial cell | none | Transmigration |
| PVR | Blood endothelial cell | DNAM1 | Transmigration |
| S1P | Lymphatic | S1P receptor 1 | Entry into afferent and efferent |
| | endothelial cell | (S1P1) | lymphatics (in peripheral or SLOs |
| | | | respectively) |

The methods described herein can be used to treat cancer in a subject by administering to the subject an effective amount of a neuromodulating agent, e.g., a neuromodulating agent described herein. The method may include administering locally (e.g., intratumorally) to the subject a neuromodulating agent described herein in a dose (e.g., effective amount) and for a time sufficient to treat the cancer.

The methods described herein can also be used to potentiate or increase an immune response in a subject in need thereof, e.g., an anti-tumor immune response. For example, the subject has cancer, such as a cancer described herein. The methods described herein can also include a step of selecting a subject in need of potentiating an immune response, e.g., selecting a subject who has cancer or is at risk of developing cancer.

The neuromodulating agent may inhibit proliferation or disrupt the function of non-neural cells associated with the cancer, e.g., the method includes administering to the subject an effective amount of a neuromodulating agent for a time sufficient to inhibit proliferation or disrupt the function of non-neural cells associated with the cancer. Non-neural cells associated with the cancer include malignant cancer cells, malignant cancer cells in necrotic and hypoxic areas, Natural Killer cells, Natural Killer T cells, macrophages, tumor associated macrophages, TH1 helper cells, TH2 helper cells, CD8 cytotoxic T cells, TH17 cells, T regulatory cells, tumor associated neutrophils, terminally differentiated myeloid dendritic cells, myeloid derived suppressor cells, T lymphocytes, adipocytes, B lymphocytes, B10 cells, Breg cells, lymphatic endothelial cells, pericytes, endothelial cells, cancer associated fibroblasts, fibroblasts, dendritic cells, mesenchymal stem cells, red blood cells, or extracellular matrix. The proliferation of non-neural cells associated with the cancer may be decreased in the subject at least 1%, 2%, 5%, 10%, 15%, 20%, 25%, 30%, 35%, 40%, 50%, 60%, 70%, 80%, 90%, 95% or more, compared to before the administration. The proliferation of non-neural cells associated with the cancer can be decreased in the subject between 5-20%, between 5-50%, between 10-50%, between 20-80%, between 20-70%.

The neuromodulating agent can be administered in an amount sufficient to treat cancer. For example, the stroma associated with the tumor, e.g., fibroblasts, is disrupted such that an essential function, e.g., the production of matrix metalloproteases, is altered to inhibit tumor survival or promote tumor control.

The neuromodulating agent can have one or more of the following activities: (a) inhibits an immune checkpoint, (b) activates anti-tumor immune response, (c) activate tumor-specific T cells from draining lymph nodes, and/or (d) stimulates a neoantigen-specific immune response. The activity can be modulated as appropriate in the subject (e.g., a human subject or animal model) at least 1%, 2%, 5%, 10%, 15%, 20%, 25%, 30%, 35%, 40%, 50%, 60%, 70%, 80%, 90%, 95% or more, compared to before the administration. The activity can be modulated as appropriate in the subject between 5-20%, between 5-50%, between 10-50%, between 20-80%, between 20-70%.

The neuromodulating agent can treat cancer by increasing cancer cell death in a subject (e.g., a human subject or animal model) or in a cancer cell culture (e.g., a culture generated from a patient tumor sample, a cancer cell line, or a repository of patient samples). A neuromodulating agent can increase cancer cell death by at least 1%, 2%, 5%, 10%, 15%, 20%, 25%, 30%, 35%, 40%, 50%, 60%, 70%, 80%, 90%, 95% or more compared to before administration to a subject or cancer cell culture. A neuromodulating agent can increase cancer cell death in a subject or cancer cell culture between 5-20%, between 5-50%, between 10-50%, between 20-80%, between 20-70%.

The neuromodulating agent can also act to inhibit cancer cell growth, proliferation, metastasis, or invasion, e.g., the method includes administering to the subject (e.g., a human subject or animal model) or a cancer cell culture (e.g., a culture generated from a patient tumor sample, a cancer cell line, or a repository of patient samples) a neuromodulating agent in an amount (e.g., an effective amount) and for a time sufficient to inhibit cancer cell growth, proliferation, metastasis, or invasion. Cancer cell growth, proliferation, metastasis, or invasion can be decreased in the subject or cancer cell culture at least 1%, 2%, 5%, 10%, 15%, 20%, 25%, 30%, 35%, 40%, 50%, 60%, 70%, 80%, 90%, 95% or more, compared to before the administration Cancer cell growth, proliferation, metastasis, or invasion can be decreased in the subject or cancer cell culture between 5-20%, between 5-50%, between 10-50%, between 20-80%, between 20-70%.

The neuromodulating agent can inhibit cancer cell invasion or metastasis along a nerve, e.g., the method includes administering to the subject (e.g., a human subject or animal model) a neuromodulating agent in an amount (e.g., an effective amount) and for a time sufficient to inhibit cancer cell invasion or metastasis along a nerve. For example, the neuromodulating agent is an antibody against a ligand selected from: Galanin; Semaphorin-4F; Caveolin-1; a chemokine such as CCL2, CCR2, CXCL12, and CXCR4; GDNF; GFRa1; NGF; neurotrophin-3 or -4; substance P; Neuropeptide Y; Peptide YY; Vasoactive intestinal peptide (VIP); or NCAM1. In other examples, the neuromodulating agent can be a receptor antagonist against the receptor for a ligand selected from: Galanin; Semaphorin-4F; Caveolin-1; a chemokine such as CCL2, CCR2, CXCL12, and CXCR4; GDNF; GFRa1; NGF; neurotrophin-3 or -4; substance P; Neuropeptide Y; Peptide YY; Vasoactive intestinal peptide (VIP); or NCAM1. The neuromodulating can decrease cancer cell invasion or metastasis along a nerve in the subject at least 1%, 2%, 5%, 10%, 15%, 20%, 25%, 30%, 35%, 40%, 50%, 60%, 70%, 80%, 90%, 95% or more, compared to before the administration. The neuromodulating agent can decrease cancer cell invasion or metastasis along a nerve in the subject between 5-20%, between 5-50%, between 10-50%, between 20-80%, between 20-70%.

The neuromodulating agent can also reduce the number of nerve fibers in the affected tissue or reduce the activity of peripheral nerve fibers in the affected tissue. For example, the method includes administering to the subject (e.g., a human subject or animal model) a neuromodulating agent in an amount (e.g., an effective amount) and for a time sufficient to reduce the number of nerve fibers in the affected tissue or reduce the activity of peripheral nerve fibers in the affected tissue. The affected tissue can be a tumor, a tumor micro-environment, or the bone marrow niche. The number of nerve fibers in the affected tissue or the activity of peripheral nerve fibers in the affected tissue can be decreased in the subject at least 1%, 2%, 5%, 10%, 15%, 20%, 25%, 30%, 35%, 40%, 50%, 60%, 70%, 80%, 90%, 95% or more, compared to before the administration. The number of nerve fibers in the affected tissue or the activity of peripheral nerve fibers in the affected tissue can be decreased in the subject between 5-20%, between 5-50%, between 10-50%, between 20-80%, between 20-70%.

The neuromodulating agent can also increase the number of nerve fibers in the affected tissue or increase the activity of peripheral nerve fibers in the affected tissue. For example, the method includes administering to the subject (e.g., a human subject or animal model) a neuromodulating agent in an amount (e.g., an effective amount) and for a time sufficient to increase the number of nerve fibers in the affected tissue or increase the activity of peripheral nerve fibers in the affected tissue. The affected tissue can be a tumor, a tumor micro-environment, or the bone marrow niche. The number of nerve fibers in the affected tissue or the activity of peripheral nerve fibers in the affected tissue can be increased in the subject at least 1%, 2%, 5%, 10%, 15%, 20%, 25%, 30%, 35%, 40%, 50%, 60%, 70%, 80% or more, compared to before the administration. The number of nerve fibers in the affected tissue or the activity of peripheral nerve fibers in the affected tissue can be increased in the subject between 5-20%, between 5-50%, between 10-50%, between 20-80%, between 20-70%.

The nerve fibers that are modulated can be part of the peripheral nervous system, e.g., a somatic nerve, an autonomic nerve, a sensory nerve, a cranial nerve, an optic nerve, an olfactory nerve, a sympathetic nerve, a parasympathetic nerve, a chemoreceptor, a photoreceptor, a mechanoreceptor, a thermoreceptor, a nociceptor, an efferent nerve fiber, or an afferent nerve fiber.

IV. Cancer

In the methods described herein relating to cancer, the cancer or neoplasm may be any solid or liquid cancer and includes benign or malignant tumors, and hyperplasias, including gastrointestinal cancer (such as non-metastatic or metastatic colorectal cancer, pancreatic cancer, gastric cancer, esophageal cancer, hepatocellular cancer, cholangiocellular cancer, oral cancer, lip cancer); urogenital cancer (such as hormone sensitive or hormone refractory prostate cancer, renal cell cancer, bladder cancer, penile cancer); gynecological cancer (such as ovarian cancer, cervical cancer, endometrial cancer); lung cancer (such as small-cell lung cancer and non-small-cell lung cancer); head and neck cancer (e.g., head and neck squamous cell cancer); CNS cancer including malignant glioma, astrocytomas, retinoblastomas and brain metastases; malignant mesothelioma; non-metastatic or metastatic breast cancer (e.g., hormone refractory metastatic breast cancer); skin cancer (such as malignant melanoma, basal and squamous cell skin cancers, Merkel Cell Carcinoma, lymphoma of the skin, Kaposi Sarcoma); thyroid cancer; bone and soft tissue sarcoma; and hematologic neoplasias (such as multiple myeloma, acute myelogenous leukemia, chronic myelogenous leukemia, myelodysplastic syndrome, acute lymphoblastic leukemia, Hodgkin's lymphoma).

Additional examples of cancers that can be treated according to the methods described herein include breast cancer, lung cancer, stomach cancer, colon cancer, liver cancer, renal cancer, colorectal cancer, prostate cancer, pancreatic cancer, cervical cancer, anal cancer, vulvar cancer, penile cancer, vaginal cancer, testicular cancer, pelvic cancer, thyroid cancer, uterine cancer, rectal cancer, brain cancer, head and neck cancer, esophageal cancer, bronchus cancer, gallbladder cancer, ovarian cancer, bladder cancer, oral cancer, oropharyngeal cancer, larynx cancer, biliary tract cancer, skin cancer, a cancer of the central nervous system, a cancer of the respiratory system, and a cancer of the urinary system. Examples of breast cancers include, but are not limited to, triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, and phyllodes tumor.

Other examples of cancers that can be treated according to the methods described herein include leukemia (e.g., B-cell leukemia, T-cell leukemia, acute myeloid leukemia (AML), chronic myeloid leukemia (CML), acute lymphocytic (lymphoblastic) leukemia (ALL), chronic lymphocytic leukemia (CLL), and erythroleukemia), sarcoma (e.g., angiosarcoma, chondrosarcoma, Ewing's sarcoma, fibrosarcoma, gastrointestinal stromal tumor, leiomyosarcoma, liposarcoma, malignant peripheral nerve sheath tumor, malignant fibrous cytoma, osteosarcoma, pleomorphic sarcoma, rhabdomyosarcoma, synovial sarcoma, vascular sarcoma, Kaposi's sarcoma, dermatofibrosarcoma, epithelioid sarcoma, leyomyosarcoma, and neurofibrosarcoma), carcinoma (e.g., basal cell carcinoma, large cell carcinoma, small cell carcinoma, non-small cell lung carcinoma, renal carcinoma, hepatocarcinoma, gastric carcinoma, choriocarcinoma, adenocarcinoma, hepatocellular carcinoma, giant (or oat) cell carcinoma, squamous cell carcinoma, adenosquamous carcinoma, anaplastmic carcinoma, adrenocortical carcinoma, cholangiocarcinoma, Merkel cell carcinoma, ductal carcinoma in situ (DCIS), and invasive ductal carcinoma), blastoma (e.g., hepatoblastoma, medulloblastoma, nephroblastoma, neuroblastoma, pancreatoblastoma, pleuropulmonary blastoma, retinoblastoma, and glioblastoma multiforme), lymphoma (e.g., Hodgkin's lymphoma, non-Hodgkin's lymphoma, and Burkitt lymphoma), myeloma (e.g., multiple myeloma, plasmacytoma, localized myeloma, and extramedullary myeloma), melanoma (e.g., superficial spreading melanoma, nodular melanoma, lentigno maligna melanoma, acral lentiginous melanoma, and amelanotic melanoma), neuroma (e.g., ganglioneuroma, Pacinian neuroma, and acoustic neuroma), glioma (e.g., astrocytoma, oligoastrocytoma, ependymoma, brainstem glioma, optic nerve glioma, and oligoastrocytoma), pheochromocytoma, meningioma, malignant mesothelioma, and virally induced cancer.

In some embodiments, the cancer is a paraneoplastic cancer (e.g., a cancer that causes a paraneoplastic syndrome). Paraneoplastic syndromes are rare disorders that are triggered by an altered immune system response to a neoplasm, and are mediated by humoral factors such as hormones, cytokines, or auto-antibodies produced by the tumor. Symptoms of paraneoplastic syndrome may be endocrine, neuromuscular, or musculoskeletal, cardiovascular, cutaneous, hematologic, gastrointestinal, renal, or neurological. Paraneoplastic syndromes commonly present with lung, breast, and ovarian cancer and cancer of the lymphatic system (e.g., lymphoma). Paraneoplastic neurological disorders are disorders that affect the central or peripheral nervous system, and can include symptoms such as ataxia (difficulty with walking and balance), dizziness, nystagmus (rapid uncontrolled eye movements), difficulty swallowing, loss of muscle tone, loss of fine motor coordination, slurred speech memory loss, vision problems, sleep disturbances, dementia, seizures, or sensory loss in the limbs. Breast, ovarian, and lung cancers are most commonly associated with paraneoplastic neurological disorders. Other common types of paraneoplastic syndromes include paraneoplastic cerebellar degeneration, paraneoplastic pemphigus, paraneoplastic autonomic neuropathy, paraneoplastic encephalomyelitis, and cancer-associated autoimmune retinopathy.

Endocrine paraneoplastic syndromes include Cushing syndrome (caused by ectopic ACTH), which is most commonly caused by small cell lung cancer, pancreatic carcinoma, neural tumors, or thymoma; SIADH (caused by antidiuretic hormone), which is most commonly caused by small cell lung cancer and CNS malignancies; hypercalcemia (caused by PTHrp, TGFα, TNF, or IL-1), which is most commonly caused by lung cancer, breast carcinoma, renal and bladder carcinoma, multiple myeloma, adult T cell leukemia/lymphoma, ovarian carcinoma, and squamous cell carcinoma (e.g., lung, head, neck, or esophagus carcinoma); hyperglycemia (caused by insulin insulin-like substance, or “big” IGF-II), which is most commonly caused by fibrosarcoma, mesenchymal sarcomas, insulinoma, and hepatocellular carcinoma; carcinoid syndrome (caused by serotonin or bradykinin), which is most commonly caused by bronchial adenoma, pancreatic carcinoma, and gastric carcinoma; and hyperaldosteronism (caused by aldosterone), which is most commonly caused by adrenal adenoma/Conn's syndrome, non-Hodgkin's lymphoma, ovarian carcinoma, and pulmonary cancer.

Neurological paraneoplastic syndromes include Lambert-Eaton myasthenic syndrome (LEMS), which is most commonly caused by small cell lung cancer; paraneoplastic cerebellar degeneration, which is most commonly caused by lung cancer, ovarian cancer, breast carcinoma, and Hodgkin's lymphoma; encephalomyelitis; limbic encephalitis, which is most commonly caused by small cell lung carcinoma; myasthenia gravis, which is most commonly caused by thymoma; brainstem encephalitis; opsoclonus myoclonus ataxia (caused by autoimmune reaction against Nova-1), which is most commonly caused by breast carcinoma, ovarian carcinoma, small cell lung carcinoma, and neuroblastoma; anti-NMDA receptor encephalitis (caused by autoimmune reaction against NMDAR subunits), which is most commonly caused by teratoma; and polymyositis, which is most commonly caused by lung cancer, bladder cancer, and non-Hodgkin's lymphoma. Mucotaneous paraneoplastic syndromes include acanthosis nigricans, which is most commonly caused by gastric carcinoma, lung carcinoma, and uterine carcinoma; dermatomyositis, which is most commonly caused by bronchogenic carcinoma, breast carcinoma, ovarian cancer, pancreatic cancer, stomach cancer, colorectal cancer, and Non-Hodgkin's lymphoma; Leser-Trelat sign; necrolytic migratory erythema, which is most commonly caused by glucoganoma; Sweet's syndrome; florid cutaneous papillomatosis; pyoderma gangrenosum; and acquired generalized hypertrichosis.

Hematological syndromes include granulocytosis (caused by G-CSF); polycythemia (caused by erythropoietin), which is commonly caused by renal carcinoma, cerebellar hemangioma, and heptatocellular carcinoma; Trousseau sign (caused by mucins), which is commonly caused by pancreatic carcinoma and bronchogenic carcinoma; nonbacterial thrombotic endocarditis, which is caused by advanced cancers; and anemia, which is most commonly caused by thymic neoplasms. Other paraneoplastic syndromes include membranous glomerular nephritis; neoplastic fever; Staffer syndrome, which is caused by renal cell carcinoma; and tumor-induced osteomalacia (caused by FGF23), which is caused by hemangiopericytoma and phosphaturic mesenchymal tumor.

In some embodiments, a subject is identified as having cancer after presenting with symptoms of a paraneoplastic syndrome. A common symptom of paraneoplastic syndrome is fever. Auto-antibodies directed against nervous system proteins are also frequently observed in patients with paraneoplastic syndromes, including anti-Hu, anti-Yo, anti-Ri, anti-amphiphysin, anti-CV2, anti-Ma2, anti-recoverin, anti-transducin, anti-carbonic anhydrase II, anti-arrestin, anti-GCAP1, anti-GCAP2, anti-HSP27, anti-Rab6A, and anti-PNR. Other symptoms that can be used to identify a patient with paraneoplastic cancer include ataxia, dizziness, nystagmus, difficulty swallowing, loss of muscle tone, loss of fine motor coordination, slurred speech memory loss, vision loss, sleep disturbances, dementia, seizures, dysgeusia, cachexia, anemia, itching, or sensory loss in the limbs. In some embodiments, a patient presents with symptoms of paraneoplastic syndrome and is then identified as having cancer based on imaging tests (e.g., CT, MRI, or PET scans).

The cancer may be highly innervated, metastatic, non-metastatic cancer, or benign (e.g., a benign tumor). The cancer may be a primary tumor or a metastasized tumor.

Subjects who can be treated with the methods disclosed herein include subjects who have had one or more tumors resected, received chemotherapy or other pharmacological treatment for the cancer, received radiation therapy, and/or received other therapy for the cancer. Subjects who have not previously been treated for cancer can also be treated with the methods disclosed herein.

V. Combination Therapies for Cancer

Combination Therapies for Cancer

A neuromodulating agent described herein can be administered in combination with a second therapeutic agent for treatment of cancer. In some embodiments, the second therapeutic agent is selected based on tumor type, tumor tissue of origin, tumor stage, or mutations in non-neurome genes expressed by the tumor.

Checkpoint Inhibitors

One type of agent that can be administered in combination with a neuromodulating agent described herein is a checkpoint inhibitor. Checkpoint inhibitors can be broken down into at least 4 major categories: i) agents such as antibodies that block an inhibitory pathway directly on T cells or natural killer (NK) cells (e.g., PD-1 targeting antibodies such as nivolumab and pembrolizumab, antibodies targeting TIM-3, and antibodies targeting LAG-3, 2B4, CD160, A2aR, BTLA, CGEN-15049, or KIR), ii) agents such as antibodies that activate stimulatory pathways directly on T cells or NK cells (e.g., antibodies targeting OX40, GITR, or 4-1 BB), iii) agents such as antibodies that block a suppressive pathway on immune cells or rely on antibody-dependent cellular cytotoxicity to deplete suppressive populations of immune cells (e.g., CTLA-4 targeting antibodies such as ipilimumab, antibodies targeting VISTA, and antibodies targeting PD-L2, Gr1, or Ly6G), and iv) agents such as antibodies that block a suppressive pathway directly on cancer cells or that rely on antibody-dependent cellular cytotoxicity to enhance cytotoxicity to cancer cells (e.g., rituximab, antibodies targeting PD-L1, and antibodies targeting B7-H3, B7-H4, Gal-9, or MUC1). Such agents described herein can be designed and produced, e.g., by conventional methods known in the art (e.g., Templeton, Gene and Cell Therapy, 2015; Green and Sambrook, Molecular Cloning, 2012).

Chemotherapy

A second type of therapeutic agent that can be administered in combination with a neuromodulating agent described herein is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed Engl. 33:183 1994); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel; chloranbucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al., Proc ASCO 18:233a, 1999, and Douillard et al., Lancet 355:1041, 2000.

Biologic Cancer Agents

Another type of therapeutic agent that can be administered in combination with a neuromodulating agent described herein is a therapeutic agent that is a biologic such a cytokine (e.g., interferon or an interleukin (e.g., IL-2)) used in cancer treatment. In other embodiments the biologic is an anti-angiogenic agent, such as an anti-VEGF agent, e.g., bevacizumab. In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include Rituximab; Daclizumab; Basiliximab; Palivizumab; Infliximab; Trastuzumab; Gemtuzumab ozogamicin; Alemtuzumab; Ibritumomab tiuxetan; Adalimumab; Omalizumab; Tositumomab-I-131; Efalizumab; Cetuximab; Bevacizumab; Natalizumab; Tocilizumab; Panitumumab; Ranibizumab; Eculizumab;

Certolizumab pegol; Golimumab; Canakinumab; Ustekinumab; Ofatumumab; Denosumab; Motavizumab; Raxibacumab; Belimumab; Ipilimumab; Brentuximab Vedotin; Pertuzumab; Ado-trastuzumab emtansine; and Obinutuzumab. Also included are antibody-drug conjugates. Examples of biologic cancer agents that can be used in combination with neuromodulating agents described herein are shown in Table 12 below. These antibodies can be administered in combination with a neuromodulating agent to promote ADCC or ADCP.

TABLE 12
APPROVED CANCER ANTIBODIES

| Antibody | Company | Antigen | Indication |
|---|---|---|---|
| ado-trastuzumab | Genentech | HER2 | Metastatic breast cancer |
| emtansine | | | |
| alemtuzumab | Genzyme | CD52 | B-cell chronic lymphocytic leukemia |
| atezolizumab | Genentech | PD-L1 | Urothelial carcinoma |
| atezolizumab | Genentech | PD-L1 | Urothelial carcinoma |
| | | | Metastatic non-small cell lung cancer |
| avelumab | EMD Serono | PD-L1 | Metastatic Merkel cell carcinoma |
| bevacizumab | Genentech | VEGF | Metastatic colorectal cancer |
| blinatumomab | Amgen | CD19 | Precursor B-cell acute lymphoblastic leukemia |
| brentuximab | Seattle Genetics | CD30 | Hodgkin lymphoma |
| vedotin | | | Anaplastic large-cell lymphoma |
| cetuximab | ImClone Systems | EGFR | Metastatic colorectal carcinoma |
| daratumumab | Janssen Biotech | CD38 | Multiple myeloma |
| dinutuximab | United Therapeutics | GD2 | Pediatric high-risk neuroblastoma |
| durvalumab | AstraZeneca | PD-L1 | Urothelial carcinoma |
| elotuzumab | Bristol-Myers | SLAMF7 | Multiple myeloma |
| | Squibb | | |
| ibritumomab | Spectrum | CD20 | Relapsed or refractory low-grade, follicular, or |
| tiuxetan | Pharmaceuticals | | transformed B-cell non-Hodgkin's lymphoma |
| ipilimumab | Bristol-Myers | CTLA-4 | Metastatic melanoma |
| | Squibb | | |
| necitumumab | Eli Lilly | EGFR | Metastatic squamous non-small cell lung |
| | | | carcinoma |
| nivolumab | Bristol-Myers | PD-1 | Metastatic melanoma |
| | Squibb | | |
| nivolumab | Bristol-Myers | PD-1 | Metastatic squamous non-small cell lung |
| | Squibb | | carcinoma |
| obinutuzumab | Genentech | CD20 | Chronic lymphocytic leukemia |
| ofatumumab | Glaxo Grp | CD20 | Chronic lymphocytic leukemia |
| olaratumab | Eli Lilly | PDGFRA | Soft tissue sarcoma |
| panitumumab | Amgen | EGFR | Metastatic colorectal cancer |
| pembrolizumab | Merck | PD-1 | Metastatic melanoma |
| pertuzumab | Genentech | HER2 | Metastatic breast cancer |
| ramucirumab | Eli Lilly | VEGFR2 | Gastric cancer |
| rituximab | Genentech | CD20 | B-cell non-Hodgkin's lymphoma |
| trastuzumab | Genentech | HER2 | Metastatic breast cancer |

Non-Drug Therapies

Another type of agent that can be administered in combination with a neuromodulating agent is a therapeutic agent that is a non-drug treatment. For example, the second therapeutic agent is radiation therapy, cryotherapy, hyperthermia and/or surgical excision of tumor tissue.

In any of the combination therapy approaches described herein, the first and second therapeutic agent (e.g., a neuromodulating agent described herein and the additional therapeutic agent) are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

VI. Methods of Treatment

Administration

An effective amount of a neuromodulating agent described herein for treatment of cancer can be administered to a subject by standard methods. For example, the agent can be administered by any of a number of different routes including, e.g., intravenous, intradermal, subcutaneous, percutaneous injection, oral, transdermal (topical), or transmucosal. The neuromodulating agent can be administered orally or administered by injection, e.g., intramuscularly, or intravenously. The most suitable route for administration in any given case will depend on the particular agent administered, the patient, the particular disease or condition being treated, pharmaceutical formulation methods, administration methods (e.g., administration time and administration route), the patients age, body weight, sex, severity of the diseases being treated, the patient's diet, and the patient's excretion rate. The agent can be encapsulated or injected, e.g., in a viscous form, for delivery to a chosen site, e.g., a tumor or a lymph node. The agent can be provided in a matrix capable of delivering the agent to the chosen site. Matrices can provide slow release of the agent and provide proper presentation and appropriate environment for cellular infiltration. Matrices can be formed of materials presently in use for other implanted medical applications. The choice of matrix material is based on any one or more of: biocompatibility, biodegradability, mechanical properties, and cosmetic appearance and interface properties. One example is a collagen matrix.

The agent (e.g., peptide, neurotransmitter, small molecule, nucleic acid, protein such as an antibody) can be incorporated into pharmaceutical compositions suitable for administration to a subject, e.g., a human. Such compositions typically include the agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a neuromodulating agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable carriers. These can be prepared according to methods known to those skilled in the art.

Nucleic acid molecule agents described herein can be administered directly (e.g., therapeutic mRNAs) or inserted into vectors used as gene therapy vectors. Gene therapy vectors can be delivered to a subject by, for example, intravenous injection, local administration (see U.S. Pat. No. 5,328,470) or by stereotactic injection (see, e.g., Chen et al., PNAS 91:3054 1994). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of formulating pharmaceutical agents are known in the art, e.g., Niazi, Handbook of Pharmaceutical Manufacturing Formulations (Second Edition), CRC Press 2009, describes formulation development for liquid, sterile, compressed, semi-compressed and OTC forms. Transdermal and mucosal delivery, lymphatic system delivery, nanoparticles, controlled drug release systems, theranostics, protein and peptide drugs, and biologics delivery are described in Wang et al., Drug Delivery: Principles and Applications (Second Edition), Wiley 2016; formulation and delivery of peptide and protein agent is described, e.g., in Banga, Therapeutic Peptides and Proteins: Formulation, Processing, and Delivery Systems (Third Edition), CRC Press 2015.

The neuromodulating agents described herein may be administered in a unit dose form. For example, the methods described herein include administration of a unit dose form of a beta-adrenergic inhibitory agent. The unit dose can be less than or more than a unit dose of the beta blocker that is FDA approved for high blood pressure, a cardiac condition, angina, essential tremor, hypertrophic subaortic stenosis, migraine prophylaxis, myocardial infarction prophylaxis, pheochromocytoma, tachyarrhythmias, or thyrotoxicosis. The beta-adrenergic blocking agent can be selected from: acebutolol, atenolol, bisoprolol, metoprolol, nadolol, and propranolol. The agent can be formulated for parenteral administration, enteral administration (e.g., oral), or local administration (e.g., epicutaneous, inhalational, intra-articular, intrathecal, intravaginal, intravitreal, intrauterine, intra-lesional or intra-tumoral administration).

The unit dose form can be a unit dose of a cholinergic inhibitory agent. The unit dose can be less than or more than a unit dose of the cholinergic blocker that is FDA approved for Alzheimer's Disease, Cardiac and Respiratory Disorders, Atony and Neurogenic Bladder, motion sickness, Myasthenia gravis, Peptic ulcer, IBD, Glaucoma, Parkinson's Disease, reflex neurogenic bladder (spinal cord injury), or Incontinence-overactive bladder. The cholinergic blocking agent can be selected from: tacrine, galantamine, rivastigmine, donepezil. The unit dose can be configured for local administration, e.g., epicutaneous, inhalational, intra-articular, intrathecal, intravaginal, intravitreal, intrauterine, intra-lesional or intra-tumoral administration.

The unit dose form can be a unit dose of a dopaminergic inhibitory agent. The unit dose can be less than or more than a unit dose of the dopamine blocker that is FDA approved for schizophrenia, bipolar disorder, or nausea and vomiting. The dopamine blocking agent can be selected from: acepromazine, amisulpride, amoxapine, asenapine, azaperone, benperidol, Bromopride, butaclamol, chlorpromazine, chlorprothixene, clopenthixol, Domperidone, droperidol, eticlopride, flupenthixol, fluphenazine, fluspirilene, haloperidol, hydroxyzine, iodobenzamide, loxapine, mesoridazine, levomepromazine, metoclopramide, nafadotride, nemonapride, olanzapine, paliperidone, penfluridol, perazine, perphenazine, pimozide, prochlorperazine, promazine, quetiapine, raclopride, remoxipride, risperidone, spiperone, spiroxatrine, stepholidine, sulpiride, sultopride, tetrahydropalmatine, thiethylperazine, thioridazine, thiothixene, tiapride, trifluoperazine, trifluperidol, triflupromazine, and ziprasidone. The unit dose can be configured for local administration, e.g., epicutaneous, inhalational, intra-articular, intrathecal, intravaginal, intravitreal, intrauterine, intra-lesional or intra-tumoral administration.

The unit dose can be a unit dose of a serotonin inhibitory agent. The unit dose can be less than or more than a unit dose of the serotonin blocker that is FDA approved for treatment of a mood disorder, e.g., major depressive disorder (MDD), anxiety disorder, obsessive-compulsive disorder (OCD), attention deficit hyperactivity disorder (ADHD), chronic neuropathic pain, fibromyalgia syndrome (FMS), or for the relief of menopausal symptoms. The serotonin blocking agent can be selected from: Venlafaxine, Desvenlafaxine, Duloxetine, Milnacipran Levomilnacipran, Sibutramine, and Atomoxetine. The unit dose can be configured for local administration, e.g., epicutaneous, inhalational, intra-articular, intrathecal, intravaginal, intravitreal, intrauterine, intra-lesional or intra-tumoral administration.

Local Administration

The neuromodulating agents described herein can be administered locally, e.g., to the site of damage or disease associated with the cancer in the subject, such as tumor or lymph node. Examples of local administration include epicutaneous, inhalational, intra-articular, intrathecal, intravaginal, intravitreal, intrauterine, intra-lesional administration, lymph node administration, intratumoral administration and administration to a mucous membrane of the subject, wherein the administration is intended to have a local and not a systemic effect. As an example, for the treatment of a cancer described herein, the neuromodulating agent may be administered locally (e.g., intratumorally) in a compound-impregnated substrate such as a wafer, microcassette, or resorbable sponge placed in direct contact with the affected tissue. Alternatively, the neuromodulating agent is infused into the brain or cerebrospinal fluid using standard methods. As yet another example, a pulmonary cancer described herein may be treated, for example, by administering the neuromodulating agent locally by inhalation, e.g., in the form of an aerosol spray from a pressured container or dispenser which contains a suitable propellant, e.g., a gas such as carbon dioxide or a nebulizer. A neuromodulating agent for use in the methods described herein can be administered into a lymph node or at the site of a tumor, e.g., intratumorally. In certain embodiments, the agent is administered to a mucous membrane of the subject.

Combination Therapy

The neuromodulating agents described herein may be administered in combination with one or more additional therapies (e.g., 1, 2, 3 or more additional therapeutic agents). The two or more agents can be administered at the same time (e.g., administration of all agents occurs within 10 minutes, 5 minutes, 2 minutes or less). The agents can also be administered simultaneously via co-formulation. The two or more agents can also be administered sequentially, such that the action of the two or more agents overlaps and their combined effect is such that the reduction in a symptom, or other parameter related to the disorder is greater than what would be observed with one agent or treatment delivered alone or in the absence of the other. The effect of the two or more treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, local routes, and direct absorption through mucous membrane tissues. The therapeutic agents can be administered by the same route or by different routes. For example, a first therapeutic agent of the combination may be administered by intravenous injection while a second therapeutic agent of the combination can be administered locally in a compound-impregnated microcassette. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

For use in treating cancer, the second agent may be a checkpoint inhibitor, a chemotherapeutic drug, a biologic drug. In one embodiment, the inhibitor of checkpoint is an inhibitory antibody (e.g., a monospecific antibody such as a monoclonal antibody). The antibody may be, e.g., humanized or fully human. In other embodiments, the inhibitor of checkpoint is a fusion protein, e.g., an Fc-receptor fusion protein. In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein. In one embodiment, the inhibitor of checkpoint is an inhibitor (e.g., an inhibitory antibody or small molecule inhibitor) of CTLA-4 (e.g., an anti-CTLA4 antibody such as ipilimumab or tremelimumab). In one embodiment, the inhibitor of checkpoint is an inhibitor (e.g., an inhibitory antibody or small molecule inhibitor) of PD-1 (e.g., nivolumab; pembrolizumab; pidilizumab/CT-011). In one embodiment, the inhibitor of checkpoint is an inhibitor (e.g., an inhibitory antibody or small molecule inhibitor) of PDL1 (e.g., MPDL3280A/RG7446; MED14736; MSB0010718C; BMS 936559). In one embodiment, the inhibitor of checkpoint is an inhibitor (e.g., an inhibitory antibody or Fc fusion or small molecule inhibitor) of PDL2 (e.g., a PDL2/Ig fusion protein such as AMP 224). In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAGS, VISTA, KIR, 2B4, CD160, CGEN-15049, CHK 1, CHK2, A2aR, B-7 family ligands, or a combination thereof. The second agent may also be an anti-angiogenic drug, e.g., an anti-VEGF antibody, or the second agent may be an oncolytic agent e.g., a chemotherapy, a drug that targets cancer metabolism, an antibody that marks a cancer cell surface for destruction, e.g., rituximab or trastuzumab an antibody-drug conjugate, e.g., trastuzumab emtansine, a cell therapy, or other commonly-used anti-neoplastic agent.

Dosing

Subjects that can be treated as described herein are subjects with cancer or at risk of developing cancer. The cancer may be a primary tumor or a metastasized tumor. Subjects who can be treated with the methods disclosed herein include subjects who have had one or more tumors resected, received chemotherapy or other pharmacological treatment for the cancer, received radiation therapy, and/or received other therapy for the cancer. Subjects who have never previously been treated for cancer can also be treated using the methods described herein.

In some embodiments, the agent is administered in an amount and for a time effective to result in one of (or more, e.g., 2 or more, 3 or more, 4 or more of): (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

The methods described herein may include a step of selecting a treatment for a patient. The method includes (a) identifying (e.g., diagnosing) a patient who has cancer or is at risk of developing cancer, and (b) selecting a neuromodulating agent, e.g., a neuromodulating agent described herein, to treat the condition in the patient. In some embodiments, the method includes administering the selected treatment to the subject. In some embodiments, a patient is identified as having cancer based on imaging (e.g., MRI, CT, or PET scan), biopsy, or blood sample (e.g., detection of blood antigen markers, circulating tumor DNA (e.g., by PCR). In some embodiments, a patient is identified as having cancer after presenting with one or more symptoms of a paraneoplastic syndrome (e.g., fever, auto-antibodies directed against nervous system proteins, ataxia, dizziness, nystagmus, difficulty swallowing, loss of muscle tone, loss of fine motor coordination, slurred speech memory loss, vision loss, sleep disturbances, dementia, seizures, dysgeusia, cachexia, anemia, itching, or sensory loss in the limbs). In some embodiments, a patient presents with symptoms of paraneoplastic syndrome and is then identified as having cancer based on imaging (e.g., CT, MRI, or PET scans).

The method may also include (a) identifying (e.g., diagnosing) a patient who has a neoplasm, (b) optionally evaluating the neoplasm for innervation, and (c) selecting a neuromodulating agent (e.g., a neuromodulating agent described herein) to treat the patient if the neoplasm is highly innervated (e.g., if the level of innervation is at least 10% higher (e.g., at least 20%, 30%, 40%, 50%, 60%, 70%, 80% higher) than the level of innervation in control tissue, e.g., non-cancerous tissue of the same subject). Innervation may be measured by staining tissue sections for neural markers e.g., immuno-histochemical staining for tyrosine hydroxylase, vesicular acetylcholine transporter; NGF-Inducible Large External glycoprotein, choline acetyltransferase, parvalbumin, neurofilament protein, Synapsin, synaptophysin, NeuN, NSE, MAP2, Beta III tubulin, 160 kD Neurofilament medium/200 kD Neurofilament Heavy, NSE, PSD93/PSD95, Doublecortin (DCX), c-fos, PSA-NCAM, NeuroD or Beta2, Tau, Calbindin-D28k, Calretinin, Neurofilament Protein (NFP), Glial fibrillary acidic protein (GFAP), S100β, Vimentin and CNPase; or by staining tissue sections with cell-identifying stains, e.g., H&E stain, Nissl Stain, Cresyl violet, Neutral red, Thionine and Toluidine blue, Luxol Fast blue stain, Weigert's Chromium hematoxylin method, Page's iron-eriochrome cyanine R, Dextran Conjugates (Fluorescein, Tetramethylrhodamine, Texas Red, Rhodamine Green), Hydrazides & Biocytins, Isolectin GS-1B4 conjugates, Golgi silver stain, or myelin stain; or by imaging the nervous system, e.g., by MRI, CT, PET, EEG, EMG, Myelogram, or magnetoencephalography. In some embodiments, the neoplasm is selected from: head and neck squamous cell carcinoma, adenoid cystic carcinoma, lymphoma, rhabdomyosarcoma, biliary tract cancer, gastric cancer, pancreatic cancer, prostate cancer, lung cancer, breast cancer, skin cancer (e.g., melanoma), renal cell carcinoma, or colorectal cancer. In some embodiments, the cancer is a cancer listed in Table 5. In some embodiments, the neoplasm is derived from a secretory tissue, glandular tissue, or endocrine or hormonal tissue.

In one embodiment, the method includes (a) identifying (e.g., diagnosing) a patient who has a neoplasm, (b) optionally evaluating the neoplasm for perineural invasion, and (c) selecting a neuromodulating agent to treat the patient if the neoplasm exhibits perineural invasion. In some embodiments, the neoplasm is selected from: head and neck squamous cell carcinoma, adenoid cystic carcinoma, lymphoma, rhabdomyosarcoma, biliary tract cancer, gastric cancer, pancreatic cancer, and prostate cancer.

In one embodiment, the method includes (a) identifying (e.g., diagnosing) a patient who has a neoplasm, (b) optionally evaluating the subject for metastasis to brain or spinal cord, and (c) selecting a neuromodulating agent to treat the patient if the neoplasm exhibits metastasis to brain or spinal cord. In some embodiments, the neoplasm is a lung cancer, breast cancer, skin cancer (e.g., melanoma), lymphoma, renal cell carcinoma, GI tract cancer, prostate cancer, or colorectal cancer.

In some embodiments, the method includes administering the selected treatment to the subject.

The method may also include a step of assessing the subject for a parameter of cancer progression or remission, e.g., assessing the subject for one or more (e.g., 2 or more, 3 or more, 4 or more) of: primary tumor size (e.g., by imaging), number of metastases (e.g., by imaging or biopsy), cell death in situ (e.g., by biopsy), blood antigen markers (e.g., by ELISA), circulating tumor DNA (e.g., by PCR), or function of the affected organ (e.g., by a test of circulating enzymes for liver, albuminuria for kidney, lung capacity for lung, etc.).

In some embodiments, a tumor is treated with a neuromodulating agent and a second therapeutic agent. The second therapeutic agent can be selected based on tumor type, tumor tissue of origin, tumor stage, or mutations in non-neurome genes expressed by the tumor.

A neuromodulating agent administered according to the methods described herein does not have a direct effect on the central nervous system (CNS) or gut. Any effect on the CNS or gut will be reduced compared to the effect observed if the neuromodulating agent is administered directly to the CNS or gut. Direct effects on the CNS or gut can be avoided by modifying the neuromodulating agent not to cross the BBB, as described herein above, or administering the agent locally to a subject.

Subjects with cancer or at risk of developing cancer are treated with an effective amount of a neuromodulating agent. The methods described herein also include contacting immune cells with an effective amount of a neuromodulating agent. In some embodiments, an effective amount of a neuromodulating agent is an amount sufficient to increase or decrease lymph node innervation, tumor innervation, the development of HEVs or TLOs, immune cell migration, proliferation, recruitment, lymph node homing, lymph node egress, differentiation, tumor homing, tumor egress, activation, polarization, cytokine production, degranulation, maturation, ADCC, ADCP, or antigen presentation. In some embodiments, an effective amount of a neuromodulating agent is an amount sufficient to increase or decrease tumor innervation or nerve activity in a tumor. In some embodiments, an effective amount of a neuromodulating agent is an amount sufficient to treat the cancer or tumor, cause remission, reduce tumor growth, volume, metastasis, invasion, proliferation, or number, increase cancer cell death, increase time to recurrence, or improve survival.

The methods described herein may also include a step of assessing the subject for a parameter of immune response, e.g., assessing the subject for one or more (e.g., 2 or more, 3 or more, 4 or more) of: Th2 cells, T cells, circulating monocytes, neutrophils, peripheral blood hematopoietic stem cells, macrophages, mast cell degranulation, activated B cells, NKT cells, macrophage phagocytosis, macrophage polarization, antigen presentation, immune cell activation, immune cell proliferation, immune cell lymph node homing or egress, T cell differentiation, immune cell recruitment, immune cell migration, lymph node innervation, dendritic cell maturation, HEV development, TLO development, or cytokine production. In embodiments, the method includes measuring a cytokine or marker associated with the particular immune cell type, as listed in Table 9 (e.g., performing an assay listed in Table 9 for the cytokine or marker). In some embodiments, the method includes measuring a chemokine, receptor, or immune cell trafficking molecule, as listed in Tables 10 and 11 (e.g., performing an assay to measure the chemokine, marker, or receptor). The assessing may be performed after the administration, before the first administration and/or during a course a treatment, e.g., after a first, second, third, fourth or later administration, or periodically over a course of treatment, e.g., once a month, or once every 3 months. In one embodiment, the method includes assessing the subject prior to treatment or first administration and using the results of the assessment to select a subject for treatment. In certain embodiments, the method also includes modifying the administering step (e.g., stopping the administration, increasing or decreasing the periodicity of administration, increasing or decreasing the dose of the neuromodulating agent) based on the results of the assessment. For example, in embodiments where increasing a parameter of immune response described herein is desired (e.g., cancer-related embodiments where, e.g., an increase in Th2 cells is desired), the method includes stopping the administration if a marker of Th2 cells is not increased at least 5%, 10%, 15%, 20%, 30%, 40%, 50% or more; or the method includes increasing the periodicity of administration if the marker of Th2 cells is not increased at least 5%, 10%, 15%, 20%, 30%, 40%, 50% or more; or the method includes increasing the dose of the neuromodulating agent if the marker of Th2 cells is not increased at least 5%, 10%, 15%, 20%, 30%, 40%, 50% or more. For example, in embodiments where decreasing a parameter of immune response described herein is desired (e.g., embodiments where a decrease in Th2 cells is desired), the method includes stopping the administration if a marker of Th2 cells is not decreased at least 5%, 10%, 15%, 20%, 30%, 40%, 50% or more; or the method includes increasing the periodicity of administration if the marker of Th2 cells is not decreased at least 5%, 10%, 15%, 20% or more; or the method includes increasing the dose of the neuromodulating agent if the marker of Th2 cells is not decreased at least 5%, 10%, 15%, 20% or more.

In certain embodiments, immune effects (e.g., immune cell activities) are modulated in a subject (e.g., a subject having a cancer or inflammatory or autoimmune condition) or in a cultured cell by at least 1%, 2%, 5%, 10%, 15%, 20%, 25%, 30%, 35%, 40%, 50%, 60%, 70%, 80%, compared to before an administration, e.g., of a dosing regimen, of a neuromodulating agent such as those described herein. In certain embodiments, the immune effects are modulated in the subject or a cultured cell between 5-20%, between 5-50%, between 10-50%, between 20-80%, between 20-70%, between 50-100%, between 100-500%. The immune effects described herein may be assessed by standard methods:

The neuromodulating agents described herein are administered in an amount (e.g., an effective amount) and for a time sufficient to effect one of the outcomes described above. The neuromodulating agent may be administered once or more than once. The neuromodulating agent may be administered once daily, twice daily, three times daily, once every two days, once weekly, twice weekly, three times weekly, once biweekly, once monthly, once bimonthly, twice a year, or once yearly. Treatment may be discrete (e.g., an injection) or continuous (e.g., treatment via an implant or infusion pump). Subjects may be evaluated for treatment efficacy 1 week, 2 weeks, 1 month, 2 months, 3 months, 4 months, 5 months, 6 months or more following administration of a neuromodulating agent depending on the neuromodulating agent and route of administration used for treatment. Depending on the outcome of the evaluation, treatment may be continued or ceased, treatment frequency or dosage may change, or the patient may be treated with a different neuromodulating agent. Subjects may be treated for a discrete period of time (e.g., 1, 2, 3, 4, 5, 6, 7, 8, 9, 10, 11, or 12 months) or until the disease or condition is alleviated, or treatment may be chronic depending on the severity and nature of the disease or condition being treated.

Kits

The invention also features a kit comprising (a) a unit dose described herein, and (b) instructions for administering the unit dose to treat cancer.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1—Screening Method for Serotonin Receptor Binders

High throughput methods for identifying compounds from libraries that bind to a target molecule have been described previously, see e.g., Janzen and Bernasconi (Eds.), High Throughput Screening: Methods and Protocols (Methods in Molecular Biology), Humana Press 2009. In brief, to identify compounds that bind to the serotonin receptor 5HT2C the following screening assay is performed:

Cell Culture & Membrane (Target Protein) Preparation:

AV12 cells are stably transfected with a eukaryotic expression vector containing the coding region for the human 5HT2C receptor (see e.g., Lucaites, V. L., et al., (1996) Life Sci. 59(13), 1081-1095). To prepare membrane protein preparations, using the technique of Bosworth and Towers, Nature 341, 167, 1989, cells are grown to a cell density of 2-3×10⁶ cells/mL, and 15 L are harvested on a daily basis by centrifugation, washed in phosphate-buffered saline (PBS), and stored as frozen cell pastes at −80° C. To loosen the frozen cell paste, 30 mL of 50 mM Tris-HCl, pH 7.4, at ambient temperature are added to 7.5 grams of pellet. The cell slurry is homogenized on ice in a 55-mL glass/teflon dounce, transferred to a 250-mL conical tube that is then filled to the neck with buffer, mixed, and centrifuged in a table top centrifuge at 200 g (1060 RPM, GH-3.7 rotor) at 4° C. for 15 min. The supernatant is collected and saved on ice. The pellet is resuspended and subjected to the homogenization and centrifugation procedure just described. The 200 g supernatant is again collected and combined with the first supernatant stored on ice. The combined supernatants are then centrifuged at 14,250 rpm in a Sorvall RC5 centrifuge (GSA SLA-1500 rotor) for 50 min at 4° C. The supernatant is gently removed and discarded, and the remaining membrane pellet is resuspended using the dounce homogenizer. The membrane protein concentration is determined (BCA kit) and aliquots of the membrane preparation are quick frozen in liquid nitrogen and stored at −80° C. The average yield is 1.2% of starting weight.

SPA-Format Receptor-Binding Assay:

Twenty microliter of test compound, unlabeled 5-HT control, or assay buffer is added to each well of a 96-well microtiter plate. Fifty microliter of 15-nM [3H]-5HT ligand (5-Hydroxy(3H)tryptamine trifluoroacetate (Code TRK1006 Amersham) at a final concentration of 5 nM/well) is then added to the wells followed by 80 μL (20 ug) of 5HT2C membranes as prepared above and the plates are shaken for 1 min. After a 30-min incubation at room temperature, 0.5 mg of Wheat Germ Agglutinin (WGA)-SPA beads (Amersham biotech) are added, plates are mixed by shaking every 30 min for 2 h and then counted in a MicroBeta Scintillation Counter (Perkin Elemer Wallac). The absence of binding of labeled 5HT ligand in a sample indicates that the test compound has successfully bound the target receptor. Test compounds that bind target receptor with greater than 100 nM EC50 (p<0.05 for at least 3 replicates) are selected for further testing.

### Example 2—Dose Finding Study for Neuromodulator Candidate

A lead candidate for treatment of a solid cancer is identified by the screening method of Example 1. Based on preclinical data from in vitro and in vivo testing of the identified lead compound, it is determined that 120 mg is a safe starting dose in humans.

A ‘3+3’ design of incremental escalation of dose in a cohort of subjects is employed to identify a Maximum Tolerated Dose (MTD) of the lead candidate. Dose escalation is determined using a Fibonacci sequence, whereby an additional 100% of the original dose is administered for the second time, 67% of the second dose for the third time, and so on, until the MTD is reached.

Three patients are given 120 mg of the identified lead compound. If none of the three patients report any dose limiting toxicity (DLT) of this first dose, then the dose is escalated for the next cohort of 3 subjects. If within any one particular cohort one of the patients reports a DLT, the study at that dose is repeated. If two of the patients report DLT, this dose is then regarded as the Maximum Tolerated Dose (MTD).

### Example 3—T Cell Activation in Culture

A high throughput antigen recall assay is used to confirm that the agents identified as described in Example 1 or Example 2 activate T cells. Determining impaired T-cell function by culturing human peripheral blood mononuclear cells (PBMC) in vitro with recall antigens has been described (see e.g., Stone et al, Clin. Immunol. 131:41, 2009). In brief, the following procedure is used for the detection of the modulation of interferon gamma secretion from T cells treated with a compound of interest:
Plates (1,536 wells) are coated with 5 μl of the first anti-gamma interferon (anti-IFN-γ) antibody at 1 μg/ml in PBS. After overnight incubation at 4° C., the plates are washed twice with PBS and saturated with 10 μl PBS-1% human serum albumin (20% solution; Kedrion, Lucca, Italy) at room temperature (RT) for 1 h. After saturation, the plates are washed twice with 13 μl Hanks' balanced salt solution and received 4 μl of complete medium.
CMV antigen is dispensed with predefined patterns in 96-well master plates and transferred into 1,536-well plates using the Hydra II liquid handler. One microliter of 10× antigen solution is dispensed into the wells that already contain 4 μl of complete medium. The plates are sealed with a plastic membrane and frozen at −80° C., ready for use.
PBMCs obtained from heparinized peripheral blood of CMV-positive donors by the conventional Ficoll gradient are brought to 2×10\hat{ }6 cells/ml in complete medium and automatically dispensed into thawed plates at 5 μl per well. The plates are incubated for 2 days in a 5% CO2 atmosphere.
During the culture, the agent of interest identified as described in Example 1 or Example 2 is administered at various concentrations to the cells in culture.
At the end of the culture, supernatants or cells can be collected for further experiments. After washing twice with PBS-Tween 20 0.05%, the plates are spun upside down on filter paper at 500×g for 2 min for complete removal of washing buffer. Then, the wells receive 5 μl biotinylated antibody at 1 μg/ml for 1 h. After washing twice with PBS-Tween and three times with PBS and drying by centrifugation, the wells receive 5 μl alkaline phosphatase-streptavidin at 1 μg/ml. After being incubated at room temperature, the plates are washed as described in the previous step and the wells receive 10 μl p-nitrophenylphosphate. After a 1-h incubation at RT, the plates are scanned on a Victor 3V (Perkin-Elmer). Results are shown as the optical density at 405 nm×1,000 or as picograms/milliliter of cytokine.

Wells in which the compound of interest induces an amount of interferon gamma as measured by optical density that is greater than 2-fold higher than the unstimulated cell control are identified as being able to induce T cell activation.

### Example 4—Identification of Novel Neurobiological Correlations with Immune Disease

To identify novel correlations of neurobiological signaling molecules and immune cells, a list of neurotransmitter and neuropeptide genes and pathways was generated using published literature and UniProt (see Table 1). These genes and pathways were used as inputs to publicly available immune cell databases (e.g., RCAI RefDIC, Reference Database of Immune Cells). Through the bioinformatics analysis, novel correlations were found of overexpression by at least two-fold of certain neurobiological signaling genes of interest in certain individual immune cells. Table 13 lists the neurobiological signaling molecules (column 1) that are targets for therapeutic intervention for immune disorders or conditions through activity on the correlated immune cells (column 2).

TABLE 13
IMMUNE CELL CORRELATIONS

| Gene | Immune Cell | Accession Number |
|---|---|---|
| ADRA2C | Dendritic Cells | P18825 |
| ADRB1 | Myeloid Cells | P08588 |
| ADRB1 | Monocytes | P08588 |
| ADRB2 | B Cells | P07550 |
| ADRB2 | T Cells | P07550 |
| ADRB2 | Myeloid Cells | P07550 |
| ADRB2 | Monocytes | P07550 |
| ADRBK1 | Dendritic Cells | P25098 |
| ADRBK1 | T Cells | P25098 |
| ADRBK1 | Myeloid Cells | P25098 |
| ADRBK1 | Monocytes | P25098 |
| ADRBK2 | Dendritic Cells | P35626 |
| ADRBK2 | B Cells | P35626 |
| ADRBK2 | T Cells | P35626 |
| ADRBK2 | Myeloid Cells | P35626 |
| ADRBK2 | Monocytes | P35626 |
| C4orf48 | Dendritic Cells | Q5BLP8 |
| C4orf48 | B Cells | Q5BLP8 |
| C4orf48 | T Cells | Q5BLP8 |
| CALCRL | Dendritic Cells | Q16602 |
| CALCRL | Myeloid Cells | Q16602 |
| CALCRL | Monocytes | Q16602 |
| CALCRL | Myeloid Cells | Q16602 |
| CALCRL | Monocytes | Q16602 |
| CHRNA2 | Dendritic Cells | Q15822 |
| CHRNA2 | B Cells | Q15822 |
| CHRNA2 | T Cells | Q15822 |
| CHRNA2 | Myeloid Cells | Q15822 |
| CHRNA7 | Dendritic Cells | P36544 |
| CHRNA7 | T Cells | P36544 |
| CHRNA7 | Myeloid Cells | P36544 |
| CHRNA7 | Monocytes | P36544 |
| CHRNB2 | Dendritic Cells | P17787 |
| CHRNB2 | B Cells | P17787 |
| CHRNB2 | T Cells | P17787 |
| CHRNB2 | Myeloid Cells | P17787 |
| CHRNB2 | Monocytes | P17787 |
| CRH | Dendritic Cells | Q13324 |
| CYSLTR2 | Monocytes | Q9NS75 |
| HTR2B | Dendritic Cells | P41595 |
| HTR2B | Myeloid Cells | P41595 |
| HTR3A | B Cells | P46098 |
| HTR4 | Monocytes | Q13639 |
| NMB | Dendritic Cells | P08949 |
| NMB | B Cells | P08949 |
| NMB | T Cells | P08949 |
| NPPA | Dendritic Cells | P01160 |
| NXPH3 | Dendritic Cells | O95157 |
| PMCH | Myeloid Cells | P20382 |
| PNOC | B Cells | Q13519 |
| UCN | Dendritic Cells | Q96RP3 |
| UCN | B Cells | Q96RP3 |
| UCN | T Cells | Q96RP3 |
| UCN | Myeloid Cells | Q96RP3 |

### Example 5—T Cell Activation and Cytokine Secretion Assay

Peripheral blood mononuclear cells (PBMCs) were isolated from whole blood by ficoll density separation. Briefly, blood was diluted 1:2 with 0.5 mM EDTA solution in PBS, loaded onto a Ficoll-filled Leucosep tube (Greiner Bio-One), and centrifuged for 20 minutes at 1000×g. After centrifugation, the leukocyte/PBMC layer on top of the separation medium was collected and sequentially washed three times with 0.5 mM EDTA solution in PBS.

T cells were isolated from PBMCs using magnetic bead-based separation following vendor specification, e.g., Biolegend MojoSort Human CD4/CD8 Naïve T Cell Isolation Kit protocol. In brief, the PBMCs were labeled with biotinylated antibodies against cell surface receptors for cells not in the population of interest. The labeled cells are then captured by streptavidin-coated magnetic beads and removed by magnetic incubation. The uncaptured cells that flow through the magnetic separation are predominantly comprised of the population of interest, in this instance CD3+ T cells. T cells were stained with a 5 μM solution of Tag-it Violet™ dye (BioLegend) for 20 minutes protected from light. The stain was quenched by incubating the cells in cell culture medium containing 10% FBS (complete media).

Stained cells were plated on tissue culture plates and sub-maximally activated with concanavalin A (con A) added to the culture medium. Cells were plated at 0.1×10⁶ cells well. Dopamine, dopaminergic agonist quinpirole, adrenergic agonist isoproterenol, adrenergic antagonist propranolol, and neuropeptide Y were added at a range of concentrations between 0.1 nM and 0.1 mM. Cells were collected at 24, 48, and 72 hrs.

Supernatants were collected at 24, 48, and 72 hrs and cytokine secretion was analyzed by flow cytometry using a LEGENDplex assay (BioLegend). In brief, following manufacturer's protocol, beads pre-coated with antibodies specific to various cytokines were incubated with cell supernatant. Cytokines in the supernatant are confirmed by adding a second detection antibody in a classic “sandwich ELISA” format. The beads were then stained and the captured cytokine composition assessed by flow cytometry.

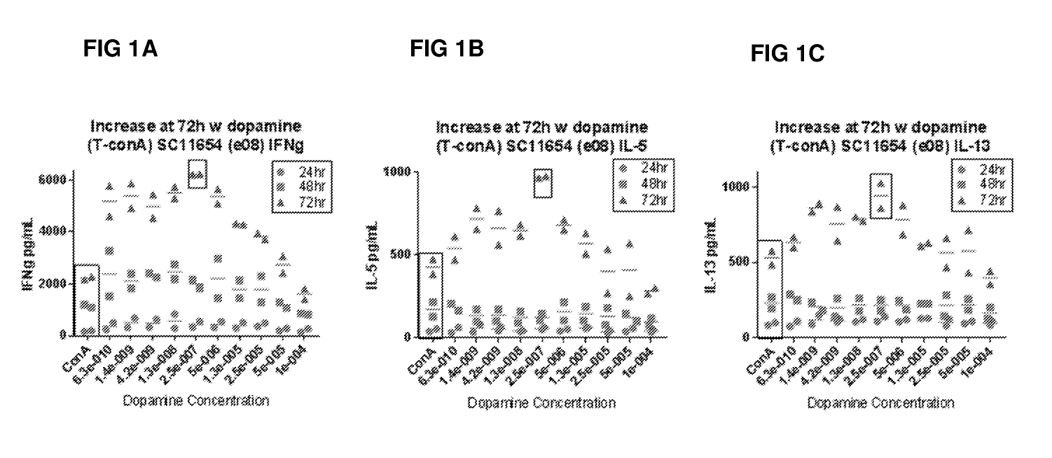

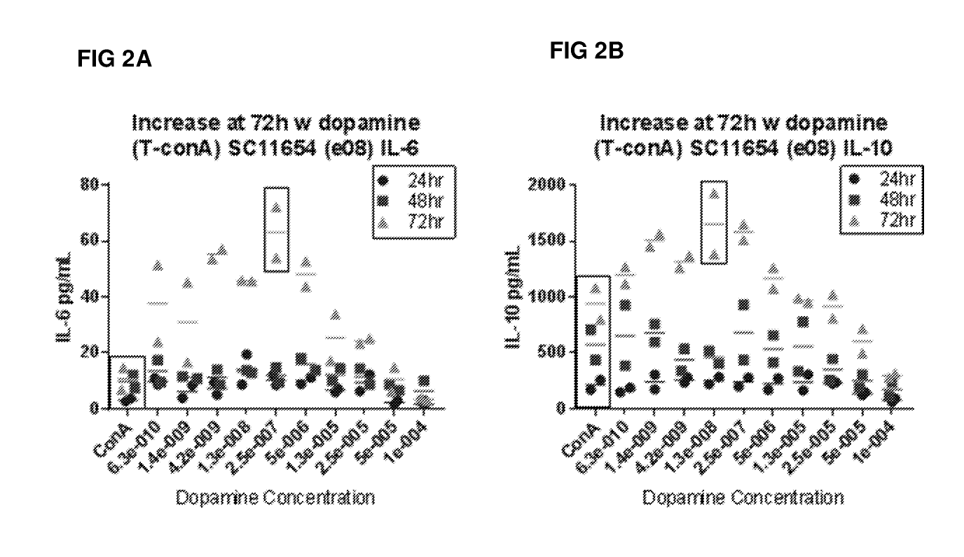

We found that dopamine stimulation at low sub-nanomolar concentrations induced an increase in the production of the pro-inflammatory cytokines IFNγ, IL-5, IL-6, IL-10, and IL-13 at 72 hours post treatment (FIGS. 1A-1C and FIGS. 2A-2B). These data suggest that stimulation of T cells with dopamine in vivo may increase production of pro-inflammatory cytokines, which can be important for promoting inflammation in the context of cancer, in which case one might want to activate T cells to treat the disease.

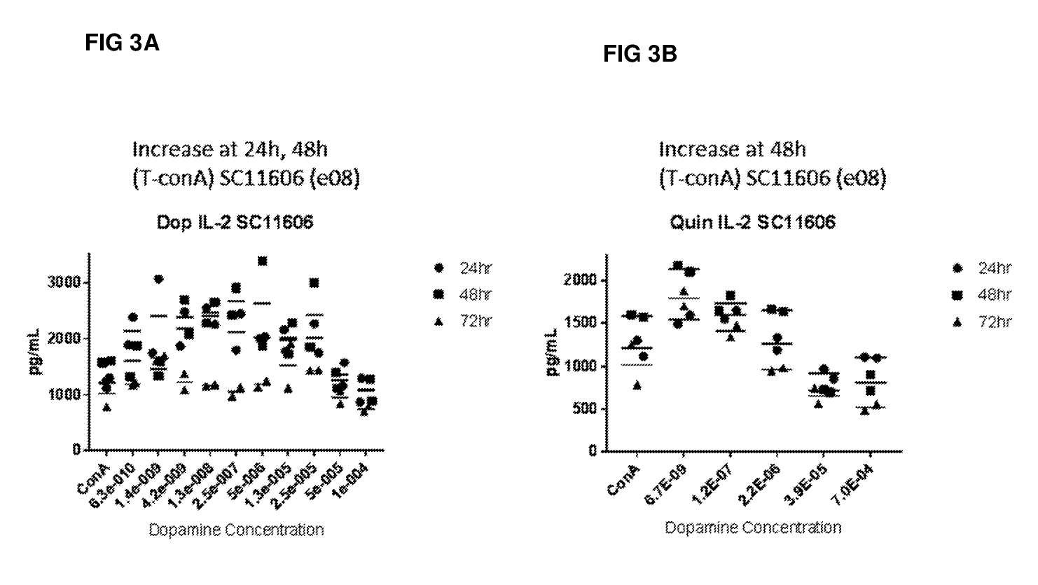

We found that stimulation of T cells with dopamine and the synthetic dopaminergic agonist, quinpirole, induced an increase in the production of the pro-survival cytokine IL-2. For dopamine the effect is observed at 24- and 48-hours post stimulation with nanomolar concentrations of the neurotransmitter. For quinpirole, the effect was seen at all time points tested, again at nanomolar concentrations of the agonist (FIGS. 3A-3B). The differences in the kinetics may be due to the differences in lability or affinity of the agents. These data suggest that stimulation of T cells with dopamine in vivo may increase proliferation of T cells, which can be a useful treatment in the context of immunotherapy for cancer by increasing the total number of T cells in the patient (analogous to IL-2 cytokine therapy). Alternatively, the data suggest that inhibition of dopaminergic signaling, for example with a small molecule or antibody antagonist, may prevent the dopamine-mediated proliferation of T cells.

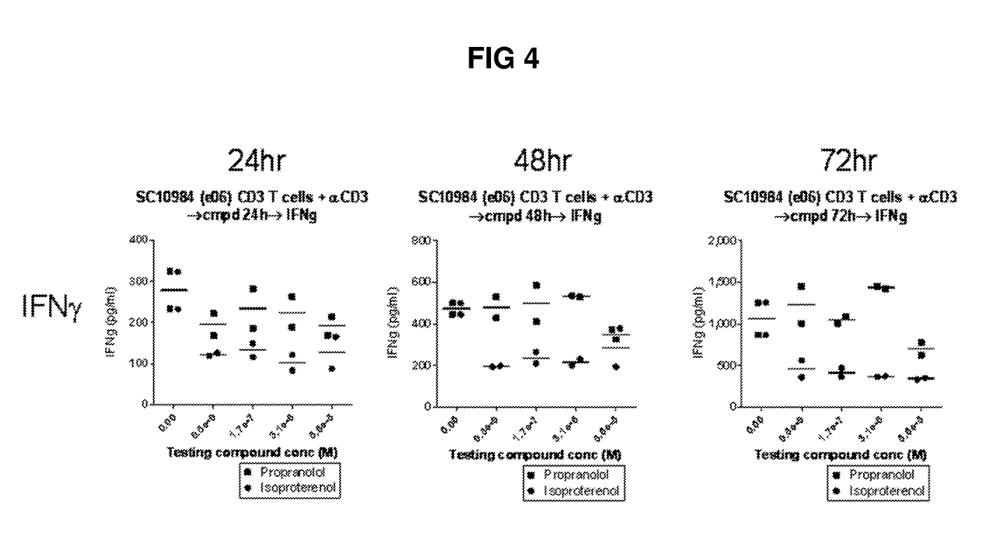

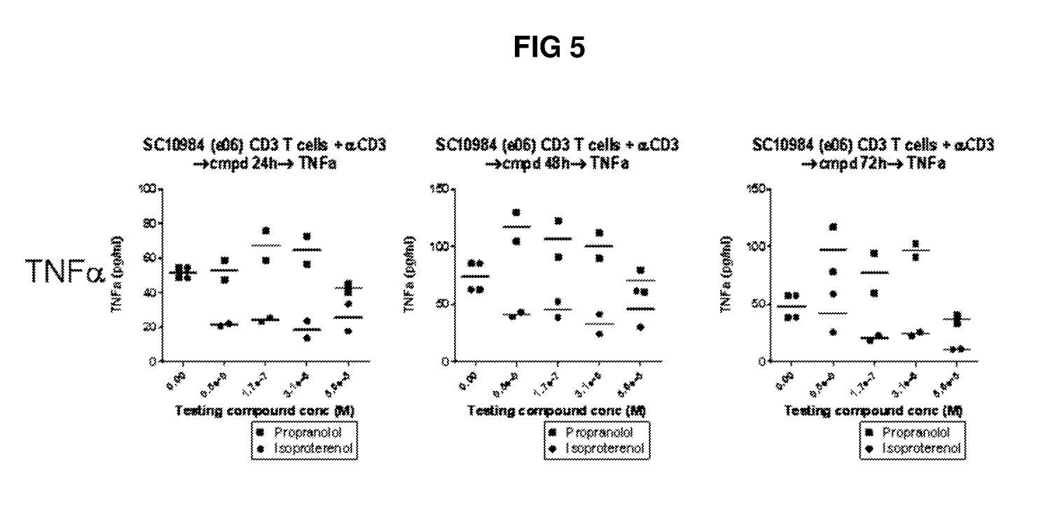

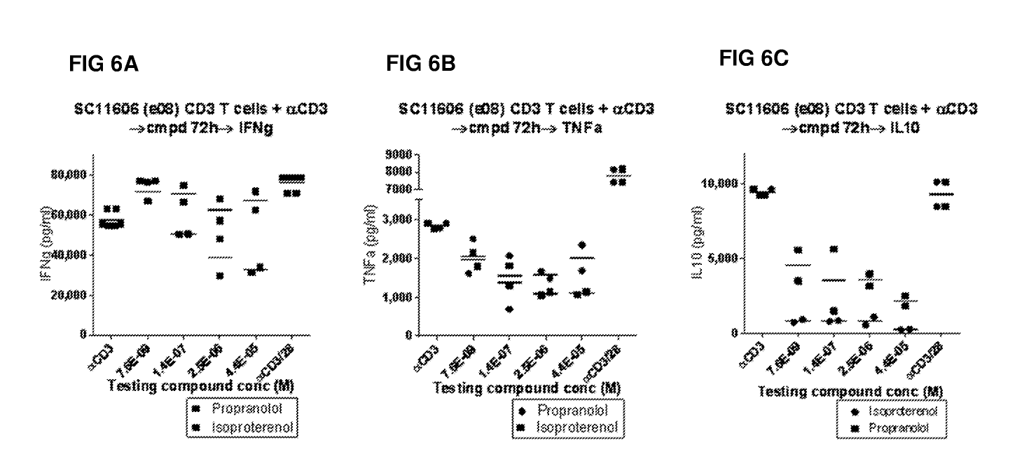

We observed that, in contrast to the dopamine data, stimulation of T cells with the adrenergic agonist isoproterenol induced a decrease in the amount of pro-inflammatory cytokines IFNγ, TNFα, and IL-10 in two different donors at multiple time points (FIG. 4, FIG. 5, and FIGS. 6A-6C). These data suggest that stimulation of T cells via agonism of adrenergic receptors in vivo may increase production of pro-inflammatory cytokines, which can be important for promoting inflammation in the context of cancer, in which case one might want to activate T cells to treat the disease. Alternatively, these data also suggest that inhibition of adrenergic signaling, for example with a small molecule adrenergic antagonist or with an antibody against the adrenergic receptor, may prevent the activation of T cells.

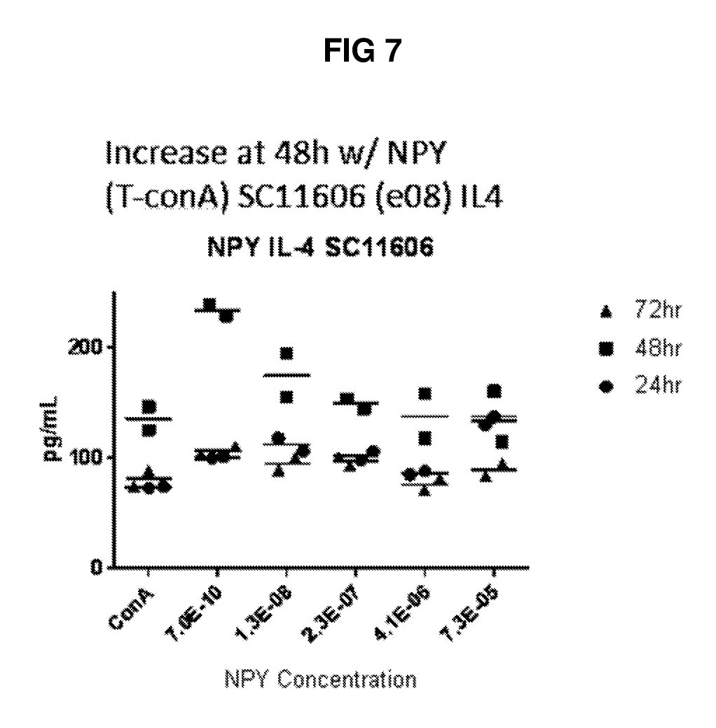

We found that stimulation of T cells with neuropeptide Y induced an increase in the cytokine IL-4 at sub-nanomolar concentrations at 48 hours post-treatment (FIG. 7). These data suggest that intervention along the neuropeptide Y signaling axis in vivo can have a useful therapeutic impact. Physiologically, Th2 cytokines are known to mediate host defense against parasites but they can also trigger disease if their activities are dysregulated. For example, inhibition of neuropeptide Y, for example with an antibody against the neuropeptide Y receptor or an inert peptide mimetic, could be a therapeutic intervention for IL-4 mediated diseases, including cancer.

### Example 6—Macrophage Activation and Cytokine Secretion Assay

Peripheral blood mononuclear cells (PBMCs) were isolated from whole blood by ficoll density separation. Briefly, blood was diluted 1:2 with 0.5 mM EDTA solution in PBS, loaded onto a Ficoll-filled Leucosep tube (Greiner Bio-One), and centrifuged for 20 minutes at 1000×g. After centrifugation, the leukocyte/PBMC layer was collected and sequentially washed three times with 0.5 mM EDTA solution in PBS.

Monocytes (CD14+) were isolated from PBMCs using magnetic bead-based separation following vendor specifications, e.g., Biolegend MojoSort Human CD14 Selection Kit protocol. In brief, PBMCs were labeled with biotinylated antibodies against cell surface receptors for cells not in the population of interest. The labeled cells are then captured by streptavidin-coated magnetic beads and removed by magnetic incubation. The uncaptured cells that flow through the magnetic separation are predominantly comprised of the population of interest, in this case CD14+ monocytes.

Monocytes were differentiated into macrophages by culturing in DMEM complete medium containing 10% FBS for seven days in the presence of 40 ng/mL human M-CSF. Media was changed on day 1 and day 4. On Day 4, macrophages were polarized to various subtypes as follows: M0—incubated with 40 ng/mL M-CSF; M1—cultured with 40 ng/mL M-CSF, 20 ng/mL IFNγ, and 50 ng/mL LPS; M2—incubated with 40 ng/mL M-CSF, 20 ng/mL IL4, 20 ng/mL IL10, and 20 ng/mL TFGB. On day 7, cells were harvested by scraping them from the tissue culture plates and transferring them to 96-well plates. Macrophages were incubated with the neuropeptide CGRP and the small molecule beta adrenergic receptor agonist isoproterenol at varying dilutions from 10 μM to 1 nM.

Supernatants were collected at 24, 48, and 72 hrs and cytokine secretion was analyzed by flow cytometry using a LEGENDplex assay (BioLegend). In brief, following manufacturer's protocol, beads that are pre-coated with antibodies specific to various cytokines were incubated with cell supernatant. Cytokines in the supernatant were confirmed by adding a second detection antibody in a classic “sandwich ELISA” format. The beads were then stained and the captured cytokine composition assessed by flow cytometry.

M1 macrophage polarization is defined as increase in production of IL-12, TNF, IL-6, IL-8, IL-1B, MCP-1 and CCL2. Additionally, markers of M1 polarization that can be detected by RNA include Nos2. M2 polarization is defined as increase in IL-10 and/or a decrease in the M1 cytokines listed above. Additionally, markers of M2 polarization that can be detected by RNA include Arg1, IDO, PF4, CCL24, IL10, and IL4Ralpha.

In this assay, macrophages incubated with the beta adrenergic receptor agonist isoproterenol are polarized toward an M2 phenotype as measured by an increase in the transcripts for Arg1 and IL-10 and a decrease in the transcript of NOS2. Conversely, macrophages stimulated with neuropeptide CGRP are polarized toward an M1 phenotype, as measured by increased secretion of TNFα.

This surprising result indicates that macrophages can be polarized toward an M1 or M2 phenotype strictly via stimulation of neurotransmitter or neuropeptide pathways. M2 polarized macrophages are anti-inflammatory and induce a broadly suppressive immunological cascade, including cytokine secretion, reduced phagocytic activity, and reduced antigen presentation. M1 polarized macrophages are pro-inflammatory and induce a broadly pro-inflammatory immunological cascade, including cytokine secretion, increased phagocytic activity, and increased antigen presentation. As such, this surprising finding indicates that substances that modulate these neurotransmitter/neuropeptide pathways could be used to treat patients with a range of immunological and inflammatory disorders, for example cancer, fibrosis, allergy, allergic dermatitis, pancreatitis, ulcerative colitis, inflammatory bowel disease, Hirschsprung's disease, NASH, fatty liver disease, atherosclerosis, hemophagocytic lymphohistiocytosis, hemophagocytic syndrome, myasthenia gravis, glomerulonephritis, and other diseases and conditions in which macrophage activation and polarization plays a role.

### Example 7—Lymph Node Remodeling by Hock Injection of Dopamine Agonist

C57BL/6J mice were injected in each hock with 50 μL of the immunostimulant CpG ODN (0.1 nmol), 50 μL dopaminergic agonist quinpirole (0.1 nmol) or with 25 μL dopaminergic antagonist (Haloperidol −48.5 nmol) followed by 25 μL quinpirole (0.1 nmol). 24 hours after hock injection, brachial lymph nodes (LN) were harvested in culture medium (RPMI+10% FBS). LNs were transferred to 24-well tissue culture plates containing 0.5 mL LN digestion buffer (RPMI, 2% FBS, collagenase D (3.3 mg/mL), and DNAse I (40 μg/mL)). LN capsules were manually opened with two syringe (26G) needles and the LNs were incubated in digestion buffer for 15 minutes. Digested LNs were filtered with a 40 μM cell strainer and tissues were smashed with the plunger of a 5 mL syringe. Collected cells were washed in culture medium and plated for assays.

Total number of viable cells were assessed by staining with viability dye eFluor 780 (eBioscience). Surface markers of various immune cell subsets were analyzed by staining the cells with antibodies for cell identity (CD3, CD4, CD8, CD19), for the inflammatory marker CD69, and the migratory marker CCR7. The cells were then assayed by flow cytometry.

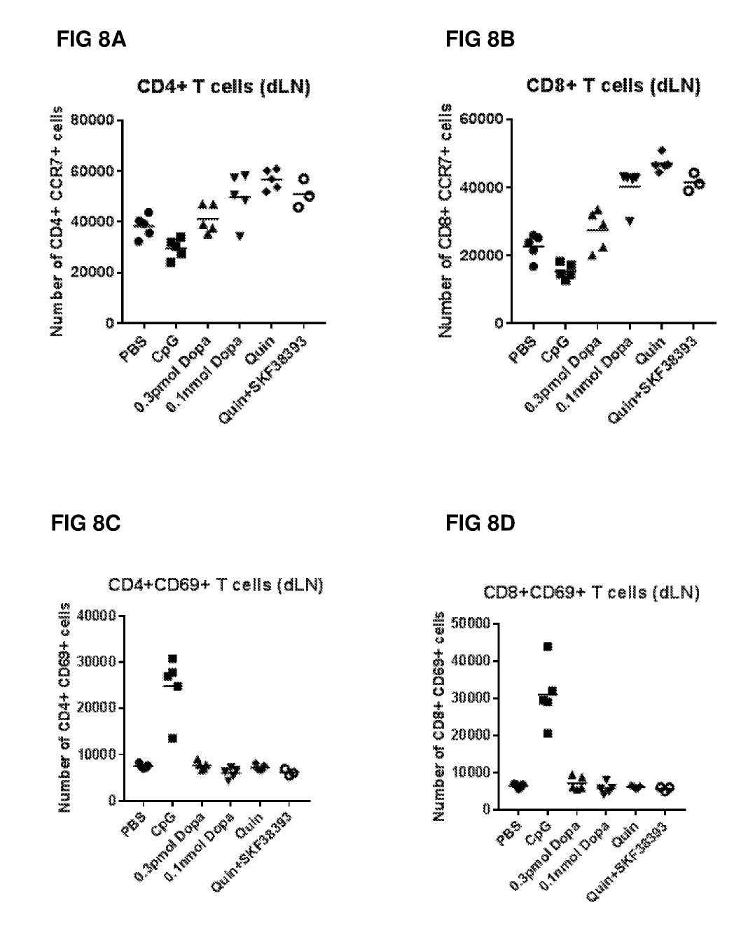

As can be seen in FIGS. 8A-8D, treatment with dopamine agonist increased the number of migratory phenotype CCR7+ T cells in the lymph node, which was inhibited by pre-treatment with a dopaminergic antagonist. In contrast, treatment with CpG ODN increased the number of inflammatory CD69+ T cells but had no effect on CCR7 expression.

CCR7 is one of the predominant chemokine receptors responsible for T cell and other immune cell homing to secondary lymphoid organs, tumors, and sites of inflammation. As such, the unexpected result described here could be useful in the treatment of multiple diseases in which immune cell migration is pathogenic or therapeutic, for example cancers in which the recruitment of immune cells to a tumor would provide therapeutic benefit.

### Example 8—NK Cell Activation to Enhance ADCC

Primary Natural Killer (NK) cells are isolated from human peripheral blood using a magnetic bead-based separation kit that negatively selects NK cells by sequestering other defined cell types (T, B, monocytes, etc.).

Isolated NK cells are incubated with a target cell line, for example a Her2 expressing cancer cell line that has been pre-coated with trastuzumab, an anti-Her2 antibody, at a range of target-to-effector cell ratios. Following antibody-dependent cell cytotoxicity (ADCC)—antibody-mediated killing of the target cells by the NK cells, the number of surviving target cells is assessed by a fluorescent viability stain.

NK cells treated with the beta adrenergic agonist metaproterenol induce significantly less ADCC than NK cells that have been pre-treated with a beta adrenergic antagonist (nadolol or propranolol) prior to exposure to the agonist. Thus, adrenergic signaling is sufficient to reduce the cytotoxic capacity of NK cells. Control of the cytotoxicity of NK cells has implications for cancer immunotherapy where activation of NK cell cytotoxicity can increase the response to treatment.

### Example 9—T Cell-Targeted Dopamine Agonism to Treat Cancer

According to the methods disclosed herein, a physician of skill in the art can treat a patient, such as a human patient with a solid tumor that is a candidate for immunotherapy (e.g., the patient has substantial T cell infiltration into the tumor as assessed by histological analysis of a biopsy), so as to inhibit solid tumor growth or reduce tumor volume. The method of treatment can include diagnosing or identifying a patient as a candidate for immunotherapy based on biopsy results conducted by the physician or a skilled laboratory technician. To treat the patient, a physician of skill in the art can administer to the human patient a neuromodulating agent that increases dopaminergic signaling (e.g., a dopamine agonist, such as dopamine, dopexamine, quinpirole, bromocriptine, lisuride, pergolide, cabergoline, quinagolide, apomorphine, ropinirole, pramipexole, or piribedil). The agent can be conjugated to an antibody that recognizes a protein expressed by a T cell (e.g., CD2, CD3, CD4, CD5, CD6, CD8, CD45, PD-1, CTLA-4, or TCR) and administered systemically (e.g., intravenous injection) or locally (e.g., intratumoral injection) to inhibit tumor growth. The neuromodulating agent-antibody conjugate is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, the neuromodulating agent-antibody conjugate is administered bimonthly, once a month, once every two weeks, or at least once a week or more (e.g., 1, 2, 3, 4, 5, 6, or 7 times a week or more).

The antibody binds to the patient's T cells, and the attached neuromodulating agent (e.g., dopamine agonist) activates the patient's T cells (e.g., increases T cell cytokine production of one or more pro-inflammatory or proliferative cytokines). The neuromodulating agent-antibody conjugate is administered to the patient in an amount sufficient to decrease tumor burden, increase progression free survival, or increase pro-inflammatory cytokine levels by 10% or more (e.g., 10%, 20%, 30%, 40%, 50%, 60%, 70%, 80%, 90%, 95% or more). Cytokine production can be assessed by collecting a blood sample from the patient and evaluating one or more pro-inflammatory cytokines (e.g., IL-2, IFNγ, IL-5, IL-6, IL-10, and IL-13). The blood sample can be collected one day or more after administration of the neuromodulating agent-antibody conjugate (e.g., 1, 2, 3, 4, 5, 6, 7, 10, 14, 21, or 30 or more days after administration). The blood sample can be compared to a blood sample collected from the patient prior to administration of the neuromodulating agent-antibody conjugate (e.g., a blood sample collected earlier the same day, 1 day, 1 week, 2 weeks, one month or more before administration of the neuromodulating agent-antibody conjugate). Tumor burden can be assessed using standard imaging methods (e.g., digital radiography, positron emission tomography (PET) scan, computed tomography (CT) scan, or magnetic resonance imaging (MRI) scan). Images from before and after administration of the neuromodulating agent-antibody conjugate can be compared to evaluate the efficacy of the treatment. A finding of a reduction in the total number of tumors, number of primary tumors, volume of tumors, positive lymph nodes, or distant metastases, or an increase in progression free survival indicates that the neuromodulating agent-antibody conjugate has successfully treated the cancer.

### Example 10—Adrenergic Antagonism to Activate T Cells to Target Tumors

According to the methods disclosed herein, a physician of skill in the art can treat a patient, such as a human patient with a solid tumor that is a candidate for immunotherapy (e.g., the patient has substantial T cell infiltration into the tumor as assessed by histological analysis of a biopsy), so as to inhibit solid tumor growth or reduce tumor volume. The method of treatment can include diagnosing or identifying a patient as a candidate for immunotherapy based on biopsy results conducted by the physician or a skilled laboratory technician. To treat the patient, a physician of skill in the art can administer to the human patient a neuromodulating agent that decreases beta adrenergic signaling (e.g., a beta adrenergic antagonist, such as propanalol, acebutol, atenolol, metoprolol, and naldol). The beta adrenergic antagonist can be administered at a dose lower or higher than that administered to a patient with high blood pressure or a cardiac condition, or it can be chemically modified (e.g., PEGylated) or delivered in a particulate formulation (e.g., a nanoparticle or microparticle) so that it does not cross the blood brain barrier. The formulation of the beta adrenergic antagonist is derived such that intravenous administration results in accumulation at the site of the tumor, based on the leakiness and enhanced permeability and retention (EPR) effect of tumor vasculature. A microparticulate formulation of propanalol is administered parenterally (e.g., intravenous injection) to inhibit tumor growth. The microparticulate formulation of propanalol is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, the microparticulate formulation of propanalol is administered bimonthly, once a month, once every two weeks, or at least once a week or more (e.g., 1, 2, 3, 4, 5, 6, or 7 times a week or more).

The beta adrenergic antagonist (e.g., propanalol) activates the patient's T cells (e.g., increases T cell cytokine production of one or more pro-inflammatory cytokines) and reverses T cell immune suppression. The beta adrenergic antagonist is administered to the patient in an amount sufficient to decrease tumor burden, increase progression free survival, or increase pro-inflammatory cytokine levels by 10% or more (e.g., 10%, 20%, 30%, 40%, 50%, 60%, 70%, 80%, 90%, 95% or more). Cytokine production can be assessed by collecting a blood sample from the patient and evaluating one or more pro-inflammatory cytokines (e.g., IFNγ, TNFα, or IL-10). The blood sample can be collected one day or more after administration of the beta adrenergic antagonist (e.g., 1, 2, 3, 4, 5, 6, 7, 10, 14, 21, or 30 or more days after administration). The blood sample can be compared to a blood sample collected from the patient prior to administration of the beta adrenergic antagonist (e.g., a blood sample collected earlier the same day, 1 day, 1 week, 2 weeks, one month or more before administration of the beta adrenergic antagonist). Tumor burden can be assessed using standard imaging methods (e.g., digital radiography, positron emission tomography (PET) scan, computed tomography (CT) scan, or magnetic resonance imaging (MRI) scan). Images from before and after administration of the beta adrenergic antagonist can be compared to evaluate the efficacy of the treatment. A finding of a reduction in the total number of tumors, number of primary tumors, volume of tumors, positive lymph nodes, or distant metastases, or an increase in progression free survival or pro-inflammatory biomarkers of immune activation indicates that the beta adrenergic antagonist has successfully improved the patient's condition and treated the cancer.

### Example 11—M1 Macrophage Polarization for Tumor Immunotherapy

According to the methods disclosed herein, a physician of skill in the art can treat a patient, such as a human patient with a solid tumor that is non-responsive to immunotherapy, so as to inhibit solid tumor growth or reduce tumor volume. A tumor can be considered non-responsive to immunotherapy if a prior course of treatment with a checkpoint inhibitor antibody, e.g., anti-PDL1, was unsuccessful, or if the tumor is categorized as “cold”, “immune excluded”, or “immune desert” based on the absence of active CD8 lymphocytes within the tumor or the presence of MO/M2 monocytes, macrophages, or myeloid-derived suppressor cells as assessed by histology or transcriptional profiling of a tumor biopsy. The method of treatment can include diagnosing or identifying a patient as having a solid tumor that is non-responsive to immunotherapy based on medical history or biopsy results conducted by the physician or a skilled laboratory technician.

To treat the patient, a physician of skill in the art can administer to the human patient a neuromodulating agent that increases macrophage polarization toward an M1 phenotype (e.g., an agent that increases macrophage antigen presentation and production of pro-inflammatory cytokines and reverses local immune suppression). The neuromodulating agent can be an agent that increases neuropeptide signaling, such as CGRP or an analog thereof. CGRP is administered locally to the tumor (e.g., intratumoral injection) to decrease tumor growth or reduce tumor burden. CGRP is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, CGRP is administered bimonthly, once a month, once every two weeks, or at least once a week or more (e.g., 1, 2, 3, 4, 5, 6, or 7 times a week or more).

CGRP increases macrophage polarization toward an M1 phenotype (e.g., increases macrophage antigen presentation and production of pro-inflammatory cytokines). CGRP is administered to the patient in an amount sufficient to decrease tumor burden, slow tumor growth, increase M1 polarization by 10% or more (e.g., 10%, 20%, 30%, 40%, 50%, 60%, 70%, 80%, 90%, 95% or more). Macrophage polarization can be assessed by collecting a tumor biopsy sample from the patient and evaluating one or more pro-inflammatory cytokines (e.g., IL-12, TNF, IL-6, IL-8, IL-1B, MCP-1 and CCL2) or antigen presentation markers (e.g., CD11c, CD11b, HLA molecules (e.g., MHC-II), CD40, B7, CD80 or CD86) using flow cytometry or immunohistochemistry. The biopsy can be collected one day or more after administration of CGRP (e.g., 1, 2, 3, 4, 5, 6, 7, 10, 14, 21, 30, or 60 or more days after administration). The biopsy can be compared to a biopsy collected from the patient prior to administration of CGRP (e.g., a biopsy collected earlier the same day, 1 day, 1 week, 2 weeks, one month or more before administration of CGRP). Tumor burden and tumor growth can be assessed using standard imaging methods (e.g., digital radiography, positron emission tomography (PET) scan, computed tomography (CT) scan, or magnetic resonance imaging (MRI) scan). Images from before and after administration of CGRP can be compared to evaluate the efficacy of the treatment. A finding of a reduction in the total number of tumors, number of primary tumors, volume of tumors, growth of tumors, positive lymph nodes, or distant metastases, or an increase in progression free survival or markers of M1 polarization indicates that CGRP has successfully improved the patient's condition and treated the cancer.

### Example 12—Neuro-Activation of Immune Cells in a Lymph Node to Treat a Tumor

According to the methods disclosed herein, a physician of skill in the art can treat a patient, such as a human patient with cancer (e.g., a solid tumor), so as to inhibit tumor growth or reduce tumor volume. Before treatment, the physician diagnoses or identifies the patient has having tumor-specific lymphocytes in the draining lymph nodes as detected by a sentinel node biopsy. The presence of tumor-specific T lymphocytes in the lymph node is confirmed by ELISPOT assay following lymphocyte pulsing with tumor lysate from the patient's own tumor biopsy. To treat the patient, a physician of skill in the art can administer to the human patient a neuromodulating agent that increases the number of CCR7+ T cells in the lymph node (e.g., a dopamine agonist, such as dopamine, dopexamine, quinpirole, bromocriptine, lisuride, pergolide, cabergoline, quinagolide, apomorphine, ropinirole, pramipexole, or piribedil). The dopamine agonist (e.g., quinpirole) is administered by subcutaneous injection proximal to the tumor draining lymph node, and can be formulated in a nanoparticle smaller than 50 nm to enhance localization to the lymph node. The patient can be treated concurrently with a checkpoint inhibitor antibody, for example anti-PDL1. Quinpirole is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, Quinpirole is administered bimonthly, once a month, once every two weeks, or at least once a week or more (e.g., 1, 2, 3, 4, 5, 6, or 7 times a week or more).

The combination of the dopamine agonist and checkpoint inhibitor increases CCR7+ T cell migration from the draining lymph node to the tumor and activates T cells (e.g., increases T cell pro-inflammatory cytokine production), thus leading to a strong immune response. Quinpirole is administered to the patient in an amount sufficient to decrease tumor burden, slow tumor growth, or increase CCR7+ T cell numbers in the lymph node or tumor by 10% or more (e.g., 10%, 20%, 30%, 40%, 50%, 60%, 70%, 80%, 90%, 95% or more). CCR7+ T cell numbers can be assessed by collecting a tumor biopsy or lymph node biopsy from the patient and evaluating CCR7+ T cells using flow cytometry. The biopsy can be collected one day or more after administration of CGRP (e.g., 1, 2, 3, 4, 5, 6, 7, 10, 14, 21, 30, or 60 or more days after administration). The biopsy can be compared to a biopsy collected from the patient prior to administration of Quinpirole (e.g., a biopsy collected earlier the same day, 1 day, 1 week, 2 weeks, one month or more before administration of Quinpirole). Tumor burden and tumor growth can be assessed using standard imaging methods (e.g., digital radiography, positron emission tomography (PET) scan, computed tomography (CT) scan, or magnetic resonance imaging (MRI) scan). Images from before and after administration of Quinpirole e can be compared to evaluate the efficacy of the treatment. A finding of a reduction in the total number of tumors, number of primary tumors, volume of tumors, growth of tumors, positive lymph nodes, or distant metastases, or an increase in progression free survival or CCR7+ T cells in the tumor or tumor draining lymph node indicates that Quinpirole has successfully improved the patient's condition and treated the cancer.

### Example 13—NK Cell Activation to Treat a Solid Tumor

According to the methods disclosed herein, a physician of skill in the art can treat a patient, such as a human patient with cancer (e.g., a solid tumor), so as to inhibit tumor growth or reduce tumor volume. Before treatment, the physician diagnoses or identifies the patient has having a tumor expressing a particular antigen that can be targeted using a therapeutic antibody (e.g., Her2-positive breast cancer). To treat the patient, a physician of skill in the art can administer to the human patient a neuromodulating agent that increases NK cell activity (e.g., restores lytic function to NK cells). The neuromodulating agent can be a beta adrenergic antagonist, such as propanalol, acebutol, atenolol, metoprolol, and naldol. The beta adrenergic antagonist (e.g., propanalol) can administered by orally at a dose lower or higher than that administered to a patient with high blood pressure or a cardiac condition, and administered in combination with an antibody that targets the antigen expressed by the tumor (e.g., trastuzumab). Propanalol is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, propanalol is administered bimonthly, once a month, once every two weeks, or at least once a week or more (e.g., 1, 2, 3, 4, 5, 6, or 7 times a week or more).

The beta adrenergic antagonist increases NK cell activity (e.g., increases NK cell cytotoxicity, such as ADCC). Propanalol is administered to the patient in an amount sufficient to decrease tumor burden, slow tumor growth, or increase NK cell activity by 10% or more (e.g., 10%, 20%, 30%, 40%, 50%, 60%, 70%, 80%, 90%, 95% or more). NK cell activity can be assessed by collecting a tumor biopsy from the patient and evaluating one or more markers of NK cell activation (e.g., CD117, NKp46, CD94, CD56, CD16, KIR, CD69, HLA-DR, CD38, KLRG1, or TIA-1) using flow cytometry or immunohistochemistry. The biopsy can be collected one day or more after administration of propanalol (e.g., 1, 2, 3, 4, 5, 6, 7, 10, 14, 21, 30, or 60 or more days after administration). The biopsy can be compared to a biopsy collected from the patient prior to administration of propanalol (e.g., a biopsy collected earlier the same day, 1 day, 1 week, 2 weeks, one month or more before administration of propanolol). Tumor burden and tumor growth can be assessed using standard imaging methods (e.g., digital radiography, positron emission tomography (PET) scan, computed tomography (CT) scan, or magnetic resonance imaging (MRI) scan). Images from before and after administration of propanolol can be compared to evaluate the efficacy of the treatment. A finding of a reduction in the total number of tumors, number of primary tumors, volume of tumors, growth of tumors, positive lymph nodes, or distant metastases, or an increase in progression free survival or NK cell activation in the tumor indicates that propanalol has successfully improved the patient's condition and treated the cancer.

### Example 14—Neuromodulation to Activate the Immune System and Inhibit a Tumor Cell

According to the methods disclosed herein, a physician of skill in the art can treat a patient, such as a human patient with a solid tumor that is a candidate for immunotherapy (e.g., the patient has substantial T cell infiltration into the tumor as assessed by histological analysis of a biopsy), so as to inhibit solid tumor growth or reduce tumor volume. The method of treatment can include diagnosing or identifying a patient as a candidate for immunotherapy based on biopsy results conducted by the physician or a skilled laboratory technician. To treat the patient, a physician of skill in the art can administer to the human patient a neuromodulating agent that decreases beta adrenergic signaling (e.g., a beta adrenergic antagonist, such as propanalol, acebutol, atenolol, metoprolol, and naldol). The beta adrenergic antagonist can be administered at a dose lower or higher than that administered to a patient with high blood pressure or a cardiac condition. Propanalol is administered parenterally (e.g., intratumorally) to inhibit tumor growth. Propanalol is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, propanalol is administered bimonthly, once a month, once every two weeks, or at least once a week or more (e.g., 1, 2, 3, 4, 5, 6, or 7 times a week or more).

The beta adrenergic antagonist (e.g., propanalol) increases macrophage polarization toward an M1 phenotype and decreases tumor growth. The beta adrenergic antagonist is administered to the patient in an amount sufficient to decrease tumor growth decrease tumor burden, increase progression free survival, or increase pro-inflammatory cytokine levels by 10% or more (e.g., 10%, 20%, 30%, 40%, 50%, 60%, 70%, 80%, 90%, 95% or more). Macrophage polarization can be assessed by collecting a tumor biopsy sample from the patient and evaluating one or more pro-inflammatory cytokines (e.g., IL-12, TNF, IL-6, IL-8, IL-1B, MCP-1 and CCL2) or antigen presentation markers (e.g., CD11c, CD11b, HLA molecules (e.g., MHC-II), CD40, B7, CD80 or CD86) using flow cytometry or immunohistochemistry. The biopsy sample can be collected one day or more after administration of the beta adrenergic antagonist (e.g., 1, 2, 3, 4, 5, 6, 7, 10, 14, 21, or 30 or more days after administration). The biopsy sample can be compared to a biopsy sample collected from the patient prior to administration of the beta adrenergic antagonist (e.g., a blood sample collected earlier the same day, 1 day, 1 week, 2 weeks, one month or more before administration of the beta adrenergic antagonist). Tumor burden can be assessed using standard imaging methods (e.g., digital radiography, positron emission tomography (PET) scan, computed tomography (CT) scan, or magnetic resonance imaging (MRI) scan). Images from before and after administration of the beta adrenergic antagonist can be compared to evaluate the efficacy of the treatment. A finding of a reduction in the total number of tumors, number of primary tumors, volume of tumors, or growth of tumors, or an increase in M1 macrophage polarization indicates that the beta adrenergic antagonist has successfully activated an immune response and treated the cancer.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of treating a subject with a disease characterized by immune dysregulation, the method comprising administering to the subject an effective amount of a neuromodulating agent selected from the group consisting of a neurotransmission modulator, a neuropeptide signaling modulator, a neuronal growth factor modulator, and a neurome gene expression modulator.

2. A method of treating a subject identified as having a disease characterized by immune dysregulation, the method comprising administering to the subject an effective amount of a neuromodulating agent selected from the group consisting of a neurotransmission modulator, a neuropeptide signaling modulator, a neuronal growth factor modulator, and a neurome gene expression modulator.

3. A method of treating a subject with a disease characterized by immune dysregulation, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

4. A method of treating a subject identified as having a disease characterized by immune dysregulation, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

5. A method of modulating an immune response in a subject, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

6. A method of modulating an immune response in a subject, the method comprising administering an effective amount of a neuromodulating agent selected from the group consisting of a neurotransmission modulator, a neuropeptide signaling modulator, a neuronal growth factor modulator, and a neurome gene expression modulator.

7. A method of modulating an immune cell activity, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

8. A method of treating a subject with cancer, the method comprising administering to the subject an effective amount of a neuromodulating agent selected from the group consisting of a neurotransmission modulator, a neuropeptide signaling modulator, a neuronal growth factor modulator, and a neurome gene expression modulator.

9. A method of treating a subject identified as having cancer, the method comprising administering to the subject an effective amount of a neuromodulating agent selected from the group consisting of a neurotransmission modulator, a neuropeptide signaling modulator, a neuronal growth factor modulator, and a neurome gene expression modulator.

10. A method of treating a subject with cancer, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

11. A method of treating a subject identified as having cancer, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

12. A method of treating a subject with a T cell-infiltrated tumor, the method comprising administering to the subject an effective amount of a neuromodulating agent selected from the group consisting of a neurotransmission modulator, a neuropeptide signaling modulator, a neuronal growth factor modulator, and a neurome gene expression modulator.

13. A method of treating a subject with a T cell-infiltrated tumor, the method comprising contacting the tumor with an effective amount of a neuromodulating agent selected from the group consisting of a neurotransmission modulator, a neuropeptide signaling modulator, a neuronal growth factor modulator, and a neurome gene expression modulator.

14. A method of treating a subject with a T cell-infiltrated tumor, the method comprising contacting a T cell in the tumor with an effective amount of a neuromodulating agent selected from the group consisting of a neurotransmission modulator, a neuropeptide signaling modulator, a neuronal growth factor modulator, and a neurome gene expression modulator.

15. The method any one of claims 1-14, wherein the method comprises contacting an immune cell from column 2 of Table 13 with an effective amount of a neuromodulating agent that modulates a corresponding gene in column 1 of Table 13.

16. The method of any one of claims 1-15, wherein the method comprises modulating an immune cell activity.

17. The method of any one of claims 1-14, wherein the method comprises modulating lymph node innervation, modulating development of high endothelial venules (HEVs), or modulating the development of ectopic or tertiary lymphoid organs (TLOs).

18. The method of claim 7 or 16, wherein the immune cell activity is activation, proliferation, phagocytosis, antibody-dependent cell-mediated cytotoxicity (ADCC), antibody-dependent cell-mediated phagocytosis (ADCP), antigen presentation, lymph node homing, lymph node egress, differentiation, degranulation, polarization, cytokine production, recruitment, or migration.

19. The method of claim 17 or 18, wherein the activation, proliferation, phagocytosis, ADCC, ADCP, antigen presentation, lymph node homing, lymph node egress, differentiation, degranulation, polarization, cytokine production, recruitment, migration, lymph node innervation, development of HEVs, or development of TLOs is increased.

20. The method of claim 17 or 18, wherein the activation, proliferation, phagocytosis, ADCC, ACCP, antigen presentation, lymph node homing, lymph node egress, differentiation, degranulation, polarization, cytokine production, recruitment, migration, lymph node innervation, development of HEVs, or development of TLOs is decreased.

21. The method of claim 19, wherein polarization toward an M1 phenotype is increased.

22. The method of claim 19, wherein polarization toward an M2 phenotype is increased.

23. The method of claim 20, wherein polarization toward an M1 phenotype is decreased.

24. The method of claim 20, wherein polarization toward an M2 phenotype is decreased.

25. The method of claim 19 or 20, wherein the cytokines are pro-inflammatory cytokines, anti-inflammatory cytokines, or proliferative cytokines.

26. The method of claim 19 or 20, wherein recruitment or migration is directed toward a tumor.

27. The method of claim 19 or 20, wherein migration is directed away from tumor.

28. The method of claim 24 or 25, wherein recruitment or migration is directed toward a lymph node or secondary lymphoid organ.

29. The method of claim 24 or 25, wherein migration is directed away from a lymph node or secondary lymphoid organ.

30. The method of any one of claims 3-29, wherein the immune cell is selected from the group consisting of a T cell, a cytotoxic T cell, a monocyte, a peripheral blood hematopoietic stem cell, a macrophage, an antigen presenting cell, a Natural Killer cell, a mast cell, a neutrophil, an eosinophil, a basophil, a Natural Killer T cell, a B cell, a dendritic cell, and a regulatory T cell.

31. A method of modulating innervation of a lymph node or lymphoid organ, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

32. A method of modulating innervation of a lymph node or lymphoid organ, the method comprising administering an effective amount of a neuromodulating agent selected from the group consisting of a neurotransmission modulator, a neuropeptide signaling modulator, a neuronal growth factor modulator, and a neurome gene expression modulator.

33. The method of claim 31 or 32, wherein innervation is increased.

34. The method of claim 31 or 32, wherein innervation is decreased.

35. A method of modulating development of high endothelial venules (HEVs) or ectopic or tertiary lymphoid organs (TLOs), the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

36. A method of modulating development of high endothelial venules (HEVs) or ectopic or tertiary lymphoid organs (TLOs), the method comprising administering with an effective amount of a neuromodulating agent selected from the group consisting of a neurotransmission modulator, a neuropeptide signaling modulator, a neuronal growth factor modulator, and a neurome gene expression modulator.

37. The method of claim 35 or 36, wherein development of high endothelial venules (HEVs) or ectopic or tertiary lymphoid organs (TLOs) is increased.

38. The method of claim 35 or 36, wherein development of high endothelial venules (HEVs) or ectopic or tertiary lymphoid organs (TLOs) is decreased.

39. A method of modulating T cell cytokine production, the method comprising contacting a T cell with an effective amount of a neuromodulating agent selected from the group consisting of a neurotransmission modulator, a neuropeptide signaling modulator, a neuronal growth factor modulator, and a neurome gene expression modulator.

40. A method of modulating T cell cytokine production, the method comprising administering an effective amount of a neuromodulating agent selected from the group consisting of a neurotransmission modulator, a neuropeptide signaling modulator, a neuronal growth factor modulator, and a neurome gene expression modulator.

41. The method of claim 39 or 40, wherein T cell cytokine production of pro-inflammatory or pro-survival cytokines is increased.

42. The method of claim 39 or 40, wherein T cell cytokine production of pro-inflammatory cytokines is decreased.

43. The method of claim 39 or 40, wherein T cell cytokine production of anti-inflammatory cytokines is increased.

44. A method of modulating macrophage polarization, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

45. A method of modulating macrophage polarization, the method comprising administering an effective amount of a neuromodulating agent selected from the group consisting of a neurotransmission modulator, a neuropeptide signaling modulator, a neuronal growth factor modulator, and a neurome gene expression modulator.

46. The method of claim 44 or 45, wherein macrophages are polarized toward an M2 phenotype.

47. The method of claim 44 or 45, wherein macrophages are polarized toward an M1 phenotype.

48. A method of increasing the number of immune cells in a tumor, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

49. A method of increasing the number of immune cells in a tumor, the method comprising administering an effective amount of a neuromodulating agent selected from the group consisting of a neurotransmission modulator, a neuropeptide signaling modulator, a neuronal growth factor modulator, and a neurome gene expression modulator.

50. The method of claim 48 or 49, wherein the method comprises increasing immune cell migration or recruitment to a tumor.

51. The method of any one of claims 48-50, wherein the immune cell is a T cell, γδ T cell, Th1 CD4+ T cell, cytotoxic CD8+ T cell, B cell, macrophage, M1 macrophage, natural killer cell, neutrophil, eosinophil, mast cell, or dendritic cell.

52. The method of claim 51, wherein the immune cell is a CCR7+ T cell.

53. A method of increasing immune cell homing to a lymph node, the method comprising administering an effective amount of a neuromodulating agent selected from the group consisting of a neurotransmission modulator, a neuropeptide signaling modulator, a neuronal growth factor modulator, and a neurome gene expression modulator.

54. A method of increasing immune cell homing to a lymph node, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

55. The method of claim 53 or 54, wherein the immune cell is a T cell, B cell, macrophage, or dendritic cell.

56. The method of claim 55, wherein the immune cell is a CCR7+ T cell.

57. A method of increasing the number of CCR7+ T cells in a lymph node, the method comprising contacting a CCR7+ T cell with an effective amount of a dopamine agonist.

58. A method of increasing the number of CCR7+ T cells in a lymph node, the method comprising administering an effective amount of a dopamine agonist.

59. The method of claim 57 or 58, wherein the method comprises increasing CCR7+ T cell proliferation.

60. The method of claim 57 or 58, wherein the method comprises increasing CCR7+ T cell lymph node homing.

61. A method of decreasing immune cell migration to a tumor, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

62. A method of decreasing immune cell migration to a tumor, the method comprising administering an effective amount of a neuromodulating agent selected from the group consisting of a neurotransmission modulator, a neuropeptide signaling modulator, a neuronal growth factor modulator, and a neurome gene expression modulator.

63. The method of claim 61 or 62, wherein the immune cell is a myeloid-derived suppressor cell (MDSC), regulatory T cell, M2 macrophage, or immature dendritic cell.

64. A method of increasing pro-inflammatory cytokine levels, the method comprising administering an effective amount of a neuromodulating agent selected from the group consisting of a neurotransmission modulator, a neuropeptide signaling modulator, a neuronal growth factor modulator, and a neurome gene expression modulator.

65. A method of increasing pro-inflammatory cytokine levels, the method comprising contacting immune cell with an effective amount of a neuromodulating agent selected from the group consisting of a neurotransmission modulator, a neuropeptide signaling modulator, a neuronal growth factor modulator, and a neurome gene expression modulator.

66. A method of increasing T cell production of pro-inflammatory or proliferative cytokines, the method comprising contacting a T cell with an effective amount of a neuromodulating agent selected from the group consisting of a neurotransmission modulator, a neuropeptide signaling modulator, a neuronal growth factor modulator, and a neurome gene expression modulator.

67. A method of increasing T cell production of pro-inflammatory or proliferative cytokines, the method comprising administering an effective amount of a neuromodulating agent selected from the group consisting of a neurotransmission modulator, a neuropeptide signaling modulator, a neuronal growth factor modulator, and a neurome gene expression modulator.

68. The method of any one of claims 64-67, wherein the pro-inflammatory cytokine is interferon gamma (IFNγ), interleukin-5 (IL-5), IL-6, IL-10, IL-13, or tumor necrosis factor alpha (TNFα).

69. A method of increasing macrophage polarization toward an M1 phenotype, the method comprising contacting a macrophage with an effective amount of a neuromodulating agent selected from the group consisting of a neurotransmission modulator, a neuropeptide signaling modulator, a neuronal growth factor modulator, and a neurome gene expression modulator.

70. A method of increasing macrophage polarization toward an M1 phenotype, the method comprising administering an effective amount of a neuromodulating agent selected from the group consisting of a neurotransmission modulator, a neuropeptide signaling modulator, a neuronal growth factor modulator, and a neurome gene expression modulator.

71. A method of increasing immune cell cytotoxicity, the method comprising contacting an immune cell with an effective amount of a neuromodulating agent selected from the group consisting of a neurotransmission modulator, a neuropeptide signaling modulator, a neuronal growth factor modulator, and a neurome gene expression modulator.

72. A method of increasing immune cell cytotoxicity, the method comprising administering an effective amount of a neuromodulating agent selected from the group consisting of a neurotransmission modulator, a neuropeptide signaling modulator, a neuronal growth factor modulator, and a neurome gene expression modulator.

73. The method of claim 71 or 72, wherein the cytotoxicity is antibody-dependent cell-mediated cytotoxicity.

74. A method of increasing Natural Killer (NK) cell activity or restoring NK cell lytic function, the method comprising contacting an NK cell with an effective amount a neuromodulating agent selected from the group consisting of a neurotransmission modulator, a neuropeptide signaling modulator, a neuronal growth factor modulator, and a neurome gene expression modulator.

75. A method of increasing Natural Killer (NK) cell activity or restoring NK cell lytic function, the method comprising administering an effective amount a neuromodulating agent selected from the group consisting of a neurotransmission modulator, a neuropeptide signaling modulator, a neuronal growth factor modulator, and a neurome gene expression modulator.

76. A method of increasing immune cell activation, the method comprising contacting an immune cell with an effective amount of a neuromodulating agent selected from the group consisting of a neurotransmission modulator, a neuropeptide signaling modulator, a neuronal growth factor modulator, and a neurome gene expression modulator, wherein the neuromodulating agent slows or prevents tumor growth.

77. A method of increasing immune cell activation, the method comprising administering an effective amount of a neuromodulating agent selected from the group consisting of a neurotransmission modulator, a neuropeptide signaling modulator, a neuronal growth factor modulator, and a neurome gene expression modulator, wherein the neuromodulating agent slows or prevents tumor growth.

78. A method of increasing immune cell polarization toward an M1 phenotype, the method comprising contacting an immune cell with an effective amount of a neuromodulating agent selected from the group consisting of a neurotransmission modulator, a neuropeptide signaling modulator, a neuronal growth factor modulator, and a neurome gene expression modulator, wherein the neuromodulating agent slows or prevents tumor growth.

79. A method of increasing immune cell polarization toward an M1 phenotype, the method comprising administering an effective amount of a neuromodulating agent selected from the group consisting of a neurotransmission modulator, a neuropeptide signaling modulator, a neuronal growth factor modulator, and a neurome gene expression modulator, wherein the neuromodulating agent slows or prevents tumor growth.

80. The method of any one of claims 76-79, wherein the immune cell is a macrophage.

81. The method of any one of claim 25, 41, 42, or 64-68, wherein the pro-inflammatory cytokine is IL-1β, IL-4, IL-5, IL-6, IL-8, IL-10, IL-12, IL-13, IL-18, TNFα, IFNγ, MCP-1, CCL2, or GMCSF.

82. The method of claim 25 or 41, wherein the pro-survival cytokine is IL-2, IL-4, IL-6, IL-7, or IL-15.

83. The method of claim 25 or 43, wherein the anti-inflammatory cytokine is IL-4, IL-10, IL-11, IL-13, IFNα, or TGFβ.

84. The method of any one of claim 8-16, 48-50, 61, or 62, wherein the cancer is gastrointestinal cancer, gastric cancer, pancreatic cancer, urogenital cancer, prostate cancer, gynecological cancer, ovarian cancer, lung cancer, small cell lung cancer, non-small cell lung cancer, head and neck cancer, esophageal cancer, CNS cancer, glioma, malignant mesothelioma, melanoma, non-metastatic or metastatic breast cancer, skin cancer, thyroid cancer, bone or soft tissue sarcoma, paraneoplastic cancer, or a hematologic neoplasia.

85. The method of any one of claims 1-84, wherein the neuromodulating agent is a dopamine agonist, adrenergic agonist, nicotinic agonist, muscarinic agonist, serotonin agonist, glutamate receptor agonist, histamine agonist, cannabinoid receptor agonist, purinergic receptor agonist, GABA agonist, neuropeptide Y receptor agonist, somatostatin receptor agonist, CGRP receptor agonist, tachykinin receptor agonist, VIP receptor agonist, opioid agonist, oxytocin receptor agonist, or vasopressin receptor agonist.

86. The method of claim 85, wherein the agonist is selected from an agonist listed in Tables 2A-2L.

87. The method of claim 86, wherein the agonist is a dopamine agonist listed in Table 2A or 2C.

88. The method of claim 87, wherein the dopamine agonist is dopamine, quinpirole dopexamine, bromocriptine, lisuride, pergolide, cabergoline, quinagolide, apomorphine, ropinirole, pramipexole, or piribedil.

89. The method of claim 86, wherein the agonist is an adrenergic agonist listed in Table 2A or 2B.

90. The method of claim 89, wherein the adrenergic agonist is isoproterenol or metaproterenol.

91. The method of any one of claims 1-84, wherein the neuromodulating agent is a dopamine antagonist, adrenergic antagonist, nicotinic antagonist, muscarinic antagonist, serotonin antagonist, glutamate receptor antagonist, histamine antagonist, cannabinoid receptor antagonist, purinergic receptor antagonist, GABA antagonist, neuropeptide Y receptor antagonist, somatostatin receptor antagonist, CGRP receptor antagonist, tachykinin receptor antagonist, VIP receptor antagonist, opioid antagonist, oxytocin receptor antagonist, or vasopressin receptor antagonist.

92. The method of claim 91, wherein the antagonist is selected from an antagonist listed in Tables 2A-2L.

93. The method of claim 92, wherein the antagonist is a dopamine antagonist listed in Table 2A or 2C.

94. The method of claim 93, wherein the dopamine antagonist is haloperidol or L-741,626.

95. The method of claim 92, wherein the antagonist is a beta adrenergic antagonist listed in Table 2A or 2B.

96. The method of claim 95, wherein the beta adrenergic antagonist is propranolol or nadolol.

97. The method of any one of claims 1-84, wherein the neuromodulating agent is neuropeptide Y, CGRP, somatostatin, bombesin, cholecystokinin, dynorphin, enkephalin, endorphin, gastrin glucagon, melatonin, motilin, neurokinin A, neurokinin B, orexin, oxytocin, pancreatic peptide, peptide YY, substance P, or vasoactive intestinal peptide.

98. The method of claim 97, wherein the neuromodulating agent is neuropeptide Y.

99. The method of claim 97, wherein the neuromodulating agent is CGRP.

100. The method of any one of claims 1-84, wherein the neuromodulating agent is a neuropeptide Y, CGRP, somatostatin, bombesin, cholecystokinin, dynorphin, enkephalin, endorphin, gastrin glucagon, melatonin, motilin, neurokinin A, neurokinin B, orexin, oxytocin, pancreatic peptide, peptide YY, substance P, or vasoactive intestinal peptide blocking antibody.

101. The method of claim 100, wherein the neuromodulating agent is a neuropeptide Y blocking antibody.

102. The method of claim 100, wherein the neuromodulating agent is a CGRP blocking antibody.

103. The method of claim 102, wherein the CGRP blocking antibody is an antibody listed in Table 4.

104. The method of any one of claims 1-103, wherein the neuromodulating agent is a neurotransmission modulator.

105. The method of claim 104, wherein the neurotransmission modulator is a neurotransmitter listed in Tables 1A-1B a neurotransmitter encoded by a gene in Table 7, an agonist or an antagonist of a neurotransmitter of neurotransmitter receptor listed in Tables 1A-1B or encoded by a gene in Table 7, a neurotransmission modulator listed in Table 2M, a modulator of a biosynthesis, channel, ligand receptor, signaling, structural, synaptic, vesicular, or transporter protein encoded by a gene in Table 7, a channel or transporter protein encoded by a gene in Table 8, or a neurotoxin listed in Table 3.

106. The method of claim 105, wherein the agonist or antagonist is an agonist or antagonist listed in Tables 2A-2K.

107. The method of any one of claims 1-103, wherein the neuromodulating agent is a neuropeptide signaling modulator.

108. The method of claim 107, wherein the neuropeptide signaling modulator is a neuropeptide listed in Tables 1A-1B or encoded by a gene in Table 7 or analog thereof, an agonist or antagonist of a neuropeptide or neuropeptide receptor listed in in Tables 1A-1B or encoded by a gene in Table 7, or a modulator of a biosynthesis, ligand, receptor, or signaling protein encoded by a gene in Table 7.

109. The method of claim 108, wherein the neuropeptide has at least 70%, 75%, 80%, 85%, 90%, 95%, 98%, or 99% identity to the neuropeptide sequence referenced by accession number or Entrez Gene ID in Tables 1A-1B or Table 7.

110. The method of claim 108, wherein the agonist or antagonist is an agonist or antagonist listed in Tables 2A or 2L.

111. The method of any one of claims 1-103, wherein the neuromodulating agent is a neuronal growth factor modulator.

112. The method of claim 111, wherein the neuronal growth factor modulator is a neuronal growth factor listed in Table 10 or encoded by a gene in Table 7 or an analog thereof, or a modulator of a ligand, receptor, structural, synaptic, or signaling protein encoded by a gene in Table 7.

113. The method of claim 112, wherein the neuronal growth factor has at least 70%, 75%, 80%, 85%, 90%, 90%, 98%, or 99% identity to the neuronal growth factor sequence referenced by accession number or Entrez Gene ID in Table 10 or Table 7

114. The method of claim 111, wherein the neuronal growth factor modulator is an antibody listed in Table 5.

115. The method of claim 111, wherein the neuronal growth factor modulator is an agonist or antagonist listed in Table 6.

116. The method of claim 111, wherein the neuronal growth factor modulator is etanercept, thalidomide, lenalidomide, pomalidomide, pentoxifylline, bupropion, DOI, disitertide, or trabedersen.

117. The method of any one of claims 1-103, wherein the neuromodulating agent is a neurome gene expression modulator.

118. The method of claim 117, wherein the neurome gene expression modulator increases or decreases the expression of a neurome gene in Table 7.

119. The method of any one of claims 1-118, wherein the neuromodulating agent modulates the expression of a neurome gene in Table 7 or the activity of a protein encoded by a neurome gene in Table 7.

120. The method of any one of claims 1-119, wherein the neuromodulating agent modulates the expression or activity of a chemokine, chemokine receptor, or immune cell trafficking molecule in Tables 10 or 11.

121. The method of any one of claims 1-120, wherein the neuromodulating agent is selected from the group consisting of a neurotransmitter, a neuropeptide, an antibody, a small molecule, a DNA molecule, a RNA molecule, a gRNA, and a viral vector.

122. The method of claim 121, wherein the antibody is a blocking antibody.

123. The method of claim 121, wherein the RNA molecule is an mRNA or an inhibitory RNA.

124. The method of claim 121, wherein the viral vector is selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a lentivirus.

125. The method of claim 124, wherein the herpes virus is a replication deficient herpes virus.

126. The method of any one of claims 1-125, wherein the neuromodulating agent does not cross the blood brain barrier.

127. The method of claim 126, wherein the neuromodulating agent has been modified to prevent blood brain barrier crossing by conjugation to a targeting moiety, formulation in a particulate delivery system, addition of a molecular adduct, or through modulation of its size, polarity, flexibility, or lipophilicity.

128. The method of any one of claims 1-127, wherein the neuromodulating agent does not have a direct effect on the central nervous system or gut.

129. The method of any one of claims 1-128, wherein the neuromodulating agent is administered locally.

130. The method of claim 129, wherein the neuromodulating agent is administered intratumorally.

131. The method of claim 129, wherein the neuromodulating agent is administered to or near a lymph node.

132. The method of any one of claims 1-131, wherein the method further comprises administering a second therapeutic agent.

133. The method of claim 132, wherein the second therapeutic agent is a checkpoint inhibitor, a chemotherapeutic agent, a biologic cancer agent, an anti-angiogenic drug, a drug that targets cancer metabolism, an antibody that marks a cancer cell surface for destruction, an antibody-drug conjugate, a cell therapy, a commonly used anti-neoplastic agent, or a non-drug therapy.

134. The method of claim 133, wherein the checkpoint inhibitor is an inhibitory antibody, a fusion protein, an agent that interacts with a checkpoint protein, an agent that interacts with the ligand of a checkpoint protein, an inhibitor of CTLA-4, an inhibitor of PD-1, an inhibitor of PD-L1, an inhibitor of PD-L2, or an inhibitor of B7-H3, B7-H4, BTLA, HVEM, TIM3, GAL9, LAGS, VISTA, KIR, 2B4, CD160, CGEN-15049, CHK 1, CHK2, A2aR, or B-7 family ligands.

135. The method of claim 133, wherein the biologic cancer agent is an antibody listed in Table 12.

136. The method of any one of claims 1-135, wherein the neuromodulating agent decreases tumor volume, tumor growth, tumor innervation, cancer cell proliferation, cancer cell invasion, or cancer cell metastasis, or increases cancer cell death.

137. The method of any one of claims 1-136, wherein the method further comprises measuring one or more of tumor volume, tumor growth, tumor innervation, cancer cell proliferation, cancer cell invasion, cancer cell metastasis, or tumor neurome gene expression after administration of the neuromodulating agent.

138. The method of any one of claims 1-137, wherein the method further comprises measuring cytokine levels after administration of the neuromodulating agent.

139. The method of any one of claims 1-138, wherein the method further comprises measuring one or more immune cell markers after administration of the neuromodulating agent.

140. The method of any one of claims 1-139, wherein the method further comprises measuring the expression of one or more neurome genes in Table 7 after administration of the neuromodulating agent.

141. The method of any one of claims 1-140, wherein the method further comprises measuring cytokine levels before administration of the neuromodulating agent.

142. The method of any one of claims 1-141, wherein the method further comprises measuring one or more immune cell markers before administration of the neuromodulating agent.

143. The method of claim 139 or 142, wherein the one or more immune cell markers is a marker listed in Table 9.

144. The method of any one of claims 1-143, wherein the method further comprises profiling an immune cell for expression of one or more neurome genes in Table 7 before administration of the neuromodulating agent.

145. The method of claim 144, wherein the method further comprises selecting a neuromodulating agent based on the profiling results.

146. The method of any one of claim 105, 108, 109, 112, 113, 118, 119, 140, or 144, wherein the one or more neurome genes in Table 7 is a channel, transporter, neurotransmitter, neuropeptide, neurotrophic, signaling, synaptic, structural, ligand, receptor, biosynthesis, other, or vesicular gene.

147. The method of any one of claims 1-146, wherein the subject is not diagnosed as having a neuropsychiatric disorder.

148. The method of any one of claims 1-147, wherein the subject is not diagnosed as having high blood pressure or a cardiac condition.

149. The method of any one of claims 1-148, wherein the neuromodulating agent is administered in an amount sufficient to increase lymph node innervation, increase tumor innervation, increase nerve activity in a lymph node, increase nerve activity in a tumor, increase the development of HEVs or TLOs, increase immune cell migration, increase immune cell proliferation, increase immune cell recruitment, increase immune cell lymph node homing, increase immune cell lymph node egress, increase immune cell tumor homing, increase immune cell tumor egress, increase immune cell differentiation, increase immune cell activation, increase immune cell polarization, increase immune cell cytokine production, increase immune cell degranulation, increase immune cell maturation, increase immune cell ADCC, increase immune cell ADCP, or increase immune cell antigen presentation.

150. The method of any one of claims 1-148, wherein the neuromodulating agent is administered in an amount sufficient to decrease lymph node innervation, decrease tumor innervation, decrease nerve activity in a tumor, decrease nerve activity in a lymph node, decrease the development of HEVs or TLOs, decrease immune cell migration, decrease immune cell proliferation, decrease immune cell recruitment, decrease immune cell lymph node homing, decrease immune cell lymph node egress, decrease immune cell tumor homing, decrease immune cell tumor egress, decrease immune cell differentiation, decrease immune cell activation, decrease immune cell polarization, decrease immune cell cytokine production, decrease immune cell degranulation, decrease immune cell maturation, decrease immune cell ADCC, decrease immune cell ADCP, or decrease immune cell antigen presentation.

151. The method of any one of claims 1-150, wherein the neuromodulating agent is administered in an amount sufficient to treat the cancer or tumor, cause remission, reduce tumor growth, reduce tumor volume, reduce tumor metastasis, reduce tumor invasion, reduce tumor proliferation, reduce tumor number, increase cancer cell death, increase time to recurrence, or improve survival.
